(19) United States
(12) Patent Application Publication
(10) Pub. No.: US 2024/0002344 A1
(43) Pub. Date: Jan. 4, 2024

(54) COMPOUNDS, COMPOSITIONS AND METHODS FOR HISTONE LYSINE DEMETHYLASE INHIBITION

(71) Applicant: FibroGen, Inc., San Francisco, CA (US)

(72) Inventors: Zuhui ZHANG, Millbrae, CA (US); Xiaoti Zhou, Millbrae, CA (US); Michael P. Arend, Belmont, CA (US); Christian Hauge Kjaergaard, Boulder Creek, CA (US)

(21) Appl. No.: 18/043,117

(22) PCT Filed: Aug. 27, 2021
(86) PCT No.: PCT/US2021/048056

§ 371 Date: Feb. 27, 2023

Related U.S. Application Data
(60) Provisional application No. 63/072,012, filed on Aug. 28, 2020.

(51) Int. Cl. C07D 213/84; C07D 213/65; C07D 401/04; C07C 255/56

(52) U.S. Cl. CPC C07D 213/84; C07C 255/56; C07D 401/04; C07D 213/65

(57) ABSTRACT

The present disclosure relates generally to compounds and pharmaceutical compositions for the selective inhibition of histone lysine demethylase5 (KDM5), particularly KDM5B, and methods of their use in treating conditions and diseases associated with KDM5 activity.

### CROSS REFERENCE TO RELATED APPLICATIONS

This application claims the benefit under 35 U.S.C. § 119(e) to U.S. Provisional Application No. 63/072,012, filed Aug. 28, 2020, which is incorporated by reference herein its entirety.

### FIELD

Provided herein are compounds and pharmaceutical compositions suitable for the selective inhibition of histone lysine demethylase-5 (KDM5), particularly KDM5B, and methods for their use in treating conditions and diseases modulated by KDM5 activity.

### BACKGROUND

Epigenetic enzymes modify DNA or histones by adding or removing chemical markers including methylation, acetylation, phosphorylation, and ubiquitination. These alterations modify the interactions between histones and DNA and lead to local changes in chromatin structure that silence or activate local genes. Histone Demethylases (HDMs) are a class of epigenetic enzyme that remove methyl groups from histone lysine residues, in particular lysine residues 4 (H3K4), 9 (H3K9), 27 (H3K27), 36 (H3K36), and 79 (H3K79) on histone 3, and lysine residue 20 (H4K20) on histone 4. Although there is some literature on the roles of HDMs in non-cancer settings (e.g. inflammation and wound healing), the vast majority of the current HDM literature is in cancer where HDMs are frequently over-expressed and where genetic approaches support pro-tumorigenic HDM functions.

At least 17 different HDMs have been identified so far. While the first and most studied HDMs belong to the lysine-specific demethylase 1 (LSD1; also known as KDM1) family of amine oxidases, the remainder are all Jumonji C (JmjC) domain containing Fe(II)-dependent and 2-oxoglutarate (2OG)-dependent dioxygenases. The JmjC domain is responsible for the demethylation activity by first hydroxylating histone lysine methylamine groups utilizing oxygen and 2-OG, which is then followed by the spontaneous loss of the unstable hydroxymethyl group. (See, e.g., Loenarz and Schofield (2008) Nat Chem Biol 4(3):152-6; Ozer et al (2007) Nat Chem Biol 3(3):144-53.)

The KDM5, or JARID1, family of JmjC HDMs includes KDM5A (JARID1A/RBP2), KDM5B (JARID1B/PLU-1), KDM5C (JARID1C/SMCX), and KDM5D (JARID1D/SMCY). These enzymes can act on di- and trimethylated H3K4 and demethylate H3K4me3/me2/me. There are numerous reports of pro-tumorigenic functions for KDM5A, KDM5B, and KDM5C. (See, e.g., Tang et al (2015) Oncotarget 6(14):12723-39.) For example, KDM5B has been implicated in the development of prostate, breast, and skin cancer and also has been associated with melanoma maintenance. (See, e.g., Han et al. (2017) Oncotarget 8(5):8980-8991.) KDM5B is also overexpressed in non-small cell lung cancer (NSCLC) cells and is associated with tumor size, lymph node metastasis, advanced stages, and poor overall survival in NSCLC patients. (Kuo et al. (2018) Clin Epigenetics 10(1):107.)

There remains a need for compounds that are effective in the treatment and prevention of conditions and disorders associated with KDM5 and, in particular KDM5B, including various cancers. The compounds provided herein inhibit KDM5 activity and can be used to treat and prevent KDM5-associated disorders such as cancer.

### SUMMARY

The present disclosure is directed to compounds, compositions comprising the same, and methods of using the compounds to selectively modulate the activity of histone demethylases (HDMs), in particular, histone lysine demethylase 5.

In one aspect, provided are compounds represented by formula I:

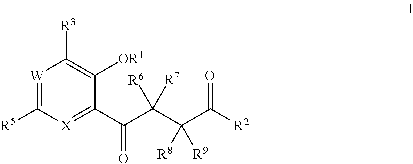

or a pharmaceutically acceptable salt, isotopically enriched analog, stereoisomer, mixture of stereoisomers or prodrug thereof, wherein:
one of W or X is N and the other of W and X is CR⁴; or W and X are CR⁴;
R¹ is hydrogen, —P(O)(OR²⁰)₂, —CH₂P(O)(OR²⁰)₂, —P(O)(R²⁰)(OR²⁰), —CH₂P(O)(R²⁰)(OR²⁰), —P(O)(N(R²⁰)₂)(OR²⁰), —CH₂P(O)(N(R²⁰)₂)(OR²⁰), —P(O)(R²⁰)(N(R²⁰)₂), —CH₂P(O)(R²⁰)(N(R²⁰)₂), —C(O)R²⁰, —C(O)N(R²¹)(R²²), —CH₂P(O)(N(R²⁰)₂)₂, or —P(O)(N(R²⁰)₂)₂;
R² is —OH, —OCH₂P(O)(OR²)₂, —OCH₂P(O)(R²⁰)(N(R′)₂), —OCH₂P(O)(R²⁰)(OR²⁰), —OCH₂P(O)(N(R²⁰)₂)(OR²⁰), —OCH₂P(O)(N(R²⁰)₂)₂, —N(R²¹)(R²²), —N(R²⁰)C(O)R²⁰, —N(R²⁰)C(O)OR², —N(R²⁰)C(O)N(R²¹)(R²¹), —N(R²)S(O)₂(R²⁰), —NR²⁰S(O)₂N(R²¹)(R²²), or —NR²⁰S(O)₂O(R²⁰);
R³ is halo, cyano, or C₁₋₆ haloalkyl;
each R⁴ is independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each alkyl, alkenyl, alkynyl, haloalkyl, cycloalkyl, cycloalkenyl, heterocyclyl, aryl, and heteroaryl of R⁴ is independently optionally substituted with 1-5 R¹⁴;
R⁵ is halo, cyano, -L-C₁₋₆ alkyl, -L-C₁₋₆ haloalkyl, -L-C₃₋₁₀ cycloalkyl, -L-C₃₋₁₀ cycloalkenyl, -L-heterocyclyl, -L-aryl, or -L-heteroaryl; wherein each alkyl, haloalkyl, cycloalkyl, cycloalkenyl, heterocyclyl, aryl, and heteroaryl of R⁵ is independently optionally substituted with 1-5 R¹⁵;
L is a bond, —C₁₋₆ alkylene, —C₁₋₆ heteroalkylene, —O—, —S—, —S(O)—, —S(O)₂—, —NR¹⁶—, —C(O)NR¹⁶—, —NR¹⁶C(O)—, —OC(O)—, or —C(O)O—;
each of R⁶, R⁷, R⁸, and R⁹ are independently hydrogen, deuterium, C₁₋₆ alkyl, or C₁₋₆ haloalkyl;
each of R¹⁴ and R¹⁵ are independently hydroxy, halo, cyano, —NO₂, C₁₋₆ alkyl, C₂₋₆ alkenyl, C₂₋₆ alkynyl, C₁₋₆haloalkyl, C₁₋₆ alkoxy, C₁₋₆ haloalkoxy, C₃₋₁₀ cycloalkyl, aryl, benzyl, heteroaryl, heterocyclyl, —N(R¹⁶)₂, —C(O)R¹⁶, —C(O)OR¹⁶, —S—R¹⁶, S(O)R¹⁶, —NR¹⁶S(O)R¹⁶, —S(O)N(R¹⁶)₂, —NR¹⁶S(O)N(R¹⁶)₂, —S(O)₂R¹⁶, —NR¹⁶S(O)₂—R¹⁶, —S(O)₂N(R¹⁶)₂, —NR¹⁶S(O)₂N(R¹⁶)₂, —NR¹⁶C(O)N(R¹⁶)₂, —C(O)N(R¹⁶)₂, —NR¹⁶C(O)R¹⁶, —OC(O)N(R¹⁶)₂, or —NR¹⁶C(O)OR¹⁶;
each R¹⁶ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆haloalkyl, C₁₋₆ heteroalkyl, C₃₋₁₀ cycloalkyl, heterocyclyl, aryl, or heteroaryl of R¹⁶ is independently optionally substituted with 1-5 halo, cyano, —NO₂, oxo, —SF₅, C₁₋₆ alkyl, C₁₋₆ haloalkyl, C₁₋₆ alkoxy, C₁₋₆ haloalkoxy, C₃₋₁₀ cycloalkyl, aryl, benzyl, heteroaryl, or heterocyclyl;
each R²⁰ is independently hydrogen, C₁₋₆ alkyl, C₂₋₆ alkenyl, C₂₋₆ alkynyl, C₃₋₁₀ cycloalkyl, aryl, heteroaryl, or heterocyclyl; wherein each alkyl, alkenyl, alkynyl, cycloalkyl, aryl, heteroaryl, or heterocyclyl is independently optionally substituted with 1-5 R³⁰;
each R²¹ and R²² is independently hydrogen, C₁₋₆ alkyl, C₂₋₆ alkenyl, C₂₋₆ alkynyl, C₃₋₁₀ cycloalkyl, aryl, heteroaryl, or heterocyclyl; wherein each alkyl, alkenyl, alkynyl, cycloalkyl, aryl, heteroaryl, or heterocyclyl is independently optionally substituted with 1-5 R³⁰ groups, or R²⁰ and R²¹ together with the nitrogen to which they are attached form a heterocyclyl; wherein said heterocyclyl is independently optionally substituted with 1-5 R³⁰;
each R³⁰ is independently oxo, thioxo, hydroxy, halo, —NO₂, —N₃, cyano, C₁₋₆ alkyl, C₂₋₆ alkenyl, C₂₋₆ alkynyl, C₃₋₁₀ cycloalkyl, C₁₋₆ haloalkyl, aryl, heteroaryl, heterocyclyl, —O(C₁₋₆ alkyl), —O(C₂₋₆ alkenyl), —O(C₂₋₆ alkynyl), —O(C₃₋₁₀ cycloalkyl), —O(C₁₋₆ haloalkyl), —O(aryl), —O(heteroaryl), —O(heterocyclyl), —NH₂, —NH(C₁₋₆ alkyl), —NH(C₂₋₆ alkenyl), —NH(C₂₋₆ alkynyl), —NH(C₃₋₁₀ cycloalkyl), —NH(C₁₋₆ haloalkyl), —NH(aryl), —NH(heteroaryl), —NH(heterocyclyl), —N(C₁₋₆ alkyl)₂, —N(C₃₋₁₀ cycloalkyl)₂, —N(C₂₋₆ alkenyl)₂, —N(C₂₋₆ alkynyl)₂, —N(C₃₋₁₀ cycloalkyl)₂, —N(C₁₋₆ haloalkyl)₂, —N(aryl)₂, —N(heteroaryl)₂, —N(heterocyclyl)₂, —N(C₁₋₆ alkyl)(C₃₋₁₀ cycloalkyl), —N(C₁₋₆ alkyl)(C₂₋₆ alkenyl), —N(C₁₋₆ alkyl)(C₂₋₆ alkynyl), —N(C₁₋₆ alkyl)(C₃₋₁₀ cycloalkyl), —N(C₁₋₆ alkyl)(C₁₋₆ haloalkyl), —N(C₁₋₆ alkyl)(aryl), —N(C₁₋₆ alkyl)(heteroaryl), —N(C₁₋₆ alkyl)(heterocyclyl), —C(O)(C₁₋₆ alkyl), —C(O)(C₂₋₆ alkenyl), —C(O)(C₂₋₆ alkynyl), —C(O)(C₃₋₁₀ cycloalkyl), —C(O)(C₁₋₆ haloalkyl), —C(O)(aryl), —C(O)(heteroaryl), —C(O)(heterocyclyl), —C(O)O(C₁₋₆ alkyl), —C(O)O(C₂₋₆ alkenyl), —C(O)O(C₂₋₆ alkynyl), —C(O)O(C₃₋₁₀ cycloalkyl), —C(O)O(C₁₋₆ haloalkyl), —C(O)O(aryl), —C(O)O(heteroaryl), —C(O)O(heterocyclyl), —C(O)NH₂, —C(O)NH(C₁₋₆ alkyl), —C(O)NH(C₂₋₆ alkenyl), —C(O)NH(C₂₋₆ alkynyl), —C(O)NH(C₃₋₁₀ cycloalkyl), —C(O)NH(C₁₋₆ haloalkyl), —C(O)NH(aryl), —C(O)NH(heteroaryl), —C(O)NH(heterocyclyl), —C(O)N(C₁₋₆ alkyl)₂, —C(O)N(C₃₋₁₀ cycloalkyl)₂, —C(O)N(C₂₋₆ alkenyl)₂, —C(O)N(C₂₋₆ alkynyl)₂, —C(O)N(C₃₋₁₀ cycloalkyl)₂, —C(O)N(C₁₋₆ haloalkyl)₂, —C(O)N(aryl)₂, —C(O)N(heteroaryl)₂, —C(O)N(heterocyclyl)₂, —NHC(O)(C₁₋₆ alkyl), —NHC(O)(C₂₋₆ alkenyl), —NHC(O)(C₂₋₆ alkynyl), —NHC(O)(C₃₋₁₀ cycloalkyl), —NHC(O)(C₁₋₆ haloalkyl), —NHC(O)(aryl), —NHC(O)(heteroaryl), —NHC(O)(heterocyclyl), —NHC(O)O(C₁₋₆ alkyl), —NHC(O)O(C₂₋₆ alkenyl), —NHC(O)O(C₂₋₆ alkynyl), —NHC(O)O(C₃₋₁₀ cycloalkyl), —NHC(O)O(C₁₋₆ haloalkyl), —NHC(O)O(aryl), —NHC(O)O(heteroaryl), —NHC(O)O(heterocyclyl), —NHC(O)NH(C₁₋₆ alkyl), —NHC(O)NH(C₂₋₆ alkenyl), —NHC(O)NH(C₂₋₆ alkynyl), —NHC(O)NH(C₃₋₁₀ cycloalkyl), —NHC(O)NH(C₁₋₆ haloalkyl), —NHC(O)NH(aryl), —NHC(O)NH(heteroaryl), —NHC(O)NH(heterocyclyl), —SH, —S(C₁₋₆ alkyl), —S(C₂₋₆ alkenyl), —S(C₂₋₆ alkynyl), —S(C₃₋₁₀ cycloalkyl), —S(C₁₋₆ haloalkyl), —S(aryl), —S(heteroaryl), —S(heterocyclyl), —NHS(O)(C₁₋₆ alkyl), —N(C₁₋₆ alkyl)(S(O)(C₁₋₆ alkyl), —S(O)N(C₁₋₆ alkyl)₂, —S(O)(C₁₋₆ alkyl), —S(O)(NH)(C₁₋₆ alkyl), —S(O)(C₂₋₆ alkenyl), —S(O)(C₂₋₆ alkynyl), —S(O)(C₃₋₁₀ cycloalkyl), —S(O)(C₁₋₆ haloalkyl), —S(O)(aryl), —S(O)(heteroaryl), —S(O)(heterocyclyl), —S(O)₂(C₁₋₆ alkyl), —S(O)₂(C₂₋₆ alkenyl), —S(O)₂(C₂₋₆ alkynyl), —S(O)₂(C₃₋₁₀ cycloalkyl), —S(O)₂(C₁₋₆ haloalkyl), —S(O)₂(aryl), —S(O)₂(heteroaryl), —S(O)₂(heterocyclyl), —S(O)₂NH(C₁₋₆ alkyl), or —S(O)₂N(C₁₋₆ alkyl)₂; wherein each alkyl, cycloalkyl, aryl, heteroaryl, or heterocyclyl of R³⁰ is optionally substituted with one to four halo, C₁₋₆ alkyl, C₁₋₆ haloalkyl, —OH, —NH₂, —NH(C₁₋₆ alkyl), —NH(C₃₋₁₀ cycloalkyl), —NH(C₁₋₆ haloalkyl), —NH(aryl), —NH(heteroaryl), —NH(heterocyclyl), —N(C₁₋₆ alkyl)₂, —N(C₃₋₁₀ cycloalkyl)₂, —NHC(O)(C₃₋₁₀ cycloalkyl), —NHC(O)(C₁₋₆ haloalkyl), —NHC(O)(aryl), —NHC(O)(heteroaryl), —NHC(O)(heterocyclyl), —NHC(O)O(C₁₋₆ alkyl), —NHC(O)O(C₂₋₆ alkynyl), —NHC(O)O(C₃₋₁₀ cycloalkyl), —NHC(O)O(C₁₋₆ haloalkyl), —NHC(O)O(aryl), —NHC(O)O(heteroaryl), —NHC(O)O(heterocyclyl), —NHC(O)NH(C₁₋₆ alkyl), —S(O)(NH)(C₁₋₆ alkyl), S(O)₂(C₁₋₆ alkyl), —S(O)₂(C₃₋₁₀ cycloalkyl), —S(O)₂(C₁₋₆ haloalkyl), —S(O)₂(aryl), —S(O)₂(heteroaryl), —S(O)₂(heterocyclyl), —S(O)₂NH(C₁₋₆ alkyl), —S(O)₂N(C₁₋₆ alkyl)₂, —O(C₃₋₁₀ cycloalkyl), —O(C₁₋₆ haloalkyl), —O(aryl), —O(heteroaryl), —O(heterocyclyl), or —O(C₁₋₆ alkyl).

In certain embodiments, when W and X are both CH, then R⁵ and R³ are not both halo. In certain embodiments, the compound is not 3-bromo-2-hydroxy-5-methoxy-y-oxo-benzenebutanoic acid.

This disclosure also provides pharmaceutical compositions comprising one or more compounds of formula I and a pharmaceutically acceptable excipient.

This disclosure is also directed to methods for inhibiting the activity of histone lysine demethylase and treating, pretreating, or delaying onset of a condition associated with histone lysine demethylase. In one aspect, provided is a method of treating, pretreating, or delaying onset of a condition associated with undesirable cellular proliferation.

### DETAILED DESCRIPTION

Before the present compounds and methods are described, it is to be understood that the disclosure is not limited to the methodologies, protocols, cell lines, assays, and reagents described, as these may vary. It is also to be understood that the terminology used herein is intended to describe embodiments of the present disclosure, and is in no way intended to limit the scope of the present disclosure as set forth in the appended claims.

### Definitions

It must be noted that as used herein, and in the appended claims, the singular forms “a,” “an,” and “the” include plural references unless the context clearly dictates otherwise.

Unless defined otherwise, all technical, and scientific terms used herein have the same meanings as commonly understood by one of ordinary skill in the art to which this disclosure pertains. Although any methods and materials similar or equivalent to those described herein can be used in the practice or testing of the present disclosure, exemplary methods, devices, and materials are now described. All publications cited herein are incorporated herein by reference in their entirety for the purpose of describing and disclosing the methodologies, reagents, and tools reported in the publications that might be used in connection with the disclosure. Nothing herein is to be construed as an admission that the disclosure is not entitled to antedate such publications by virtue of prior invention.

The practice of the present disclosure will employ, unless otherwise indicated, conventional methods of chemistry, biochemistry, molecular biology, cell biology, genetics, immunology, and pharmacology, within the skill of the art. Such techniques are explained fully in the literature. (See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; D. M. Weir, and C. C. Blackwell, eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton & Graham eds. (1997) PCR (Introduction to Biotechniques Series), 2nd ed., Springer Verlag.

The terms “disorders,” “diseases,” and “conditions” are used inclusively and refer to any condition deviating from normal.

The term “alkyl” refers to saturated monovalent straight or branched chain hydrocarbyl groups having from 1 to 10 carbon atoms, from 1 to 6 carbon atoms, or 1 to 3 carbon atoms. This term is exemplified by groups such as methyl, ethyl, n-propyl, iso-propyl, n-butyl, t-butyl, n-pentyl, and the like.

The term “alkoxy” refers to —O-alkyl, where alkyl is as defined above.

The term “alkenyl” refers to a vinyl unsaturated monovalent hydrocarbyl group having from 2 to 6 carbon atoms, or from 2 to 4 carbon atoms, and having at least 1, or from 1 to 2 sites of vinyl (>C═C<) unsaturation. Such groups are exemplified by vinyl (ethen-1-yl), allyl, but-3-enyl and the like.

The term “alkynyl” refers to an acetylinic unsaturated monovalent hydrocarbyl groups having from 2 to 6 carbon atoms, or 2 to 3 carbon atoms, and having at least 1, or from 1 to 2 sites of acetylenic (—C≡C—) unsaturation. This group is exemplified by ethyn-1-yl, propyn-1-yl, propyn-2-yl, and the like.

The term “aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, benzo[1,3]-dioxol-5-yl, 2,3-dihydro-benzo[1,4]dioxin-6-yl, 2,3-dihydro-benzofuran-5-yl, dibenzofuran-4-yl, and the like) provided that the point of attachment is the aryl group.

The term “cycloalkyl” refers to cyclic alkyl groups of from 3 to 10 carbon atoms having single or multiple cyclic rings including, by way of example, adamantyl, cyclopropyl, cyclobutyl, cyclopentyl, cyclooctyl, and the like.

The term “cycloalkenyl” refers to cyclic alkenyl (but not aromatic) groups of from 5 to 10 carbon atoms having single or multiple cyclic rings and having at least one site of vinyl (>C═C<) unsaturation within the ring including, by way of example, cyclopentenyl, cyclooctenyl, and the like.

The term “halo” or “halogen” refers to fluoro, chloro, bromo, and iodo, and in certain embodiments, is fluoro, chloro or bromo.

The term “heteroaryl” refers to an aromatic group of from 1 to 15 carbon atoms, or from 1 to 10 carbon atoms, and 1 to 4 heteroatoms selected from oxygen, nitrogen, and sulfur within the ring. Such heteroaryl groups can have a single ring (e.g., pyridinyl, furyl, or thienyl) or multiple condensed rings (e.g., indolizinyl or benzothienyl). The nitrogen and/or sulfur ring atoms can optionally be oxidized to provide for the N-oxide or the sulfoxide, and sulfone derivatives. Exemplary heteroaryls include pyridinyl, pyrrolyl, indolyl, thiophenyl, thienyl, and furyl.

The term “haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

The term “haloalkoxy” refers to —O-haloalkyl, where haloalkyl is as defined above.

The term “heteroalkylene” refers to a linear, divalent C₁₋₆ alkyl group (i.e., C₁₋₆ alkylene) in which one or two of the carbon atoms (and any associated hydrogen atoms) are each independently replaced with the same or different heteroatomic group. Heteroatomic groups include, but are not limited to, —NH—, —O—, —S—, —S(O)—, —S(O)₂—, and the like.

The term “heterocyclyl” or “heterocyclic” refers to a saturated or unsaturated (but not aromatic) group having a single ring or multiple condensed rings, from 1 to 10 carbon atoms, and from 1 to 4 hetero atoms selected from nitrogen, sulfur or oxygen within the ring wherein, in fused ring systems, one or more of the rings can be aryl or heteroaryl provided that the point of attachment is at the heterocycle. The nitrogen and/or sulfur ring atoms can optionally be oxidized to provide for the N-oxide or the sulfoxide, and sulfone derivatives.

The term “substituted heterocyclyl” or “substituted heterocyclic” refers to heterocycle groups that are substituted with from 1 to 3 of the same substituents as defined for substituted cycloalkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

The term “oxo” refers to the atom (═O) or (—O⁻).

The term “amino acid” refers to any of the naturally occurring amino acids, as well as synthetic analogs (e.g., D-stereoisomers of the naturally occurring amino acids, such as D-threonine), and derivatives thereof. α-Amino acids comprise a carbon atom to which is bonded an amino group, a carboxyl group, a hydrogen atom, and a distinctive group referred to as a “side chain.” The side chains of naturally occurring amino acids are well known in the art, and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine, and tryptophan), substituted arylalkyl (e.g., as in tyrosine), and heteroarylalkyl (e.g., as in histidine). Unnatural amino acids are also known in the art, as set forth in, for example, Williams, ed. (1989) Synthesis of Optically Active α-Amino Acids, Pergamon Press; Evans et al. (1990) J. Amer. Chem. Soc. 112:4011-4030; Pu et al. (1991) J. Amer. Chem. Soc. 56:1280-1283; Williams et al. (1991) J. Amer. Chem. Soc. 113:9276-9286; and all references cited therein.

The term “pharmaceutically acceptable salt” refers to a pharmaceutically acceptable salt of a compound, which salt can be derived from a variety of organic, and inorganic counter ions well known in the art, and include, by way of example only, sodium, potassium, calcium, magnesium, ammonium, tetraalkylammonium, and the like; and when the molecule contains a basic functionality, a salt of an organic or inorganic acid, such as hydrochloride, hydrobromide, tartrate, mesylate, acetate, maleate, oxalate, and the like.

The term “excipient” as used herein means an inert or inactive substance used in the production of pharmaceutical products or other tablets, including without limitation any substance used as a binder, disintegrant, coating, compression/encapsulation aid, cream or lotion, lubricant, parenteral, sweetener or flavoring, suspending/gelling agent, or wet granulation agent.

It is understood that the substituents as defined herein are not intended to include impermissible substitution patterns (e.g., methyl substituted with 5 fluoro groups or a hydroxy group attached to an ethenylic or acetylenic carbon atom). Such impermissible substitution patterns are well known to the skilled artisan.

### 2. Compounds

Provided herein are compounds of formula I:

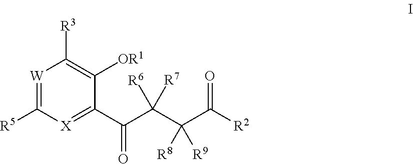

or a pharmaceutically acceptable salt, isotopically enriched analog, stereoisomer, mixture of stereoisomers or prodrug thereof, wherein:
one of W or X is N and the other of W and X is CR⁴; or W and X are CR⁴;
R¹ is hydrogen, —P(O)(OR²⁰)₂, —CH₂P(O)(OR²⁰)₂, —P(O)(R²)(OR²), —CH₂P(O)(R²⁰)(OR²⁰), —P(O)(N(R²⁰)₂)(OR²⁰), —CH₂P(O)(N(R²⁰)₂)(OR²⁰), —P(O)(R²⁰)(N(R²⁰)₂), —CH₂P(O)(R²⁰)(N(R²⁰)₂), —C(O)R²⁰, —C(O)N(R²¹)(R²²), —CH₂P(O)(N(R²⁰)₂)₂, or —P(O)(N(R²⁰)₂)₂;
R² is —OH, —OCH₂P(O)(OR²⁰)₂, —OCH₂P(O)(R²⁰)(N(R)₂), —OCH₂P(O)(R²⁰)(OR²⁰), —OCH₂P(O)(N(R²⁰)₂)(OR²), —OCH₂P(O)(N(R²)₂)₂, —N(R²¹)(R²²), —N(R²⁰)C(O)R²⁰, —N(R²)C(O)OR², —N(R²⁰)C(O)N(R²¹)(R²¹), —N(R²⁰)S(O)₂(R²⁰), —NR²⁰S(O)₂N(R²¹)(R²²), or —NR²⁰S(O)₂O(R²⁰) R³ is halo, cyano, or C₁₋₆ haloalkyl;
each R⁴ is independently hydrogen, halo, cyano, C₁₋₆ alkyl, C₂₋₆ alkenyl, C₂₋₆ alkynyl,
C₁₋₆ haloalkyl, C₃₋₁₀ cycloalkyl, C₃₋₁₀ cycloalkenyl, heterocyclyl, aryl, or heteroaryl; wherein each alkyl, alkenyl, alkynyl, haloalkyl, cycloalkyl, cycloalkenyl, heterocyclyl, aryl, and heteroaryl of R⁴ is independently optionally substituted with 1-5 R¹⁴;
R⁵ is halo, cyano, -L-C₁₋₆ alkyl, -L-C₁₋₆ haloalkyl, -L-C₃₋₁₀ cycloalkyl, -L-C₃₋₁₀ cycloalkenyl, -L-heterocyclyl, -L-aryl, or -L-heteroaryl; wherein each alkyl, haloalkyl, cycloalkyl, cycloalkenyl, heterocyclyl, aryl, and heteroaryl of R⁵ is independently optionally substituted with 1-5 R¹⁵;
L is a bond, —C₁₋₆ alkylene, —C₁₋₆ heteroalkylene, —O—, —S—, —S(O)—, —S(O)₂—, —NR¹⁶—, —C(O)NR¹⁶—, —NR¹⁶C(O)—, —OC(O)—, or —C(O)O—;
each of R⁶, R⁷, R⁸, and R⁹ are independently hydrogen, deuterium, C₁₋₆ alkyl, or C₁₋₆ haloalkyl;
each of R¹⁴ and R¹⁵ are independently hydroxy, halo, cyano, —NO₂, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, aryl, benzyl, heteroaryl, heterocyclyl, —N(R¹⁶)₂, —C(O)R¹⁶, —C(O)OR¹⁶, —S—R¹⁶, S(O)R¹⁶, —NR¹⁶S(O)R¹⁶, —S(O)N(R¹⁶)₂, —NR¹⁶S(O)N(R¹⁶)₂, —S(O)₂R¹⁶, —NR¹⁶S(O)₂—R¹⁶, —S(O)₂N(R¹⁶)₂, —NR¹⁶S(O)₂N(R¹⁶)₂, —NR¹⁶C(O)N(R¹⁶)₂, —C(O)N(R¹⁶)₂, —NR¹⁶C(O)R¹⁶, —OC(O)N(R¹⁶)₂, or —NR¹⁶C(O)OR¹⁶;
each R¹⁶ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆haloalkyl, C₁₋₆ heteroalkyl, C₃₋₁₀ cycloalkyl, heterocyclyl, aryl, or heteroaryl of R¹⁶ is independently optionally substituted with 1-5 halo, cyano, —NO₂, oxo, —SF₅, C₁₋₆ alkyl, C₁₋₆ haloalkyl, C₁₋₆ alkoxy, C₁₋₆ haloalkoxy, C₃₋₁₀ cycloalkyl, aryl, benzyl, heteroaryl, or heterocyclyl;
each R²⁰ is independently hydrogen, C₁₋₆ alkyl, C₂₋₆ alkenyl, C₂₋₆ alkynyl, C₃₋₁₀ cycloalkyl, aryl, heteroaryl, or heterocyclyl; wherein each alkyl, alkenyl, alkynyl, cycloalkyl, aryl, heteroaryl, or heterocyclyl is independently optionally substituted with 1-5 R³⁰;
each R²¹ and R²² is independently hydrogen, C₁₋₆ alkyl, C₂₋₆ alkenyl, C₂₋₆ alkynyl, C₃₋₁₀ cycloalkyl, aryl, heteroaryl, or heterocyclyl; wherein each alkyl, alkenyl, alkynyl, cycloalkyl, aryl, heteroaryl, or heterocyclyl is independently optionally substituted with 1-5 R³⁰ groups, or R²⁰ and R²¹ together with the nitrogen to which they are attached form a heterocyclyl; wherein said heterocyclyl is independently optionally substituted with 1-5 R³⁰;
each R³⁰ is independently oxo, thioxo, hydroxy, halo, —NO₂, —N₃, cyano, C₁₋₆ alkyl, C₂₋₆ alkenyl, C₂₋₆ alkynyl, C₃₋₁₀ cycloalkyl, C₁₋₆ haloalkyl, aryl, heteroaryl, heterocyclyl, —O(C₁₋₆ alkyl), —O(C₂₋₆ alkenyl), —O(C₂₋₆ alkynyl), —O(C₃₋₁₀ cycloalkyl), —O(C₁₋₆ haloalkyl), —O(aryl), —O(heteroaryl), —O(heterocyclyl), —NH₂, —NH(C₁₋₆ alkyl), —NH(C₂₋₆ alkenyl), —NH(C₂₋₆ alkynyl), —NH(C₃₋₁₀ cycloalkyl), —NH(C₁₋₆ haloalkyl), —NH(aryl), —NH(heteroaryl), —NH(heterocyclyl), —N(C₁₋₆ alkyl)₂, —N(C₃₋₁₀ cycloalkyl)₂, —N(C₂₋₆ alkenyl)₂, —N(C₂₋₆ alkynyl)₂, —N(C₃₋₁₀ cycloalkyl)₂, —N(C₁₋₆ haloalkyl)₂, —N(aryl)₂, —N(heteroaryl)₂, —N(heterocyclyl)₂, —N(C₁₋₆ alkyl)(C₃₋₁₀ cycloalkyl), —N(C₁₋₆ alkyl)(C₂₋₆ alkenyl), —N(C₁₋₆ alkyl)(C₂₋₆ alkynyl), —N(C₁₋₆ alkyl)(C₃₋₁₀ cycloalkyl), —N(C₁₋₆ alkyl)(C₁₋₆ haloalkyl), —N(C₁₋₆ alkyl)(aryl), —N(C₁₋₆ alkyl)(heteroaryl), —N(C₁₋₆ alkyl)(heterocyclyl), —C(O)(C₁₋₆ alkyl), —C(O)(C₂₋₆ alkenyl), —C(O)(C₂₋₆ alkynyl), —C(O)(C₃₋₁₀ cycloalkyl), —C(O)(C₁₋₆ haloalkyl), —C(O)(aryl), —C(O)(heteroaryl), —C(O)(heterocyclyl), —C(O)O(C₁₋₆ alkyl), —C(O)O(C₂₋₆ alkenyl), —C(O)O(C₂₋₆ alkynyl), —C(O)O(C₃₋₁₀ cycloalkyl), —C(O)O(C₁₋₆ haloalkyl), —C(O)O(aryl), —C(O)O(heteroaryl), —C(O)O(heterocyclyl), —C(O)NH₂, —C(O)NH(C₁₋₆ alkyl), —C(O)NH(C₂₋₆ alkenyl), —C(O)NH(C₂₋₆ alkynyl), —C(O)NH(C₃₋₁₀ cycloalkyl), —C(O)NH(C₁₋₆ haloalkyl), —C(O)NH(aryl), —C(O)NH(heteroaryl), —C(O)NH(heterocyclyl), —C(O)N(C₁₋₆ alkyl)₂, —C(O)N(C₃₋₁₀ cycloalkyl)₂, —C(O)N(C₂₋₆ alkenyl)₂, —C(O)N(C₂₋₆ alkynyl)₂, —C(O)N(C₃₋₁₀ cycloalkyl)₂, —C(O)N(C₁₋₆ haloalkyl)₂, —C(O)N(aryl)₂, —C(O)N(heteroaryl)₂, —C(O)N(heterocyclyl)₂, —NHC(O)(C₁₋₆ alkyl), —NHC(O)(C₂₋₆ alkenyl), —NHC(O)(C₂₋₆ alkynyl), —NHC(O)(C₃₋₁₀ cycloalkyl), —NHC(O)(C₁₋₆ haloalkyl), —NHC(O)(aryl), —NHC(O)(heteroaryl), —NHC(O)(heterocyclyl), —NHC(O)O(C₁₋₆ alkyl), —NHC(O)O(C₂₋₆ alkenyl), —NHC(O)O(C₂₋₆ alkynyl), —NHC(O)O(C₃₋₁₀ cycloalkyl), —NHC(O)O(C₁₋₆ haloalkyl), —NHC(O)O(aryl), —NHC(O)O(heteroaryl), —NHC(O)O(heterocyclyl), —NHC(O)NH(C₁₋₆ alkyl), —NHC(O)NH(C₂₋₆ alkenyl), —NHC(O)NH(C₂₋₆ alkynyl), —NHC(O)NH(C₃₋₁₀ cycloalkyl), —NHC(O)NH(C₁₋₆ haloalkyl), —NHC(O)NH(aryl), —NHC(O)NH(heteroaryl), —NHC(O)NH(heterocyclyl), —SH, —S(C₁₋₆ alkyl), —S(C₂₋₆ alkenyl), —S(C₂₋₆ alkynyl), —S(C₃₋₁₀ cycloalkyl), —S(C₁₋₆ haloalkyl), —S(aryl), —S(heteroaryl), —S(heterocyclyl), —NHS(O)(C₁₋₆ alkyl), —N(C₁₋₆ alkyl)(S(O)(C₁₋₆ alkyl), —S(O)N(C₁₋₆ alkyl)₂, —S(O)(C₁₋₆ alkyl), —S(O)(NH)(C₁₋₆ alkyl), —S(O)(C₂₋₆ alkenyl), —S(O)(C₂₋₆ alkynyl), —S(O)(C₃₋₁₀ cycloalkyl), —S(O)(C₁₋₆ haloalkyl), —S(O)(aryl), —S(O)(heteroaryl), —S(O)(heterocyclyl), —S(O)₂(C₁₋₆ alkyl), —S(O)₂(C₂₋₆ alkenyl), —S(O)₂(C₂₋₆ alkynyl), —S(O)₂(C₃₋₁₀ cycloalkyl), —S(O)₂(C₁₋₆ haloalkyl), —S(O)₂(aryl), —S(O)₂(heteroaryl), —S(O)₂(heterocyclyl), —S(O)₂NH(C₁₋₆ alkyl), or —S(O)₂N(C₁₋₆ alkyl)₂; wherein each alkyl, cycloalkyl, aryl, heteroaryl, or heterocyclyl of R³⁰ is optionally substituted with one to four halo, C₁₋₆ alkyl, C₁₋₆ haloalkyl, —OH, —NH₂, —NH(C₁₋₆ alkyl), —NH(C₃₋₁₀ cycloalkyl), —NH(C₁₋₆ haloalkyl), —NH(aryl), —NH(heteroaryl), —NH(heterocyclyl), —N(C₁₋₆ alkyl)₂, —N(C₃₋₁₀ cycloalkyl)₂, —NHC(O)(C₃₋₁₀ cycloalkyl), —NHC(O)(C₁₋₆ haloalkyl), —NHC(O)(aryl), —NHC(O)(heteroaryl), —NHC(O)(heterocyclyl), —NHC(O)O(C₁₋₆ alkyl), —NHC(O)O(C₂₋₆ alkynyl), —NHC(O)O(C₃₋₁₀ cycloalkyl), —NHC(O)O(C₁₋₆ haloalkyl), —NHC(O)O(aryl), —NHC(O)O(heteroaryl), —NHC(O)O(heterocyclyl), —NHC(O)NH(C₁₋₆ alkyl), —S(O)(NH)(C₁₋₆ alkyl), S(O)₂(C₁₋₆ alkyl), —S(O)₂(C₃₋₁₀ cycloalkyl), —S(O)₂(C₁₋₆ haloalkyl), —S(O)₂(aryl), —S(O)₂(heteroaryl), —S(O)₂(heterocyclyl), —S(O)₂NH(C₁₋₆ alkyl), —S(O)₂N(C₁₋₆ alkyl)₂, —O(C₃₋₁₀ cycloalkyl), —O(C₁₋₆ haloalkyl), —O(aryl), —O(heteroaryl), —O(heterocyclyl), or —O(C₁₋₆ alkyl);
   provided that:
when W and X are both CH, then R⁵ and R³ are not both halo; and
the compound is not 3-bromo-2-hydroxy-5-methoxy-y-oxo-benzenebutanoic acid.

In certain embodiments, provided herein are compounds of formula I:

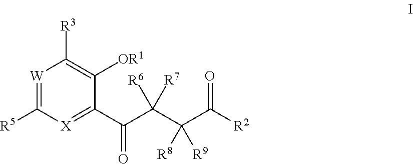

or a pharmaceutically acceptable salt, isotopically enriched analog, stereoisomer, mixture of stereoisomers or prodrug thereof, wherein:
one of W or X is N and the other of W and X is CR⁴; or W and X are CR⁴;
R¹ is hydrogen, —P(O)(OR²⁰)₂, —CH₂P(O)(OR²⁰)₂, —P(O)(R²⁰)(OR²⁰), —CH₂P(O)(R²⁰)(OR²⁰), —P(O)(N(R²⁰)₂)(OR²⁰), —CH₂P(O)(N(R²⁰)₂)(OR²⁰), —P(O)(R²⁰)(N(R²⁰)₂), —CH₂P(O)(R²⁰)(N(R²⁰)₂), —C(O)R²⁰, —C(O)N(R²¹)(R²²), —CH₂P(O)(N(R²⁰)₂)₂, or —P(O)(N(R²⁰)₂)₂;
R² is —OH, —OCH₂P(O)(OR²⁰)₂, —OCH₂P(O)(R²⁰)(N(R²⁰)₂), —OCH₂P(O)(R²⁰)(OR²⁰), —OCH₂P(O)(N(R²⁰)₂)(OR²⁰), —OCH₂P(O)(N(R²⁰)₂)₂, —N(R²¹)(R²²), —N(R²⁰)C(O)R²⁰, —N(R²⁰)C(O)OR²⁰, —N(R²⁰)C(O)N(R²¹)(R²¹), —N(R²⁰)S(O)₂(R²⁰), —NR²⁰S(O)₂N(R²¹)(R²²), or —NR²⁰S(O)₂O(R²⁰)
R³ is halo, cyano, or C₁₋₆ haloalkyl;
each R⁴ is independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each alkyl, alkenyl, alkynyl, haloalkyl, cycloalkyl, cycloalkenyl, heterocyclyl, aryl, and heteroaryl of R⁴ is independently optionally substituted with 1-5 R¹⁴;
R⁵ is halo, cyano, -L-C₁₋₆ alkyl, -L-C₁₋₆ haloalkyl, -L-C₃₋₁₀ cycloalkyl, -L-C₃₋₁₀ cycloalkenyl, -L-heterocyclyl, -L-aryl, or -L-heteroaryl; wherein each alkyl, haloalkyl, cycloalkyl, cycloalkenyl, heterocyclyl, aryl, and heteroaryl of R⁵ is independently optionally substituted with 1-5 R¹⁵;
L is a bond, —C₁₋₆ alkylene, —C₁₋₆ heteroalkylene, —O—, —S—, —S(O)—, —S(O)₂—, —NR¹⁶—, —C(O)NR¹⁶—, —NR¹⁶C(O)—, —OC(O)—, or —C(O)O—;
each of R⁶, R⁷, R⁸, and R⁹ are independently hydrogen, deuterium, C₁₋₆ alkyl, or C₁₋₆ haloalkyl;
each of R¹⁴ and R¹⁵ are independently halo, cyano, —NO₂, C₁₋₆ alkyl, C₂₋₆ alkenyl, C₂₋₆ alkynyl, C₁₋₆haloalkyl, C₁₋₆ alkoxy, C₁₋₆ haloalkoxy, —N(R¹⁶)₂, —C(O)R¹⁶, —C(O)OR¹⁶, —S—R¹⁶, S(O)R¹⁶, —NR¹⁶S(O)R¹⁶, —S(O)N(R¹⁶)₂, —NR¹⁶S(O)N(R¹⁶)₂, —S(O)₂R¹⁶, —NR¹⁶S(O)₂—R¹⁶, —S(O)₂N(R¹⁶)₂, —NR¹⁶S(O)₂N(R¹⁶)₂, —NR¹⁶C(O)N(R¹⁶)₂, —C(O)N(R¹⁶)₂, —NR¹⁶C(O)R¹⁶, —OC(O)N(R¹⁶)₂, or —NR¹⁶C(O)OR¹⁶;
each R¹⁶ is independently hydrogen, C₁₋₆ alkyl, C₂₋₆ alkenyl, C₂₋₆ alkynyl, C₁₋₆haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆haloalkyl, C₁₋₆ heteroalkyl, C₃₋₁₀ cycloalkyl, heterocyclyl, aryl, or heteroaryl of R¹⁶ is independently optionally substituted with 1-5 halo, cyano, —NO₂, oxo, —SF₅, C₁₋₆ alkyl, C₁₋₆ haloalkyl, C₁₋₆ alkoxy, C₁₋₆ haloalkoxy, C₃₋₁₀ cycloalkyl, aryl, benzyl, heteroaryl, or heterocyclyl;
each R²⁰ is independently hydrogen, C₁₋₆ alkyl, C₂₋₆ alkenyl, C₂₋₆ alkynyl, C₃₋₁₀ cycloalkyl, aryl, heteroaryl, or heterocyclyl; wherein each alkyl, alkenyl, alkynyl, cycloalkyl, aryl, heteroaryl, or heterocyclyl is independently optionally substituted with 1-5 R³⁰;
each R²¹ and R²² is independently hydrogen, C₁₋₆ alkyl, C₂₋₆ alkenyl, C₂₋₆ alkynyl, C₃₋₁₀ cycloalkyl, aryl, heteroaryl, or heterocyclyl; wherein each alkyl, alkenyl, alkynyl, cycloalkyl, aryl, heteroaryl, or heterocyclyl is independently optionally substituted with 1-5 R³⁰ groups, or R²⁰ and R²¹ together with the nitrogen to which they are attached form a heterocyclyl; wherein said heterocyclyl is independently optionally substituted with 1-5 R³⁰;
each R³⁰ is independently oxo, thioxo, hydroxy, halo, —NO₂, —N₃, cyano, C₁₋₆ alkyl, C₂₋₆ alkenyl, C₂₋₆ alkynyl, C₃₋₁₀ cycloalkyl, C₁₋₆ haloalkyl, aryl, heteroaryl, heterocyclyl, —O(C₁₋₆ alkyl), —O(C₂₋₆ alkenyl), —O(C₂₋₆ alkynyl), —O(C₃₋₁₀ cycloalkyl), —O(C₁₋₆ haloalkyl), —O(aryl), —O(heteroaryl), —O(heterocyclyl), —NH₂, —NH(C₁₋₆ alkyl), —NH(C₂₋₆ alkenyl), —NH(C₂₋₆ alkynyl), —NH(C₃₋₁₀ cycloalkyl), —NH(C₁₋₆ haloalkyl), —NH(aryl), —NH(heteroaryl), —NH(heterocyclyl), —N(C₁₋₆ alkyl)₂, —N(C₃₋₁₀ cycloalkyl)₂, —N(C₂₋₆ alkenyl)₂, —N(C₂₋₆ alkynyl)₂, —N(C₃₋₁₀ cycloalkyl)₂, —N(C₁₋₆ haloalkyl)₂, —N(aryl)₂, —N(heteroaryl)₂, —N(heterocyclyl)₂, —N(C₁₋₆ alkyl)(C₃₋₁₀ cycloalkyl), —N(C₁₋₆ alkyl)(C₂₋₆ alkenyl), —N(C₁₋₆ alkyl)(C₂₋₆ alkynyl), —N(C₁₋₆ alkyl)(C₃₋₁₀ cycloalkyl), —N(C₁₋₆ alkyl)(C₁₋₆ haloalkyl), —N(C₁₋₆ alkyl)(aryl), —N(C₁₋₆ alkyl)(heteroaryl), —N(C₁₋₆ alkyl)(heterocyclyl), —C(O)(C₁₋₆ alkyl), —C(O)(C₂₋₆ alkenyl), —C(O)(C₂₋₆ alkynyl), —C(O)(C₃₋₁₀ cycloalkyl), —C(O)(C₁₋₆ haloalkyl), —C(O)(aryl), —C(O)(heteroaryl), —C(O)(heterocyclyl), —C(O)O(C₁₋₆ alkyl), —C(O)O(C₂₋₆ alkenyl), —C(O)O(C₂₋₆ alkynyl), —C(O)O(C₃₋₁₀ cycloalkyl), —C(O)O(C₁₋₆ haloalkyl), —C(O)O(aryl), —C(O)O(heteroaryl), —C(O)O(heterocyclyl), —C(O)NH₂, —C(O)NH(C₁₋₆ alkyl), —C(O)NH(C₂₋₆ alkenyl), —C(O)NH(C₂₋₆ alkynyl), —C(O)NH(C₃₋₁₀ cycloalkyl), —C(O)NH(C₁₋₆ haloalkyl), —C(O)NH(aryl), —C(O)NH(heteroaryl), —C(O)NH(heterocyclyl), —C(O)N(C₁₋₆ alkyl)₂, —C(O)N(C₃₋₁₀ cycloalkyl)₂, —C(O)N(C₂₋₆ alkenyl)₂, —C(O)N(C₂₋₆ alkynyl)₂, —C(O)N(C₃₋₁₀ cycloalkyl)₂, —C(O)N(C₁₋₆ haloalkyl)₂, —C(O)N(aryl)₂, —C(O)N(heteroaryl)₂, —C(O)N(heterocyclyl)₂, —NHC(O)(C₁₋₆ alkyl), —NHC(O)(C₂₋₆ alkenyl), —NHC(O)(C₂₋₆ alkynyl), —NHC(O)(C₃₋₁₀ cycloalkyl), —NHC(O)(C₁₋₆ haloalkyl), —NHC(O)(aryl), —NHC(O)(heteroaryl), —NHC(O)(heterocyclyl), —NHC(O)O(C₁₋₆ alkyl), —NHC(O)O(C₂₋₆ alkenyl), —NHC(O)O(C₂₋₆ alkynyl), —NHC(O)O(C₃₋₁₀ cycloalkyl), —NHC(O)O(C₁₋₆ haloalkyl), —NHC(O)O(aryl), —NHC(O)O(heteroaryl), —NHC(O)O(heterocyclyl), —NHC(O)NH(C₁₋₆ alkyl), —NHC(O)NH(C₂₋₆ alkenyl), —NHC(O)NH(C₂₋₆ alkynyl), —NHC(O)NH(C₃₋₁₀ cycloalkyl), —NHC(O)NH(C₁₋₆ haloalkyl), —NHC(O)NH(aryl), —NHC(O)NH(heteroaryl), —NHC(O)NH(heterocyclyl), —SH, —S(C₁₋₆ alkyl), —S(C₂₋₆ alkenyl), —S(C₂₋₆ alkynyl), —S(C₃₋₁₀ cycloalkyl), —S(C₁₋₆ haloalkyl), —S(aryl), —S(heteroaryl), —S(heterocyclyl), —NHS(O)(C₁₋₆ alkyl), —N(C₁₋₆ alkyl)(S(O)(C₁₋₆ alkyl), —S(O)N(C₁₋₆ alkyl)₂, —S(O)(C₁₋₆ alkyl), —S(O)(NH)(C₁₋₆ alkyl), —S(O)(C₂₋₆ alkenyl), —S(O)(C₂₋₆ alkynyl), —S(O)(C₃₋₁₀ cycloalkyl), —S(O)(C₁₋₆ haloalkyl), —S(O)(aryl), —S(O)(heteroaryl), —S(O)(heterocyclyl), —S(O)₂(C₁₋₆ alkyl), —S(O)₂(C₂₋₆ alkenyl), —S(O)₂(C₂₋₆ alkynyl), —S(O)₂(C₃₋₁₀ cycloalkyl), —S(O)₂(C₁₋₆ haloalkyl), —S(O)₂(aryl), —S(O)₂(heteroaryl), —S(O)₂(heterocyclyl), —S(O)₂NH(C₁₋₆ alkyl), or —S(O)₂N(C₁₋₆ alkyl)₂; wherein each alkyl, cycloalkyl, aryl, heteroaryl, or heterocyclyl of R³⁰ is optionally substituted with one to four halo, C₁₋₆ alkyl, C₁₋₆ haloalkyl, —OH, —NH₂, —NH(C₁₋₆ alkyl), —NH(C₃₋₁₀ cycloalkyl), —NH(C₁₋₆ haloalkyl), —NH(aryl), —NH(heteroaryl), —NH(heterocyclyl), —N(C₁₋₆ alkyl)₂, —N(C₃₋₁₀ cycloalkyl)₂, —NHC(O)(C₃₋₁₀ cycloalkyl), —NHC(O)(C₁₋₆ haloalkyl), —NHC(O)(aryl), —NHC(O)(heteroaryl), —NHC(O)(heterocyclyl), —NHC(O)O(C₁₋₆ alkyl), —NHC(O)O(C₂₋₆ alkynyl), —NHC(O)O(C₃₋₁₀ cycloalkyl), —NHC(O)O(C₁₋₆ haloalkyl), —NHC(O)O(aryl), —NHC(O)O(heteroaryl), —NHC(O)O(heterocyclyl), —NHC(O)NH(C₁₋₆ alkyl), —S(O)(NH)(C₁₋₆ alkyl), S(O)₂(C₁₋₆ alkyl), —S(O)₂(C₃₋₁₀ cycloalkyl), —S(O)₂(C₁₋₆ haloalkyl), —S(O)₂(aryl), —S(O)₂(heteroaryl), —S(O)₂(heterocyclyl), —S(O)₂NH(C₁₋₆ alkyl), —S(O)₂N(C₁₋₆ alkyl)₂, —O(C₃₋₁₀ cycloalkyl), —O(C₁₋₆ haloalkyl), —O(aryl), —O(heteroaryl), —O(heterocyclyl), or —O(C₁₋₆ alkyl);
   provided that:
when W and X are both CH, then R¹ and R³ are not both halo; and
the compound is not 3-bromo-2-hydroxy-5-methoxy-y-oxo-benzenebutanoic acid.

In certain embodiments, provided is a compound of formula II, or a pharmaceutically acceptable salt, isotopically enriched analog, stereoisomer, mixture of stereoisomers or prodrug thereof:

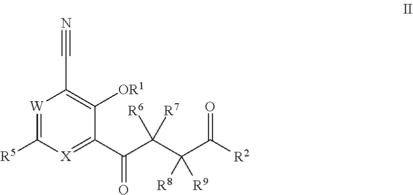

wherein each of W, X, R¹, R², R⁵, R⁶, R⁷, R⁸ and R⁹ are independently as defined herein.

In certain embodiments, X is N and W is CR⁴.

In certain embodiments, W is N and X is CR⁴.

In certain embodiments, X is N and W is CH.

In certain embodiments, W is N and X is CH.

In certain embodiments, W and X are CH.

In certain embodiments, provided is a compound of formula III, or a pharmaceutically acceptable salt, isotopically enriched analog, stereoisomer, mixture of stereoisomers or prodrug thereof:

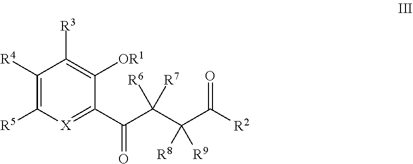

wherein each of X, R¹, R², R³, R⁴, R⁵, R⁶, R⁷, R⁸ and R⁹ are independently as defined herein.

In certain embodiments, R⁶, R⁷, R⁸, and R⁹ are hydrogen.

In certain embodiments, provided is a compound of formula IV, or a pharmaceutically acceptable salt, isotopically enriched analog, stereoisomer, mixture of stereoisomers or prodrug thereof:

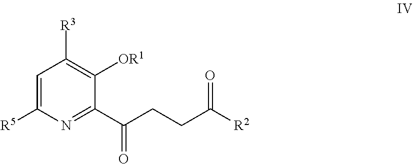

wherein each of R¹, R², R³ and R⁵ are independently as defined herein.

In certain embodiments, provided is a compound of formula V, or a pharmaceutically acceptable salt, isotopically enriched analog, stereoisomer, mixture of stereoisomers or prodrug thereof:

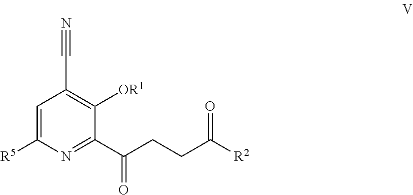

wherein each of R¹, R², and R⁵ are independently as defined herein.

In certain embodiments, R¹ is H.

In certain embodiments, R² is —OH.

In certain embodiments, R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵.

In certain embodiments, R⁵ is -L-aryl or -L-heteroaryl; wherein each aryl and heteroaryl of R⁵ is optionally substituted with 1-3 R¹⁵.

In certain embodiments, each of R¹⁴ and R¹⁵ are independently halo, cyano, —NO₂, C₁₋₆ alkyl, C₂₋₆ alkenyl, C₂₋₆ alkynyl, C₁₋₆haloalkyl, C₁₋₆ alkoxy, C₁₋₆ haloalkoxy, —N(R¹⁶)₂, —C(O)R¹⁶, —C(O)OR¹⁶, —S—R¹⁶, S(O)R¹⁶, —NR¹⁶S(O)R¹⁶, —S(O)N(R¹⁶)₂, —NR¹⁶S(O)N(R¹⁶)₂, —S(O)₂R¹⁶, —NR¹⁶S(O)₂—R¹⁶, —S(O)₂N(R¹⁶)₂, —NR¹⁶S(O)₂N(R¹⁶)₂, —NR¹⁶C(O)N(R¹⁶)₂, —C(O)N(R¹⁶)₂, —NR¹⁶C(O)R¹⁶, —OC(O)N(R¹⁶)₂, or —NR¹⁶C(O)OR¹⁶.

In certain embodiments, each R¹⁵ is independently halo, cyano, —NO₂, C₁₋₆ alkyl, C₂₋₆ alkenyl, C₂₋₆ alkynyl, C₁₋₆haloalkyl, C₁₋₆ alkoxy, C₁₋₆ haloalkoxy, aryl, benzyl, —N(R¹⁶)₂, —C(O)R¹⁶, —C(O)OR¹⁶, —S—R¹⁶, S(O)R¹⁶, —NR¹⁶S(O)R¹⁶, —S(O)N(R¹⁶)₂, —NR¹⁶S(O)N(R¹⁶)₂, —S(O)₂R¹⁶, —NR¹⁶S(O)₂—R¹⁶, —S(O)₂N(R¹⁶)₂, —NR¹⁶S(O)₂N(R¹⁶)₂, —NR¹⁶C(O)N(R¹⁶)₂, —C(O)N(R¹⁶)₂, —NR¹⁶C(O)R¹⁶, —OC(O)N(R¹⁶)₂, or —NR¹⁶C(O)OR¹⁶.

In certain embodiments, L is a bond, —C₁₋₅ alkylene, —C₁₋₅ heteroalkylene, —O—, —S—, —NR¹⁶—, or —C(O)NR¹⁶—; or L is a bond, —C₁₋₅ alkylene, —C₁₋₅ heteroalkylene, —O—, —S—, —S(O)—, —S(O)₂—, —C(O)NR¹⁶—, —NR¹⁶C(O)—, —OC(O)—, or —C(O)O—.

In certain embodiments, L is a bond, ethylene, methylene, —O—, —S—, —C(O)NH—, or —C(O)NCH₃—. In certain embodiments, L is a bond, methylene, or —O—. In certain embodiments, L is a bond or —C₁₋₅ alkylene. In certain embodiments, L is a bond. In certain embodiments, L is —C₁₋₅ alkylene.

In certain embodiments, R⁵ is bromo, cyano, —CF₃, —S—CH₃, methyl, phenyl, —O—CH₃, benzyl, 2-pyridinyl, 3-pyridinyl, 4-pyridinyl, 2-methoxy-phenyl, phenethyl, 1-methyl-1H-pyrazol-4-yl, 4-chloro-benzyl, 3-chloro-benzyl, 4-fluoro-phenyl, 3-fluoro-phenyl, 2-chloro-benzyl, 4-chloro-phenyl, 3-chloro-phenyl, naphthalen-2-ylmethyl, cyclohexyl, 4-trifluoromethyl-phenyl, 4-methoxy-benzyl, 3-trifluoromethyl-phenyl, 3-trifluoromethyl-benzyl, 2-chloro-6-fluoro-benzyl, 3,5-dichloro-benzyl, 2,6-dichloro-benzyl, cyclohexylmethyl, naphthalen-1-ylmethyl, 2-methyl-benzyl, 3-methyl-benzyl, 4-methyl-benzyl, 4-trifluoromethyl-benzyl, 4-fluoro-benzyl, 2-trifluoromethyl-benzyl, 3-fluoro-benzyl, 2-fluoro-benzyl, 4-cyano-benzyl, 3-cyano-benzyl, 2-cyano-benzyl, 4-trifluoromethoxy-benzyl, 3-trifluoromethoxy-benzyl, 2-trifluoromethoxy-benzyl, biphenyl-4-ylmethyl, biphenyl-3-ylmethyl, biphenyl-2-ylmethyl, 2,6-difluoro-benzyl, 2,6-dimethyl-benzyl, 2,4,6-trifluoro-benzyl, 3-chloro-2,6-difluoro-benzyl, 2,3,6-trifluoro-benzyl, 2-chloro-6-cyano-benzyl, 2-chloro-6-trifluoromethyl-benzyl, 2-fluoro-6-trifluoromethyl-benzyl, 2-methoxy-6-trifluoromethyl-benzyl, 2-methyl-6-trifluoromethyl-benzyl, 2,5-dichloro-benzyl, 2,4-dichloro-benzyl, 2,3-dichloro-benzyl, 3-trifluoromethoxy-phenyl, 4-trifluoromethoxy-phenyl, naphthalen-1-yl, 2-fluoro-phenyl, 2-chloro-phenyl, 2-methyl-phenyl, 3-methyl-phenyl, 4-methyl-phenyl, biphenyl-3-yl, biphenyl-4-yl, phenoxy, naphthalen-2-yl, phenylsulfanyl, 4-fluoro-phenoxy, 3-fluoro-2-methyl-phenyl, 2-chloro-4-methoxy-phenyl, 5-fluoro-2-methyl-phenyl, 2-chloro-4-fluoro-phenyl, 2-chloro-4-methyl-phenyl, 2-ethyl-phenyl, 4-fluoro-2-methyl-phenyl, 2-cyano-4-fluoro-phenyl, 2-fluoro-6-methyl-phenyl, 3-chloro-2-methyl-phenyl, 4-chloro-2-methyl-phenyl, 5-chloro-2-methyl-phenyl, 2,3-dimethyl-phenyl, 2,4-dimethyl-phenyl, 2,5-dimethyl-phenyl, 2-methyl-3-trifluoromethyl-phenyl, 2-methyl-4-trifluoromethyl-phenyl, 2-methyl-5-trifluoromethyl-phenyl, 3-cyano-2-methyl-phenyl, 4-cyano-2-methyl-phenyl, 5-cyano-2-methyl-phenyl, 2-chloro-3-methyl-phenyl, 2-chloro-5-methyl-phenyl, 2-chloro-3-trifluoromethyl-phenyl, 2-cyano-5-trifluoromethyl-phenyl, 2-chloro-5-methoxy-phenyl, 2-chloro-3-fluoro-phenyl, 2-chloro-5-fluoro-phenyl, 4-fluoro-naphthalen-1-yl, 4-chloro-naphthalen-1-yl, 4-phenyl-naphthalen-1-yl, quinolin-5-yl, 4-methyl-naphthalen-1-yl, 2-chloro-3-methoxy-phenyl, 2-chloro-4-trifluoromethyl-phenyl, quinolin-8-yl, 2-chloro-phenyl, 2-chloro-6-methyl-benzyl, 2,4,6-trimethyl-benzyl, 2-chloro-6-methoxy-benzyl, 2,6-dichloro-4-fluoro-benzyl, 2,6-dichloro-4-methyl-benzyl, 2,4,6-trichloro-benzyl, 2,6-dichloro-3-fluoro-benzyl, 2-chloro-phenoxy, 4-chloro-phenoxy, 3-chloro-phenoxy, p-tolyloxy-phenyl, o-tolyloxy-phenyl, 4-methoxy-phenoxy, 2,6-dichloro-4-trifluoromethoxy-benzyl, 2,6-dichloro-4-trifluoromethyl-benzyl, 2-benzyl-benzyl, 2,6-dichloro-4-methoxy-benzyl, 2-fluoro-6-methyl-benzyl, 2,6-dichloro-3-fluoro-benzyl, 2-fluoro-6-methoxy-benzyl, 4-fluoro-2,6-dimethyl-benzyl, 4-chloro-2,6-dimethyl-benzyl, 4-cyano-2,6-dimethyl-benzyl, 3,5-dimethyl-isoxazol-4-ylmethyl, 2,6-dichloro-3-methyl-benzyl, 2,3,6-trichloro-benzyl, benzoylamino, benzoyl-methyl-amino, (2,6-dimethyl-benzoyl)-methyl-amino, 2,6-dimethyl-benzoylamino, or 2,6-dichloro-benzoylamino.

In certain embodiments, one of W or X is N and the other of W and X is CR⁴; R¹ is H; R² is H; R³ is halo, cyano, C₁₋₆ alkyl, or C₁₋₆ haloalkyl; R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵; each of R⁶, R⁷, R⁸, and R⁹ is hydrogen; and L is a bond, —C₁₋₅ alkylene, —C₁₋₅ heteroalkylene, —O—, —S—, —NR¹⁶—, or —C(O)NR¹⁶—.

In certain embodiments, X is N and W is CR⁴; R¹ is H; R² is H; R³ is halo, cyano, C₁₋₆ alkyl, or C₁₋₆ haloalkyl; R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵; each of R⁶, R⁷, R⁸, and R⁹ is hydrogen; and L is a bond, —C₁₋₅ alkylene, —C₁₋₅ heteroalkylene, —O—, —S—, —NR¹⁶—, or —C(O)NR¹⁶—.

In certain embodiments, X is N and W is CH; R¹ is H; R² is H; R³ is halo, cyano, methyl, or —CF₃; R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵; each of R⁶, R⁷, R⁸, and R⁹ is hydrogen; and L is a bond, methylene, or —O—.

In certain embodiments, X and W are CH; R¹ is H; R² is H; R³ is halo, cyano, methyl, or —CF₃; R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵; each of R⁶, R⁷, R⁸, and R⁹ is hydrogen; and L is a bond, methylene, or —O—.

In certain embodiments, provided is a compound selected from Table 1, or a pharmaceutically acceptable salt, isotopically enriched analog, stereoisomer, mixture of stereoisomers or prodrug thereof:

TABLE 1

| No | Structure | Name |
|---|---|---|
| 1 | 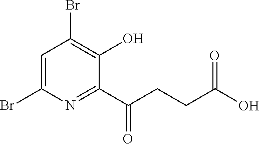 [SMILES: O=C(O)CCC(=O)c1nc(Br)cc(Br)c1O] | 4-(4,6-Dibromo-3-hydroxy-pyridin-2-yl)-4- oxo-butyric acid |
| 2 | 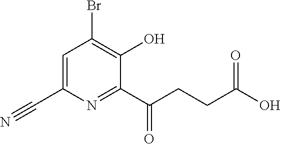 [SMILES: N#Cc1cc(Br)c(O)c(C(=O)CCC(=O)O)n1] | 4-(4-Bromo-6-cyano-3-hydroxy-pyridin-2- yl)-4-oxo-butyric acid |
| 3 | 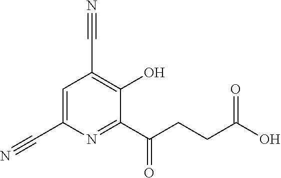 [SMILES: N#Cc1cc(C#N)c(O)c(C(=O)CCC(=O)O)n1] | 4-(4,6-Dicyano-3-hydroxy-pyridin-2-yl)-4- oxo-butyric acid |
| 4 | 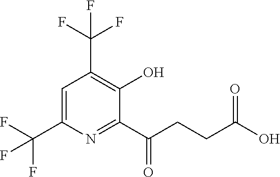 [SMILES: O=C(O)CCC(=O)c1nc(C(F)(F)F)cc(C(F)(F)F)c1O] | 4-(3-Hydroxy-4,6-bis-trifluoromethyl- pyridin-2-yl)-4-oxo-butyric acid |
| 5 | 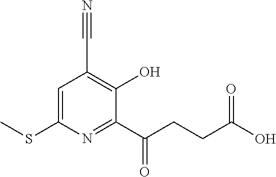 [SMILES: CSc1cc(C#N)c(O)c(C(=O)CCC(=O)O)n1] | 4-(4-Cyano-3-hydroxy-6-methylsulfanyl- pyridin-2-yl)-4-oxo-butyric acid |
| 6 | 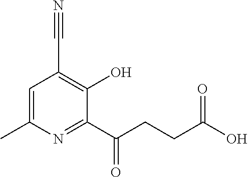 [SMILES: Cc1cc(C#N)c(O)c(C(=O)CCC(=O)O)n1] | 4-(4-Cyano-3-hydroxy-6-methyl-pyridin-2- yl)-4-oxo-butyric acid |
| 7 | 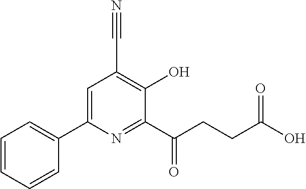 [SMILES: N#Cc1cc(-c2ccccc2)nc(C(=O)CCC(=O)O)c1O] | 4-(4-Cyano-3-hydroxy-6-phenyl-pyridin-2- yl)-4-oxo-butyric acid |
| 8 | 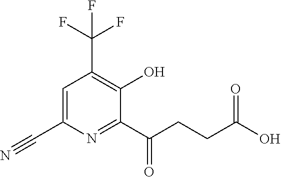 [SMILES: N#Cc1cc(C(F)(F)F)c(O)c(C(=O)CCC(=O)O)n1] | 4-(6-Cyano-3-hydroxy-4-trifluoromethyl- pyridin-2-yl)-4-oxo-butyric acid |
| 9 | 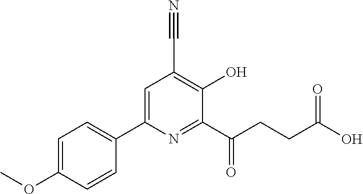 [SMILES: COc1ccc(-c2cc(C#N)c(O)c(C(=O)CCC(=O)O)n2)cc1] | 4-[4-Cyano-3-hydroxy-6-(4-methoxy- phenyl)-pyridin-2-yl]-4-oxo-butyric acid |
| 10 | 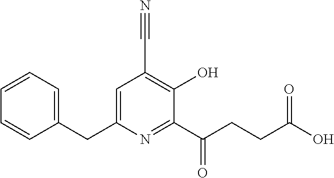 [SMILES: N#Cc1cc(Cc2ccccc2)nc(C(=O)CCC(=O)O)c1O] | 4-(6-Benzyl-4-cyano-3-hydroxy-pyridin-2- yl)-4-oxo-butyric acid |
| 11 | 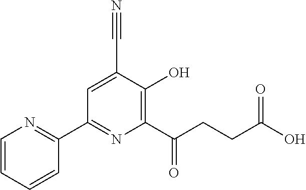 [SMILES: N#Cc1cc(-c2ccccn2)nc(C(=O)CCC(=O)O)c1O] | 4-(4-Cyano-5-hydroxy-[2,2′]bipyridinyl-6- yl)-4-oxo-butyric acid |
| 12 | 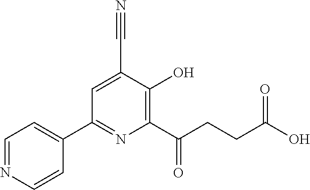 [SMILES: N#Cc1cc(-c2ccncc2)nc(C(=O)CCC(=O)O)c1O] | 4-(4-Cyano-5-hydroxy-[2,4′]bipyridinyl-6- yl)-4-oxo-butyric acid |
| 13 | 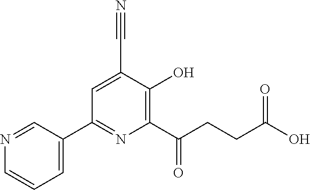 [SMILES: N#Cc1cc(-c2cccnc2)nc(C(=O)CCC(=O)O)c1O] | 4-(4-Cyano-5-hydroxy-[2,3′]bipyridinyl-6- yl)-4-oxo-butyric acid |
| 14 | 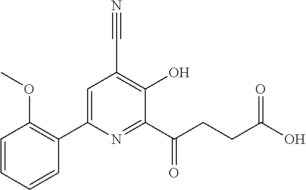 [SMILES: COc1ccccc1-c1cc(C#N)c(O)c(C(=O)CCC(=O)O)n1] | 4-[4-Cyano-3-hydroxy-6-(2-methoxy- phenyl)-pyridin-2-yl]-4-oxo-butyric acid |
| 15 | 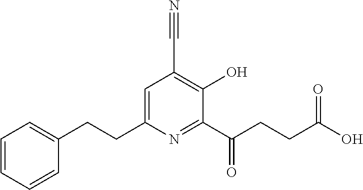 [SMILES: N#Cc1cc(CCc2ccccc2)nc(C(=O)CCC(=O)O)c1O] | 4-(4-Cyano-3-hydroxy-6-phenethyl-pyridin- 2-yl)-4-oxo-butyric acid |
| 16 | 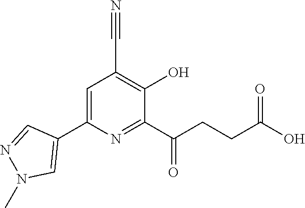 [SMILES: Cn1cc(-c2cc(C#N)c(O)c(C(=O)CCC(=O)O)n2)cn1] | 4-[4-Cyano-3-hydroxy-6-(1-methyl-1H- pyrazol-4-yl)-pyridin-2-yl]-4-oxo-butyric acid |
| 17 | 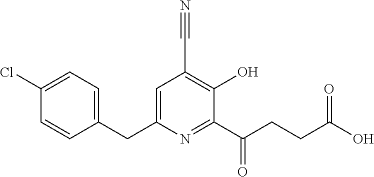 [SMILES: N#Cc1cc(Cc2ccc(Cl)cc2)nc(C(=O)CCC(=O)O)c1O] | 4-[6-(4-Chloro-benzyl)-4-cyano-3-hydroxy- pyridin-2-yl]-4-oxo-butyric acid |
| 18 | 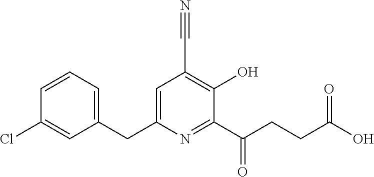 [SMILES: N#Cc1cc(Cc2cccc(Cl)c2)nc(C(=O)CCC(=O)O)c1O] | 4-[6-(3-Chloro-benzyl)-4-cyano-3-hydroxy- pyridin-2-yl]-4-oxo-butyric acid |
| 19 | 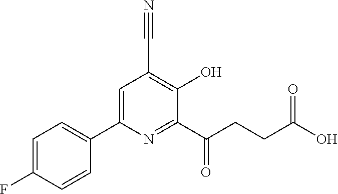 [SMILES: N#Cc1cc(-c2ccc(F)cc2)nc(C(=O)CCC(=O)O)c1O] | 4-[4-Cyano-6-(4-fluoro-phenyl)-3-hydroxy- pyridin-2-yl]-4-oxo-butyric acid |
| 20 | 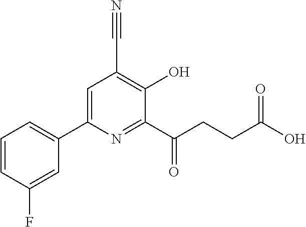 [SMILES: N#Cc1cc(-c2cccc(F)c2)nc(C(=O)CCC(=O)O)c1O] | 4-[4-Cyano-6-(3-fluoro-phenyl)-3-hydroxy- pyridin-2-yl]-4-oxo-butyric acid |
| 21 | 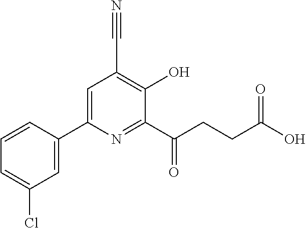 [SMILES: N#Cc1cc(-c2cccc(Cl)c2)nc(C(=O)CCC(=O)O)c1O] | 4-[6-(2-Chloro-benzyl)-4-cyano-3-hydroxy- pyridin-2-yl]-4-oxo-butyric acid |
| 22 | 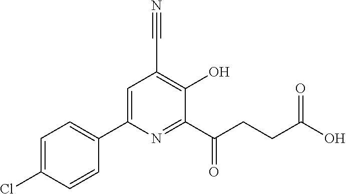 [SMILES: N#Cc1cc(-c2ccc(Cl)cc2)nc(C(=O)CCC(=O)O)c1O] | 4-[6-(4-Chloro-phenyl)-4-cyano-3-hydroxy- pyridin-2-yl]-4-oxo-butyric acid |
| 23 | 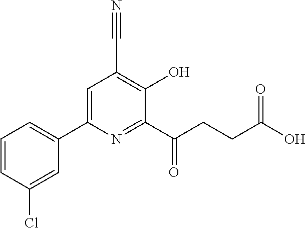 [SMILES: N#Cc1cc(-c2cccc(Cl)c2)nc(C(=O)CCC(=O)O)c1O] | 4-[6-(3-Chloro-phenyl)-4-cyano-3-hydroxy- pyridin-2-yl]-4-oxo-butyric acid |
| 24 | 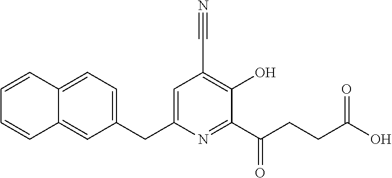 [SMILES: N#Cc1cc(Cc2ccc3ccccc3c2)nc(C(=O)CCC(=O)O)c1O] | 4-(4-Cyano-3-hydroxy-6-naphthalen-2- ylmethyl-pyridin-2-yl)-4-oxo-butyric acid |
| 25 | 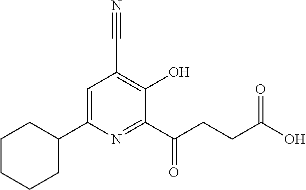 [SMILES: N#Cc1cc(C2CCCCC2)nc(C(=O)CCC(=O)O)c1O] | 4-(4-Cyano-6-cyclohexyl-3-hydroxy- pyridin-2-yl)-4-oxo-butyric acid |
| 26 | 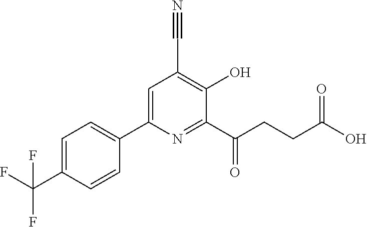 [SMILES: N#Cc1cc(-c2ccc(C(F)(F)F)cc2)nc(C(=O)CCC(=O)O)c1O] | 4-[4-Cyano-3-hydroxy-6-(4-trifluoromethyl- phenyl)-pyridin-2-yl]-4-oxo-butyric acid |
| 27 | 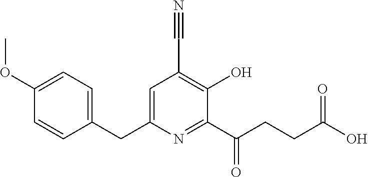 [SMILES: COc1ccc(Cc2cc(C#N)c(O)c(C(=O)CCC(=O)O)n2)cc1] | 4-[4-Cyano-3-hydroxy-6-(4-methoxy- benzyl)-pyridin-2-yl]-4-oxo-butyric acid |
| 28 | 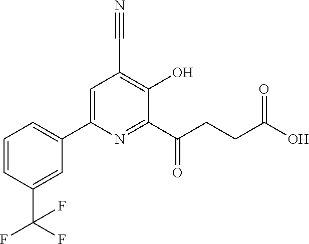 [SMILES: N#Cc1cc(-c2cccc(C(F)(F)F)c2)nc(C(=O)CCC(=O)O)c1O] | 4-[4-Cyano-3-hydroxy-6-(3-trifluoromethyl- phenyl)-pyridin-2-yl]-4-oxo-butyric acid |
| 29 | 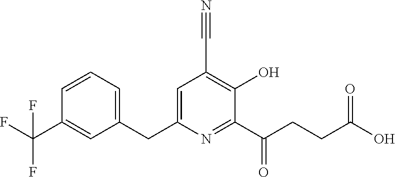 [SMILES: N#Cc1cc(Cc2cccc(C(F)(F)F)c2)nc(C(=O)CCC(=O)O)c1O] | 4-[4-Cyano-3-hydroxy-6-(3-trifluoromethyl- benzyl)-pyridin-2-yl]-4-oxo-butyric acid |
| 30 | 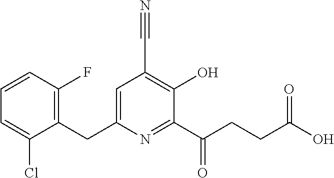 [SMILES: N#Cc1cc(Cc2c(F)cccc2Cl)nc(C(=O)CCC(=O)O)c1O] | 4-[6-(2-Chloro-6-fluoro-benzyl)-4-cyano-3- hydroxy-pyridin-2-yl]-4-oxo-butyric acid |
| 31 | 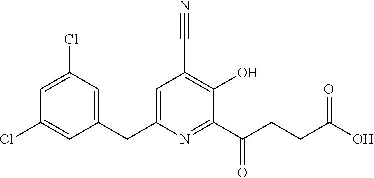 [SMILES: N#Cc1cc(Cc2cc(Cl)cc(Cl)c2)nc(C(=O)CCC(=O)O)c1O] | 4-[4-Cyano-6-(3,5-dichloro-benzyl)-3- hydroxy-pyridin-2-yl]-4-oxo-butyric acid |
| 32 | 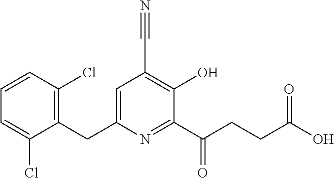 [SMILES: N#Cc1cc(Cc2c(Cl)cccc2Cl)nc(C(=O)CCC(=O)O)c1O] | 4-[4-Cyano-6-(2,6-dichloro-benzyl)-3- hydroxy-pyridin-2-yl]-4-oxo-butyric acid |
| 33 | 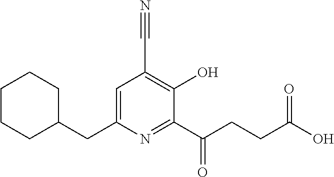 [SMILES: N#Cc1cc(CC2CCCCC2)nc(C(=O)CCC(=O)O)c1O] | 4-(4-Cyano-6-cyclohexylmethyl-3-hydroxy- pyridin-2-yl)-4-oxo-butyric acid |
| 34 | 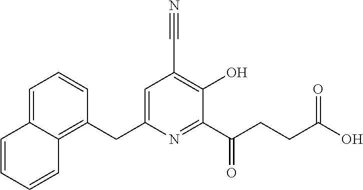 [SMILES: N#Cc1cc(Cc2cccc3ccccc23)nc(C(=O)CCC(=O)O)c1O] | 4-(4-Cyano-3-hydroxy-6-naphthalen-1- ylmethyl-pyridin-2-yl)-4-oxo-butyric acid |
| 35 | 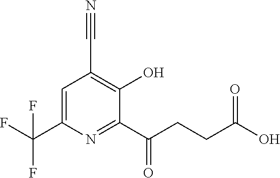 [SMILES: N#Cc1cc(C(F)(F)F)nc(C(=O)CCC(=O)O)c1O] | 4-(4-Cyano-3-hydroxy-6-trifluoromethyl- pyridin-2-yl)-4-oxo-butyric acid |
| 36 | 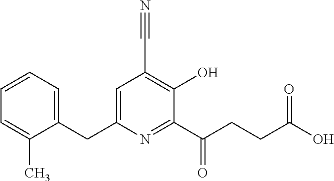 [SMILES: Cc1ccccc1Cc1cc(C#N)c(O)c(C(=O)CCC(=O)O)n1] | 4-[4-Cyano-3-hydroxy-6-(2-methyl-benzyl)- pyridin-2-yl]-4-oxo-butyric acid |
| 37 | 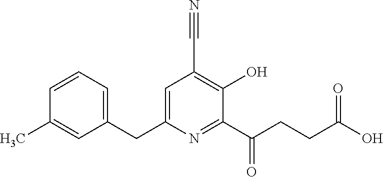 [SMILES: Cc1cccc(Cc2cc(C#N)c(O)c(C(=O)CCC(=O)O)n2)c1] | 4-[4-Cyano-3-hydroxy-6-(3-methyl-benzyl)- pyridin-2-yl]-4-oxo-butyric acid |
| 38 | 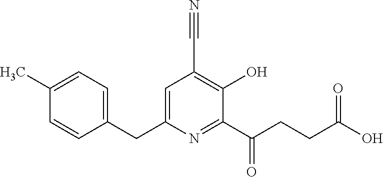 [SMILES: Cc1ccc(Cc2cc(C#N)c(O)c(C(=O)CCC(=O)O)n2)cc1] | 4-[4-Cyano-3-hydroxy-6-(4-methyl-benzyl)- pyridin-2-yl]-4-oxo-butyric acid |
| 39 | 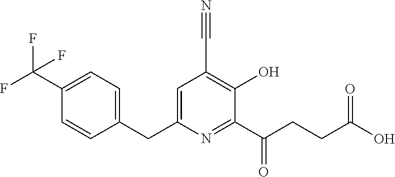 [SMILES: N#Cc1cc(Cc2ccc(C(F)(F)F)cc2)nc(C(=O)CCC(=O)O)c1O] | 4-[4-Cyano-3-hydroxy-6-(4-trifluoromethyl- benzyl)-pyridin-2-yl]-4-oxo-butyric acid |
| 40 | 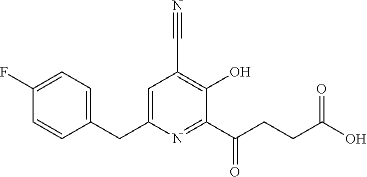 [SMILES: N#Cc1cc(Cc2ccc(F)cc2)nc(C(=O)CCC(=O)O)c1O] | 4-[4-Cyano-6-(4-fluoro-benzyl)-3-hydroxy- pyridin-2-yl]-4-oxo-butyric acid |
| 41 | 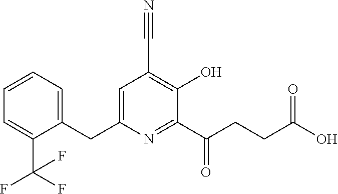 [SMILES: N#Cc1cc(Cc2ccccc2C(F)(F)F)nc(C(=O)CCC(=O)O)c1O] | 4-[4-Cyano-3-hydroxy-6-(2-trifluoromethyl- benzyl)-pyridin-2-yl]-4-oxo-butyric acid |
| 42 | 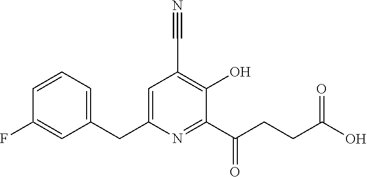 [SMILES: N#Cc1cc(Cc2cccc(F)c2)nc(C(=O)CCC(=O)O)c1O] | 4-[4-Cyano-6-(3-fluoro-benzyl)-3-hydroxy- pyridin-2-yl]-4-oxo-butyric acid |
| 43 | 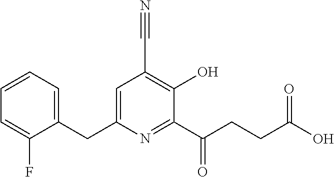 [SMILES: N#Cc1cc(Cc2ccccc2F)nc(C(=O)CCC(=O)O)c1O] | 4-[4-Cyano-6-(2-fluoro-benzyl)-3-hydroxy- pyridin-2-yl]-4-oxo-butyric acid |
| 44 | 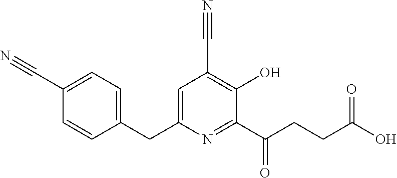 [SMILES: N#Cc1ccc(Cc2cc(C#N)c(O)c(C(=O)CCC(=O)O)n2)cc1] | 4-[4-Cyano-6-(4-cyano-benzyl)-3-hydroxy- pyridin-2-yl]-4-oxo-butyric acid |
| 45 | 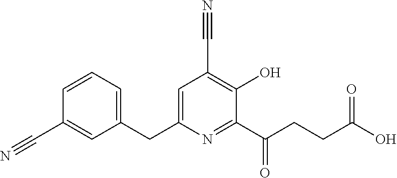 [SMILES: N#Cc1cccc(Cc2cc(C#N)c(O)c(C(=O)CCC(=O)O)n2)c1] | 4-[4-Cyano-6-(3-cyano-benzyl)-3-hydroxy- pyridin-2-yl]-4-oxo-butyric acid |
| 46 | 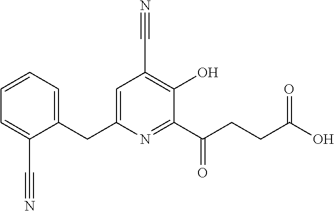 [SMILES: N#Cc1ccccc1Cc1cc(C#N)c(O)c(C(=O)CCC(=O)O)n1] | 4-[4-Cyano-6-(2-cyano-benzyl)-3-hydroxy- pyridin-2-yl]-4-oxo-butyric acid |
| 47 | 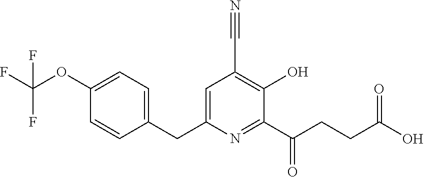 [SMILES: N#Cc1cc(Cc2ccc(OC(F)(F)F)cc2)nc(C(=O)CCC(=O)O)c1O] | 4-[4-Cyano-3-hydroxy-6-(4- trifluoromethoxy-benzyl)-pyridin-2-yl]-4- oxo-butyric acid |
| 48 | 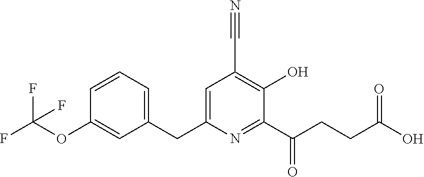 [SMILES: N#Cc1cc(Cc2cccc(OC(F)(F)F)c2)nc(C(=O)CCC(=O)O)c1O] | 4-[4-Cyano-3-hydroxy-6-(3- trifluoromethoxy-benzyl)-pyridin-2-yl]-4- oxo-butyric acid |
| 49 | 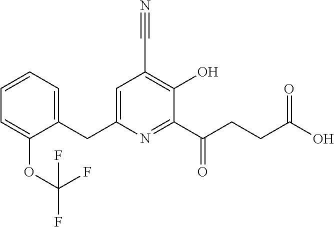 [SMILES: N#Cc1cc(Cc2ccccc2OC(F)(F)F)nc(C(=O)CCC(=O)O)c1O] | 4-[4-Cyano-3-hydroxy-6-(2- trifluoromethoxy-benzyl)-pyridin-2-yl]-4- oxo-butyric acid |
| 50 | 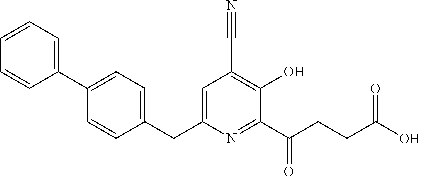 [SMILES: N#Cc1cc(Cc2ccc(-c3ccccc3)cc2)nc(C(=O)CCC(=O)O)c1O] | 4-(6-Biphenyl-4-ylmethyl-4-cyano-3- hydroxy-pyridin-2-yl)-4-oxo-butyric acid |
| 51 | 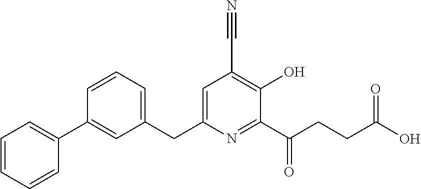 [SMILES: N#Cc1cc(Cc2cccc(-c3ccccc3)c2)nc(C(=O)CCC(=O)O)c1O] | 4-(6-Biphenyl-3-ylmethyl)-4-cyano-3- hydroxy-pyridin-2-yl)-4-oxo-butyric acid |
| 52 | 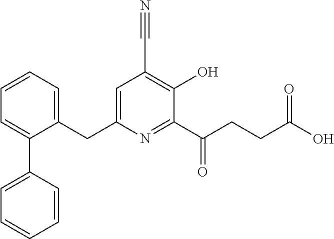 [SMILES: N#Cc1cc(Cc2ccccc2-c2ccccc2)nc(C(=O)CCC(=O)O)c1O] | 4-(6-Biphenyl-2-ylmethyl-4-cyano-3- hydroxy-pyridin-2-yl)-4-oxo-butyric acid |
| 53 | 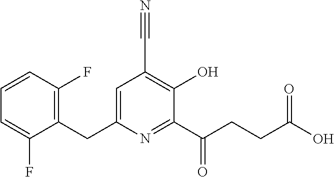 [SMILES: N#Cc1cc(Cc2c(F)cccc2F)nc(C(=O)CCC(=O)O)c1O] | 4-[4-Cyano-6-(2,6-difluoro-benzyl)-3- hydroxy-pyridin-2-yl]-4-oxo-butyric acid |
| 54 | 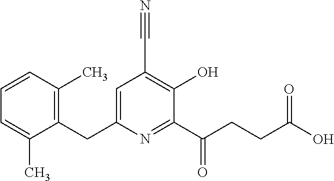 [SMILES: Cc1cccc(C)c1Cc1cc(C#N)c(O)c(C(=O)CCC(=O)O)n1] | 4-[4-Cyano-6-(2,6-dimethyl-benzyl)-3- hydroxy-pyridin-2-yl]-4-oxo-butyric acid |
| 55 | 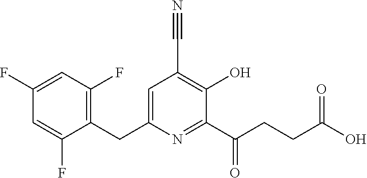 [SMILES: N#Cc1cc(Cc2c(F)cc(F)cc2F)nc(C(=O)CCC(=O)O)c1O] | 4-[4-Cyano-3-hydroxy-6-(2,4,6-trifluoro- benzyl)-pyridin-2-yl]-4-oxo-butyric acid |
| 56 | 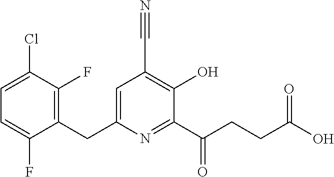 [SMILES: N#Cc1cc(Cc2c(F)ccc(Cl)c2F)nc(C(=O)CCC(=O)O)c1O] | 4-[6-(3-Chloro-2,6-difluoro-benzyl)-4- cyano-3-hydroxy-pyridin-2-yl]-4-oxo- butyric acid |
| 57 | 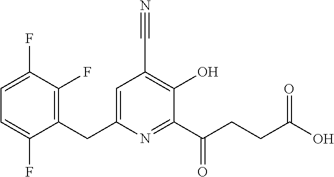 [SMILES: N#Cc1cc(Cc2c(F)ccc(F)c2F)nc(C(=O)CCC(=O)O)c1O] | 4-[4-Cyano-3-hydroxy-6-(2,3,6-trifluoro- benzyl)-pyridin-2-yl]-4-oxo-butyric acid |
| 58 | 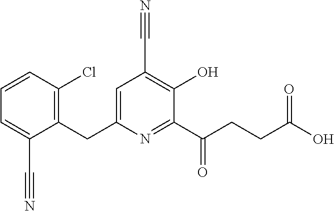 [SMILES: N#Cc1cc(Cc2c(Cl)cccc2C#N)nc(C(=O)CCC(=O)O)c1O] | 4-[6-(2-Chloro-6-cyano-benzyl)-4-cyano-3- hydroxy-pyridin-2-yl]-4-oxo-butyric acid |
| 59 | 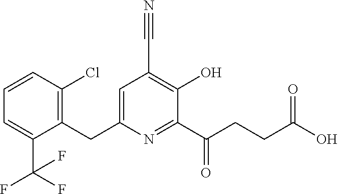 [SMILES: N#Cc1cc(Cc2c(Cl)cccc2C(F)(F)F)nc(C(=O)CCC(=O)O)c1O] | 4-[6-(2-Chloro-6-trifluoromethyl-benzyl)-4- cyano-3-hydroxy-pyridin-2-yl]-4-oxo- butyric acid |
| 60 | 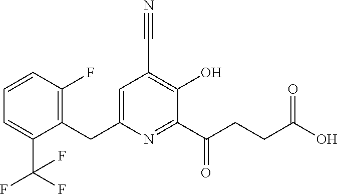 [SMILES: N#Cc1cc(Cc2c(F)cccc2C(F)(F)F)nc(C(=O)CCC(=O)O)c1O] | 4-[6-(2-Fluoro-6-trifluoromethyl-benzyl)-4- cyano-3-hydroxy-pyridin-2-yl]-4-oxo- butyric acid |
| 61 | 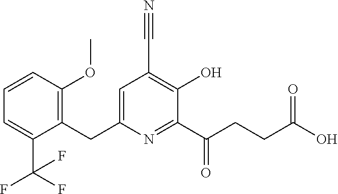 [SMILES: COc1cccc(C(F)(F)F)c1Cc1cc(C#N)c(O)c(C(=O)CCC(=O)O)n1] | 4-[4-Cyano-3-hydroxy-6-(2-methoxy-6- trifluoromethyl-benzyl)-pyridin-2-yl]-4-oxo- butyric acid |
| 62 | 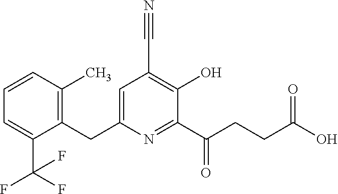 [SMILES: Cc1cccc(C(F)(F)F)c1Cc1cc(C#N)c(O)c(C(=O)CCC(=O)O)n1] | 4-[4-Cyano-3-hydroxy-6-(2-methyl-6- trifluoromethyl-benzyl)-pyridin-2-yl]-4-oxo- butyric acid |
| 63 | 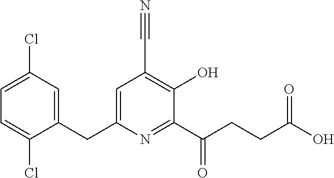 [SMILES: N#Cc1cc(Cc2cc(Cl)ccc2Cl)nc(C(=O)CCC(=O)O)c1O] | 4-[4-Cyano-6-(2,5-dichloro-benzyl)-3- hydroxy-pyridin-2-yl]-4-oxo-butyric acid |
| 64 | 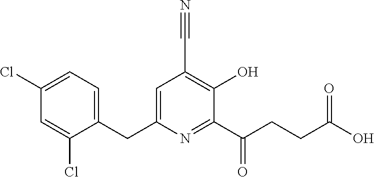 [SMILES: N#Cc1cc(Cc2ccc(Cl)cc2Cl)nc(C(=O)CCC(=O)O)c1O] | 4-[4-Cyano-6-(2,4-dichloro-benzyl)-3- hydroxy-pyridin-2-yl]-4-oxo-butyric acid |
| 65 | 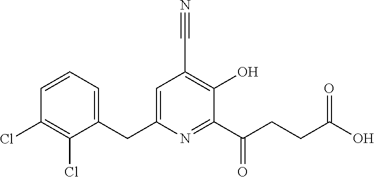 [SMILES: N#Cc1cc(Cc2cccc(Cl)c2Cl)nc(C(=O)CCC(=O)O)c1O] | 4-[4-Cyano-6-(2,3-dichloro-benzyl)-3- hydroxy-pyridin-2-yl]-4-oxo-butyric acid |
| 66 | 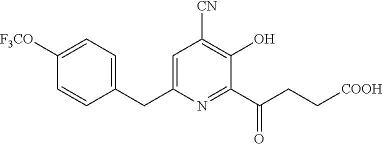 [SMILES: N#Cc1cc(Cc2ccc(OC(F)(F)F)cc2)nc(C(=O)CCC(=O)O)c1O] | 4-[4-Cyano-3-hydroxy-6-(3- trifluoromethoxy-phenyl)-pyridin-2-yl]-4- oxo-butyric acid |
| 67 | 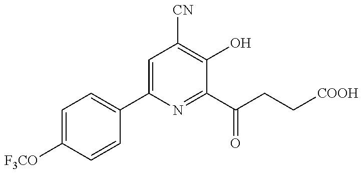 [SMILES: N#Cc1cc(-c2ccc(OC(F)(F)F)cc2)nc(C(=O)CCC(=O)O)c1O] | 4-[4-Cyano-3-hydroxy-6-(4- trifluoromethoxy-phenyl)-pyridin-2-yl]-4- oxo-butyric acid |
| 68 | 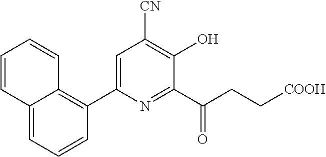 [SMILES: N#Cc1cc(-c2cccc3ccccc23)nc(C(=O)CCC(=O)O)c1O] | 4-(4-Cyano-3-hydroxy-6-naphthalen-1-yl- pyridin-2-yl)-4-oxo-butyric acid |
| 69 | 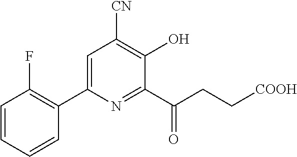 [SMILES: N#Cc1cc(-c2ccccc2F)nc(C(=O)CCC(=O)O)c1O] | 4-[4-Cyano-6-(2-fluoro-phenyl)-3-hydroxy- pyridin-2-yl]-4-oxo-butyric acid |
| 70 | 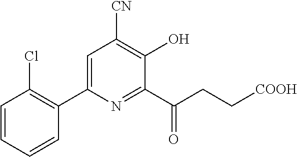 [SMILES: N#Cc1cc(-c2ccccc2Cl)nc(C(=O)CCC(=O)O)c1O] | 4-[6-(2-Chloro-phenyl)-4-cyano-3-hydroxy- pyridin-2-yl]-4-oxo-butyric acid |
| 71 | 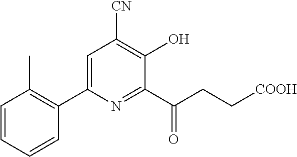 [SMILES: Cc1ccccc1-c1cc(C#N)c(O)c(C(=O)CCC(=O)O)n1] | 4-(4-Cyano-3-hydroxy-6-o-tolyl-pyridin-2- yl)-4-oxo-butyric acid |
| 72 | 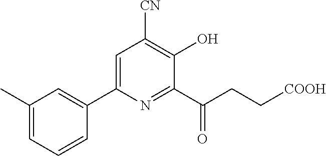 [SMILES: Cc1cccc(-c2cc(C#N)c(O)c(C(=O)CCC(=O)O)n2)c1] | 4-(4-Cyano-3-hydroxy-6-m-tolyl-pyridin-2- yl)-4-oxo-butyric acid |
| 73 | 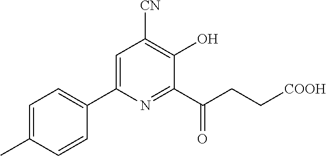 [SMILES: Cc1ccc(-c2cc(C#N)c(O)c(C(=O)CCC(=O)O)n2)cc1] | 4-(4-Cyano-3-hydroxy-6-p-tolyl-pyridin-2- yl)-4-oxo-butyric acid |
| 74 | 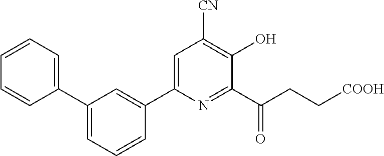 [SMILES: N#Cc1cc(-c2cccc(-c3ccccc3)c2)nc(C(=O)CCC(=O)O)c1O] | 4-(6-Biphenyl-3-yl-4-cyano-3-hydroxy- pyridin-2-yl)-4-oxo-butyric acid |
| 75 | 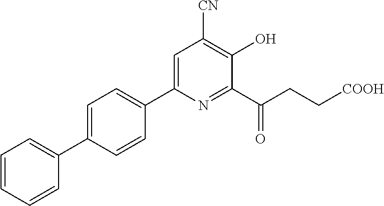 [SMILES: N#Cc1cc(-c2ccc(-c3ccccc3)cc2)nc(C(=O)CCC(=O)O)c1O] | 4-(6-Biphenyl-4-yl-4-cyano-3-hydroxy- pyridin-2-yl)-4-oxo-butyric acid |
| 76 | 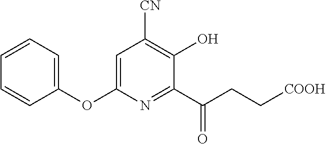 [SMILES: N#Cc1cc(Oc2ccccc2)nc(C(=O)CCC(=O)O)c1O] | 4-(4-Cyano-3-hydroxy-6-phenoxy-pyridin- 2-yl)-4-oxo-butyric acid |
| 77 | 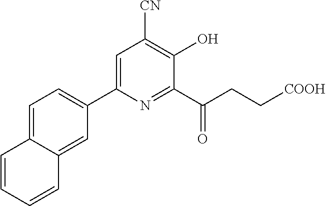 [SMILES: N#Cc1cc(-c2ccc3ccccc3c2)nc(C(=O)CCC(=O)O)c1O] | 4-(4-Cyano-3-hydroxy-6-naphthlane-2-yl- pyridin-2-yl)-4-oxo-butyric acid |
| 78 | 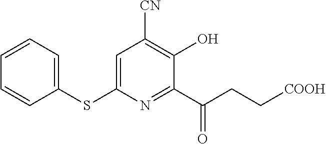 [SMILES: N#Cc1cc(Sc2ccccc2)nc(C(=O)CCC(=O)O)c1O] | 4-(4-Cyano-3-hydroxy-6-phenylsulfanyl- pyridin-2-yl)-4-oxo-butyric acid |
| 79 | 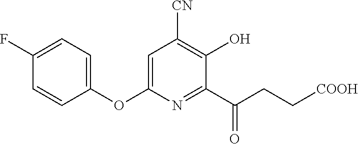 [SMILES: N#Cc1cc(Oc2ccc(F)cc2)nc(C(=O)CCC(=O)O)c1O] | 4-[4-Cyano-6-(4-fluoro-phenoxy)-3- hydroxy-pyridin-2-yl]-4-oxo-butyric acid |
| 80 | 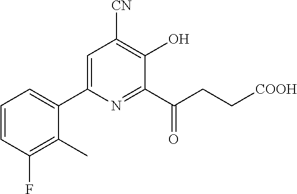 [SMILES: Cc1c(F)cccc1-c1cc(C#N)c(O)c(C(=O)CCC(=O)O)n1] | 4-[4-Cyano-6-(3-fluoro-2-methyl-phenyl)-3- hydroxy-pyridin-2-yl]-4-oxo-butyric acid |
| 81 | 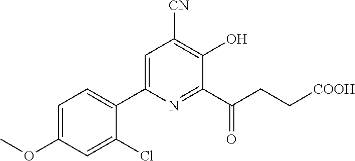 [SMILES: COc1ccc(-c2cc(C#N)c(O)c(C(=O)CCC(=O)O)n2)c(Cl)c1] | 4-[6-(2-Chloro-4-methoxy-phenyl)-4-cyano- 3-hydroxy-pyridin-2-yl]-4-oxo-butyric acid |
| 82 | 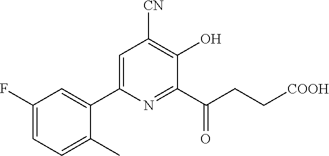 [SMILES: Cc1ccc(F)cc1-c1cc(C#N)c(O)c(C(=O)CCC(=O)O)n1] | 4-[4-Cyano-6-(5-fluoro-2-methyl-phenyl)-3- hydroxy-pyridin-2-yl]-4-oxo-butyric acid |
| 83 | 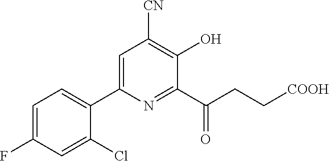 [SMILES: N#Cc1cc(-c2ccc(F)cc2Cl)nc(C(=O)CCC(=O)O)c1O] | 4-[6-(2-Chloro-4-fluoro-phenyl)-4-cyano-3- hydroxy-pyridin-2-yl]-4-oxo-butyric acid |
| 84 | 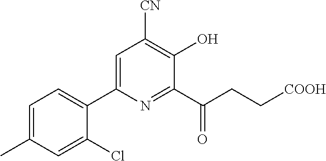 [SMILES: Cc1ccc(-c2cc(C#N)c(O)c(C(=O)CCC(=O)O)n2)c(Cl)c1] | 4-[6-(2-Chloro-4-methyl-phenyl)-4-cyano-3- hydroxy-pyridin-2-yl]-4-oxo-butyric acid |
| 85 | 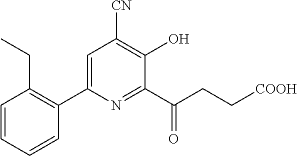 [SMILES: CCc1ccccc1-c1cc(C#N)c(O)c(C(=O)CCC(=O)O)n1] | 4-[4-Cyano-6-(2-ethyl-phenyl)-3-hydroxy- pyridin-2-yl]-4-oxo-butyric acid |
| 86 | 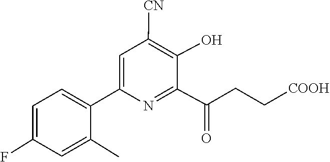 [SMILES: Cc1cc(F)ccc1-c1cc(C#N)c(O)c(C(=O)CCC(=O)O)n1] | 4-[4-Cyano-6-(4-fluoro-2-methyl-phenyl)-3- hydroxy-pyridin-2-yl]-4-oxo-butyric acid |
| 87 | 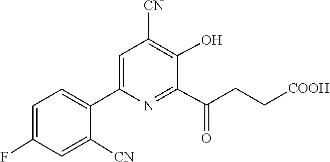 [SMILES: N#Cc1cc(F)ccc1-c1cc(C#N)c(O)c(C(=O)CCC(=O)O)n1] | 4-[4-Cyano-6-(2-cyano-4-fluoro-phenyl)-3- hydroxy-pyridin-2-yl]-4-oxo-butyric acid |
| 88 | 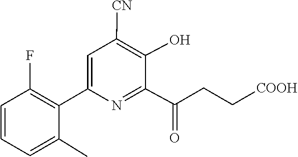 [SMILES: Cc1cccc(F)c1-c1cc(C#N)c(O)c(C(=O)CCC(=O)O)n1] | 4-[4-Cyano-6-(2-fluoro-6-methyl-phenyl)-3- hydroxy-pyridin-2-yl]-4-oxo-butyric acid |
| 89 | 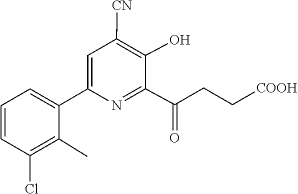 [SMILES: Cc1c(Cl)cccc1-c1cc(C#N)c(O)c(C(=O)CCC(=O)O)n1] | 4-[6-(3-Chloro-2-methyl-phenyl)-4-cyano-3- hydroxy-pyridin-2-yl]-4-oxo-butyric acid |
| 90 | 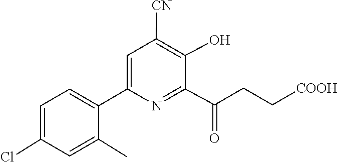 [SMILES: Cc1cc(Cl)ccc1-c1cc(C#N)c(O)c(C(=O)CCC(=O)O)n1] | 4-[6-(4-Chloro-2-methyl-phenyl)-4-cyano-3- hydroxy-pyridin-2-yl]-4-oxo-butyric acid |
| 91 | 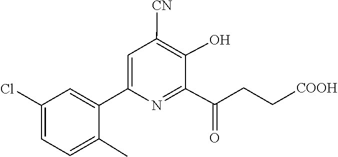 [SMILES: Cc1ccc(Cl)cc1-c1cc(C#N)c(O)c(C(=O)CCC(=O)O)n1] | 4-[6-(5-Chloro-2-methyl-phenyl)-4-cyano-3- hydroxy-pyridin-2-yl]-4-oxo-butyric acid |
| 92 | 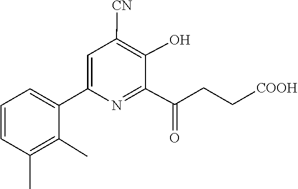 [SMILES: Cc1cccc(-c2cc(C#N)c(O)c(C(=O)CCC(=O)O)n2)c1C] | 4-[4-Cyano-6-(2,3-dimethyl-phenyl)-3- hydroxy-pyridin-2-yl]-4-oxo-butyric acid |
| 93 | 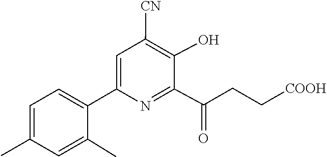 [SMILES: Cc1ccc(-c2cc(C#N)c(O)c(C(=O)CCC(=O)O)n2)c(C)c1] | 4-[4-Cyano-6-(2,4-dimethyl-phenyl)-3- hydroxy-pyridin-2-yl]-4-oxo-butyric acid |
| 94 | 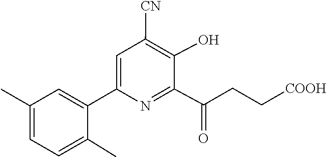 [SMILES: Cc1ccc(C)c(-c2cc(C#N)c(O)c(C(=O)CCC(=O)O)n2)c1] | 4-[4-Cyano-6-(2,5-dimethyl-phenyl)-3- hydroxy-pyridin-2-yl]-4-oxo-butyric acid |
| 95 | 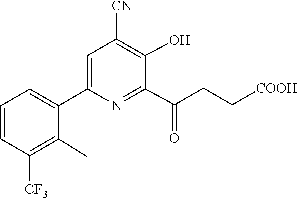 [SMILES: Cc1c(-c2cc(C#N)c(O)c(C(=O)CCC(=O)O)n2)cccc1C(F)(F)F] | 4-[4-Cyano-3-hydroxy-6-(2-methyl-3- trifluoromethyl-phenyl)-pyridin-2-yl]-4-oxo- butyric acid |
| 96 | 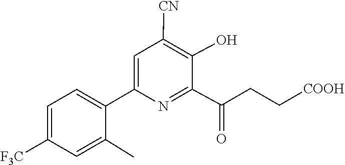 [SMILES: Cc1cc(C(F)(F)F)ccc1-c1cc(C#N)c(O)c(C(=O)CCC(=O)O)n1] | 4-[4-Cyano-3-hydroxy-6-(2-methyl-4- trifluoromethyl-phenyl)-pyridin-2-yl]-4-oxo- butyric acid |
| 97 | 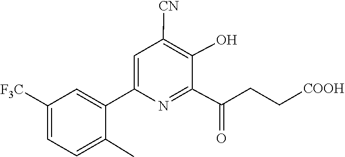 [SMILES: Cc1ccc(C(F)(F)F)cc1-c1cc(C#N)c(O)c(C(=O)CCC(=O)O)n1] | 4-[4-Cyano-3-hydroxy-6-(2-methyl-5- trifluoromethyl-phenyl)-pyridin-2-yl]-4-oxo- butyric acid |
| 98 | 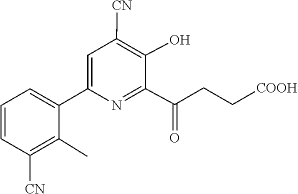 [SMILES: Cc1c(C#N)cccc1-c1cc(C#N)c(O)c(C(=O)CCC(=O)O)n1] | 4-[4-Cyano-6-(3-cyano-2-methyl-phenyl)-3- hydroxy-pyridin-2-yl]-4-oxo-butyric acid |
| 99 | 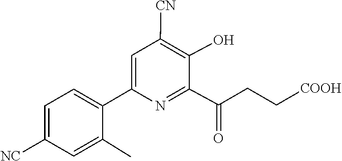 [SMILES: Cc1cc(C#N)ccc1-c1cc(C#N)c(O)c(C(=O)CCC(=O)O)n1] | 4-[4-Cyano-6-(4-cyano-2-methyl-phenyl)-3- hydroxy-pyridin-2-yl]-4-oxo-butyric acid |
| 100 | 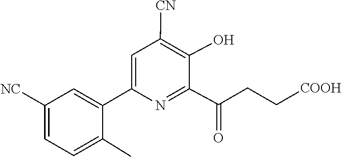 [SMILES: Cc1ccc(C#N)cc1-c1cc(C#N)c(O)c(C(=O)CCC(=O)O)n1] | 4-[4-Cyano-6-(5-cyano-2-methyl-phenyl)-3- hydroxy-pyridin-2-yl]-4-oxo-butyric acid |
| 101 | 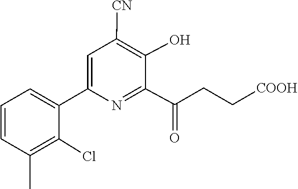 [SMILES: Cc1cccc(-c2cc(C#N)c(O)c(C(=O)CCC(=O)O)n2)c1Cl] | 4-[6-(2-Chloro-3-methyl-phenyl)-4-cyano-3- hydroxy-pyridin-2-yl]-4-oxo-butyric acid |
| 102 | 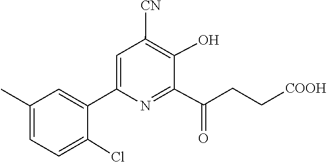 [SMILES: Cc1ccc(Cl)c(-c2cc(C#N)c(O)c(C(=O)CCC(=O)O)n2)c1] | 4-[6-(2-Chloro-4-methyl-phenyl)-4-cyano-3- hydroxy-pyridin-2-yl]-4-oxo-butyric acid |
| 103 | 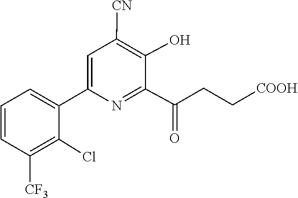 [SMILES: N#Cc1cc(-c2cccc(C(F)(F)F)c2Cl)nc(C(=O)CCC(=O)O)c1O] | 4-[6-(2-Chloro-3-trifluoromethyl-phenyl)-4- cyano-3-hydroxy-pyridin-2-yl]-4-oxo- butyric acid |
| 104 | 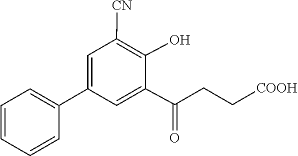 [SMILES: N#Cc1cc(-c2ccccc2)cc(C(=O)CCC(=O)O)c1O] | 4-(5-Cyano-4-hydroxy-biphenyl-3-yl)-4- oxo-butyric acid |
| 105 | 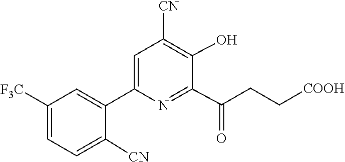 [SMILES: N#Cc1ccc(C(F)(F)F)cc1-c1cc(C#N)c(O)c(C(=O)CCC(=O)O)n1] | 4-[4-Cyano-6-(2-cyano-5-trifluoromethyl- phenyl)-3-hydroxy-pyridin-2-yl]-4-oxo- butyric acid |
| 106 | 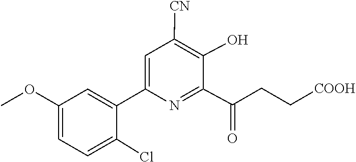 [SMILES: COc1ccc(Cl)c(-c2cc(C#N)c(O)c(C(=O)CCC(=O)O)n2)c1] | 4-[6-(2-Chloro-5-methoxy-phenyl)-4-cyano- 3-hydroxy-pyridin-2-yl]-4-oxo-butyric acid |
| 107 | 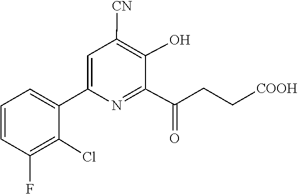 [SMILES: N#Cc1cc(-c2cccc(F)c2Cl)nc(C(=O)CCC(=O)O)c1O] | 4-[6-(2-Chloro-3-fluoro-phenyl)-4-cyano-3- hydroxy-pyridin-2-yl]-4-oxo-butyric acid |
| 108 | 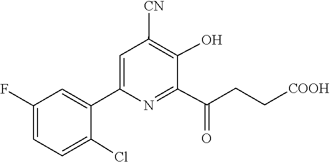 [SMILES: N#Cc1cc(-c2cc(F)ccc2Cl)nc(C(=O)CCC(=O)O)c1O] | 4-[6-(2-Chloro-5-fluoro-phenyl)-4-cyano-3- hydroxy-pyridin-2-yl]-4-oxo-butyric acid |
| 109 | 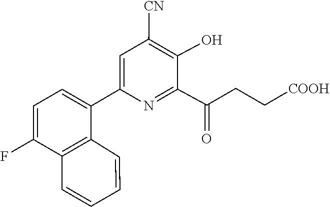 [SMILES: N#Cc1cc(-c2ccc(F)c3ccccc23)nc(C(=O)CCC(=O)O)c1O] | 4-[4-Cyano-6-(4-fluoro-naphthalen-1-yl)-3- hydroxy-pyridin-2-yl]-4-oxo-butyric acid |
| 110 | 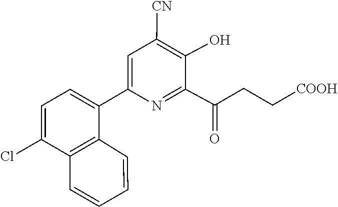 [SMILES: N#Cc1cc(-c2ccc(Cl)c3ccccc23)nc(C(=O)CCC(=O)O)c1O] | 4-[6-(4-Chloro-naphthalen-1-yl)-4-cyano-3- hydroxy-pyridin-2-yl]-4-oxo-butyric acid |
| 111 | 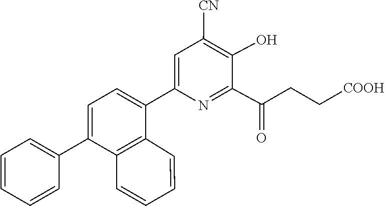 [SMILES: N#Cc1cc(-c2ccc(-c3ccccc3)c3ccccc23)nc(C(=O)CCC(=O)O)c1O] | 4-[4-Cyano-3-hydroxy-6-(4-phenyl- naphthalen-1-yl)-pyridin-2-yl]-4-oxo-butyric acid |
| 112 | 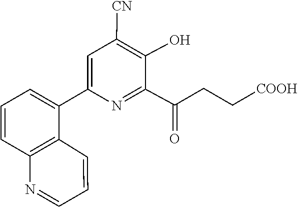 [SMILES: N#Cc1cc(-c2cccc3ncccc23)nc(C(=O)CCC(=O)O)c1O] | 4-(4-Cyano-3-hydroxy-6-quinolin-5-yl- pyridin-2-yl)-4-oxo-butyric acid |
| 113 | 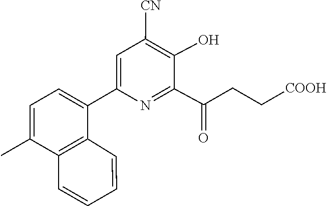 [SMILES: Cc1ccc(-c2cc(C#N)c(O)c(C(=O)CCC(=O)O)n2)c2ccccc12] | 4-[4-Cyano-3-hydroxy-6-(4-methyl- naphthalen-1-yl)-pyridin-2-yl]-4-oxo-butyric acid |
| 114 | 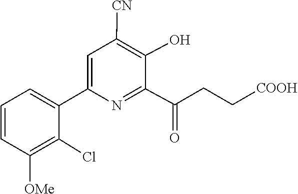 [SMILES: COc1cccc(-c2cc(C#N)c(O)c(C(=O)CCC(=O)O)n2)c1Cl] | 4-[6-(2-Chloro-3-methoxy-phenyl)-4-cyano- 3-hydroxy-pyridin-2-yl]-4-oxo-butyric acid |
| 115 | 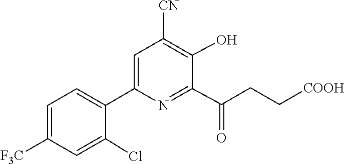 [SMILES: N#Cc1cc(-c2ccc(C(F)(F)F)cc2Cl)nc(C(=O)CCC(=O)O)c1O] | 4-[6-(2-Chloro-4-trifluoromethyl-phenyl)-4- cyano-3-hydroxy-pyridin-2-yl]-4-oxo- butyric acid |
| 116 | 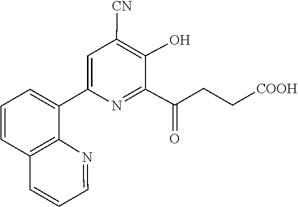 [SMILES: N#Cc1cc(-c2cccc3cccnc23)nc(C(=O)CCC(=O)O)c1O] | 4-(4-Cyano-3-hydroxy-6-quinolin-8-yl- pyridin-2-yl)-4-oxo-butyric acid |
| 117 | 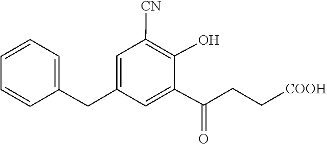 [SMILES: N#Cc1cc(Cc2ccccc2)cc(C(=O)CCC(=O)O)c1O] | 4-(5-Benzyl-3-cyano-2-hydroxy-phenyl)-4- oxo-butyric acid |
| 118 | 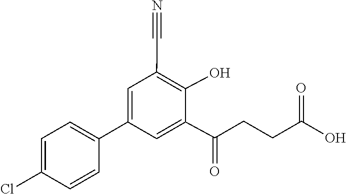 [SMILES: N#Cc1cc(-c2ccc(Cl)cc2)cc(C(=O)CCC(=O)O)c1O] | 4-(4′-Chloro-5-cyano-4-hydroxy-biphenyl-3- yl)-4-oxo-butyric acid |
| 119 | 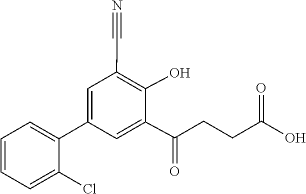 [SMILES: N#Cc1cc(-c2ccccc2Cl)cc(C(=O)CCC(=O)O)c1O] | 4-(2′-Chloro-5-cyano-4-hydroxy-biphenyl-3- yl)-4-oxo-butyric acid |
| 120 | 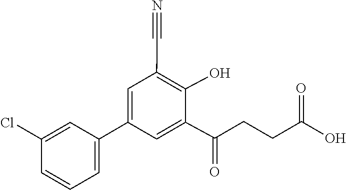 [SMILES: N#Cc1cc(-c2cccc(Cl)c2)cc(C(=O)CCC(=O)O)c1O] | 4-(3′-Chloro-5-cyano-4-hydroxy-biphenyl-3- yl)-4-oxo-butyric acid |
| 121 | 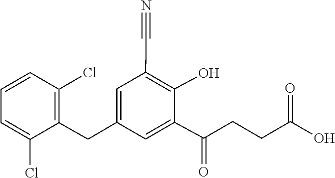 [SMILES: N#Cc1cc(Cc2c(Cl)cccc2Cl)cc(C(=O)CCC(=O)O)c1O] | 4-[3-Cyano-5-(2,6-dichloro-benzyl)-2- hydroxy-phenyl]-4-oxo-butyric acid |
| 122 | 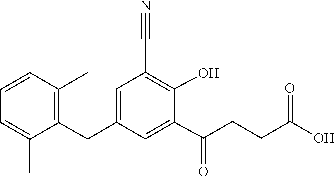 [SMILES: Cc1cccc(C)c1Cc1cc(C#N)c(O)c(C(=O)CCC(=O)O)c1] | 4-[3-Cyano-5-(2,6-dimethyl-benzyl)-2- hydroxy-phenyl]-4-oxo-butyric acid |
| 123 | 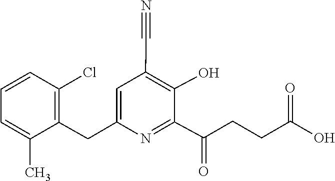 [SMILES: Cc1cccc(Cl)c1Cc1cc(C#N)c(O)c(C(=O)CCC(=O)O)n1] | 4-[6-(2-Chloro-6-methyl-benzyl)-4-cyano-3- hydroxy-pyridin-2-yl]-4-oxo-butyric acid |
| 124 | 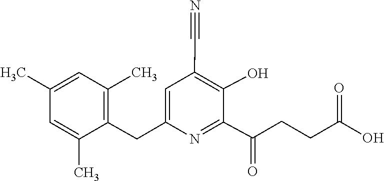 [SMILES: Cc1cc(C)c(Cc2cc(C#N)c(O)c(C(=O)CCC(=O)O)n2)c(C)c1] | 4-[4-Cyano-3-hydroxy-6-(2,4,6-trimethyl- benzyl)-pyridin-2-yl]-4-oxo-butyric acid |
| 125 | 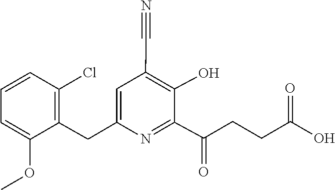 [SMILES: COc1cccc(Cl)c1Cc1cc(C#N)c(O)c(C(=O)CCC(=O)O)n1] | 4-[6-(2-Chloro-6-methoxy-benzyl)-4-cyano- 3-hydroxy-pyridin-2-yl]-4-oxo-butyric acid |
| 126 | 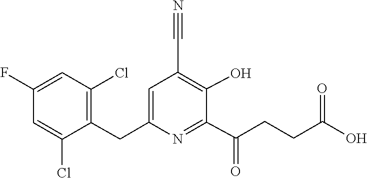 [SMILES: N#Cc1cc(Cc2c(Cl)cc(F)cc2Cl)nc(C(=O)CCC(=O)O)c1O] | 4-[4-Cyano-6-(2,6-dichloro-4-fluoro- benzyl)-3-hydroxy-pyridin-2-yl]-4-oxo- butyric acid |
| 127 | 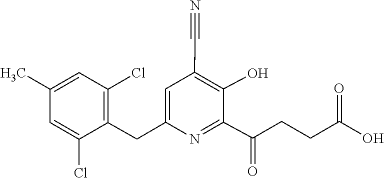 [SMILES: Cc1cc(Cl)c(Cc2cc(C#N)c(O)c(C(=O)CCC(=O)O)n2)c(Cl)c1] | 4-[4-Cyano-6-(2,6-dichloro-4-methyl- benzyl)-3-hydroxy-pyridin-2-yl]-4-oxo- butyric acid |
| 128 | 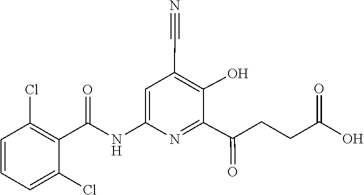 [SMILES: N#Cc1cc(NC(=O)c2c(Cl)cccc2Cl)nc(C(=O)CCC(=O)O)c1O] | 4-[4-Cyano-6-(2,6-dichloro-benzoylamino)- 3-hydroxy-pyridin-2-yl]-4-oxo-butyric acid |
| 129 | 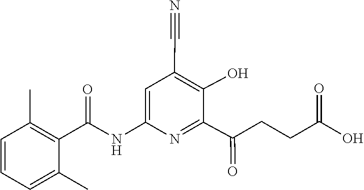 [SMILES: Cc1cccc(C)c1C(=O)Nc1cc(C#N)c(O)c(C(=O)CCC(=O)O)n1] | 4-[4-Cyano-6-(2,6-dimethyl-benzoylamino)- 3-hydroxy-pyridin-2-yl]-4-oxo-butyric acid |
| 130 | 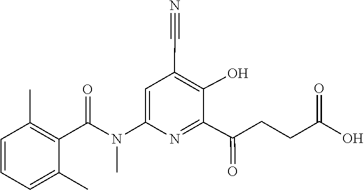 [SMILES: Cc1cccc(C)c1C(=O)N(C)c1cc(C#N)c(O)c(C(=O)CCC(=O)O)n1] | 4-{4-Cyano-6-[2,6-dimethyl-benzoyl)-N- methyl-amino]-3-hydroxy-pyridin-2-yl}-4- oxo-butyric acid |
| 131 | 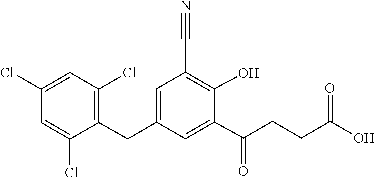 [SMILES: N#Cc1cc(Cc2c(Cl)cc(Cl)cc2Cl)cc(C(=O)CCC(=O)O)c1O] | 4-[3-Cyano-2-hydroxy-5-(2,4,6-trichloro- benzyl)-phenyl]-4-oxo-butyric acid |
| 132 | 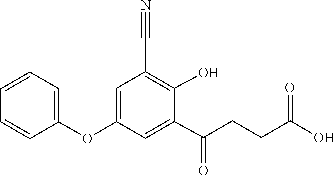 [SMILES: N#Cc1cc(Oc2ccccc2)cc(C(=O)CCC(=O)O)c1O] | 4-(3-Cyano-2-hydroxy-5-phenoxy-phenyl)- 4-oxo-butyric acid |
| 133 | 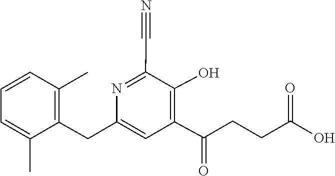 [SMILES: Cc1cccc(C)c1Cc1cc(C(=O)CCC(=O)O)c(O)c(C#N)n1] | 4-[2-Cyano-6-(2,6-dimethyl-benzyl)-3- hydroxy-pyridin-4-yl]-4-oxo-butyric acid |
| 134 | 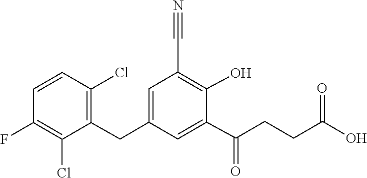 [SMILES: N#Cc1cc(Cc2c(Cl)ccc(F)c2Cl)cc(C(=O)CCC(=O)O)c1O] | 4-[3-Cyano-5-(2,6-dichloro-3-fluoro- benzyl)-2-hydroxy-phenyl]-4-oxo-butyric acid |
| 135 | 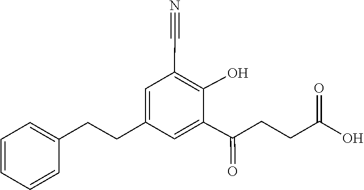 [SMILES: N#Cc1cc(CCc2ccccc2)cc(C(=O)CCC(=O)O)c1O] | 4-(3-Cyano-2-hydroxy-5-phenethyl-phenyl)- 4-oxo-butyric acid |
| 136 | 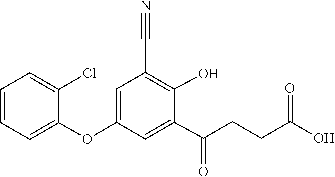 [SMILES: N#Cc1cc(Oc2ccccc2Cl)cc(C(=O)CCC(=O)O)c1O] | 4-[5-(2-Chloro-phenoxy)-3-cyano-2- hydroxy-phenyl]-4-oxo-butyric acid |
| 137 | 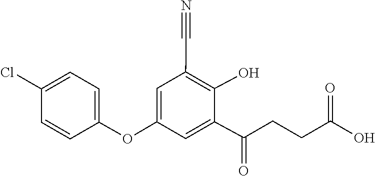 [SMILES: N#Cc1cc(Oc2ccc(Cl)cc2)cc(C(=O)CCC(=O)O)c1O] | 4-[5-(4-Chloro-phenoxy)-3-cyano-2- hydroxy-phenyl]-4-oxo-butyric acid |
| 138 | 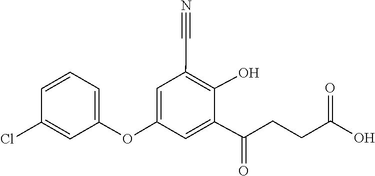 [SMILES: N#Cc1cc(Oc2cccc(Cl)c2)cc(C(=O)CCC(=O)O)c1O] | 4-[5-(3-Chloro-phenoxy)-3-cyano-2- hydroxy-phenyl]-4-oxo-butyric acid |
| 139 | 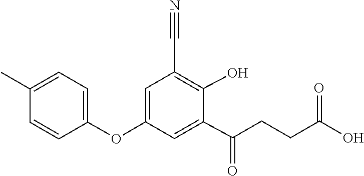 [SMILES: Cc1ccc(Oc2cc(C#N)c(O)c(C(=O)CCC(=O)O)c2)cc1] | 4-(3-Cyano-2-hydroxy-5-p-tolyloxy- phenyl)-4-oxo-butyric acid |
| 140 | 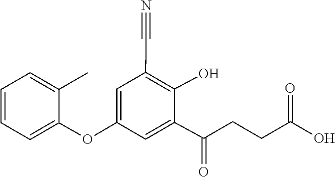 [SMILES: Cc1ccccc1Oc1cc(C#N)c(O)c(C(=O)CCC(=O)O)c1] | 4-(3-Cyano-2-hydroxy-5-o-tolyloxy- phenyl)-4-oxo-butyric acid |
| 141 | 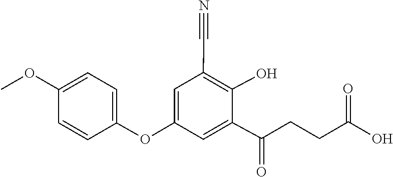 [SMILES: COc1ccc(Oc2cc(C#N)c(O)c(C(=O)CCC(=O)O)c2)cc1] | 4-[3-Cyano-2-hydroxy-5-(4-methoxy- phenoxy)-phenyl]-4-oxo-butyric acid |
| 142 | 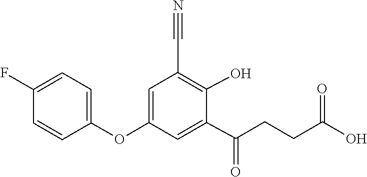 [SMILES: N#Cc1cc(Oc2ccc(F)cc2)cc(C(=O)CCC(=O)O)c1O] | 4-[3-Cyano-5-(4-fluoro-phenoxy)-2- hydroxy-phenyl]-4-oxo-butyric acid |
| 143 | 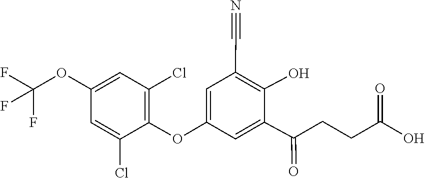 [SMILES: N#Cc1cc(Oc2c(Cl)cc(OC(F)(F)F)cc2Cl)cc(C(=O)CCC(=O)O)c1O] | 4-[4-Cyano-6-(2,6-dichloro-4- trifluoromethoxy-benzyl)-3-hydroxy- pyridin-2-yl]-4-oxo-butyric acid |
| 144 | 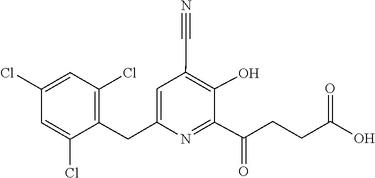 [SMILES: N#Cc1cc(Cc2c(Cl)cc(Cl)cc2Cl)nc(C(=O)CCC(=O)O)c1O] | 4-[4-Cyano-3-hydroxy-6-(2,4,6-trichloro- benzyl)-pyridin-2-yl]-4-oxo-butyric acid |
| 145 | 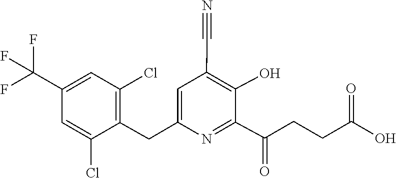 [SMILES: N#Cc1cc(Cc2c(Cl)cc(C(F)(F)F)cc2Cl)nc(C(=O)CCC(=O)O)c1O] | 4-[4-Cyano-6-(2,6-dichloro-4- trifluoromethyl-benzyl)-3-hydroxy-pyridin- 2-yl]-4-oxo-butyric acid |
| 146 | 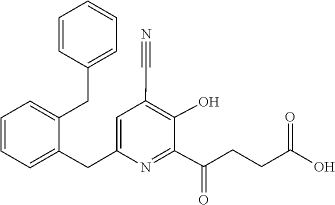 [SMILES: N#Cc1cc(Cc2ccccc2Cc2ccccc2)nc(C(=O)CCC(=O)O)c1O] | 4-[6-(2-Benzyl-benzyl)-4-cyano-3-hydroxy- pyridin-2-yl]-4-oxo-butyric acid |
| 147 | 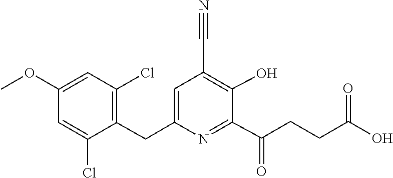 [SMILES: COc1cc(Cl)c(Cc2cc(C#N)c(O)c(C(=O)CCC(=O)O)n2)c(Cl)c1] | 4-[4-Cyano-6-(2,6-dichloro-4-methoxy- benzyl)-3-hydroxy-pyridin-2-yl]-4-oxo- butyric acid |
| 148 | 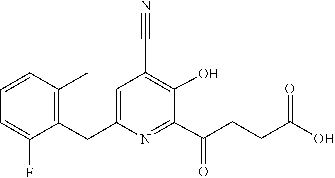 [SMILES: Cc1cccc(F)c1Cc1cc(C#N)c(O)c(C(=O)CCC(=O)O)n1] | 4-[4-Cyano-6-(2-fluoro-6-methyl-benzyl)-3- hydroxy-pyridin-2-yl]-4-oxo-butyric acid |
| 149 | 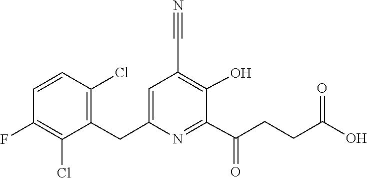 [SMILES: N#Cc1cc(Cc2c(Cl)ccc(F)c2Cl)nc(C(=O)CCC(=O)O)c1O] | 4-[4-Cyano-6-(2,6-dichloro-3-fluoro- benzyl)-3-hydroxy-pyridin-2-yl]-4-oxo- butyric acid |
| 150 | 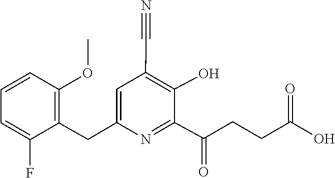 [SMILES: COc1cccc(F)c1Cc1cc(C#N)c(O)c(C(=O)CCC(=O)O)n1] | 4-[4-Cyano-6-(2-fluoro-6-methoxy-benzyl)- 3-hydroxy-pyridin-2-yl]-4-oxo-butyric acid |
| 151 | 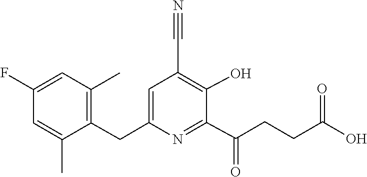 [SMILES: Cc1cc(F)cc(C)c1Cc1cc(C#N)c(O)c(C(=O)CCC(=O)O)n1] | 4-[4-Cyano-6-(4-fluoro-2,6-dimethyl- benzyl)-3-hydroxy-pyridin-2-yl]-4-oxo- butyric acid |
| 152 | 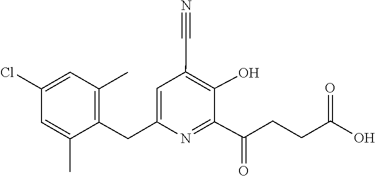 [SMILES: Cc1cc(Cl)cc(C)c1Cc1cc(C#N)c(O)c(C(=O)CCC(=O)O)n1] | 4-[4-Cyano-6-(4-chloro-2,6-dimethyl- benzyl)-3-hydroxy-pyridin-2-yl]-4-oxo- butyric acid |
| 153 | 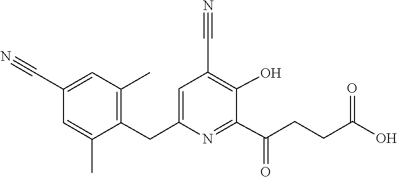 [SMILES: Cc1cc(C#N)cc(C)c1Cc1cc(C#N)c(O)c(C(=O)CCC(=O)O)n1] | 4-[4-Cyano-6-(4-cyano-2,6-dimethyl- benzyl)-3-hydroxy-pyridin-2-yl]--4-oxo- butyric acid |
| 154 | 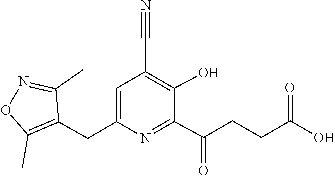 [SMILES: Cc1noc(C)c1Cc1cc(C#N)c(O)c(C(=O)CCC(=O)O)n1] | 4-[4-Cyano-6-(3,5-dimethyl-isoxazol-4- ylmethyl)-3-hydroxy-pyridin-2-yl]-4-oxo- butyric acid |
| 155 | 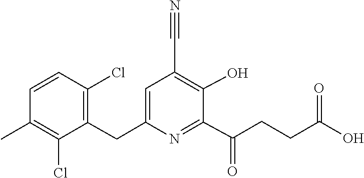 [SMILES: Cc1ccc(Cl)c(Cc2cc(C#N)c(O)c(C(=O)CCC(=O)O)n2)c1Cl] | 4-[4-Cyano-6-(2,6-dichloro-3-methyl- benzyl)-3-hydroxy-pyridin-2-yl]-4-oxo- butyric acid |
| 156 | 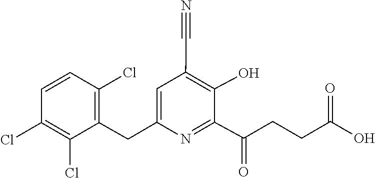 [SMILES: N#Cc1cc(Cc2c(Cl)ccc(Cl)c2Cl)nc(C(=O)CCC(=O)O)c1O] | 4-[4-Cyano-3-hydroxy-6-(2,3,6-trichloro- benzyl)-pyridin-2-yl]-4-oxo-butyric acid |
| 157 | 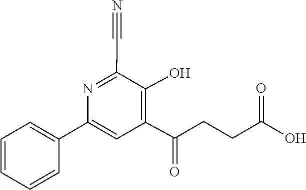 [SMILES: N#Cc1nc(-c2ccccc2)cc(C(=O)CCC(=O)O)c1O] | 4-(2-Cyano-3-hydroxy-6-phenyl-pyridin-4- yl)-4-oxo-butyric acid |
| 158 | 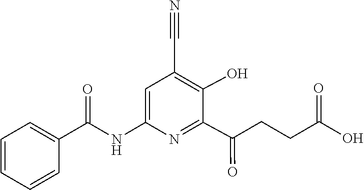 [SMILES: N#Cc1cc(NC(=O)c2ccccc2)nc(C(=O)CCC(=O)O)c1O] | 4-(6-Benzoylamino-4-cyano-3-hydroxy- pyridin-2-yl)-4-oxo-butyric acid |
| 159 | 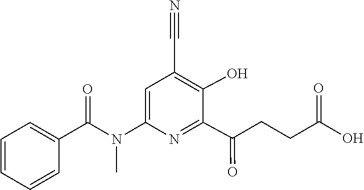 [SMILES: CN(C(=O)c1ccccc1)c1cc(C#N)c(O)c(C(=O)CCC(=O)O)n1] | 4-[6-(Benzoyl-N-methyl-amino)-4-cyano-3- hydroxy-pyridin-2-yl]-4-oxo-butyric acid |
| 160 | 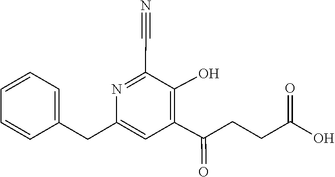 [SMILES: N#Cc1nc(Cc2ccccc2)cc(C(=O)CCC(=O)O)c1O] | 4-(6-Benzyl-2-cyano-3-hydroxy-pyridin-4- yl)-4-oxo-butyric acid |

### 3. Compositions and Methods

This disclosure provides compounds, compositions and methods of inhibiting the activity of a histone lysine demethylase-5 (KDM5) enzyme, as well as compounds and compositions for the manufacture of a medicament, for use in treating various conditions or disorders as described herein. The compound or composition can be used in methods to treat, pretreat, or delay progression or onset of a condition associated with a KDM5, particularly KDM5B. In certain embodiments, the composition is a pharmaceutical composition comprising a pharmaceutically acceptable excipient or carrier, and a therapeutically effective amount of one or more compounds of formula I In the present disclosure, each of the various embodiments above also relate to a pharmaceutically acceptable salt, isotopically enriched analog, stereoisomer, mixture of stereoisomers or prodrug of the compound (e.g., a compound of formula I).

In certain embodiments, provided is a method of inhibiting the activity of a KDM5, particularly KDM5B, comprising bringing into contact the KDM5 and an inhibitory-effective amount of a compound or pharmaceutical composition disclosed herein.

In certain embodiments, provided is a method of treating, pretreating, or delaying onset of a condition associated with KDM5, particularly KDM5B, the method comprising administering to a patient a therapeutically effective amount of a compound or pharmaceutical composition disclosed herein.

In certain embodiments, provided is a method of treating, pretreating, or delaying onset of a condition associated with undesirable cellular proliferation, the method comprising administering to a patient a therapeutically effective amount of a compound or pharmaceutical composition disclosed herein.

In certain embodiments, the condition is cancer. Exemplary cancers include, but are not limited to, acoustic neuroma; adenocarcinoma; adrenal gland cancer; anal cancer; angiosarcoma (e.g., lymphangio sarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM), myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In certain embodiments, the condition is a neoplasm, a tumor, or leukemia. In certain embodiments, the condition is histocytoma, glioma, astrocytoma, osteoma, lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, or melanoma. In certain embodiments, the cancer is embryonic carcinoma, teratoma, seminoma, germ cell tumors, prostate cancer, breast cancer, stomach cancer, gastrointestinal cancer, neuroblastoma, choriocarcinoma, yolk sac tumors, ovarian cancer, endometrial cancer, cervical cancer, retinoblastoma, kidney cancer, liver cancer, gastric cancer, brain cancer, medulloblastoma, medulloepithelioma, glioma, glioblastoma, multiple myeloma, lung cancer, bronchial cancer, mesothelioma, skin cancer, colon and rectal cancer, bladder cancer, pancreatic cancer, lip and oral cancer, laryngeal and pharyngeal cancer, melanoma, pituitary cancer, penile cancer, parathyroid cancer, thyroid cancer, pheochromocytoma and paraganglioma, thymoma and thymic carcinoma, leukemia, lymphoma, plasma cell neoplasms, myeloproliferative disorders, islet cell tumor, small intestine cancer, transitional cell cancer, pleuropulmonary blastoma, gestational trophoblastic cancer, esophageal cancer, central nervous system cancer, head and neck cancer, endocrine cancer, cardiovascular cancer, rhabdomyosarcoma, soft tissue carcinomas, carcinomas of bone, cartilage, fat, vascular, neural, and hematopoietic tissues or an AIDS-related cancer.

In certain embodiments, provided is a method of treating, pretreating, or delaying onset of a hematologic cancer, in particular a B-cell acute lymphocytic leukemia (B-ALL) or B-cell lymphoma, comprising administering a therapeutically effective amount of a compound or composition disclosed herein to a patient in need thereof. In certain embodiments, provided is a method of treating, pretreating, or delaying onset of a lung cancer, in particular a non-small cell lung cancer (NSCLC), comprising administering a therapeutically effective amount of a compound or composition disclosed herein to a patient in need thereof. In certain embodiments, provided is a method of treating, pretreating, or delaying onset of a breast cancer, in particular an estrogen receptor positive (ER+) breast cancer, comprising administering a therapeutically effective amount of a compound or composition disclosed herein to a patient in need thereof.

In certain embodiments, provided is a method of preventing or treating a viral infection, the method comprising administering to a patient a therapeutically effective amount of a compound or pharmaceutical composition disclosed herein.

In certain embodiments, the patient has a viral infection or is at risk for viral infection but is free from cancer. The viral infection may be due to a nuclear DNA viral infection such as a herpes viral infection. The herpesvirus may be, e.g., herpes simplex virus (HSV) type 1, herpes simplex virus type 2, varicella zoster virus (VZV), or cytomegalovirus (CMV). The herpesvirus may be Epstein-Barr virus (EBV), Kaposi's Sarcoma-Associated herpesvirus, herpes simiae virus, herpes lymphotropic virus, human herpesvirus-7 (HHMV-7), or human herpesvirus-8 (HHMV-8). Viral infections especially pose a threat to individuals that have suppressed (immunosuppressed) or otherwise compromised (immunocompromised) immune systems. For example, individuals with HIV/AIDS, diabetes, or cancer often have reduced ability to ward off additional and/or opportunistic viral infections due to immune systems that are adversely affected by the underlying, primary infection or condition. Therefore, preventing or treating viral infection or re-activation is especially important for these individuals.

In certain embodiments, the viral infection involves reactivation of a virus after latency in the patient. In certain embodiments, the patient has undergone, is undergoing, or will undergo, immunosuppression. In certain embodiments, the method prevents or treats viral-induced encephalitis, viral-induced keratitis, or reduces the severity of infection. In certain embodiments, the patient is an immunocompromised mammal.

In certain embodiments, provided is a method for treating a hepatitis B virus (HBV) infection, comprising administering a therapeutically effective amount of a compound or composition disclosed herein to a patient in need thereof. In certain embodiments, provided is a method for treating a hepatocellular carcinoma derived from persistent HBV or HCV infection, comprising administering a therapeutically effective amount of a compound or composition disclosed herein to a patient in need thereof.

In certain embodiments, the condition is cardiovascular disease. In certain embodiments, the cardiovascular disease is heart disease. In certain embodiments, the cardiovascular disease is coronary heart disease. In certain embodiments, the cardiovascular disease is stroke or cerebrovascular disease. In certain embodiments, the cardiovascular disease is a congenital heart defect. In certain embodiments, the cardiovascular disease is peripheral artery disease. In certain embodiments, the cardiovascular disease is heart disease associated with atherosclerosis. In certain embodiments, the cardiovascular disease is ischemic heart disease. In certain embodiments, the cardiovascular disease is hypertensive heart disease. In certain embodiments, the cardiovascular disease is cardiac arrhythmia. In certain embodiments, the cardiovascular disease is heart failure, congenital heart disease. In certain embodiments, the cardiovascular disease is inflammatory heart disease. In certain embodiments, the cardiovascular disease is cardiomyopathy.

In certain embodiments, the compound is administered in combination with one or more additional pharmaceutical agents described herein. The additional pharmaceutical agent may be an anti-proliferative agent. In certain embodiments, the additional pharmaceutical agent is an anti-cancer agent. The additional pharmaceutical agent may also be a kinase inhibitor. In certain embodiments, the additional pharmaceutical agent is an inhibitor of histone lysine demethylase. In certain embodiments, the additional pharmaceutical agent includes an anti-cancer agent, anti-inflammatory agent, steroids, immunosuppressant, radiation therapy, or other agents. In certain embodiments, the additional pharmaceutical agent is an anti-proliferative agent. In certain embodiments, the additional pharmaceutical agent is a non-selective inhibitor of a histone demethylase. In certain embodiments, the additional pharmaceutical agent is an immunotherapy agent. In certain embodiments, the additional pharmaceutical agent is an immune checkpoint inhibitor. In certain embodiments, the anti-cancer agent is a chemotherapeutic. In certain embodiments, the immunotherapy agent is a PD1 inhibitor. In certain embodiments, the immunotherapy agent is a PDL1 inhibitor. In certain embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In certain embodiments, the additional pharmaceutical agent is etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. Exemplary chemotherapeutic agents include alkylating agents such as nitrogen mustards, ethylenimines, methylmelamines, alkyl sulfonates, nitrosoureas, and triazenes; antimetabolites such as folic acid analogs, pyrimidine analogs, in particular fluorouracil and cytosine arabinoside, and purine analogs; natural products such as vinca alkaloids epi-podophyllotoxins, antibiotics, enzymes, and biological response modifiers; and miscellaneous products such as platinum coordination complexes, anthracenedione, substituted urea such as hydroxyurea, methyl hydrazine derivatives, and adrenocorticoid suppressant. Exemplary chemotherapeutic agents also include anthracycline antibiotics, actinomycin D, plicamycin, puromycin, gramicidin D, paclitaxel, colchicine, cytochalasin B, emetine, maytansine, amsacrine, cisplatin, carboplatin, mitomycin, altretamine, cyclophosphamide, lomustine, and carmustine. In certain embodiments, a pharmaceutical composition described herein further comprises a combination of the additional pharmaceutical agents described herein.

### 4. General Synthetic Methods

The compounds of this disclosure can be prepared from readily available starting materials using, for example, the following general methods, and procedures. It will be appreciated that where certain process conditions (i.e., reaction temperatures, times, mole ratios of reactants, solvents, pressures, etc.) are given, other process conditions can also be used unless otherwise stated. Optimum reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting certain functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance CA USA), EMKA-Chemie Gmbh & Co. KG (Eching Germany), or Millipore Sigma (Burlington MA USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

In certain embodiments, provided is a method of preparing a compound of Formula I, comprising contacting a compound of Formula I-1:

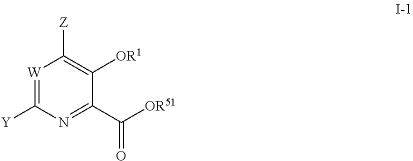

with a compound of Formula I-2:

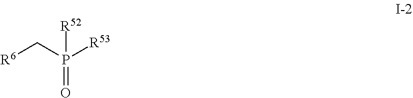

under conditions sufficient to produce a compound of Formula I-3:

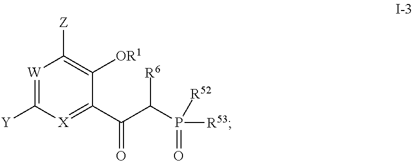

contacting a compound of Formula I-3 with a compound of Formula I-4:

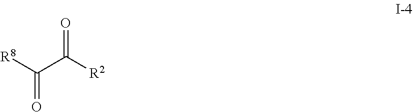

under suitable conditions to produce a compound of Formula I-5:

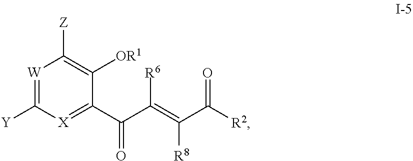

and
reducing and, when R⁷ and R⁹ are other than hydrogen, optionally further derivatizing a compound of Formula I-5 to produce a compound of Formula I-6:

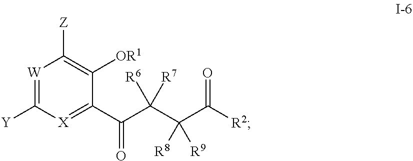

and optionally further coupling a compound of Formula I-6 with a compound of Formula I-7:

R⁵-A I-7
under appropriate coupling conditions to provide a compound of Formula I, wherein W, X, R¹, R², R³, R⁵, R⁶, R⁷, R⁸, and R⁹ are defined herein, Y and A, and Z and B are complimentary cross-coupling substituents, R⁵¹ is C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl, and R⁵² and R⁵³ are independently C₁₋₆ alkoxy, —O—C₂₋₆ alkenyl, —O—C₂₋₆ alkynyl, —O—C₁₋₆ haloalkyl, —O—C₃₋₁₀ cycloalkyl, —O—C₃₋₁₀ cycloalkenyl, —O-heterocyclyl, —O-aryl, or —O-heteroaryl. In certain embodiments, Y and Z are hydrogen or leaving groups (e.g.; halo, such as Cl, Br, or I, or a pseudohalide, such as a triflate, sulfonate, or phosphate), the transformation of which is known to those of skill in the art, and A and B are boronic acid, zinc(II) halide (e.g., —ZnBr or —ZnCl), trialkyltin (e.g., —SnBu₃), fluorosulfonyl esters, tin, zinc, sodium, or hydrogen. In certain embodiments, the coupling takes place in the presence of a catalyst (e.g., Pd). In certain embodiments, the method further comprises converting one or more substituents from one functional group to another. For example, in certain embodiments, the method further comprises converting R¹ from hydrogen to an optionally substituted alkyl.

Scheme I illustrates a general method which can be employed for the synthesis of compounds described herein, where W, X, R¹, R², R³, R⁵, R⁶, R⁷, R⁸, and R⁹ are defined herein, Y and Z are hydrogen or leaving groups (e.g.; halo, such as Cl, Br, or I, or a pseudohalide, such as a triflate, sulfonate, or phosphate), the transformation of which is known to those of skill in the art, A and B are boronic acid, zinc(II) halide (e.g., —ZnBr or —ZnCl), trialkyltin (e.g., —SnBu₃), fluorosulfonyl esters, tin, zinc, sodium, or hydrogen, R⁵¹ is C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl, and R⁵² and R⁵³ are each independently C₁₋₆ alkoxy, —O—C₂₋₆ alkenyl, —O—C₂₋₆ alkynyl, —O—C₁₋₆ haloalkyl, —O—C₃₋₁₀ cycloalkyl, —O—C₃₋₁₀ cycloalkenyl, —O— heterocyclyl, —O-aryl, or —O-heteroaryl.

In Scheme I, Y and A, and Z and B are complimentary cross-coupling substituents, and W, X, R¹, R², R³, R⁵, R⁶, R⁷, R⁸, R⁹, R⁵¹, R⁵², and R⁵³ are defined herein.

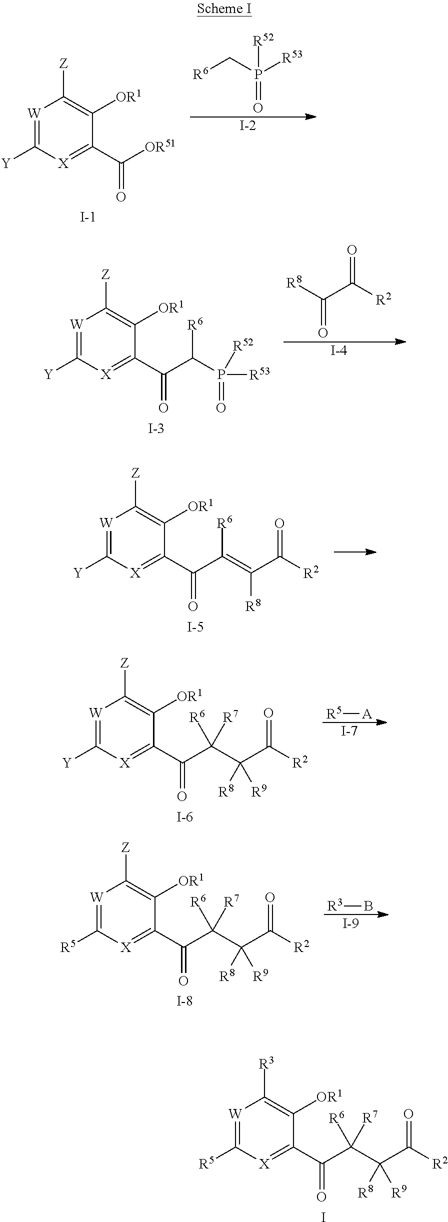

Referring to Scheme I, a compound of Formula I-2 is exposed to a base, for example n-butyl lithium, then reacted with a compound of Formula I-1 to produce phosphine oxides I-3. The compound of Formula I-3 can then be treated with a compound of Formula I-4, to provide compounds of Formula I-5. In certain embodiments, when control of stereochemistry is desired, proper control of reaction conditions and selection of substituents for the reagents and compounds of Formula I-3 and Formula I-4 can at least partially dictate the formation of E or Z isomers of Formula I-5, allowing for the stereocontrol of substituents R⁶, R⁷, R⁸, and R⁹ on subsequent fully-saturated compounds of Formula I-6. Further derivatization of α,β-unsaturated dioxo compounds of Formula I-5, transformations which are known to those of skill in the art, or exposure to standard reducing conditions, provides compounds of Formula I-6. A compound of Formula I-6 is coupled to compounds of Formula I-7 under standard cross coupling conditions to produce compounds of Formula I-8, which can be further coupled to compounds of Formula I-9 under standard cross coupling conditions to produce compounds of Formula I. Compounds of Formula I can be transesterified or hydrolyzed using methods known to one of skill in the art. For example, compounds of Formula I-8 are prepared by contacting compounds of Formula I-6, wherein Y is a leaving group (e.g., halo, such as Cl, Br, or I, or a pseudohalide, such as a triflate, sulfonate, or phosphate), with compounds of Formula I-7, wherein A is a suitable functional group such as, but not limited to, a boronic acid or a derivative thereof, such as a boronic ester, zinc or magnesium halide, an organotin compound, such as tributylstannane or trimethylstannane, fluorosulfonyl esters, tin, sodium, hydrogen, and the like. Similarly, compounds of Formula I are prepared by contacting compounds of Formula I-8, wherein Z is a leaving group (e.g., halo, such as Cl, Br, or I, or a pseudohalide, such as a triflate, sulfonate, or phosphate), with compounds of Formula I-9, wherein B is a suitable functional group such as, but not limited to, a boronic acid or a derivative thereof, such as a boronic ester, zinc or magnesium halide, an organotin compound, such as tributylstannane or trimethylstannane, fluorosulfonyl esters, tin, sodium, hydrogen, and the like. These reactions are typically conducted in the presence of suitable catalyst such as, but not limited to, a palladium catalyst including [1,1′-bis(diphenylphosphino)ferrocene]palladium(II) dichloride, Pd(OAc)₂, Pd(PPh₃)₄, PdCl₂(PPh₃)₂ or tris(dibenzylideneacetone)dipalladium(0), and the like, or a copper catalyst such as CuCl or CuI, and if required suitable mediator, co-catalyst and/or base known to one skilled in the art using suitable solvents/solvent mixtures. Upon reaction completion, compounds of Formula I can be recovered by conventional techniques such as neutralization, extraction, precipitation, chromatography, filtration and the like.

In some embodiments, the various substituents of compound I-1, I-2, I-3, I-4, I-5, I-6, I-7, 1-8, and I-9 (e.g., W, X, R¹, R², R³, R⁵, R⁶, R⁷, R⁸, R⁹, Y, Z, A, B, L, R⁵¹, R⁵², and R⁵³) are as defined for formula I. However, derivatization of compounds I-1, I-2, I-3, I-4, I-5, I-6, I-7, I-8, and I-9 prior to coupling and/or further derivatization of the resulting coupling product provides various compounds of formula I. Appropriate starting materials and reagents can be purchased or prepared by methods known to one of skill in the art.

Scheme II illustrates various general methods which can be employed for the synthesis of compounds described herein. In Scheme II, W, X, R¹, R², R³, R⁴, R⁵, R⁶, R⁷, R⁸, and R⁹ are independently as defined throughout, and each R⁵⁰ is hydrogen, alkyl, or together with the atoms to which they are attached, form a cyclic boronic ester.

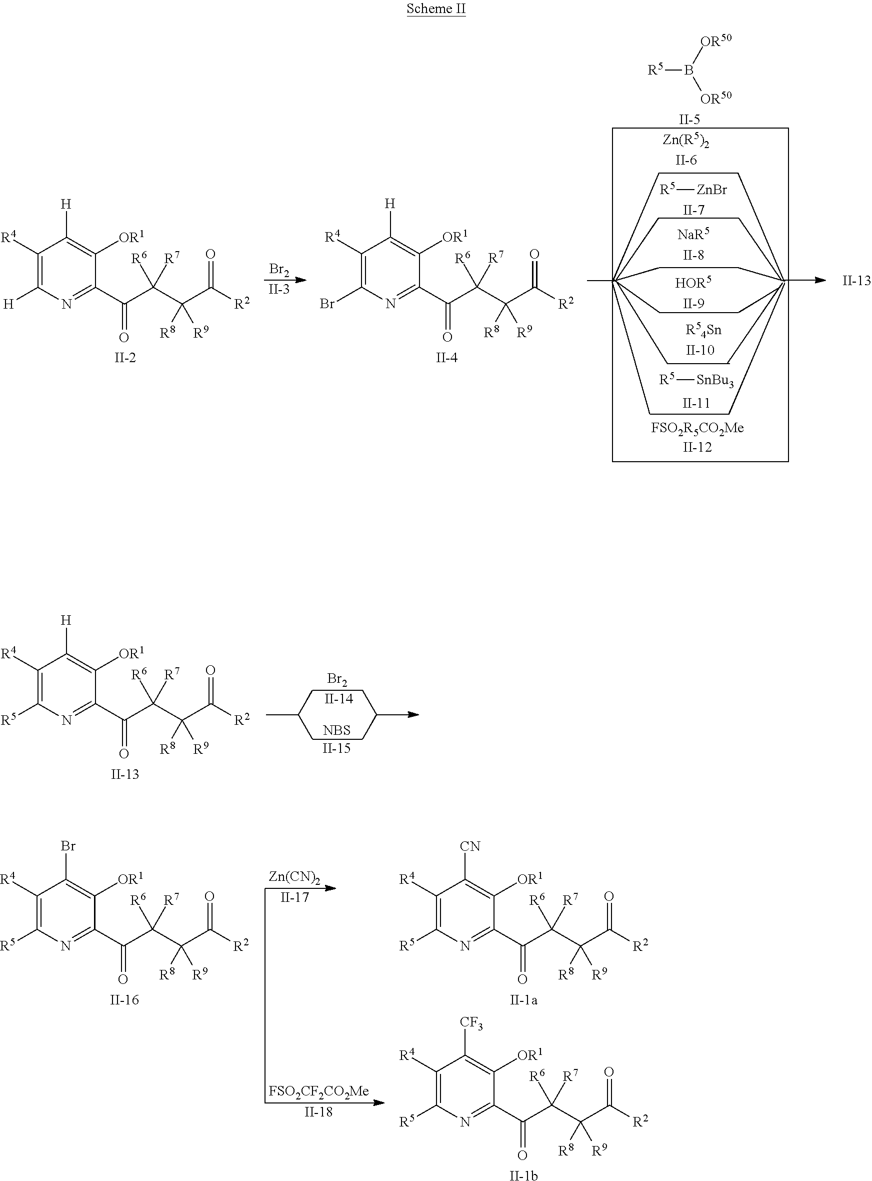

Exemplary compounds of Formula I, where W is CR⁴, X is N and R³ is cyano (Formula II-1a) or trifluoromethyl (Formula II-1b), can be prepared by reacting pyridine ester compounds of Formula II-2 with bromine under appropriate solvent and reaction conditions, to provide compounds of Formula II-4. An appropriately halogenated starting compound of Formula II-4 can then be coupled with a boronic acid or ester compound of Formula II-5 under suitable coupling conditions (e.g., Suzuki coupling), or can be coupled with an organozinc compound of Formula II-6 under suitable coupling conditions, or can be coupled with a zinc halide of Formula II-7 under suitable coupling conditions (e.g., Negishi coupling), or can be reacted with a sodium alkylthiolate or sodium alkoxide compound of Formula II-8 under suitable reaction conditions, or can be reacted with an alcohol compound of Formula II-9 under suitable reaction conditions, or can be coupled with a tetraorganotin compound of Formula II-10 under suitable coupling conditions, or can be coupled with an organotin compound of Formula II-11 under suitable coupling conditions (e.g., Stille coupling), or can be reacted with a fluorosulfonyl ester compound of Formula II-12 under suitable reaction conditions, to provide compounds of Formula II-13. The compounds of Formula II-13 can be further derivatized by brominating with bromine or N-bromosuccinimide under appropriate solvent and reaction conditions, to provide compounds of Formula II-16. Coupling appropriately halogenated starting compounds of Formula II-16 with zinc cyanide (Formula II-17) under suitable coupling conditions provides compounds of Formula II-la. Similarly, treating appropriately halogenated starting compounds of Formula II-16 with methyl 2,2-difluoro-2-(fluorosulfonyl)acetate (Formula II-18) under suitable reaction conditions provides compounds of Formula II-1b.

Scheme III further illustrates various general methods which can be employed for the synthesis of compounds described herein. In Scheme III, W, X, R¹, R², R³, R⁴, R⁵, R⁶, R⁷, R⁸, and R⁹ are independently as defined throughout, and each R⁵⁰ is hydrogen, alkyl, or together with the atoms to which they are attached, form a cyclic boronic ester.

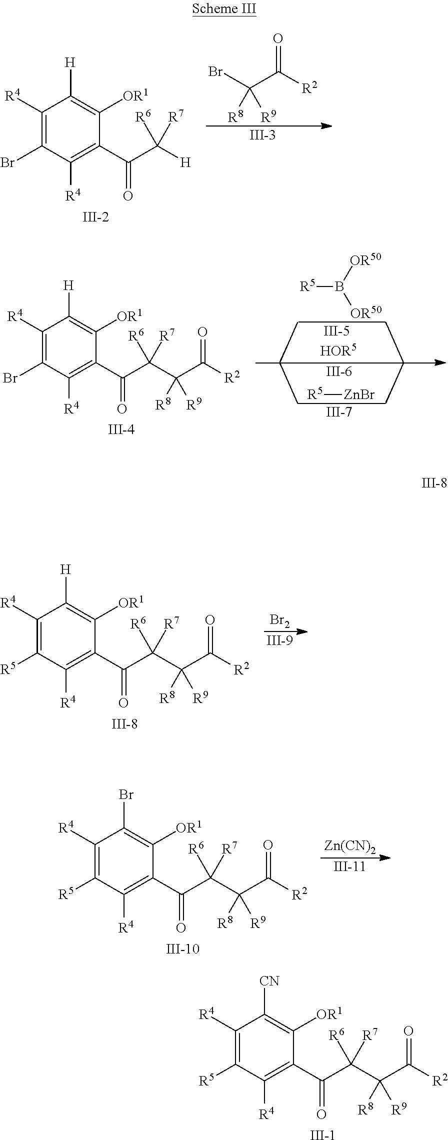

Further exemplary compounds of formula I, where W and X are CR⁴ and R³ is cyano, can be prepared by reacting substituted phenyl ketone compounds of Formula III-2 with bromoacetate compounds of Formula III-3 under appropriate solvent and reaction conditions, to provide compounds of Formula III-4. An appropriately halogenated starting compound of Formula III-4 can then be coupled with a boronic acid or ester compound of Formula III-5 under suitable coupling conditions (e.g., Suzuki coupling), or can be reacted with an alcohol compound of Formula III-6 under suitable reaction conditions, or can be coupled with a zinc halide of Formula III-7 under suitable coupling conditions (e.g., Negishi coupling), to provide compounds of Formula III-8. The compounds of Formula III-8 can be further derivatized by brominating with bromine under appropriate solvent and reaction conditions, to provide compounds of Formula III-10. Coupling appropriately halogenated starting compounds of Formula III-10 with zinc cyanide (Formula III-11) under suitable coupling conditions provides compounds of Formula III-1.

Scheme IV further illustrates various general methods which can be employed for the synthesis of compounds described herein. In Scheme IV, W, X, R¹, R², R³, R⁴, R⁵, R⁶, R⁷, R¹, R⁹, R⁵¹, R⁵², and R⁵³ are independently as defined throughout, and each R⁵⁰ is hydrogen, alkyl, or together with the atoms to which they are attached, form a cyclic boronic ester.

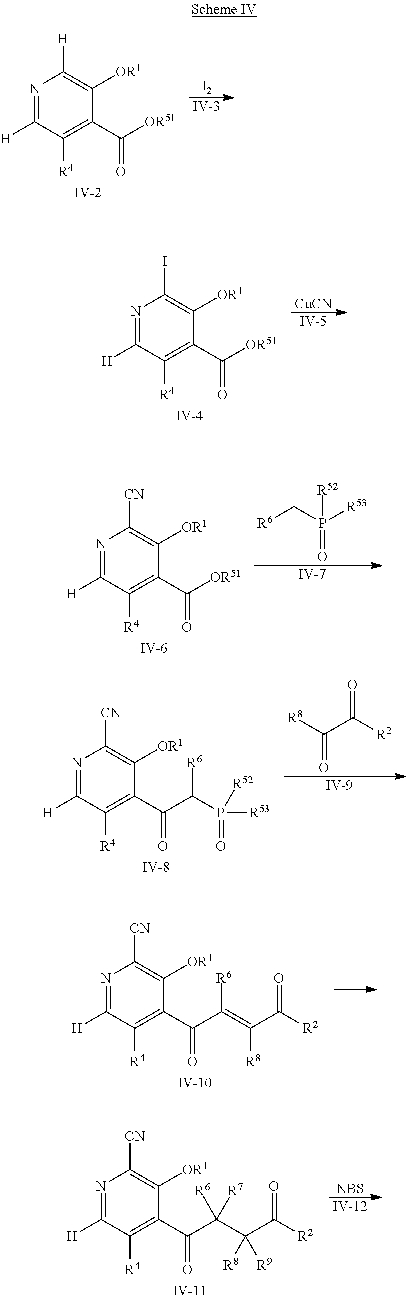

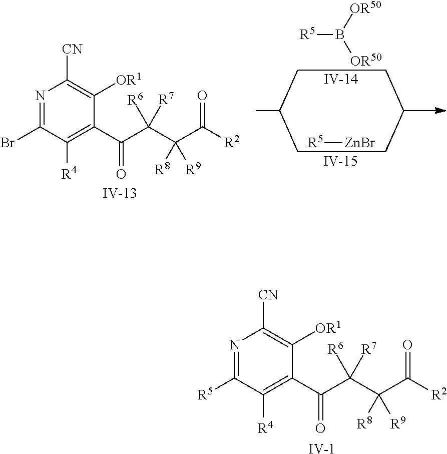

Further exemplary compounds of Formula I, where W is N, X is CR⁴ and R³ is cyano, can be prepared by reacting isonicotinic acid ester compounds of Formula IV-2 with iodine under appropriate solvent and reaction conditions, to provide compounds of Formula IV-4. An appropriately halogenated starting compound of Formula IV-4 can then be coupled with copper cyanide (Formula IV-5) under suitable coupling conditions to provide cyano isonicotinic acid ester compounds of Formula IV-6. A compound of Formula IV-7 is then exposed to a base, for example sodium bis(trimethylsilyl)amide, then reacted with cyano isonicotinic acid ester compounds of Formula IV-6 to produce phosphine oxide compounds of Formula IV-8. The compound of Formula IV-8 can then be treated with a compound of Formula IV-9, to provide compounds of Formula IV-10. In certain embodiments, when control of stereochemistry is desired, proper control of reaction conditions and selection of substituents for the reagents and compounds of Formula IV-8 and Formula IV-9 can at least partially dictate the formation of E or Z isomers of Formula IV-10, allowing for the stereocontrol of substituents R⁶, R⁷, R⁸, and R⁹ on subsequent fully-saturated compounds of Formula IV-11. Further derivatization of α,β-unsaturated dioxo compounds of Formula IV-10, transformations which are known to those of skill in the art, or exposure to standard reducing conditions, provides compounds of Formula IV-11. The compounds of Formula IV-11 can be further derivatized by brominating with N-bromosuccinimide (Formula IV-12) under appropriate solvent and reaction conditions, to provide compounds of Formula IV-13. An appropriately halogenated starting compound of Formula IV-13 can then be coupled with a boronic acid or ester compound of Formula IV-14 under suitable coupling conditions (e.g., Suzuki coupling), or can be coupled with a zinc halide of Formula IV-15 under suitable coupling conditions (e.g., Negishi coupling), to provide compounds of Formula IV-1.

Appropriate starting materials and reagents can be purchased or prepared by methods known to one of skill in the art. Upon each reaction completion, each of the intermediate or final compounds can be recovered, and optionally purified, by conventional techniques such as neutralization, extraction, precipitation, chromatography, filtration, and the like.

Other modifications to arrive at compounds of this disclosure are within the skill of the art.

### 5. Formulations and Administration

The compositions of the present disclosure can be delivered directly or in pharmaceutical compositions along with suitable carriers or excipients, as is well known in the art. Present methods of treatment can comprise administration of an effective amount of a compound of the disclosure to a subject in need; e.g., a subject having or at risk for a hyperproliferative disease or cancer. In certain embodiments, the subject is a mammalian subject. In certain embodiments, the subject is a human subject.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences, supra.

Suitable routes of administration may, for example, include oral, rectal, topical, nasal, pulmonary, ocular, intestinal, and parenteral administration. The indication to be treated, along with the physical, chemical, and biological properties of the drug, dictate the type of formulation and the route of administration to be used, as well as whether local or systemic delivery would be preferred.

Pharmaceutical compositions are often composed of the drug and an excipient(s). Pharmaceutical dosage forms are often composed of the drug, an excipient(s), and a container/closure system. One or multiple excipients, also referred to as inactive ingredients, can be added to a compound of the disclosure to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott NY; etc.)

Pharmaceutical dosage forms of a compound of the present disclosure may be manufactured by any of the methods well-known in the art, such as, for example, by conventional mixing, sieving, dissolving, melting, granulating, dragee-making, tableting, suspending, extruding, spray-drying, levigating, emulsifying, (nano/micro-) encapsulating, entrapping, or lyophilization processes. As noted above, the compositions of the present disclosure can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the disclosure, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are generally preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages typically fall within a range of circulating concentrations that includes the ED₅₀ with little or no toxicity. Dosages may vary within this range depending upon the dosage form employed and/or the route of administration utilized. Dosages are typically expressed as a number of milligrams of a compound described herein per kilogram of the subject's body weight (mg/kg). Dosages of between about 0.1 and 900 mg/kg may be appropriate. In some embodiments, about 1 and 500 mg/kg may be appropriate. In other embodiments a dosage of between 10 and 250 mg/kg may be appropriate. In some embodiments, a dosage of from about 1 to about 100 mg per kg of body weight per day, from about 1 to about 50 mg of compound per kg of body weight, or from about 1 to about 10 mg of compound per kg of body weight may be appropriate. In some embodiments, a dosage of from about 25 to about 500 mg per kg of body weight per day, from about 50 to about 500 mg of compound per kg of body weight, or from about 25 to about 250 mg of compound per kg of body weight may be appropriate. In other embodiments a dosage of between 10 and 250 mg/kg may be appropriate. In some embodiments, a dosage of from about 1 to about 100 mg per kg of body weight, from about 1 to about 50 mg of compound per kg of body weight, or from about 1 to about 10 mg of compound per kg of body weight may be appropriate. In some embodiments, a dosage of from about 25 to about 500 mg per kg of body weight, from about 50 to about 500 mg of compound per kg of body weight, or from about 25 to about 250 mg of compound per kg of body weight may be appropriate. The exact formulation, route of administration, dosage, and dosage interval should be chosen according to methods known in the art, in view of the specifics of a subject's condition.

The amount of agent or composition administered may be dependent on a variety of factors, including the sex, age, and weight of the subject being treated, the severity of the affliction, the manner of administration, and the judgment of the prescribing physician.

These and other embodiments of the present disclosure will readily occur to those of ordinary skill in the art in view of the disclosure herein and are specifically contemplated.

### EXAMPLES

This disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

### Example 1

### 4-(4,6-Dibromo-3-hydroxy-pyridin-2-yl)-4-oxo-butyric acid

### a) 3-Hydroxy-pyridine-2-carboxylic acid ethyl ester

To the solution of 3-hydroxy-pyridine-2-carboxylic acid (15.0 g, 108 mmol) in anhydrous ethanol (300 mL) was added 98% sulfuric acid (17.0 mL, 324 mmol, 3.0 eq.). The reaction was refluxed for 24 hours. After the solvents were evaporated, the residue was dissolved in 300 mL of water, neutralized with saturated sodium bicarbonate solution and extracted with ethyl acetate (200 mL×3). The combined organics were washed with brine (200 mL), dried over sodium sulfate, filtered and evaporated in vacuo to afford the title compound; MS-(+)-ion, M+1=168.18.

### b) 3-Benzyloxy-pyridine-2-carboxylic acid ethyl ester

Benzyl bromide (7.35 mL, 10.57 g, 60.7 mmol, 1.4 eq.) was added to a mixture of 3-hydroxy-pyridine-2-carboxylic acid ethyl ester (7.25 g, 46.5 mmol, 1.0 eq., see Example 1a) and cesium carbonate (17.0 g, 52.1 mmol, 1.2 eq.) in anhydrous DMF (60 mL) at room temperature. After 3 hours at room temperature, TLC shows the completion of the reaction. The reaction mixture was diluted with water (200 mL) and extracted with ethyl acetate (4×150 mL). The combined extracts were washed with brine (2×200 mL), dried over sodium sulfate, filtered and evaporated in vacuo to afford the crude product. This material was purified by flash chromatography eluting with ethyl acetate/hexane (0%-70%) to give the title compound. ¹H NMR (CDCl₃, 200 MHz) δ=8.29 (t, J=2.8 Hz, 1H), 7.44-7.24 (m, 7H), 5.20 (s, 2H), 4.46 (q, J=7.0 Hz, 2H), 1.40 (t, J=7.0 Hz, 3H).

### c) [2-(3-Benzyloxy-pyridin-2-yl)-2-oxo-ethyl]-phosphonic acid dimethyl ester

At −78° C., to a solution of dimethyl methylphosphonate (10.0 mL, 92 mmol, 2.2 eq.) in THF (200 mL) was added n-BuLi (2.5 M in hexanes, 33.5 mL, 83.6 mmol, 2.0 eq) over 20 min under N₂ atmosphere. After 30 min, a solution of 3-benzyloxy-pyridine-2-carboxylic acid ethyl ester (11.41 g, 92 mmol, see Example 1b) in THF (50 mL) was added slowly over 20 min. After stirring for 1 h at −78° C., the mixture was treated with half saturated aq. NH₄Cl (200 mL) and extracted with ethyl acetate (4×150 mL). The combined extracts were dried over sodium sulfate, filtered and evaporated in vacuo to afford the crude product. This material was purified by flash chromatography eluting with MeOH/DCM (0%-8%) to give the title compound. ¹H NMR (200 MHz, CDCl₃) δ=8.27 (dd, J=3.2 Hz, 2.4 Hz, 1H), 7.46-7.26 (m, 7H), 5.23 9 (s, 2H), 3.98 (d, J=22.2 Hz, 2H), 3.77 (d, J=1.2 Hz, 3H), 3.71 (d, J=1.2 Hz, 3H). MS-(+)-ion, M+1=336.35.

### d) 4-(3-Benzyloxy-pyridin-2-yl)-4-oxo-but-2-enoic acid ethyl ester

To a solution of [2-(3-benzyloxy-pyridin-2-yl)-2-oxo-ethyl]-phosphonic acid dimethyl ester (10.0 g, 30.0 mmol, see Example 1c) in THF (120 mL) at −78° C. was added t-BuOK (1 M in THF, 36 mmol, 36 mL, 1.2 eq). After stirring at −78° C. for 10 min, 8.6 mL of ethyl glyoxalate (50 wt % in toluene, 12.24 g, 60.0 mmol, 2.0 eq.) was added dropwise over 20 min via a dropping funnel. The mixture was stirred at −78° C. for 3 h, then was treated with half saturated aq. NH₄Cl (200 mL) and extracted with ethyl acetate (4×150 mL). The combined extracts were dried over sodium sulfate, filtered and evaporated in vacuo to afford the crude product. This material was purified by flash chromatography eluting with EtOAc/hexane (0%-30%) to give the title compound. ¹H NMR (200 MHz, CDCl₃) δ=8.27 (t, J=7.8 Hz, 1H), 8.11 (d, J=19.5 Hz, 1H), 7.48-7.32 (m, 7H), 6.85 (d, J=19.5 Hz, 1H), 5.23 (s, 2H), 4.28 (q, J=9.0 Hz, 2H), 1.34 (t, J=9.0 Hz, 3H). MS-(+)-ion, M+23=344.40.

### e) 4-(3-Hydroxy-pyridin-2-yl)-4-oxo-butyric acid ethyl ester

To a solution of 4-(3-benzyloxy-pyridin-2-yl)-4-oxo-but-2-enoic acid ethyl ester (3.3 g, 10.6 mmol, see Example 1d) in ethyl acetate (60 mL) was added Pd/C (200 mg, 0.01 eq, 10 wt. %, wet, contains ˜51% water). The mixture was vacuumed/refilled with hydrogen gas three times. After stirring for 16 hr at room temperature, the reaction mixture was filtered through celite. The filtrate was evaporated in vacuo to afford the crude product. This material was purified by flash chromatography eluting with EtOAc/hexane (0%-20%) to give the title compound. ¹H NMR (200 MHz, CDCl₃) δ=11.6 (s, 1H), 8.23 (dd, J=4.4 Hz, 1.6 Hz, 1H), 7.41-7.35 (m, 2H), 4.16 (q, J=7.4 Hz, 2H), 3.62 (t, J=6.6 Hz, 2H), 2.75 (t, J=6.6 Hz, 2H), 1.26 (t, J=7.4 Hz, 3H). MS-(+)-ion, M+1=223.96.

### f) 4-(4,6-Dibromo-3-hydroxy-pyridin-2-yl)-4-oxo-butyric acid ethyl ester

At room temperature, to a solution of 4-(3-hydroxy-pyridin-2-yl)-4-oxo-butyric acid ethyl ester (7.9 g, 47.3 mmol, see Example 1e) and sodium acetate (8.1 g, 99 mmol) in anhydrous CHCl₃ (150 mL) was added bromine (5.0 mL, 99 mmol). After 20 hours at room temperature, the reaction was quenched with saturated NaHSO₃ aqueous solution and extracted with DCM (15 mL×3). The combined organic layers were washed with aqueous sodium bicarbonate and brine, then dried over sodium sulfate, filtered and evaporated in vacuo to afford the crude product. This material was purified by flash chromatography eluting with ethyl acetate/hexane (0%-10%) to give the title compound. ¹H NMR (CDCl₃, 200 MHz) δ=12.21 (s, 1H), 8.16 (s, 1H), 7.97 (dd, J=8.2 Hz, 1.8 Hz, 2H), 7.49-7.44 (m, 3H), 4.17 (q, J=7.0 Hz, 2H), 3.75 (t, J=6.6 Hz, 2H), 2.80 (t, J=6.6 Hz, 2H), 1.29 (t, J=7.0 Hz, 3H). MS-(+)-ion, M+23=380.46.

### g) 4-(4,6-Dibromo-3-hydroxy-pyridin-2-yl)-4-oxo-butyric acid

At room temperature, to a solution of 4-(4,6-dibromo-3-hydroxy-pyridin-2-yl)-4-oxo-butyric acid ethyl ester (19 mg, 0.05 mmol, see Example if) in THF/H₂O (1 mL/0.5 mL) was added lithium hydroxide monohydrate (12 mg, 0.3 mmol). After 20 hours at room temperature, the reaction was diluted with 15 mL of water and extracted with ethyl acetate (10 mL×3). The resulting aqueous layer was treated with 1N HCl to pH=4, followed by extraction with ethyl acetate (10 mL×3). The combined organic layers were washed with brine and dried over sodium sulfate, filtered and evaporated in vacuo to afford the title compound. MS-(+)-ion, M+H=353.68.

### Example 2

### 4-(4-Bromo-6-cyano-3-hydroxy-pyridin-2-yl)-4-oxo-butyric acid

### a) 4-(6-Bromo-3-hydroxy-pyridin-2-yl)-4-oxo-butyric acid ethyl ester

To a solution of 4-(3-hydroxy-pyridin-2-yl)-4-oxo-butyric acid ethyl ester (2.0 g, 9.0 mmol, see Example 1e) in DCM (9 mL) was added 90 mL H₂O. Bromine (0.51 mL, 1.1 eq., 9.9 mmol) was then added slowly over 5 min to the mixture at room temperature. The reaction flask was wrapped in aluminum foil. After 1 hour at room temperature, a second portion of bromine (0.10 mL, 0.2 eq., 1.8 mmol) was added to the reaction as TLC shows starting material remains. After another 2 hours, the reaction was quenched with 100 mL of saturated NaHSO₃ aqueous solution and extracted with DCM (100 mL×3). The combined organic layers were washed with brine (200 mL) and dried over sodium sulfate, filtered and evaporated in vacuo to afford the crude product. This material was purified by flash chromatography eluting with ethyl acetate/hexane (0%-10%) to give the title compound. ¹H NMR (CDCl₃, 200 MHz) δ=11.58 (s, 1H), 7.53 (d, J=9.0 Hz, 1H), 7.23 (d, J=9.0 Hz, 1H, merged with CHCl₃), 4.16 (q, J=7.4 Hz, 2H), 3.56 (t, J=6.2 Hz, 2H), 2.73 (t, J=6.2 Hz, 2H), 1.26 (t, J=7.4 Hz, 3H). MS-(+)-ion, M+H=303.77.

### b) 4-(6-Cyano-3-hydroxy-pyridin-2-yl)-4-oxo-butyric acid ethyl ester

A mixture of 4-(6-bromo-3-hydroxy-pyridin-2-yl)-4-oxo-butyric acid ethyl ester (606 mg, 2.0 mmol, see Example 2a), zinc cyanide (234 mg, 2.0 mmol), tris(dibenzylideneacetone)dipalladium(0) (183 mg, 0.2 mmol), 1,1′-Bis(diphenylphosphino)ferrocene (dppf, 222 mg, 0.4 mmol), and zinc dust (26 mg, 0.4 mmol) in anhydrous dimethylacetamide (10 mL) was heated al 100° C. under N₂ atmosphere for 3 hours. After cooling to room temperature, the reaction mixture was diluted with water (50 mL) and ethyl acetate (50 mL). 5 mL of 1 N HCl was added to the mixture and it was allowed to stir for 30 min at room temperature. The organic layer was collected. The aqueous layer was further extracted with ethyl acetate (2×50 mL). The combined extracts were washed with brine (100 mL), dried over sodium sulfate, filtered and evaporated in vacuo to afford the crude product. This material was purified by flash chromatography eluting with ethyl acetate/hexane (0%-30%) to give the title compound. MS-(−)-ion, M+H=247.44.

### c) 4-(4-Bromo-6-cyano-3-hydroxy-pyridin-2-yl)-4-oxo-butyric acid ethyl ester

At room temperature, to a solution of 4-(6-cyano-3-hydroxy-pyridin-2-yl)-4-oxo-butyric acid ethyl ester (360 mg, 1.44 mmol, see Example 2b) and sodium acetate (142 mg, 1.73 mmol) in anhydrous CHCl₃ (10 mL) was added bromine (88 uL, 275 mg, 1.73 mmol). The reaction flask was wrapped in aluminum foil. After 20 hours at room temperature, the reaction was quenched with 15 mL saturated NaHSO₃ aqueous solution and extracted with DCM (15 mL×3). The combined organic layers were washed with brine (20 mL) and dried over sodium sulfate, filtered and evaporated in vacuo to afford the crude product. This material was purified by flash chromatography eluting with ethyl acetate/hexane (0%-10%) to give the title compound. MS-(+)-ion, M+H=324.88, 326.88.

### d) 4-(4-Bromo-6-cyano-3-hydroxy-pyridin-2-yl)-4-oxo-butyric acid

The title compound was prepared from 4-(4-bromo-6-cyano-3-hydroxy-pyridin-2-yl)-4-oxo-butyric acid ethyl ester (see example 2c) in analogy to example 1g, MS-(−)-ion, M−H=296.87, 298.87.

### Example 3

### 4-(4,6-Dicyano-3-hydroxy-pyridin-2-yl)-4-oxo-butyric acid

### a) 4-(4,6-Dicyano-3-hydroxy-pyridin-2-yl)-4-oxo-butyric acid ethyl ester

A mixture of 4-(4,6-dibromo-3-hydroxy-pyridin-2-yl)-4-oxo-butyric acid ethyl ester (190 mg, 0.5 mmol, see Example 1f), zinc cyanide (88 mg, 0.75 mmol), tris(dibenzylideneacetone)dipalladium(0) (46 mg, 0.05 mmol), 1,1′-Bis(diphenylphosphino)ferrocene (dppf, 55 mg, 0.1 mmol), and zinc dust (9.8 mg, 0.15 mmol) in anhydrous dimethylacetamide (8 mL) was heated at 100° C. under N₂ atmosphere for 3 hours. After cooling to room temperature, the reaction mixture was diluted with water (30 mL) and ethyl acetate (30 mL). 3 mL of 1 N HCl was added to the mixture followed by stirring for 30 min at room temperature. The organic layer was collected. The aqueous layer was further extracted with ethyl acetate (2×30 mL). The combined extracts were washed with brine (100 mL), dried over sodium sulfate, filtered and evaporated in vacuo to afford the crude product. This material was purified by flash chromatography eluting with ethyl acetate/hexane (0%-70%) to give the title compound. MS-(+)-ion, M+H=272.40.

### b) 4-(4,6-Dicyano-3-hydroxy-pyridin-2-yl)-4-oxo-butyric acid

The title compound was prepared from 4-(4,6-dicyano-3-hydroxy-pyridin-2-yl)-4-oxo-butyric acid ethyl ester (see example 3a) in analogy to example 1g, MS-(−)-ion, M−H=244.28.

### Example 4

### 4-(3-Hydroxy-4,6-bis-trifluoromethyl-pyridin-2-yl)-4-oxo-butyric acid

### a) 4-(3-Benzyloxy-4,6-dibromo-pyridin-2-yl)-4-oxo-butyric acid ethyl ester

The title compound was prepared from 4-(4,6-dibromo-3-hydroxy-pyridin-2-yl)-4-oxo-butyric acid ethyl ester (see Example if) and benzyl bromide in analogy to example 1b, MS-(+)-ion, M+Na=494.28.

### b) 4-(3-Benzyloxy-4,6-bis-trifluoromethyl-pyridin-2-yl)-4-oxo-butyric acid ethyl ester

A mixture of 4-(3-benzyloxy-4,6-dibromo-pyridin-2-yl)-4-oxo-butyric acid ethyl ester (71 mg, 0.15 mmol, see Example 4a), methyl 2,2-difluoro-2-(fluorosulfonyl)acetate (287 mg, 1.5 mmol), and copper(I) iodide (171 mg, 0.9 mmol), in anhydrous 1-methyl-2-pyrrolidone (3 mL) was heated at 100° C. under N₂ atmosphere for 3 hours. After cooling to room temperature, the reaction mixture was diluted with water (30 mL), quenched with 5 mL of 1 N HCl and extracted with ethyl acetate (3×30 mL). The combined extracts were washed with brine (50 mL), dried over sodium sulfate, filtered and evaporated in vacuo to afford the crude product. This material was purified by flash chromatography eluting with ethyl acetate/hexane (0%-40%) to give the title compound. MS-(+)-ion, M+Na=472.39.

### c) 4-(3-Hydroxy-4,6-bis-trifluoromethyl-pyridin-2-yl)-4-oxo-butyric acid ethyl ester

A solution of 4-(3-benzyloxy-4,6-bis-trifluoromethyl-pyridin-2-yl)-4-oxo-butyric acid ethyl ester (15 mg, 0.033 mmol, see Example 4b) in 1 mL trifluoroacetic acid was heated at 70° C. for 2 hours. After cooling down to room temperature, the reaction was slowly quenched with aqueous sodium bicarbonate solution to pH=7-8. The mixture was extracted with ethyl acetate, dried over sodium sulfate, filtered and evaporated in vacuo to afford the title compound, which was used directly in the next step. MS-(−)-ion, M−H=358.40.

### d) 4-(3-Hydroxy-4,6-bis-trifluoromethyl-pyridin-2-yl)-4-oxo-butyric acid

The title compound was prepared from 4-(3-hydroxy-4,6-bis-trifluoromethyl-pyridin-2-yl)-4-oxo-butyric acid ethyl ester (see example 4c) in analogy to example 1g, MS-(−)-ion, M−H=330.20.

### Example 5

### 4-(4-Cyano-3-hydroxy-6-methylsulfanyl-pyridin-2-yl)-4-oxo-butyric acid

### a) 4-(4-Bromo-3-hydroxy-6-methylsulfanyl-pyridin-2-yl)-4-oxo-butyric acid ethyl ester

To a round-bottom-flask were added 4-(4,6-dibromo-3-hydroxy-pyridin-2-yl)-4-oxo-butyric acid ethyl ester (267 mg, 0.7 mmol, see Example if), Pd₂(dba)₃ (25 mg, 0.035 mmol), Xantphos (40 mg, 0.07 mmol), sodium thiomethoxide (147 mg, 2.1 mmol), and N,N-Diisopropylethylamine (0.49 mL, 2.8 mmol) in anhydrous 1,4-dioxane (7 mL). The mixture was heated at 105° C. under N₂ atmosphere for 3 hours. The reaction mixture was then allowed to reach ambient temperature, diluted in 30 mL H₂O, treated with 1N HCl to pH=3, and extracted with ethyl acetate (50 mL×3). The combined organics were washed with brine (100 mL) and dried over sodium sulfate, filtered and evaporated in vacuo to afford the crude product. This material was purified by flash chromatography eluting with ethyl acetate/hexane (0%-15%) to give the title compound (4-(4-bromo-3-hydroxy-6-methylsulfanyl-pyridin-2-yl)-4-oxo-butyric acid ethyl ester, MS-(−)-ion, M−H=348.26, 346.32) and (4-(3-hydroxy-4,6-bis-methylsulfanyl-pyridin-2-yl)-4-oxo-butyric acid ethyl ester, MS-(+)-ion, M+H=316.34).

### b) 4-(4-Cyano-3-hydroxy-6-methylsulfanyl-pyridin-2-yl)-4-oxo-butyric acid ethyl ester

The title compound was prepared from 4-(4-bromo-3-hydroxy-6-methylsulfanyl-pyridin-2-yl)-4-oxo-butyric acid ethyl ester (see example 5a) and zinc(II) cyanide in analogy to example 2b, MS-(−)-ion, M−H=293.35.

### c) 4-(4-Cyano-3-hydroxy-6-methylsulfanyl-pyridin-2-yl)-4-oxo-butyric acid

The title compound was prepared from 4-(4-cyano-3-hydroxy-6-methylsulfanyl-pyridin-2-yl)-4-oxo-butyric acid ethyl ester (see example 5b) in analogy to example 1g, MS-(−)-ion, M−H=265.25.

### Example 6

### 4-(4-Cyano-3-hydroxy-6-methyl-pyridin-2-yl)-4-oxo-butyric acid

### a) 4-(3-Hydroxy-6-methyl-pyridin-2-yl)-4-oxo-butyric acid ethyl ester

To a round-bottom-flask were added 4-(6-bromo-3-hydroxy-pyridin-2-yl)-4-oxo-butyric acid ethyl ester (403 mg, 1.33 mmol, see Example 2a), PdCl₂(PPh₃)₂(142 mg, 0.20 mmol), and tetramethyltin (0.55 mL, 4.0 mmol) in anhydrous DMF (10 mL). The mixture was heated at 120° C. under N₂ atmosphere for 2 hours. The reaction mixture was then allowed to reach ambient temperature, diluted in 50 mL of H₂O, and extracted with ethyl acetate (50 mL×3). The combined organics were washed with brine (100 mL) and dried over sodium sulfate, filtered and evaporated in vacuo to afford the crude product. This material was purified by flash chromatography eluting with ethyl acetate/hexane (0%-10%) to give the title compound. MS-(+)-ion, M+H=238.37.

### b) 4-(4-Bromo-3-hydroxy-6-methyl-pyridin-2-yl)-4-oxo-butyric acid ethyl ester

The title compound was prepared from 4-(3-Hydroxy-6-methyl-pyridin-2-yl)-4-oxo-butyric acid ethyl ester (see example 6a) and bromine in analogy to example 2c, MS-(−)-ion, M−H=314.35.

### c) 4-(4-Cyano-3-hydroxy-6-methyl-pyridin-2-yl)-4-oxo-butyric acid ethyl ester

The title compound was prepared from 4-(4-Bromo-3-hydroxy-6-methyl-pyridin-2-yl)-4-oxo-butyric acid ethyl ester (see example 6b) and zinc(II) cyanide in analogy to example 2b, MS-(−)-ion, M−H=261.30.

### d) 4-(4-Cyano-3-hydroxy-6-methyl-pyridin-2-yl)-4-oxo-butyric acid

The title compound was prepared from 4-(4-Cyano-3-hydroxy-6-methyl-pyridin-2-yl)-4-oxo-butyric acid ethyl ester (see example 6c) in analogy to example 1g, MS-(−)-ion, M−H=234.36.

### Example 7

### 4-(4-Cyano-3-hydroxy-6-phenyl-pyridin-2-yl)-4-oxo-butyric acid

### a) 4-(3-Hydroxy-6-phenyl-pyridin-2-yl)-4-oxo-butyric acid ethyl ester

The title compound was prepared from 4-(6-Bromo-3-hydroxy-pyridin-2-yl)-4-oxo-butyric acid ethyl ester (410 mg, 1.35 mmol, see Example 2a), and tributylphenylstannane (2.70 mmol, 2.0 eq, 0.88 mL) in analogy to example 6a, MS-(−)-ion, M−H=298.46.

### b) 4-(4-Bromo-3-hydroxy-6-phenyl-pyridin-2-yl)-4-oxo-butyric acid ethyl ester

The title compound was prepared from 4-(3-Hydroxy-6-phenyl-pyridin-2-yl)-4-oxo-butyric acid ethyl ester (see example 7a) and bromine in analogy to example 2c, MS-(+)-ion, M+H=378.39, 380.46.

### c) 4-(4-Cyano-3-hydroxy-6-phenyl-pyridin-2-yl)-4-oxo-butyric acid ethyl ester

The title compound was prepared from 4-(4-Bromo-3-hydroxy-6-phenyl-pyridin-2-yl)-4-oxo-butyric acid ethyl ester (see example 7b) and zinc(II) cyanide in analogy to example 2b, MS-(+)-ion, M+H=325.40.

### d) 4-(4-Cyano-3-hydroxy-6-phenyl-pyridin-2-yl)-4-oxo-butyric acid

The title compound was prepared from 4-(4-Cyano-3-hydroxy-6-phenyl-pyridin-2-yl)-4-oxo-butyric acid ethyl ester (see example 7c) in analogy to example 1g, MS-(+)-ion, M+H=297.70.

### Example 8

### 4-(6-Cyano-3-hydroxy-4-trifluoromethyl-pyridin-2-yl)-4-oxo-butyric acid

### a) 4-(3-Benzyloxy-4-bromo-6-cyano-pyridin-2-yl)-4-oxo-butyric acid ethyl ester

The title compound was prepared from 4-(4-Bromo-6-cyano-3-hydroxy-pyridin-2-yl)-4-oxo-butyric acid ethyl ester (see Example 2c) and benzyl bromide in analogy to example 1b. ¹H NMR (200 MHz, CDCl₃) δ=8.05 (s, 1H), 7.48-7.37 (m, 6H), 5.15 (s, 2H), 4.14 (q, J=7.2 Hz, 2H), 3.34 (t, J=6.6 Hz, 2H), 2.74 (t, J=6.6 Hz, 2H), 1.26 (t, J=7.2 Hz, 3H).

### b) 4-(3-Benzyloxy-6-cyano-4-trifluoromethyl-pyridin-2-yl)-4-oxo-butyric acid ethyl ester

The title compound was prepared from 4-(3-Benzyloxy-4-bromo-6-cyano-pyridin-2-yl)-4-oxo-butyric acid ethyl ester (see example 8a) and 2,2-difluoro-2-(fluorosulfonyl)acetate in analogy to example 4b. ¹H NMR (200 MHz, CDCl₃) δ=8.02 (s, 1H), 7.40-7.38 (m, 6H), 5.14 (s, 2H), 4.14 (q, J=7.2 Hz, 2H), 3.45 (t, J=6.6 Hz, 2H), 2.79 (t, J=6.6 Hz, 2H), 1.25 (t, J=7.2 Hz, 3H).

### c) 4-(6-Cyano-3-hydroxy-4-trifluoromethyl-pyridin-2-yl)-4-oxo-butyric acid ethyl ester

The title compound was prepared from 4-(3-Benzyloxy-6-cyano-4-trifluoromethyl-pyridin-2-yl)-4-oxo-butyric acid ethyl ester (see example 8b) in analogy to example 4c, MS-(−)-ion, M−H=315.52.

### d) 4-(6-Cyano-3-hydroxy-4-trifluoromethyl-pyridin-2-yl)-4-oxo-butyric acid

The title compound was prepared from 4-(6-Cyano-3-hydroxy-4-trifluoromethyl-pyridin-2-yl)-4-oxo-butyric acid ethyl ester (see example 8c) in analogy to example 1g, MS-(+)-ion, M+H=287.33.

### Example 9

### 4-[4-Cyano-3-hydroxy-6-(4-methoxy-phenyl)-pyridin-2-yl]-4-oxo-butyric acid

### a) 4-[3-Hydroxy-6-(4-methoxy-phenyl)-pyridin-2-yl]-4-oxo-butyric acid ethyl ester

A round bottom flask was charged with 4-(6-bromo-3-hydroxy-pyridin-2-yl)-4-oxo-butyric acid ethyl ester (151 mg, 0.50 mmol, see Example 2a), 4-methoxyphenylboronic acid (114 mg, 0.75 mmol, 1.5 eq), S-Phos (12.3 mg, 0.06 eq, 0.03 mmol), palladium acetate (9.0 mg, 0.04 mmol, 0.08 eq), and tripotassium phosphate (212 mg, 1.0 mmol, 2 eq). The flask was evacuated and backfilled with nitrogen three times. Anhydrous toluene (3 mL) and water (18 mg, 1.0 mmol, 2.0 eq) were added to the reaction. The reaction was heated at 100° C. for 2 hours, until TLC shows the completion of the reaction. After cooling back to room temperature, the reaction was diluted with water (20 mL) and acidified to pH=4 with 1N HCl. The mixture was extracted with ethyl acetate (3×15 mL). The combined extracts were washed with brine (20 mL), dried over sodium sulfate, filtered and evaporated in vacuo to afford the crude product. This material was purified by flash chromatography eluting with ethyl acetate/hexane (0%-15%) to give the title compound. MS-(+)-ion, M+H=330.23.

### b) 4-[4-Bromo-3-hydroxy-6-(4-methoxy-phenyl)-pyridin-2-yl]-4-oxo-butyric acid ethyl ester

The title compound was prepared from 4-[3-Hydroxy-6-(4-methoxy-phenyl)-pyridin-2-yl]-4-oxo-butyric acid ethyl ester (see example 9a) and bromine in analogy to example 2c, MS-(+)-ion, M+H=410.10.

### c) 4-[4-Cyano-3-hydroxy-6-(4-methoxy-phenyl)-pyridin-2-yl]-4-oxo-butyric acid ethyl ester

The title compound was prepared from 4-[4-Bromo-3-hydroxy-6-(4-methoxy-phenyl)-pyridin-2-yl]-4-oxo-butyric acid ethyl ester (see example 9b) and zinc(II) cyanide in analogy to example 2b, MS-(−)-ion, M−H=353.30.

### d) 4-[4-Cyano-3-hydroxy-6-(4-methoxy-phenyl)-pyridin-2-yl]-4-oxo-butyric acid

The title compound was prepared from 4-[4-Cyano-3-hydroxy-6-(4-methoxy-phenyl)-pyridin-2-yl]-4-oxo-butyric acid ethyl ester (see example 9c) in analogy to example 1g, MS-(+)-ion, M+H=325.34.

### Example 10

### 4-(6-Benzyl-4-cyano-3-hydroxy-pyridin-2-yl)-4-oxo-butyric acid

### a) 4-(6-Benzyl-3-hydroxy-pyridin-2-yl)-4-oxo-butyric acid ethyl ester

At 0° C., to a solution of 4-(6-Bromo-3-hydroxy-pyridin-2-yl)-4-oxo-butyric acid ethyl ester (121 mg, 0.4 mmol, see Example 2a), Pd(OAc)₂ (4.5 mg, 0.02 mmol) and S-Phos (16.4 mg, 0.04 mmol) in dry THF (3 mL) was added dropwise a solution of benzylzinc(II) bromide in THF (1.0 mmol, 2 mL, 0.5 M) under nitrogen atmosphere. The reaction mixture was stirred at room temperature for 18 hours, then quenched by half saturated NH₄Cl aqueous solution (20 mL) and extracted with ethyl acetate (20 mL×3). The combined organic layers were dried (Na₂SO₄), concentrated under reduced pressure, and purified by flash column chromatography to give the 4-(6-benzyl-3-hydroxy-pyridin-2-yl)-4-oxo-butyric acid ethyl ester. MS-(+)-ion, M+H=314.39.

### b) 4-(6-Benzyl-4-bromo-3-hydroxy-pyridin-2-yl)-4-oxo-butyric acid ethyl ester

The title compound was prepared from 4-(6-Benzyl-3-hydroxy-pyridin-2-yl)-4-oxo-butyric acid ethyl ester (see example 10a) and bromine in analogy to example 2c, MS-(+)-ion, M+H=394.11.

### c) 4-(6-Benzyl-4-cyano-3-hydroxy-pyridin-2-yl)-4-oxo-butyric acid ethyl ester

The title compound was prepared from 4-(6-Benzyl-4-bromo-3-hydroxy-pyridin-2-yl)-4-oxo-butyric acid ethyl ester (see example 10b) and zinc(II) cyanide in analogy to example 2b, MS-(−)-ion, M−H=339.25.

### d) 4-(6-Benzyl-4-cyano-3-hydroxy-pyridin-2-yl)-4-oxo-butyric acid

The title compound was prepared from 4-(6-Benzyl-4-cyano-3-hydroxy-pyridin-2-yl)-4-oxo-butyric acid ethyl ester (see example 10c) in analogy to example 1g, MS-(−)-ion, M−H=309.30.

### Example 11

### 4-(4-Cyano-5-hydroxy-[2,2′]bipyridinyl-6-yl)-4-oxo-butyric acid

### a) 4-(5-Hydroxy-[2,2]bipyridinyl-6-yl)-4-oxo-butyric acid ethyl ester

The title compound was prepared from 4-(6-Bromo-3-hydroxy-pyridin-2-yl)-4-oxo-butyric acid ethyl ester (see Example 2a) and 2-tributylstannanyl-pyridine in analogy to example 6a, MS-(+)-ion, M+H=301.26.

### b) 4-(4-Bromo-5-hydroxy-[2,2′]bipyridinyl-6-yl)-4-oxo-butyric acid ethyl ester

The title compound was prepared from 4-(5-Hydroxy-[2,2′]bipyridinyl-6-yl)-4-oxo-butyric acid ethyl ester (see example 11a) and bromine in analogy to example 2c, MS-(+)-ion, M+H=381.12, 379.14.

### c) 4-(4-Cyano-5-hydroxy-[2,2′]bipyridinyl-6-yl)-4-oxo-butyric acid ethyl ester

The title compound was prepared from 4-(4-Bromo-5-hydroxy-[2,2′]bipyridinyl-6-yl)-4-oxo-butyric acid ethyl ester (see example 11b) and zinc(II) cyanide in analogy to example 2b, MS-(+)-ion, M+H=326.25.

### d) 4-(4-Cyano-5-hydroxy-[2,2′]bipyridinyl-6-yl)-4-oxo-butyric acid

The title compound was prepared from 4-(4-Cyano-5-hydroxy-[2,2′]bipyridinyl-6-yl)-4-oxo-butyric acid ethyl ester (see example 11c) in analogy to example 1g, MS-(+)-ion, M+H=298.18.

### Example 12

### 4-(4-Cyano-5-hydroxy-[2,4′]bipyridinyl-6-yl)-4-oxo-butyric acid

### a) 4-(5-Hydroxy-[2,4]bipyridinyl-6-yl)-4-oxo-butyric acid ethyl ester

The title compound was prepared from 4-(6-Bromo-3-hydroxy-pyridin-2-yl)-4-oxo-butyric acid ethyl ester (see Example 2a) and 4-tributylstannanyl-pyridine in analogy to example 6a, MS-(+)-ion, M+H=301.26.

### b) 4-(4-Bromo-5-hydroxy-[2,4′]bipyridinyl-6-yl)-4-oxo-butyric acid ethyl ester

The title compound was prepared from 4-(5-Hydroxy-[2,4′]bipyridinyl-6-yl)-4-oxo-butyric acid ethyl ester (see example 12a) and bromine in analogy to example 2c, MS-(+)-ion, M+H=381.14.

### c) 4-(4-Cyano-5-hydroxy-[2,4]bipyridinyl-6-yl)-4-oxo-butyric acid ethyl ester

The title compound was prepared from 4-(4-Bromo-5-hydroxy-[2,4′]bipyridinyl-6-yl)-4-oxo-butyric acid ethyl ester (see example 12b) and zinc(II) cyanide in analogy to example 2b, MS-(+)-ion, M+H=326.29.

### d) 4-(4-Cyano-5-hydroxy-[2,4′]bipyridinyl-6-yl)-4-oxo-butyric acid

The title compound was prepared from 4-(4-Cyano-5-hydroxy-[2,4′]bipyridinyl-6-yl)-4-oxo-butyric acid ethyl ester (see example 12c) in analogy to example 1g, MS-(+)-ion, M+H=298.20.

### Example 13

### 4-(4-Cyano-5-hydroxy-[2,3′]bipyridinyl-6-yl)-4-oxo-butyric acid

### a) 4-(5-Hydroxy-[2,3]bipyridinyl-6-yl)-4-oxo-butyric acid ethyl ester

The title compound was prepared from 4-(6-Bromo-3-hydroxy-pyridin-2-yl)-4-oxo-butyric acid ethyl ester (see Example 2a) and 3-tributylstannanyl-pyridine in analogy to example 6a, MS-(+)-ion, M+H=301.26.

### b) 4-(4-Bromo-5-hydroxy-[2,3]bipyridinyl-6-yl)-4-oxo-butyric acid ethyl ester

The title compound was prepared from 4-(5-Hydroxy-[2,4′]bipyridinyl-6-yl)-4-oxo-butyric acid ethyl ester (see example 13a) and bromine in analogy to example 2c, MS-(+)-ion, M+H=379.11, 381.12.

### c) 4-(4-Cyano-5-hydroxy-[2,3]bipyridinyl-6-yl)-4-oxo-butyric acid ethyl ester

The title compound was prepared from 4-(4-Bromo-5-hydroxy-[2,3′]bipyridinyl-6-yl)-4-oxo-butyric acid ethyl ester (see example 13b) and zinc(II) cyanide in analogy to example 2b, MS-(+)-ion, M+H=326.22.

### d) 4-(4-Cyano-5-hydroxy-[2,3′]bipyridinyl-6-yl)-4-oxo-butyric acid

The title compound was prepared from 4-(4-Cyano-5-hydroxy-[2,3′]bipyridinyl-6-yl)-4-oxo-butyric acid ethyl ester (see example 13c) in analogy to example 1g, MS-(+)-ion, M+H=298.18.

### Example 14

### 4-[4-Cyano-3-hydroxy-6-(2-methoxy-phenyl)-pyridin-2-yl]-4-oxo-butyric acid

### a) 4-[3-Hydroxy-6-(2-methoxy-phenyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-methoxyphenylboronic acid in analogy to example 9a, MS-(+)-ion, M+H=301.26.

### b) 4-[4-Bromo-3-hydroxy-6-(2-methoxy-phenyl)-pyridin-2-yl]-4-oxo-butyric acid ethyl ester

The title compound was prepared from 4-[3-Hydroxy-6-(2-methoxy-phenyl)-pyridin-2-yl]-4-oxo-butyric acid ethyl ester (see example 14a) and bromine in analogy to example 2c, MS-(+)-ion, M+H=410.10.

### c) 4-[4-Cyano-3-hydroxy-6-(2-methoxy-phenyl)-pyridin-2-yl]-4-oxo-butyric acid ethyl ester

The title compound was prepared from 4-[4-Bromo-3-hydroxy-6-(2-methoxy-phenyl)-pyridin-2-yl]-4-oxo-butyric acid ethyl ester (see example 14b) and zinc(II) cyanide in analogy to example 2b, MS-(+)-ion, M+H=355.21.

### d) 4-[4-Cyano-3-hydroxy-6-(2-methoxy-phenyl)-pyridin-2-yl]-4-oxo-butyric acid

The title compound was prepared from 4-[4-Cyano-3-hydroxy-6-(2-methoxy-phenyl)-pyridin-2-yl]-4-oxo-butyric acid ethyl ester (see example 14c) in analogy to example 1g, MS-(+)-ion, M+H=327.19.

### Example 15

### 4-(4-Cyano-3-hydroxy-6-phenethyl-pyridin-2-yl)-4-oxo-butyric acid

### a) 4-(3-Hydroxy-6-phenylethynyl-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and tributyl-phenylethynyl-stannane in analogy to example 6a, MS-(+)-ion, M+H=324.27.

### b) 4-(3-Hydroxy-6-phenethyl-pyridin-2-yl)-4-oxo-butyric acid ethyl ester

The title compound was prepared from 4-(3-Hydroxy-6-phenylethynyl-pyridin-2-yl)-4-oxo-butyric acid ethyl ester (see Example 15a) and Pd/C in analogy to example 1e, MS-(+)-ion, M+H=328.29.

### c) 4-(4-Bromo-3-hydroxy-6-phenethyl-pyridin-2-yl)-4-oxo-butyric acid ethyl ester

The title compound was prepared from 4-(3-Hydroxy-6-phenethyl-pyridin-2-yl)-4-oxo-butyric acid ethyl ester (see example 15b) and bromine in analogy to example 2c, MS-(+)-ion, M+H=406.14, 408.09.

### d) 4-(4-Cyano-3-hydroxy-6-phenethyl-pyridin-2-yl)-4-oxo-butyric acid ethyl ester

The title compound was prepared from 4-(4-Bromo-3-hydroxy-6-phenethyl-pyridin-2-yl)-4-oxo-butyric acid ethyl ester (see example 15c) and zinc(II) cyanide in analogy to example 2b, MS-(+)-ion, M+H=353.25.

### e) 4-(4-Cyano-3-hydroxy-6-phenethyl-pyridin-2-yl)-4-oxo-butyric acid

The title compound was prepared from 4-(4-Cyano-3-hydroxy-6-phenethyl-pyridin-2-yl)-4-oxo-butyric acid ethyl ester (see example 15d) in analogy to example 1g, MS-(+)-ion, M+H=325.25.

### Example 16

### 4-[4-Cyano-3-hydroxy-6-(1-methyl-1H-pyrazol-4-yl)-pyridin-2-yl]-4-oxo-butyric acid

### a) 4-[3-Hydroxy-6-(1-methyl-1H-pyrazol-4-yl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 1-methyl-1H-pyrazole-4-boronic acid in analogy to example 9a, MS-(+)-ion, M+H=304.24.

### b) 4-[4-Bromo-3-hydroxy-6-(1-methyl-1H-pyrazol-4-yl)-pyridin-2-yl]-4-oxo-butyric acid ethyl ester

The title compound was prepared from 4-[3-Hydroxy-6-(1-methyl-1H-pyrazol-4-yl)-pyridin-2-yl]-4-oxo-butyric acid ethyl ester (see example 16a) and bromine in analogy to example 2c, MS-(+)-ion, M+H=382.09, 384.10.

### c) 4-[4-Cyano-3-hydroxy-6-(1-methyl-1H-pyrazol-4-yl)-pyridin-2-yl]-4-oxo-butyric acid ethyl ester

The title compound was prepared from 4-[4-Bromo-3-hydroxy-6-(1-methyl-1H-pyrazol-4-yl)-pyridin-2-yl]-4-oxo-butyric acid ethyl ester (see example 16b) and zinc(II) cyanide in analogy to example 2b, MS-(+)-ion, M+H=329.24.

### d) 4-[4-Cyano-3-hydroxy-6-(1-methyl-1H-pyrazol-4-yl)-pyridin-2-yl]-4-oxo-butyric acid

The title compound was prepared from 4-[4-Cyano-3-hydroxy-6-(1-methyl-1H-pyrazol-4-yl)-pyridin-2-yl]-4-oxo-butyric acid ethyl ester (see example 16c) in analogy to example 1g, MS-(+)-ion, M+H=301.22.

### Example 17

### 4-[6-(4-Chloro-benzyl)-4-cyano-3-hydroxy-pyridin-2-yl]-4-oxo-butyric acid

### a) 4-[6-(4-Chloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-chloro-benzylzinc(II) chloride in THF (0.5 M) in analogy to example 10a, MS-(+)-ion, M+H=348.26.

### b) 4-[4-Bromo-6-(4-chloro-benzyl)-3-hydroxy-pyridin-2-yl]-4-oxo-butyric acid ethyl ester

The title compound was prepared from 4-[6-(4-Chloro-benzyl)-3-hydroxy-pyridin-2-yl]-4-oxo-butyric acid ethyl ester (see example 17a) and bromine in analogy to example 2c, MS-(+)-ion, M+H=426.04, 428.05.

### c) 4-[6-(4-Chloro-benzyl)-4-cyano-3-hydroxy-pyridin-2-yl]-4-oxo-butyric acid ethyl ester

The title compound was prepared from 4-[4-Bromo-6-(4-chloro-benzyl)-3-hydroxy-pyridin-2-yl]-4-oxo-butyric acid ethyl ester (see example 17b) and zinc(II) cyanide in analogy to example 2b, MS-(+)-ion, M+H=373.15.

### d) 4-[6-(4-Chloro-benzyl)-4-cyano-3-hydroxy-pyridin-2-yl]-4-oxo-butyric acid

The title compound was prepared from 4-[6-(4-Chloro-benzyl)-4-cyano-3-hydroxy-pyridin-2-yl]-4-oxo-butyric acid ethyl ester (see example 17c) in analogy to example 1g, MS-(+)-ion, M+H=345.15.

### Example 18

### 4-[6-(3-Chloro-benzyl)-4-cyano-3-hydroxy-pyridin-2-yl]-4-oxo-butyric acid

### a) 4-[6-(3-Chloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-chloro-benzylzinc(II) chloride in THF (0.5 M) in analogy to example 10a, MS-(+)-ion, M+H=348.19.

### b) 4-[4-Bromo-6-(3-chloro-benzyl)-3-hydroxy-pyridin-2-yl]-4-oxo-butyric acid ethyl ester

The title compound was prepared from 4-[6-(3-Chloro-benzyl)-3-hydroxy-pyridin-2-yl]-4-oxo-butyric acid ethyl ester (see example 18a) and bromine in analogy to example 2c, MS-(+)-ion, M+H=428.05, 426.04.

### c) 4-[6-(3-Chloro-benzyl)-4-cyano-3-hydroxy-pyridin-2-yl]-4-oxo-butyric acid ethyl ester

The title compound was prepared from 4-[4-Bromo-6-(3-chloro-benzyl)-3-hydroxy-pyridin-2-yl]-4-oxo-butyric acid ethyl ester (see example 18b) and zinc(II) cyanide in analogy to example 2b, MS-(+)-ion, M+H=373.15.

### d) 4-[6-(3-Chloro-benzyl)-4-cyano-3-hydroxy-pyridin-2-yl]-4-oxo-butyric acid

The title compound was prepared from 4-[6-(3-Chloro-benzyl)-4-cyano-3-hydroxy-pyridin-2-yl]-4-oxo-butyric acid ethyl ester (see example 18c) in analogy to example 1g, MS-(−)-ion, M−H=343.14.

### Example 19

### 4-[4-Cyano-6-(4-fluoro-phenyl)-3-hydroxy-pyridin-2-yl]-4-oxo-butyric acid

### a) 4-[6-(4-Fluoro-phenyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-fluorophenylboronic acid in analogy to example 9a, MS-(+)-ion, M+H=318.

### b) 4-[4-Bromo-6-(4-fluoro-phenyl)-3-hydroxy-pyridin-2-yl]-4-oxo-butyric acid ethyl ester

The title compound was prepared from 4-[6-(4-Fluoro-phenyl)-3-hydroxy-pyridin-2-yl]-4-oxo-butyric acid ethyl ester (see example 19a) and bromine in analogy to example 2c, MS-(+)-ion, M+H=396, 398.

### c) 4-[4-Cyano-6-(4-fluoro-phenyl)-3-hydroxy-pyridin-2-yl]-4-oxo-butyric acid ethyl ester

The title compound was prepared from 4-[4-Bromo-6-(4-fluoro-phenyl)-3-hydroxy-pyridin-2-yl]-4-oxo-butyric acid ethyl ester (see example 19b) and zinc(II) cyanide in analogy to example 2b, MS-(+)-ion, M+H=343.13.

### d) 4-[4-Cyano-6-(4-fluoro-phenyl)-3-hydroxy-pyridin-2-yl]-4-oxo-butyric acid

The title compound was prepared from 4-[4-Cyano-6-(4-fluoro-phenyl)-3-hydroxy-pyridin-2-yl]-4-oxo-butyric acid ethyl ester (see example 19c) in analogy to example 1g, MS-(−)-ion, M−H=313.19.

### Example 20

### 4-[4-Cyano-6-(3-fluoro-phenyl)-3-hydroxy-pyridin-2-yl]-4-oxo-butyric acid

### a) 4-[6-(3-Fluoro-phenyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-fluorophenylboronic acid in analogy to example 9a, MS-(+)-ion, M+H=318.

### b) 4-[4-Bromo-6-(3-fluoro-phenyl)-3-hydroxy-pyridin-2-yl]-4-oxo-butyric acid ethyl ester

The title compound was prepared from 4-[6-(3-Fluoro-phenyl)-3-hydroxy-pyridin-2-yl]-4-oxo-butyric acid ethyl ester (see example 20a) and bromine in analogy to example 2c, MS-(+)-ion, M+H=396, 398.

### c) 4-[4-Cyano-6-(3-fluoro-phenyl)-3-hydroxy-pyridin-2-yl]-4-oxo-butyric acid ethyl ester

The title compound was prepared from 4-[4-Bromo-6-(3-fluoro-phenyl)-3-hydroxy-pyridin-2-yl]-4-oxo-butyric acid ethyl ester (see example 20b) and zinc(II) cyanide in analogy to example 2b, MS-(+)-ion, M+H=343.13.

### d) 4-[4-Cyano-6-(4-fluoro-phenyl)-3-hydroxy-pyridin-2-yl]-4-oxo-butyric acid

The title compound was prepared from 4-[4-Cyano-6-(3-fluoro-phenyl)-3-hydroxy-pyridin-2-yl]-4-oxo-butyric acid ethyl ester (see example 20c) in analogy to example 1g, MS-(−)-ion, M−H=313.19.

### Example 21

### 4-[6-(2-Chloro-benzyl)-4-cyano-3-hydroxy-pyridin-2-yl]-4-oxo-butyric acid

### a) 4-[6-(2-Chloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-chloro-benzylzinc(II) chloride in THF (0.5 M) in analogy to example 10a. ¹H NMR (200 MHz, CDCl₃) δ=11.46 (s, 1H), 7.38-7.20 (m, 6H), 4.24 (s, 2H), 4.16 (q, J=7.2 Hz, 2H), 3.56 (t, J=6.6 Hz, 2H), 2.71 (t, J=6.6 Hz, 2H), 1.26 (t, J=7.2 Hz, 3H).

### b) 4-[4-Bromo-6-(2-chloro-benzyl)-3-hydroxy-pyridin-2-yl]-4-oxo-butyric acid ethyl ester

The title compound was prepared from 4-[6-(2-Chloro-benzyl)-3-hydroxy-pyridin-2-yl]-4-oxo-butyric acid ethyl ester (see example 21a) and bromine in analogy to example 2c, MS-(+)-ion, M+H=427.92, 428.91.

### c) 4-[6-(2-Chloro-benzyl)-4-cyano-3-hydroxy-pyridin-2-yl]-4-oxo-butyric acid ethyl ester

The title compound was prepared from 4-[4-Bromo-6-(2-chloro-benzyl)-3-hydroxy-pyridin-2-yl]-4-oxo-butyric acid ethyl ester (see example 21b) and zinc(II) cyanide in analogy to example 2b, MS-(+)-ion, M+H=373.08.

### d) 4-[6-(2-Chloro-benzyl)-4-cyano-3-hydroxy-pyridin-2-yl]-4-oxo-butyric acid

The title compound was prepared from 4-[6-(2-Chloro-benzyl)-4-cyano-3-hydroxy-pyridin-2-yl]-4-oxo-butyric acid ethyl ester (see example 21c) in analogy to example 1g, MS-(+)-ion, M+H=345.12.

### Example 22

### 4-[6-(4-Chloro-phenyl)-4-cyano-3-hydroxy-pyridin-2-yl]-4-oxo-butyric acid

### a) 4-[6-(4-Chloro-phenyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-chlorophenylboronic acid in analogy to example 9a, MS-(+)-ion, M+H=334.

### b) 4-[4-Bromo-6-(4-chloro-phenyl)-3-hydroxy-pyridin-2-yl]-4-oxo-butyric acid ethyl ester

The title compound was prepared from 4-[6-(4-Chloro-phenyl)-3-hydroxy-pyridin-2-yl]-4-oxo-butyric acid ethyl ester (see example 22a) and bromine in analogy to example 2c, MS-(+)-ion, M+H=412, 414.

### c) 4-[6-(4-Chloro-phenyl)-4-cyano-3-hydroxy-pyridin-2-yl]-4-oxo-butyric acid ethyl ester

The title compound was prepared from 4-[4-Bromo-6-(4-chloro-phenyl)-3-hydroxy-pyridin-2-yl]-4-oxo-butyric acid ethyl ester (see example 22b) and zinc(II) cyanide in analogy to example 2b, MS-(+)-ion, M+H=373.15.

### d) 4-[6-(4-Chloro-phenyl)-4-cyano-3-hydroxy-pyridin-2-yl]-4-oxo-butyric acid

The title compound was prepared from 4-[6-(4-Chloro-phenyl)-4-cyano-3-hydroxy-pyridin-2-yl]-4-oxo-butyric acid ethyl ester (see example 22c) in analogy to example 1g, MS-(−)-ion, M−H=329.13.

### Example 23

### 4-[6-(3-Chloro-phenyl)-4-cyano-3-hydroxy-pyridin-2-yl]-4-oxo-butyric acid

### a) 4-[6-(3-chloro-phenyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-chlorophenylboronic acid in analogy to example 9a, MS-(+)-ion, M+H=334.

### b) 4-[4-Bromo-6-(3-chloro-phenyl)-3-hydroxy-pyridin-2-yl]-4-oxo-butyric acid ethyl ester

The title compound was prepared from 4-[6-(3-chloro-phenyl)-3-hydroxy-pyridin-2-yl]-4-oxo-butyric acid ethyl ester (see example 23a) and bromine in analogy to example 2c, MS-(+)-ion, M+H=412, 414.

### c) 4-[6-(3-Chloro-phenyl)-4-cyano-3-hydroxy-pyridin-2-yl]-4-oxo-butyric acid ethyl ester

The title compound was prepared from 4-[4-Bromo-6-(3-chloro-phenyl)-3-hydroxy-pyridin-2-yl]-4-oxo-butyric acid ethyl ester (see example 23b) and zinc(II) cyanide in analogy to example 2b, MS-(+)-ion, M+H=343.13.

### d) 4-[6-(3-Chloro-phenyl)-4-cyano-3-hydroxy-pyridin-2-yl]-4-oxo-butyric acid

The title compound was prepared from 4-[6-(3-Chloro-phenyl)-4-cyano-3-hydroxy-pyridin-2-yl]-4-oxo-butyric acid ethyl ester (see example 23c) in analogy to example 1g, MS-(−)-ion, M−H=329.14.

### Example 24

### 4-(4-Cyano-3-hydroxy-6-naphthalen-2-ylmethyl-pyridin-2-yl)-4-oxo-butyric acid

### a) 4-(3-Hydroxy-6-naphthalen-2-ylmethyl-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and (2-naphthylmethyl)zinc bromide solution in THF (0.5 M) in analogy to example 10a, MS-(+)-ion, M+H=364.14.

### b) 4-(4-Bromo-3-hydroxy-6-naphthalen-2-ylmethyl-pyridin-2-yl)-4-oxo-butyric acid ethyl ester

The title compound was prepared from 4-(3-Hydroxy-6-naphthalen-2-ylmethyl-pyridin-2-yl)-4-oxo-butyric acid ethyl ester (see example 24a) and bromine in analogy to example 2c. ¹H NMR (200 MHz, CDCl₃) δ=12.14 (s, 1H), 7.84-7.34 (m, 8H), 4.22 (s, 2H), 4.16 (q, J=7.2 Hz, 2H), 3.65 (t, J=6.6 Hz, 2H), 2.75 (t, J=6.6 Hz, 2H), 1.28 (t, J=7.2 Hz, 3H).

### c) 4-(4-Cyano-3-hydroxy-6-naphthalen-2-ylmethyl-pyridin-2-yl)-4-oxo-butyric acid ethyl ester

The title compound was prepared from 4-(4-Bromo-3-hydroxy-6-naphthalen-2-ylmethyl-pyridin-2-yl)-4-oxo-butyric acid ethyl ester (see example 24b) and zinc(II) cyanide in analogy to example 2b, MS-(+)-ion, M+H=389.11.

### d) 4-(4-Cyano-3-hydroxy-6-naphthalen-2-ylmethyl-pyridin-2-yl)-4-oxo-butyric acid

The title compound was prepared from 4-(4-Cyano-3-hydroxy-6-naphthalen-2-ylmethyl-pyridin-2-yl)-4-oxo-butyric acid ethyl ester (see example 24c) in analogy to example 1g, MS-(−)-ion, M−H=361.10.

### Example 25

### 4-(4-Cyano-6-cyclohexyl-3-hydroxy-pyridin-2-yl)-4-oxo-butyric acid

### a) 4-(6-Cyclohex-1-enyl-3-hydroxy-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and (cyclohex-1-en-1-yl)boronic acid in analogy to example 9a.

¹H NMR (200 MHz, CDCl₃) δ=11.98 (s, 1H), 7.79-7.05 (m, 3H), 4.16 (q, J=7.0 Hz, 2H), 3.61 (t, J=6.6 Hz, 2H), 2.76 (t, J=6.6 Hz, 2H), 2.70-2.60 (m, 1H), 1.96-1.23 (m, 10H).

### b) 4-(6-Cyclohexyl-3-hydroxy-pyridin-2-yl)-4-oxo-butyric acid ethyl ester

The title compound was prepared from 4-(6-Cyclohex-1-enyl-3-hydroxy-pyridin-2-yl)-4-oxo-butyric acid ethyl ester (see Example 25a) and Pd/C in analogy to example 1e, MS-(+)-ion, M+H=306.25.

### c) 4-(4-Bromo-6-cyclohexyl-3-hydroxy-pyridin-2-yl)-4-oxo-butyric acid ethyl ester

The title compound was prepared from 4-(6-Cyclohexyl-3-hydroxy-pyridin-2-yl)-4-oxo-butyric acid ethyl ester (see example 25b) and bromine in analogy to example 2c, MS-(+)-ion, M+H=386.05.

### d) 4-(4-Cyano-6-cyclohexyl-3-hydroxy-pyridin-2-yl)-4-oxo-butyric acid ethyl ester

The title compound was prepared from 4-(4-Bromo-6-cyclohexyl-3-hydroxy-pyridin-2-yl)-4-oxo-butyric acid ethyl ester (see example 25c) and zinc(II) cyanide in analogy to example 2b, MS-(+)-ion, M+H=331.21.

### e) 4-(4-Cyano-6-cyclohexyl-3-hydroxy-pyridin-2-yl)-4-oxo-butyric acid

The title compound was prepared from 4-(4-Cyano-6-cyclohexyl-3-hydroxy-pyridin-2-yl)-4-oxo-butyric acid ethyl ester (see example 25d) in analogy to example 1g, MS-(−)-ion, M−H=303.20.

### Example 26

### 4-[4-Cyano-3-hydroxy-6-(4-trifluoromethyl-phenyl)-pyridin-2-yl]-4-oxo-butyric acid

### a) 4-[3-Hydroxy-6-(4-trifluoromethyl-phenyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-trifluoromethyl phenylboronic acid in analogy to example 9a, MS-(+)-ion, M+H=368.

### b) 4-[4-Bromo-3-hydroxy-6-(4-trifluoromethyl-phenyl)-pyridin-2-yl]-4-oxo-butyric acid ethyl ester

The title compound was prepared from 4-[3-Hydroxy-6-(4-trifluoromethyl-phenyl)-pyridin-2-yl]-4-oxo-butyric acid ethyl ester (see example 26a) and bromine in analogy to example 2c, MS-(+)-ion, M+H=446, 448.

### c) 4-[4-Cyano-3-hydroxy-6-(4-trifluoromethyl-phenyl)-pyridin-2-yl]-4-oxo-butyric acid ethyl ester

The title compound was prepared from 4-[4-Bromo-3-hydroxy-6-(4-trifluoromethyl-phenyl)-pyridin-2-yl]-4-oxo-butyric acid ethyl ester (see example 26b) and zinc(II) cyanide in analogy to example 2b, MS-(+)-ion, M+H=393.11.

### d) 4-[4-Cyano-3-hydroxy-6-(4-trifluoromethyl-phenyl)-pyridin-2-yl]-4-oxo-butyric acid

The title compound was prepared from 4-[4-Cyano-3-hydroxy-6-(4-trifluoromethyl-phenyl)-pyridin-2-yl]-4-oxo-butyric acid ethyl ester (see example 26c) in analogy to example 1g, MS-(+)-ion, M+H=365.11.

### Example 27

### 4-[4-Cyano-3-hydroxy-6-(4-methoxy-benzyl)-pyridin-2-yl]-4-oxo-butyric acid

### a) 4-[3-Hydroxy-6-(4-methoxy-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-methoxy-benzylzinc(II) chloride in THF (0.5 M) in analogy to example 10a, MS-(+)-ion, M+H=344.24.

### b) 4-[4-Bromo-3-hydroxy-6-(4-methoxy-benzyl)-pyridin-2-yl]-4-oxo-butyric acid ethyl ester

At room temperature, to a solution of 4-[3-hydroxy-6-(4-methoxy-benzyl)-pyridin-2-yl]-4-oxo-butyric acid ethyl ester (146 mg, 0.43 mmol, see Example 27a) in anhydrous CH₂Cl₂ (5 mL) was added N-bromosuccinimide (83 mg, 0.47 mmol). After 20 h at room temperature, the reaction was quenched by 25 mL of saturated NaHSO₃ aqueous solution and extracted with DCM (25 mL×3). The combined organic layers were washed with brine (20 mL), dried over sodium sulfate, filtered and evaporated in vacuo to afford the crude product. This material was purified by flash chromatography eluting with EtOAc/hexane (0%-10%) to give the title compound. MS-(+)-ion, M+H=424.03.

### c) 4-[4-Cyano-3-hydroxy-6-(4-methoxy-benzyl)-pyridin-2-yl]-4-oxo-butyric acid ethyl ester

The title compound was prepared from 4-[4-Bromo-3-hydroxy-6-(4-methoxy-benzyl)-pyridin-2-yl]-4-oxo-butyric acid ethyl ester (see example 27b) and zinc(II) cyanide in analogy to example 2b, MS-(+)-ion, M+H=369.13.

### d) 4-[4-Cyano-3-hydroxy-6-(4-methoxy-benzyl)-pyridin-2-yl]-4-oxo-butyric acid

The title compound was prepared from 4-[4-Cyano-3-hydroxy-6-(4-methoxy-benzyl)-pyridin-2-yl]-4-oxo-butyric acid ethyl ester (see example 27c) in analogy to example 1g, MS-(+)-ion, M+H=341.13.

### Example 28

### 4-[4-Cyano-3-hydroxy-6-(3-trifluoromethyl-phenyl)-pyridin-2-yl]-4-oxo-butyric acid

### a) 4-[3-Hydroxy-6-(3-trifluoromethyl-phenyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-trifluoromethyl phenylboronic acid in analogy to example 9a, MS-(+)-ion, M+H=368.

### b) 4-[4-Bromo-3-hydroxy-6-(3-trifluoromethyl-phenyl)-pyridin-2-yl]-4-oxo-butyric acid ethyl ester

The title compound was prepared from 4-[3-Hydroxy-6-(4-trifluoromethyl-phenyl)-pyridin-2-yl]-4-oxo-butyric acid ethyl ester (see example 28a) and bromine in analogy to example 2c, MS-(+)-ion, M+H=446, 448.

### c) 4-[4-Cyano-3-hydroxy-6-(3-trifluoromethyl-phenyl)-pyridin-2-yl]-4-oxo-butyric acid ethyl ester

The title compound was prepared from 4-[4-Bromo-3-hydroxy-6-(3-trifluoromethyl-phenyl)-pyridin-2-yl]-4-oxo-butyric acid ethyl ester (see example 28b) and zinc(II) cyanide in analogy to example 2b, MS-(+)-ion, M+H=393.11.

### d) 4-[4-Cyano-3-hydroxy-6-(3-trifluoromethyl-phenyl)-pyridin-2-yl]-4-oxo-butyric acid

The title compound was prepared from 4-[4-Cyano-3-hydroxy-6-(3-trifluoromethyl-phenyl)-pyridin-2-yl]-4-oxo-butyric acid ethyl ester (see example 28c) in analogy to example 1g, MS-(+)-ion, M+H=365.07.

### Example 29

### 4-[4-Cyano-3-hydroxy-6-(3-trifluoromethyl-benzyl)-pyridin-2-yl]-4-oxo-butyric acid

### a) 4-[3-Hydroxy-6-(3-trifluoromethyl-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-trifluoromethyl-benzylzinc(II) chloride in THF (0.5 M) in analogy to example 10a, MS-(+)-ion, M+H=382.22.

### b) 4-[4-Bromo-3-hydroxy-6-(3-trifluoromethyl-benzyl)-pyridin-2-yl]-4-oxo-butyric acid ethyl ester

The title compound was prepared from 4-[3-Hydroxy-6-(3-trifluoromethyl-benzyl)-pyridin-2-yl]-4-oxo-butyric acid ethyl ester (see example 29a) and bromine in analogy to example 2c, MS-(+)-ion, M+H=459.94, 461.89.

### c) 4-[4-Cyano-3-hydroxy-6-(3-trifluoromethyl-benzyl)-pyridin-2-yl]-4-oxo-butyric acid ethyl ester

The title compound was prepared from 4-[4-Bromo-3-hydroxy-6-(3-trifluoromethyl-benzyl)-pyridin-2-yl]-4-oxo-butyric acid ethyl ester (see example 29b) and zinc(II) cyanide in analogy to example 2b, MS-(+)-ion, M+H=407.05.

### d) 4-[4-Cyano-3-hydroxy-6-(3-trifluoromethyl-benzyl)-pyridin-2-yl]-4-oxo-butyric acid

The title compound was prepared from 4-[4-Cyano-3-hydroxy-6-(3-trifluoromethyl-benzyl)-pyridin-2-yl]-4-oxo-butyric acid ethyl ester (see example 29c) in analogy to example 1g, MS-(−)-ion, M−H=377.10.

### Example 30

### 4-[6-(2-Chloro-6-fluoro-benzyl)-4-cyano-3-hydroxy-pyridin-2-yl]-4-oxo-butyric acid

### a) 4-[6-(2-Chloro-6-fluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-chloro-6-fluoro-benzylzinc(II) chloride in THF (0.5 M) in analogy to example 10a, MS-(+)-ion, M+H=366.08.

### b) 4-[4-Bromo-6-(2-chloro-6-fluoro-benzyl)-3-hydroxy-pyridin-2-yl]-4-oxo-butyric acid ethyl ester

The title compound was prepared from 4-[6-(2-Chloro-6-fluoro-benzyl)-3-hydroxy-pyridin-2-yl]-4-oxo-butyric acid ethyl ester (see example 30a) and bromine in analogy to example 2c, MS-(+)-ion, M+H=445.88.

### c) 4-[6-(2-Chloro-6-fluoro-benzyl)-4-cyano-3-hydroxy-pyridin-2-yl]-4-oxo-butyric acid ethyl ester

The title compound was prepared from 4-[4-Bromo-6-(2-chloro-6-fluoro-benzyl)-3-hydroxy-pyridin-2-yl]-4-oxo-butyric acid ethyl ester (see example 30b) and zinc(II) cyanide in analogy to example 2b, MS-(+)-ion, M+H=391.04.

### d) 4-[6-(2-Chloro-6-fluoro-benzyl)-4-cyano-3-hydroxy-pyridin-2-yl]-4-oxo-butyric acid

The title compound was prepared from 4-[6-(2-Chloro-6-fluoro-benzyl)-4-cyano-3-hydroxy-pyridin-2-yl]-4-oxo-butyric acid ethyl ester (see example 30c) in analogy to example 1g, MS-(-)-ion, M−H=361.15.

### Example 31

### 4-[4-Cyano-6-(3,5-dichloro-benzyl)-3-hydroxy-pyridin-2-yl]-4-oxo-butyric acid

### a) 4-[6-(3,5-Dichloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3,5-dichloro-benzylzinc(II) chloride in THF (0.5 M) in analogy to example 10a, MS-(+)-ion, M+H=382.03.

### b) 4-[4-Bromo-6-(3,5-dichloro-benzyl)-3-hydroxy-pyridin-2-yl]-4-oxo-butyric acid ethyl ester

The title compound was prepared from 4-[6-(3,5-Dichloro-benzyl)-3-hydroxy-pyridin-2-yl]-4-oxo-butyric acid ethyl ester (see example 31a) and bromine in analogy to example 2c, MS-(+)-ion, M+H=461.95.

### c) 4-[4-Cyano-6-(3,5-dichloro-benzyl)-3-hydroxy-pyridin-2-yl]-4-oxo-butyric acid ethyl ester

The title compound was prepared from 4-[4-Bromo-6-(3,5-dichloro-benzyl)-3-hydroxy-pyridin-2-yl]-4-oxo-butyric acid ethyl ester (see example 31b) and zinc(II) cyanide in analogy to example 2b, MS-(+)-ion, M+H=407.05.

### d) 4-[4-Cyano-6-(3,5-dichloro-benzyl)-3-hydroxy-pyridin-2-yl]-4-oxo-butyric acid

The title compound was prepared from 4-[4-Cyano-6-(3,5-dichloro-benzyl)-3-hydroxy-pyridin-2-yl]-4-oxo-butyric acid ethyl ester (see example 31c) in analogy to example 1g, MS-(−)-ion, M−H=377.08.

### Example 32

### 4-[4-Cyano-6-(2,6-dichloro-benzyl)-3-hydroxy-pyridin-2-yl]-4-oxo-butyric acid

### a) 4-[6-(2,6-Dichloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2,6-dichloro-benzylzinc(II) chloride in THF (0.5 M) in analogy to example 10a, MS-(+)-ion, M+H=382.09.

### b) 4-[4-Bromo-6-(2,6-dichloro-benzyl)-3-hydroxy-pyridin-2-yl]-4-oxo-butyric acid ethyl ester

The title compound was prepared from 4-[6-(2,6-Dichloro-benzyl)-3-hydroxy-pyridin-2-yl]-4-oxo-butyric acid ethyl ester (see example 32a) and bromine in analogy to example 2c, MS-(+)-ion, M+H=461.95.

### c) 4-[4-Cyano-6-(2,6-dichloro-benzyl)-3-hydroxy-pyridin-2-yl]-4-oxo-butyric acid ethyl ester

The title compound was prepared from 4-[4-Bromo-6-(2,6-dichloro-benzyl)-3-hydroxy-pyridin-2-yl]-4-oxo-butyric acid ethyl ester (see example 32b) and zinc(II) cyanide in analogy to example 2b, MS-(+)-ion, M+H=407.05.

### d) 4-[4-Cyano-6-(2,6-dichloro-benzyl)-3-hydroxy-pyridin-2-yl]-4-oxo-butyric acid

The title compound was prepared from 4-[4-Cyano-6-(2,6-dichloro-benzyl)-3-hydroxy-pyridin-2-yl]-4-oxo-butyric acid ethyl ester (see example 32c) in analogy to example 1g, MS-(−)-ion, M−H=377.10.

### Example 33

### 4-(4-Cyano-6-cyclohexylmethyl-3-hydroxy-pyridin-2-yl)-4-oxo-butyric acid

### a) 4-(6-Cyclohexylmethyl-3-hydroxy-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and (cyclohexylmethyl)zinc bromide in THF (0.5 M) in analogy to example 10a, MS-(+)-ion, M+H=320.25.

### b) 4-(4-Bromo-6-cyclohexylmethyl-3-hydroxy-pyridin-2-yl)-4-oxo-butyric acid ethyl ester

The title compound was prepared from 4-(6-Cyclohexylmethyl-3-hydroxy-pyridin-2-yl)-4-oxo-butyric acid ethyl ester (see example 33a) and bromine in analogy to example 2c, MS-(+)-ion, M+H=398.10, 400.10.

### c) 4-(4-Cyano-6-cyclohexylmethyl-3-hydroxy-pyridin-2-yl)-4-oxo-butyric acid ethyl ester

The title compound was prepared from 4-(4-Bromo-6-cyclohexylmethyl-3-hydroxy-pyridin-2-yl)-4-oxo-butyric acid ethyl ester (see example 33b) and zinc(II) cyanide in analogy to example 2b, MS-(+)-ion, M+H=345.21.

### d) 4-(4-Cyano-6-cyclohexylmethyl-3-hydroxy-pyridin-2-yl)-4-oxo-butyric acid

The title compound was prepared from 4-(4-Cyano-6-cyclohexylmethyl-3-hydroxy-pyridin-2-yl)-4-oxo-butyric acid ethyl ester (see example 33c) in analogy to example 1g, MS-(+)-ion, M+H=317.26.

### Example 34

### 4-(4-Cyano-3-hydroxy-6-naphthalen-1-ylmethyl-pyridin-2-yl)-4-oxo-butyric acid

### a) 4-(3-Hydroxy-6-naphthalen-1-ylmethyl-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and (1-naphthyl)methylzinc chloride in THF (0.5 M) in analogy to example 10a, MS-(+)-ion, M+H=364.20.

### b) 4-(4-Bromo-3-hydroxy-6-naphthalen-1-ylmethyl-pyridin-2-yl)-4-oxo-butyric acid ethyl ester

The title compound was prepared from 4-(6-Cyclohexylmethyl-3-hydroxy-pyridin-2-yl)-4-oxo-butyric acid ethyl ester (see example 33a) and bromine in analogy to example 2c, MS-(+)-ion, M+H=444.03.

### c) 4-(4-Cyano-3-hydroxy-6-naphthalen-1-ylmethyl-pyridin-2-yl)-4-oxo-butyric acid ethyl ester

The title compound was prepared from 4-(4-Bromo-3-hydroxy-6-naphthalen-1-ylmethyl-pyridin-2-yl)-4-oxo-butyric acid ethyl ester (see example 34b) and zinc(II) cyanide in analogy to example 2b, MS-(+)-ion, M+H=389.16.

### d) 4-(4-Cyano-3-hydroxy-6-naphthalen-1-ylmethyl-pyridin-2-yl)-4-oxo-butyric acid

The title compound was prepared from 4-(4-Cyano-3-hydroxy-6-naphthalen-1-ylmethyl-pyridin-2-yl)-4-oxo-butyric acid ethyl ester (see example 34c) in analogy to example 1g, MS-(−)-ion, M−H=359.20.

### Example 35

### 4-(4-Cyano-3-hydroxy-6-trifluoromethyl-pyridin-2-yl)-4-oxo-butyric acid

### a) 4-(3-Benzyloxy-6-bromo-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and benzyl bromide in analogy to example 1b. MS-(+)-ion, M+H=394.03.

### b) 4-(3-Benzyloxy-6-trifluoromethyl-pyridin-2-yl)-4-oxo-butyric acid ethyl ester

The title compound was prepared from 4-(3-Benzyloxy-6-bromo-pyridin-2-yl)-4-oxo-butyric acid ethyl ester (see example 35a) and 2,2-difluoro-2-(fluorosulfonyl)acetate in analogy to example 4b. MS-(+)-ion, M+H=382.13.

### c) 4-(3-Hydroxy-6-trifluoromethyl-pyridin-2-yl)-4-oxo-butyric acid ethyl ester

The title compound was prepared from 4-(3-Benzyloxy-6-trifluoromethyl-pyridin-2-yl)-4-oxo-butyric acid ethyl ester (see example 35b) and trifluoroacetic acid in analogy to example 4c, MS-(-)-ion, M−H=290.20.

### d) 4-(4-Bromo-3-hydroxy-6-trifluoromethyl-pyridin-2-yl)-4-oxo-butyric acid ethyl ester

The title compound was prepared from 4-(3-Hydroxy-6-trifluoromethyl-pyridin-2-yl)-4-oxo-butyric acid ethyl ester (see example 35c) and bromine in analogy to example 2c, MS-(+)-ion, M+H=370.05.

### e) 4-(4-Cyano-3-hydroxy-6-trifluoromethyl-pyridin-2-yl)-4-oxo-butyric acid ethyl ester

The title compound was prepared from 4-(4-Bromo-3-hydroxy-6-trifluoromethyl-pyridin-2-yl)-4-oxo-butyric acid ethyl ester (see example 35d) and zinc(II) cyanide in analogy to example 2b, MS-(−)-ion, M−H=315.20.

### f) 4-(4-Cyano-3-hydroxy-6-trifluoromethyl-pyridin-2-yl)-4-oxo-butyric acid

The title compound was prepared from 4-(4-Cyano-3-hydroxy-6-trifluoromethyl-pyridin-2-yl)-4-oxo-butyric acid ethyl ester (see example 35e) in analogy to example 1g, MS-(−)-ion, M−H=287.13.

### Example 36

### 4-[4-Cyano-3-hydroxy-6-(2-methyl-benzyl)-pyridin-2-yl]-4-oxo-butyric acid

### a) 4-[3-Hydroxy-6-(2-methyl-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-methyl-benzylzinc(II) chloride in THF (0.5 M) in analogy to example 10a, MS-(+)-ion, M+H=328.16.

### b) 4-[4-Bromo-3-hydroxy-6-(2-methyl-benzyl)-pyridin-2-yl]-4-oxo-butyric acid ethyl ester

The title compound was prepared from 4-[3-Hydroxy-6-(2-methyl-benzyl)-pyridin-2-yl]-4-oxo-butyric acid methyl ester (see example 36a) and bromine in analogy to example 2c, MS-(+)-ion, M+H=408.02.

### c) 4-[4-Cyano-3-hydroxy-6-(2-methyl-benzyl)-pyridin-2-yl]-4-oxo-butyric acid ethyl ester

The title compound was prepared from 4-[4-Bromo-3-hydroxy-6-(2-methyl-benzyl)-pyridin-2-yl]-4-oxo-butyric acid ethyl ester (see example 36c) and zinc(II) cyanide in analogy to example 2b, MS-(−)-ion, M−H=353.15.

### d) 4-[4-Cyano-3-hydroxy-6-(2-methyl-benzyl)-pyridin-2-yl]-4-oxo-butyric acid

The title compound was prepared from 4-[4-Cyano-3-hydroxy-6-(2-methyl-benzyl)-pyridin-2-yl]-4-oxo-butyric acid ethyl ester (see example 36c) in analogy to example 1g, MS-(−)-ion, M−H=323.18.

### Example 37

### 4-[4-Cyano-3-hydroxy-6-(3-methyl-benzyl)-pyridin-2-yl]-4-oxo-butyric acid

### a) 4-[3-Hydroxy-6-(3-methyl-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-methyl-benzylzinc(II) chloride in THF (0.5 M) in analogy to example 10a, MS-(+)-ion, M+H=328.16.

### b) 4-[4-Bromo-3-hydroxy-6-(3-methyl-benzyl)-pyridin-2-yl]-4-oxo-butyric acid ethyl ester

The title compound was prepared from 4-[3-Hydroxy-6-(3-methyl-benzyl)-pyridin-2-yl]-4-oxo-butyric acid ethyl ester (see example 37a) and bromine in analogy to example 2c, MS-(+)-ion, M+H=408.02.

### c) 4-[4-Cyano-3-hydroxy-6-(2-methyl-benzyl)-pyridin-2-yl]-4-oxo-butyric acid ethyl ester

The title compound was prepared from 4-[4-Bromo-3-hydroxy-6-(3-methyl-benzyl)-pyridin-2-yl]-4-oxo-butyric acid ethyl ester (see example 37b) and zinc(II) cyanide in analogy to example 2b, MS-(+)-ion, M+H=353.15.

### d) 4-[4-Cyano-3-hydroxy-6-(3-methyl-benzyl)-pyridin-2-yl]-4-oxo-butyric acid

The title compound was prepared from 4-[4-Cyano-3-hydroxy-6-(3-methyl-benzyl)-pyridin-2-yl]-4-oxo-butyric acid ethyl ester (see example 37c) in analogy to example 1g, MS-(−)-ion, M−H=323.19.

### Example 38

### 4-[4-Cyano-3-hydroxy-6-(4-methyl-benzyl)-pyridin-2-yl]-4-oxo-butyric acid

### a) 4-[3-Hydroxy-6-(4-methyl-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-methyl-benzylzinc(II) chloride in THF (0.5 M) in analogy to example 10a, MS-(+)-ion, M+H=328.16.

### b) 4-[4-Bromo-3-hydroxy-6-(4-methyl-benzyl)-pyridin-2-yl]-4-oxo-butyric acid ethyl ester

The title compound was prepared from 4-[3-Hydroxy-6-(4-methyl-benzyl)-pyridin-2-yl]-4-oxo-butyric acid ethyl ester (see example 38a) and bromine in analogy to example 2c, MS-(+)-ion, M+H=408.02.

### c) 4-[4-Cyano-3-hydroxy-6-(4-methyl-benzyl)-pyridin-2-yl]-4-oxo-butyric acid ethyl ester

The title compound was prepared from 4-[4-Bromo-3-hydroxy-6-(4-methyl-benzyl)-pyridin-2-yl]-4-oxo-butyric acid ethyl ester (see example 38b) and zinc(II) cyanide in analogy to example 2b, MS-(+)-ion, M+H=353.18.

### d) 4-[4-Cyano-3-hydroxy-6-(4-methyl-benzyl)-pyridin-2-yl]-4-oxo-butyric acid

The title compound was prepared from 4-[4-Cyano-3-hydroxy-6-(4-methyl-benzyl)-pyridin-2-yl]-4-oxo-butyric acid ethyl ester (see example 38c) in analogy to example 1g, MS-(+)-ion, M+H=325.04.

### Example 39

### 4-[4-Cyano-3-hydroxy-6-(4-trifluoromethyl-benzyl)-pyridin-2-yl]-4-oxo-butyric acid

### a) 4-trifluoromethyl-benzylzinc(II) bromide

At 65° C., to a suspension mixture of zinc dust (520 mg, 8 mmol, Sigma, catalog #209988, <10 uM) in dry THF (10 mL) was added 1,2-dibromoethane (14 uL, 0.16 mmol) under nitrogen atmosphere, followed by the addition of chlorotrimethylsilane (82 uL, 0.64 mmol). (Note: Bubbles will be observed after 10-20 seconds of the addition of chlorotrimethylsilane, which indicates the initiation of the reaction. If no bubbles formed, additional chlorotrimethylsilane should be added until the observation of bubbles). The mixture was then stirred at 65° C. for another 30 min. After cooling to room temperature, a THF solution (4 mL) of 4-trifluoromethyl-benzyl bromide (4 mmol) was added dropwise, and the suspension was stirred at room temperature for another 1 h. The reaction mixture was directly used in the next step.

### b) 4-[3-Hydroxy-6-(4-trifluoromethyl-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-trifluoromethyl-benzylzinc(II) bromide in THF (see Example 39a) in analogy to example 10a, MS-(+)-ion, M+H=382.16.

### c) 4-[4-Bromo-3-hydroxy-6-(4-trifluoromethyl-benzyl)-pyridin-2-yl]-4-oxo-butyric acid ethyl ester

The title compound was prepared from 4-[3-Hydroxy-6-(4-trifluoromethyl-benzyl)-pyridin-2-yl]-4-oxo-butyric acid ethyl ester (see example 39b) and bromine in analogy to example 2c, MS-(+)-ion, M+H=460.01.

### d) 4-[4-Cyano-3-hydroxy-6-(4-trifluoromethyl-benzyl)-pyridin-2-yl]-4-oxo-butyric acid ethyl ester

The title compound was prepared from 4-[4-Bromo-3-hydroxy-6-(4-trifluoromethyl-benzyl)-pyridin-2-yl]-4-oxo-butyric acid ethyl ester (see example 39c) and zinc(II) cyanide in analogy to example 2b, MS-(+)-ion, M+H=406.96.

### e) 4-[4-Cyano-3-hydroxy-6-(4-trifluoromethyl-benzyl)-pyridin-2-yl]-4-oxo-butyric acid

The title compound was prepared from 4-[4-Cyano-3-hydroxy-6-(4-trifluoromethyl-benzyl)-pyridin-2-yl]-4-oxo-butyric acid ethyl ester (see example 39d) in analogy to example 1g, MS-(+)-ion, M+H=379.05.

### Example 40

### 4-[4-Cyano-6-(4-fluoro-benzyl)-3-hydroxy-pyridin-2-yl]-4-oxo-butyric acid

### a) 4-[6-(4-Fluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-fluoro-benzylzinc(II) chloride in THF (0.5 M) in analogy to example 10a, MS-(+)-ion, M+H=331.74.

### b) 4-[4-Bromo-6-(4-fluoro-benzyl)-3-hydroxy-pyridin-2-yl]-4-oxo-butyric acid ethyl ester

The title compound was prepared from 4-[3-Hydroxy-6-(4-fluoro-benzyl)-pyridin-2-yl]-4-oxo-butyric acid ethyl ester (see example 40a) and bromine in analogy to example 2c, MS-(+)-ion, M+H=411.96.

### c) 4-[4-Cyano-6-(4-fluoro-benzyl)-3-hydroxy-pyridin-2-yl]-4-oxo-butyric acid ethyl ester

The title compound was prepared from 4-[4-Bromo-3-hydroxy-6-(4-fluoro-benzyl)-pyridin-2-yl]-4-oxo-butyric acid ethyl ester (see example 40b) and zinc(II) cyanide in analogy to example 2b, MS-(+)-ion, M+H=357.14.

### d) 4-[4-Cyano-6-(4-fluoro-benzyl)-3-hydroxy-pyridin-2-yl]-4-oxo-butyric acid

The title compound was prepared from 4-[4-Cyano-3-hydroxy-6-(4-fluoro-benzyl)-pyridin-2-yl]-4-oxo-butyric acid ethyl ester (see example 40c) in analogy to example 1g, MS-(+)-ion, M+H=328.94.

### Example 41

### 4-[4-Cyano-3-hydroxy-6-(2-trifluoromethyl-benzyl)-pyridin-2-yl]-4-oxo-butyric acid

### a) 2-trifluoromethyl-benzylzinc(II) bromide

The title compound was prepared from 4-trifluoromethyl-benzyl bromide and zinc dust in analogy to example 39a.

### b) 4-[3-Hydroxy-6-(2-trifluoromethyl-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-trifluoromethyl-benzylzinc(II) bromide in THF (see Example 41a) in analogy to example 10a, MS-(+)-ion, M+H=382.05.

### c) 4-[4-Bromo-3-hydroxy-6-(2-trifluoromethyl-benzyl)-pyridin-2-yl]-4-oxo-butyric acid ethyl ester

The title compound was prepared from 4-[3-Hydroxy-6-(2-trifluoromethyl-benzyl)-pyridin-2-yl]-4-oxo-butyric acid ethyl ester (see example 41b) and bromine in analogy to example 2c, MS-(+)-ion, M+H=461.97.

### d) 4-[4-Cyano-3-hydroxy-6-(2-trifluoromethyl-benzyl)-pyridin-2-yl]-4-oxo-butyric acid ethyl ester

The title compound was prepared from 4-[4-Bromo-3-hydroxy-6-(2-trifluoromethyl-benzyl)-pyridin-2-yl]-4-oxo-butyric acid ethyl ester (see example 41c) and zinc(II) cyanide in analogy to example 2b, MS-(+)-ion, M+H=407.06.

### e) 4-[4-Cyano-3-hydroxy-6-(2-trifluoromethyl-benzyl)-pyridin-2-yl]-4-oxo-butyric acid

The title compound was prepared from 4-[4-Cyano-3-hydroxy-6-(2-trifluoromethyl-benzyl)-pyridin-2-yl]-4-oxo-butyric acid ethyl ester (see example 41c) in analogy to example 1g, MS-(+)-ion, M+H=378.95.

### Example 42

### 4-[4-Cyano-6-(3-fluoro-benzyl)-3-hydroxy-pyridin-2-yl]-4-oxo-butyric acid

### a) 4-[6-(3-Fluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-fluoro-benzylzinc(II) chloride in THF (0.5 M) in analogy to example 10a, MS-(+)-ion, M+H=331.84.

### b) 4-[4-Bromo-6-(3-fluoro-benzyl)-3-hydroxy-pyridin-2-yl]-4-oxo-butyric acid ethyl ester

The title compound was prepared from 4-[6-(3-Fluoro-benzyl)-3-hydroxy-pyridin-2-yl]-4-oxo-butyric acid ethyl ester (see example 42a) and bromine in analogy to example 2c, MS-(+)-ion, M+H=409.96.

### c) 4-[4-Cyano-6-(3-fluoro-benzyl)-3-hydroxy-pyridin-2-yl]-4-oxo-butyric acid ethyl ester

The title compound was prepared from 4-[4-Bromo-3-hydroxy-6-(3-fluoro-benzyl)-pyridin-2-yl]-4-oxo-butyric acid ethyl ester (see example 42b) and zinc(II) cyanide in analogy to example 2b, MS-(+)-ion, M+H=357.04.

### d) 4-[4-Cyano-6-(3-fluoro-benzyl)-3-hydroxy-pyridin-2-yl]-4-oxo-butyric acid

The title compound was prepared from 4-[4-Cyano-3-hydroxy-6-(3-fluoro-benzyl)-pyridin-2-yl]-4-oxo-butyric acid ethyl ester (see example 42c) in analogy to example 1g, MS-(+)-ion, M+H=328.94.

### Example 43

### 4-[4-Cyano-6-(2-fluoro-benzyl)-3-hydroxy-pyridin-2-yl]-4-oxo-butyric acid

### a) 4-[6-(2-Fluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-fluoro-benzylzinc(II) chloride in THF (0.5 M) in analogy to example 10a, MS-(+)-ion, M+H=331.84.

### b) 4-[4-Bromo-6-(2-fluoro-benzyl)-3-hydroxy-pyridin-2-yl]-4-oxo-butyric acid ethyl ester

The title compound was prepared from 4-[3-Hydroxy-6-(2-fluoro-benzyl)-pyridin-2-yl]-4-oxo-butyric acid ethyl ester (see example 43a) and bromine in analogy to example 2c, MS-(+)-ion, M+H=409.96, 411.76.

### c) 4-[4-Cyano-6-(2-fluoro-benzyl)-3-hydroxy-pyridin-2-yl]-4-oxo-butyric acid ethyl ester

The title compound was prepared from 4-[4-Bromo-3-hydroxy-6-(2-fluoro-benzyl)-pyridin-2-yl]-4-oxo-butyric acid ethyl ester (see example 43b) and zinc(II) cyanide in analogy to example 2b, MS-(+)-ion, M+H=357.04.

### d) 4-[4-Cyano-6-(2-fluoro-benzyl)-3-hydroxy-pyridin-2-yl]-4-oxo-butyric acid

The title compound was prepared from 4-[4-Cyano-3-hydroxy-6-(2-fluoro-benzyl)-pyridin-2-yl]-4-oxo-butyric acid ethyl ester (see example 43c) in analogy to example 1g, MS-(+)-ion, M+H=329.04.

### Example 44

### 4-[4-Cyano-6-(4-cyano-benzyl)-3-hydroxy-pyridin-2-yl]-4-oxo-butyric acid

### a) 4-cyano-benzylzinc(II) bromide

The title compound was prepared from 4-cyano-benzyl bromide and zinc dust in analogy to example 39a.

### b) 4-[6-(4-Cyano-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-cyano-benzylzinc(II) bromide in THF (see Example 44a) in analogy to example 10a, MS-(+)-ion, M+H=338.84.

### c) 4-[4-Bromo-6-(4-cyano-benzyl)-3-hydroxy-pyridin-2-yl]-4-oxo-butyric acid ethyl ester

The title compound was prepared from 4-[6-(4-Cyano-benzyl)-3-hydroxy-pyridin-2-yl]-4-oxo-butyric acid ethyl ester (see example 44b) and bromine in analogy to example 2c, MS-(+)-ion, M+H=416.86, 418.86.

### d) 4-[4-Cyano-6-(4-cyano-benzyl)-3-hydroxy-pyridin-2-yl]-4-oxo-butyric acid ethyl ester

The title compound was prepared from 4-[4-Bromo-6-(4-cyano-benzyl)-3-hydroxy-pyridin-2-yl]-4-oxo-butyric acid ethyl ester (see example 44c) and zinc(II) cyanide in analogy to example 2b, MS-(+)-ion, M+H=364.15.

### e) 4-[4-Cyano-6-(4-cyano-benzyl)-3-hydroxy-pyridin-2-yl]-4-oxo-butyric acid

The title compound was prepared from 4-[4-Cyano-6-(4-cyano-benzyl)-3-hydroxy-pyridin-2-yl]-4-oxo-butyric acid ethyl ester (see example 44d) in analogy to example 1g, MS-(+)-ion, M+H=335.54.

### Example 45

### 4-[4-Cyano-6-(3-cyano-benzyl)-3-hydroxy-pyridin-2-yl]-4-oxo-butyric acid

### a) 3-cyano-benzylzinc(II) bromide

The title compound was prepared from 3-cyano-benzyl bromide and zinc dust in analogy to example 39a.

### b) 4-[6-(3-Cyano-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-cyano-benzylzinc(II) bromide in THF (see Example 45a) in analogy to example 10a, MS-(+)-ion, M+H=338.74.

### c) 4-[4-Bromo-6-(3-cyano-benzyl)-3-hydroxy-pyridin-2-yl]-4-oxo-butyric acid ethyl ester

The title compound was prepared from 4-[6-(3-Cyano-benzyl)-3-hydroxy-pyridin-2-yl]-4-oxo-butyric acid ethyl ester (see example 45b) and bromine in analogy to example 2c, MS-(+)-ion, M+H=418.76.

### d) 4-[4-Cyano-6-(3-cyano-benzyl)-3-hydroxy-pyridin-2-yl]-4-oxo-butyric acid ethyl ester

The title compound was prepared from 4-[4-Bromo-6-(3-cyano-benzyl)-3-hydroxy-pyridin-2-yl]-4-oxo-butyric acid ethyl ester (see example 45c) and zinc(II) cyanide in analogy to example 2b, MS-(+)-ion, M+H=363.95.

### e) 4-[4-Cyano-6-(3-cyano-benzyl)-3-hydroxy-pyridin-2-yl]-4-oxo-butyric acid

The title compound was prepared from 4-[4-Cyano-6-(3-cyano-benzyl)-3-hydroxy-pyridin-2-yl]-4-oxo-butyric acid ethyl ester (see example 45d) in analogy to example 1g, MS-(+)-ion, M+H=336.14.

### Example 46

### 4-[4-Cyano-6-(2-cyano-benzyl)-3-hydroxy-pyridin-2-yl]-4-oxo-butyric acid

### a) 2-cyano-benzylzinc(II) bromide

The title compound was prepared from 2-cyano-benzyl bromide and zinc dust in analogy to example 39a.

### b) 4-[6-(2-Cyano-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-cyano-benzylzinc(II) bromide in THF (see Example 46a) in analogy to example 10a, MS-(+)-ion, M+H=338.64.

### c) 4-[4-Bromo-6-(2-cyano-benzyl)-3-hydroxy-pyridin-2-yl]-4-oxo-butyric acid ethyl ester

The title compound was prepared from 4-[6-(2-Cyano-benzyl)-3-hydroxy-pyridin-2-yl]-4-oxo-butyric acid ethyl ester (see example 46b) and bromine in analogy to example 2c, MS-(+)-ion, M+H=416.96.

### d) 4-[4-Cyano-6-(2-cyano-benzyl)-3-hydroxy-pyridin-2-yl]-4-oxo-butyric acid ethyl ester

The title compound was prepared from 4-[4-Bromo-6-(2-cyano-benzyl)-3-hydroxy-pyridin-2-yl]-4-oxo-butyric acid ethyl ester (see example 46c) and zinc(II) cyanide in analogy to example 2b, MS-(+)-ion, M+H=364.05.

### e) 4-[4-Cyano-6-(2-cyano-benzyl)-3-hydroxy-pyridin-2-yl]-4-oxo-butyric acid

The title compound was prepared from 4-[4-Cyano-6-(2-cyano-benzyl)-3-hydroxy-pyridin-2-yl]-4-oxo-butyric acid ethyl ester (see example 46d) in analogy to example 1g, MS-(+)-ion, M+H=336.04.

### Example 47

### 4-[4-Cyano-3-hydroxy-6-(4-trifluoromethoxy-benzyl)-pyridin-2-yl]-4-oxo-butyric acid

### a) 4-trifluoromethoxy-benzylzinc(II) bromide

The title compound was prepared from 4-trifluoromethoxy-benzyl bromide and zinc dust in analogy to example 39a.

### b) 4-[6-(4-trifluoromethoxy-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 4-trifluoromethoxy-benzylzinc(II) bromide in THF (see Example 47a) in analogy to example 10a, MS-(+)-ion, M+H=397.86.

### c) 4-[4-Bromo-6-(4-trifluoromethoxy-benzyl)-3-hydroxy-pyridin-2-yl]-4-oxo-butyric acid ethyl ester

The title compound was prepared from 4-[6-(4-trifluoromethoxy-benzyl)-3-hydroxy-pyridin-2-yl]-4-oxo-butyric acid ethyl ester (see example 47b) and bromine in analogy to example 2c, MS-(+)-ion, M+H=475.81, 477.66.

### d) 4-[4-Cyano-6-(4-trifluoromethoxy-benzyl)-3-hydroxy-pyridin-2-yl]-4-oxo-butyric acid ethyl ester

The title compound was prepared from 4-[4-Bromo-6-(4-trifluoromethoxy-benzyl)-3-hydroxy-pyridin-2-yl]-4-oxo-butyric acid ethyl ester (see example 47c) and zinc(II) cyanide in analogy to example 2b, MS-(+)-ion, M+H=422.95.

### e) 4-[4-Cyano-6-(4-trifluoromethoxy-benzyl)-3-hydroxy-pyridin-2-yl]-4-oxo-butyric acid

The title compound was prepared from 4-[4-Cyano-6-(4-trifluoromethoxy-benzyl)-3-hydroxy-pyridin-2-yl]-4-oxo-butyric acid ethyl ester (see example 47d) in analogy to example 1g, MS-(+)-ion, M+H=394.94.

### Example 48

### 4-[4-Cyano-3-hydroxy-6-(3-trifluoromethoxy-benzyl)-pyridin-2-yl]-4-oxo-butyric acid

### a) 3-trifluoromethoxy-benzylzinc(II) bromide

The title compound was prepared from 3-trifluoromethoxy-benzyl bromide and zinc dust in analogy to example 39a.

### b) 4-[6-(3-trifluoromethoxy-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-trifluoromethoxy-benzylzinc(II) bromide in THF (see Example 48a) in analogy to example 10a, MS-(+)-ion, M+H=397.99.

### c) 4-[4-Bromo-6-(3-trifluoromethoxy-benzyl)-3-hydroxy-pyridin-2-yl]-4-oxo-butyric acid ethyl ester

The title compound was prepared from 4-[6-(3-trifluoromethoxy-benzyl)-3-hydroxy-pyridin-2-yl]-4-oxo-butyric acid ethyl ester (see example 48b) and bromine in analogy to example 2c, MS-(+)-ion, M+H=475.81, 477.76.

### d) 4-[4-Cyano-6-(3-trifluoromethoxy-benzyl)-3-hydroxy-pyridin-2-yl]-4-oxo-butyric acid ethyl ester

The title compound was prepared from 4-[4-Bromo-6-(3-trifluoromethoxy-benzyl)-3-hydroxy-pyridin-2-yl]-4-oxo-butyric acid ethyl ester (see example 48c) and zinc(II) cyanide in analogy to example 2b, MS-(+)-ion, M+H=422.95.

### e) 4-[4-Cyano-6-(3-trifluoromethoxy-benzyl)-3-hydroxy-pyridin-2-yl]-4-oxo-butyric acid

The title compound was prepared from 4-[4-Cyano-6-(3-trifluoromethoxy-benzyl)-3-hydroxy-pyridin-2-yl]-4-oxo-butyric acid ethyl ester (see example 48d) in analogy to example 1g, MS-(+)-ion, M+H=394.84.

### Example 49

### 4-[4-Cyano-3-hydroxy-6-(2-trifluoromethoxy-benzyl)-pyridin-2-yl]-4-oxo-butyric acid

### a) 2-trifluoromethoxy-benzylzinc(II) bromide

The title compound was prepared from 2-trifluoromethoxy-benzyl bromide and zinc dust in analogy to example 39a.

### b) 4-[6-(2-trifluoromethoxy-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-trifluoromethoxy-benzylzinc(II) bromide in THF (see Example 49a) in analogy to example 10a, MS-(+)-ion, M+H=397.99.

### c) 4-[4-Bromo-6-(2-trifluoromethoxy-benzyl)-3-hydroxy-pyridin-2-yl]-4-oxo-butyric acid ethyl ester

The title compound was prepared from 4-[6-(2-trifluoromethoxy-benzyl)-3-hydroxy-pyridin-2-yl]-4-oxo-butyric acid ethyl ester (see example 49b) and bromine in analogy to example 2c, MS-(+)-ion, M+H=475.76.

### d) 4-[4-Cyano-6-(2-trifluoromethoxy-benzyl)-3-hydroxy-pyridin-2-yl]-4-oxo-butyric acid ethyl ester

The title compound was prepared from 4-[4-Bromo-6-(2-trifluoromethoxy-benzyl)-3-hydroxy-pyridin-2-yl]-4-oxo-butyric acid ethyl ester (see example 49c) and zinc(II) cyanide in analogy to example 2b, MS-(+)-ion, M+H=422.95.

### e) 4-[4-Cyano-6-(2-trifluoromethoxy-benzyl)-3-hydroxy-pyridin-2-yl]-4-oxo-butyric acid

The title compound was prepared from 4-[4-Cyano-6-(2-trifluoromethoxy-benzyl)-3-hydroxy-pyridin-2-yl]-4-oxo-butyric acid ethyl ester (see example 49d) in analogy to example 1g, MS-(+)-ion, M+H=394.94.

### Example 50

### 4-(6-Biphenyl-4-ylmethyl-4-cyano-3-hydroxy-pyridin-2-yl)-4-oxo-butyric acid

### a) 4-phenyl-benzylzinc(II) bromide

The title compound was prepared from 4-bromomethyl-biphenyl and zinc dust in analogy to example 39a.

### b) 4-(6-Biphenyl-4-ylmethyl-3-hydroxy-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and 4-phenyl-benzylzinc(II) bromide in THF (see Example 50a) in analogy to example 10a, MS-(+)-ion, M+H=390.04.

### c) 4-(6-Biphenyl-4-ylmethyl-4-bromo-3-hydroxy-pyridin-2-yl)-4-oxo-butyric acid ethyl ester

The title compound was prepared from 4-(6-Biphenyl-4-ylmethyl-3-hydroxy-pyridin-2-yl)-4-oxo-butyric acid ethyl ester (see example 50b) and bromine in analogy to example 2c, MS-(+)-ion, M+H=467.90, 469.80.

### d) 4-(6-Biphenyl-4-ylmethyl-4-cyano-3-hydroxy-pyridin-2-yl)-4-oxo-butyric acid ethyl ester

The title compound was prepared from 4-(6-Biphenyl-4-ylmethyl-4-bromo-3-hydroxy-pyridin-2-yl)-4-oxo-butyric acid ethyl ester (see example 50c) and zinc(II) cyanide in analogy to example 2b, MS-(+)-ion, M+H=415.04.

### e) 4-(6-Biphenyl-4-ylmethyl-4-cyano-3-hydroxy-pyridin-2-yl)-4-oxo-butyric acid

The title compound was prepared from 4-(6-Biphenyl-4-ylmethyl-4-cyano-3-hydroxy-pyridin-2-yl)-4-oxo-butyric acid ethyl ester (see example 50d) in analogy to example 1g, MS-(+)-ion, M+H=386.99.

### Example 51

### 4-(6-Biphenyl-3-ylmethyl-4-cyano-3-hydroxy-pyridin-2-yl)-4-oxo-butyric acid

### a) 3-phenyl-benzylzinc(II) bromide

The title compound was prepared from 3-bromomethyl-biphenyl and zinc dust in analogy to example 39a.

### b) 4-(6-Biphenyl-3-ylmethyl-3-hydroxy-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and 3-phenyl-benzylzinc(II) bromide in THF (see Example 51a) in analogy to example 10a, MS-(+)-ion, M+H=390.09.

### c) 4-(6-Biphenyl-3-ylmethyl-4-bromo-3-hydroxy-pyridin-2-yl)-4-oxo-butyric acid ethyl ester

The title compound was prepared from 4-(6-Biphenyl-3-ylmethyl-3-hydroxy-pyridin-2-yl)-4-oxo-butyric acid ethyl ester (see example 51b) and bromine in analogy to example 2c, MS-(+)-ion, M+H=467.90, 469.80.

### d) 4-(6-Biphenyl-3-ylmethyl-4-cyano-3-hydroxy-pyridin-2-yl)-4-oxo-butyric acid ethyl ester

The title compound was prepared from 4-(6-Biphenyl-3-ylmethyl-4-bromo-3-hydroxy-pyridin-2-yl)-4-oxo-butyric acid ethyl ester (see example 51c) and zinc(II) cyanide in analogy to example 2b, MS-(+)-ion, M+H=415.09.

### e) 4-(6-Biphenyl-3-ylmethyl-4-cyano-3-hydroxy-pyridin-2-yl)-4-oxo-butyric acid

The title compound was prepared from 4-(6-Biphenyl-3-ylmethyl-4-cyano-3-hydroxy-pyridin-2-yl)-4-oxo-butyric acid ethyl ester (see example 51d) in analogy to example 1g, MS-(+)-ion, M+H=387.04.

### Example 52

### 4-(6-Biphenyl-2-ylmethyl-4-cyano-3-hydroxy-pyridin-2-yl)-4-oxo-butyric acid

### a) 2-phenyl-benzylzinc(II) bromide

The title compound was prepared from 2-bromomethyl-biphenyl and zinc dust in analogy to example 39a.

### b) 4-(6-Biphenyl-2-ylmethyl-3-hydroxy-pyridin-2-yl)-4-oxo-butyric acid ethyl ester

The title compound was prepared from 4-(6-Bromo-3-hydroxy-pyridin-2-yl)-4-oxo-butyric acid ethyl ester (see Example 2a) and 2-phenyl-benzylzinc(II) bromide in THF (see Example 52a) in analogy to example 10a, MS-(+)-ion, M+H=390.09.

### c) 4-(6-Biphenyl-2-ylmethyl-4-bromo-3-hydroxy-pyridin-2-yl)-4-oxo-butyric acid ethyl ester

The title compound was prepared from 4-(6-Biphenyl-2-ylmethyl-3-hydroxy-pyridin-2-yl)-4-oxo-butyric acid ethyl ester (see example 52b) and bromine in analogy to example 2c, MS-(+)-ion, M+H=467.85, 469.75.

### d) 4-(6-Biphenyl-2-ylmethyl-4-cyano-3-hydroxy-pyridin-2-yl)-4-oxo-butyric acid ethyl ester

The title compound was prepared from 4-(6-Biphenyl-2-ylmethyl-4-bromo-3-hydroxy-pyridin-2-yl)-4-oxo-butyric acid ethyl ester (see example 52c) and zinc(II) cyanide in analogy to example 2b, MS-(+)-ion, M+H=415.09.

### e) 4-(6-Biphenyl-2-ylmethyl-4-cyano-3-hydroxy-pyridin-2-yl)-4-oxo-butyric acid

The title compound was prepared from 4-(6-Biphenyl-2-ylmethyl-4-cyano-3-hydroxy-pyridin-2-yl)-4-oxo-butyric acid ethyl ester (see example 52d) in analogy to example 1g, MS-(+)-ion, M+H=387.04.

### Example 53

### 4-[4-Cyano-6-(2,6-difluoro-benzyl)-3-hydroxy-pyridin-2-yl]-4-oxo-butyric acid

### a) 2,6-Difluoro-benzylzinc(II) bromide

The title compound was prepared from 2-bromomethyl-1,3-difluoro-benzene and zinc dust in analogy to example 39a.

### b) 4-[6-(2,6-Difluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2,6-Difluoro-benzylzinc(II) bromide in THF (see Example 53a) in analogy to example 10a, MS-(+)-ion, M+H=349.93.

### c) 4-[4-Bromo-6-(2,6-difluoro-benzyl)-3-hydroxy-pyridin-2-yl]-4-oxo-butyric acid ethyl ester

The title compound was prepared from 4-[6-(2,6-Difluoro-benzyl)-3-hydroxy-pyridin-2-yl]-4-oxo-butyric acid ethyl ester (see example 53b) and bromine in analogy to example 2c, MS-(+)-ion, M+H=427.85, 429.75.

### d) 4-[4-Cyano-6-(2,6-difluoro-benzyl)-3-hydroxy-pyridin-2-yl]-4-oxo-butyric acid ethyl ester

The title compound was prepared from 4-[4-Bromo-6-(2,6-difluoro-benzyl)-3-hydroxy-pyridin-2-yl]-4-oxo-butyric acid ethyl ester (see example 53c) and zinc(II) cyanide in analogy to example 2b, MS-(+)-ion, M+H=374.94.

### e) 4-[4-Cyano-6-(2,6-difluoro-benzyl)-3-hydroxy-pyridin-2-yl]-4-oxo-butyric acid

The title compound was prepared from 4-[4-Cyano-6-(2,6-difluoro-benzyl)-3-hydroxy-pyridin-2-yl]-4-oxo-butyric acid ethyl ester (see example 53d) in analogy to example 1g, MS-(+)-ion, M+H=346.88.

### Example 54

### 4-[4-Cyano-6-(2,6-dimethyl-benzyl)-3-hydroxy-pyridin-2-yl]-4-oxo-butyric acid

### a) 2,6-Dimethyl-benzylzinc(II) bromide

The title compound was prepared from 2-Bromomethyl-1,3-dimethyl-benzene and zinc dust in analogy to example 39a.

### b) 4-[6-(2,6-Dimethyl-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2,6-Dimethyl-benzylzinc(II) bromide in THF (see Example 54a) in analogy to example 10a, MS-(+)-ion, M+H=342.03.

### c) 4-[4-Bromo-6-(2,6-dimethyl-benzyl)-3-hydroxy-pyridin-2-yl]-4-oxo-butyric acid ethyl ester

The title compound was prepared from 4-[6-(2,6-Dimethyl-benzyl)-3-hydroxy-pyridin-2-yl]-4-oxo-butyric acid ethyl ester (see example 54b) and bromine in analogy to example 2c, MS-(+)-ion, M+H=419.95, 421.90.

### d) 4-[4-Cyano-6-(2,6-dimethyl-benzyl)-3-hydroxy-pyridin-2-yl]-4-oxo-butyric acid ethyl ester

The title compound was prepared from 4-[4-Bromo-6-(2,6-dimethyl-benzyl)-3-hydroxy-pyridin-2-yl]-4-oxo-butyric acid ethyl ester (see example 54c) and zinc(II) cyanide in analogy to example 2b, MS-(+)-ion, M+H=366.98.

### e) 4-[4-Cyano-6-(2,6-dimethyl-benzyl)-3-hydroxy-pyridin-2-yl]-4-oxo-butyric acid

The title compound was prepared from 4-[4-Cyano-6-(2,6-dimethyl-benzyl)-3-hydroxy-pyridin-2-yl]-4-oxo-butyric acid ethyl ester (see example 54d) in analogy to example 1g, MS-(+)-ion, M+H=338.98.

### Example 55

### 4-[4-Cyano-3-hydroxy-6-(2,4,6-trifluoro-benzyl)-pyridin-2-yl]-4-oxo-butyric acid

### a) 2,4,6-Trifluoro-benzylzinc(II) bromide

The title compound was prepared from 2-Bromomethyl-1,3,5-trifluoro-benzene and zinc dust in analogy to example 39a.

### b) 4-[6-(2,4,6-Trifluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2,4,6-Trifluoro-benzylzinc(II) bromide in THF (see Example 55a) in analogy to example 10a, MS-(+)-ion, M+H=367.98.

### c) 4-[4-Bromo-6-(2,4,6-trifluoro-benzyl)-3-hydroxy-pyridin-2-yl]-4-oxo-butyric acid ethyl ester

The title compound was prepared from 4-[6-(2,4,6-Trifluoro-benzyl)-3-hydroxy-pyridin-2-yl]-4-oxo-butyric acid ethyl ester (see example 55b) and bromine in analogy to example 2c, MS-(+)-ion, M+H=445.80, 447.75.

### d) 4-[4-Cyano-3-hydroxy-6-(2,4,6-trifluoro-benzyl)-pyridin-2-yl]-4-oxo-butyric acid ethyl ester

The title compound was prepared from 4-[4-Bromo-6-(2,4,6-trifluoro-benzyl)-3-hydroxy-pyridin-2-yl]-4-oxo-butyric acid ethyl ester (see example 55c) and zinc(II) cyanide in analogy to example 2b, MS-(+)-ion, M+H=392.94.

### e) 4-[4-Cyano-6-(2,4,6-trifluoro-benzyl)-3-hydroxy-pyridin-2-yl]-4-oxo-butyric acid

The title compound was prepared from 4-[4-Cyano-6-(2,4,6-trifluoro-benzyl)-3-hydroxy-pyridin-2-yl]-4-oxo-butyric acid ethyl ester (see example 55d) in analogy to example 1g, MS-(+)-ion, M+H=364.88.

### Example 56

### 4-[6-(3-Chloro-2,6-difluoro-benzyl)-4-cyano-3-hydroxy-pyridin-2-yl]-4-oxo-butyric acid

### a) 3-Chloro-2,6-difluoro-benzylzinc(II) bromide

The title compound was prepared from 2-Bromomethyl-4-chloro-1,3-difluoro-benzene and zinc dust in analogy to example 39a.

### b) 4-[6-(3-Chloro-2,6-difluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 3-Chloro-2,6-difluoro-benzylzinc(II) bromide in THF (see Example 56a) in analogy to example 10a, MS-(+)-ion, M+H=383.89.

### c) 4-[4-Bromo-6-(3-chloro-2,6-difluoro-benzyl)-3-hydroxy-pyridin-2-yl]-4-oxo-butyric acid ethyl ester

The title compound was prepared from 4-[6-(3-Chloro-2,6-difluoro-benzyl)-3-hydroxy-pyridin-2-yl]-4-oxo-butyric acid ethyl ester (see example 56b) and bromine in analogy to example 2c, MS-(+)-ion, M+H=463.70.

### d) 4-[6-(3-Chloro-2,6-difluoro-benzyl)-4-cyano-3-hydroxy-pyridin-2-yl]-4-oxo-butyric acid ethyl ester

The title compound was prepared from 4-[4-Bromo-6-(3-chloro-2,6-difluoro-benzyl)-3-hydroxy-pyridin-2-yl]-4-oxo-butyric acid ethyl ester (see example 56c) and zinc(II) cyanide in analogy to example 2b, MS-(+)-ion, M+H=408.99.

### e) 4-[6-(3-Chloro-2,6-difluoro-benzyl)-4-cyano-3-hydroxy-pyridin-2-yl]-4-oxo-butyric acid

The title compound was prepared from 4-[6-(3-Chloro-2,6-difluoro-benzyl)-4-cyano-3-hydroxy-pyridin-2-yl]-4-oxo-butyric acid ethyl ester (see example 56d) in analogy to example 1g, MS-(+)-ion, M+H=380.89.

### Example 57

### 4-[4-Cyano-3-hydroxy-6-(2,3,6-trifluoro-benzyl)-pyridin-2-yl]-4-oxo-butyric acid

### a) 2,3,6-Trifluoro-benzylzinc(II) bromide

The title compound was prepared from 2-Bromomethyl-1,3,4-trifluoro-benzene and zinc dust in analogy to example 39a.

### b) 4-[6-(2,3,6-Trifluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2,3,6-Trifluoro-benzylzinc(II) bromide in THF (see Example 57a) in analogy to example 10a, MS-(+)-ion, M+H=367.93.

### c) 4-[4-Bromo-6-(2,3,6-trifluoro-benzyl)-3-hydroxy-pyridin-2-yl]-4-oxo-butyric acid ethyl ester

The title compound was prepared from 4-[6-(2,4,6-Trifluoro-benzyl)-3-hydroxy-pyridin-2-yl]-4-oxo-butyric acid ethyl ester (see example 57b) and bromine in analogy to example 2c, MS-(+)-ion, M+H=445.75, 447.75.

### d) 4-[4-Cyano-3-hydroxy-6-(2,3,6-trifluoro-benzyl)-pyridin-2-yl]-4-oxo-butyric acid ethyl ester

The title compound was prepared from 4-[4-Bromo-6-(2,3,6-trifluoro-benzyl)-3-hydroxy-pyridin-2-yl]-4-oxo-butyric acid ethyl ester (see example 57c) and zinc(II) cyanide in analogy to example 2b, MS-(+)-ion, M+H=392.99.

### e) 4-[4-Cyano-6-(2,4,6-trifluoro-benzyl)-3-hydroxy-pyridin-2-yl]-4-oxo-butyric acid

The title compound was prepared from 4-[4-Cyano-6-(2,3,6-trifluoro-benzyl)-3-hydroxy-pyridin-2-yl]-4-oxo-butyric acid ethyl ester (see example 57d) in analogy to example 1g, MS-(+)-ion, M+H=364.93.

### Example 58

### 4-[6-(2-Chloro-6-cyano-benzyl)-4-cyano-3-hydroxy-pyridin-2-yl]-4-oxo-butyric acid

### a) 2-Chloro-6-cyano-benzylzinc(II) bromide

The title compound was prepared from 2-Bromomethyl-3-chloro-benzonitrile and zinc dust in analogy to example 39a.

### b) 4-[6-(2-Chloro-6-cyano-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-Chloro-6-cyano-benzylzinc(II) bromide in THF (see Example 58a) in analogy to example 10a, MS-(+)-ion, M+H=372.99.

### c) 4-[4-Bromo-6-(2-chloro-6-cyano-benzyl)-3-hydroxy-pyridin-2-yl]-4-oxo-butyric acid ethyl ester

The title compound was prepared from 4-[4-Bromo-6-(2-chloro-6-cyano-benzyl)-3-hydroxy-pyridin-2-yl]-4-oxo-butyric acid ethyl ester (see example 58b) and bromine in analogy to example 2c, MS-(+)-ion, M+H=452.75.

### d) 4-[6-(2-Chloro-6-cyano-benzyl)-4-cyano-3-hydroxy-pyridin-2-yl]-4-oxo-butyric acid ethyl ester

The title compound was prepared from 4-[4-Bromo-6-(2-chloro-6-cyano-benzyl)-3-hydroxy-pyridin-2-yl]-4-oxo-butyric acid ethyl ester (see example 58c) and zinc(II) cyanide in analogy to example 2b, MS-(+)-ion, M+H=397.99.

### e) 4-[6-(2-Chloro-6-cyano-benzyl)-4-cyano-3-hydroxy-pyridin-2-yl]-4-oxo-butyric acid

The title compound was prepared from 4-[6-(2-Chloro-6-cyano-benzyl)-4-cyano-3-hydroxy-pyridin-2-yl]-4-oxo-butyric acid ethyl ester (see example 58d) in analogy to example 1g, MS-(+)-ion, M+H=369.88.

### Example 59

### 4-[6-(2-Chloro-6-trifluoromethyl-benzyl)-4-cyano-3-hydroxy-pyridin-2-yl]-4-oxo-butyric acid

### a) 2-Chloro-6-trifluoromethyl-benzylzinc(II) bromide

The title compound was prepared from 2-Bromomethyl-1-chloro-3-trifluoromethyl-benzene and zinc dust in analogy to example 39a.

### b) 4-[6-(2-Chloro-6-trifluoromethyl-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-Chloro-6-trifluoromethyl-benzylzinc(II) bromide in THF (see Example 59a) in analogy to example 10a, MS-(+)-ion, M+H=415.89.

### c) 4-[4-Bromo-6-(2-chloro-6-trifluoromethyl-benzyl)-3-hydroxy-pyridin-2-yl]-4-oxo-butyric acid ethyl ester

The title compound was prepared from 4-[6-(2-Chloro-6-trifluoromethyl-benzyl)-3-hydroxy-pyridin-2-yl]-4-oxo-butyric acid ethyl ester (see example 59b) and bromine in analogy to example 2c, MS-(+)-ion, M+H=495.66.

### d) 4-[6-(2-Chloro-6-trifluoromethyl-benzyl)-4-cyano-3-hydroxy-pyridin-2-yl]-4-oxo-butyric acid ethyl ester

The title compound was prepared from 4-[4-Bromo-6-(2-chloro-6-trifluoromethyl-benzyl)-3-hydroxy-pyridin-2-yl]-4-oxo-butyric acid ethyl ester (see example 59c) and zinc(II) cyanide in analogy to example 2b, MS-(+)-ion, M+H=440.85.

### e) 4-[6-(2-Chloro-6-trifluoromethyl-benzyl)-4-cyano-3-hydroxy-pyridin-2-yl]-4-oxo-butyric acid

The title compound was prepared from 4-[6-(2-Chloro-6-trifluoromethyl-benzyl)-4-cyano-3-hydroxy-pyridin-2-yl]-4-oxo-butyric acid ethyl ester (see example 59d) in analogy to example 1g, MS-(+)-ion, M+H=412.94.

### Example 60

### 4-[6-(2-Fluoro-6-trifluoromethyl-benzyl)-4-cyano-3-hydroxy-pyridin-2-yl]-4-oxo-butyric acid

### a) 2-Fluoro-6-trifluoromethyl-benzylzinc(II) bromide

The title compound was prepared from 2-Bromomethyl-1-fluoro-3-trifluoromethyl-benzene and zinc dust in analogy to example 39a.

### b) 4-[6-(2-Fluoro-6-trifluoromethyl-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-Fluoro-6-trifluoromethyl-benzylzinc(II) bromide in THF (see Example 60a) in analogy to example 10a, MS-(+)-ion, M+H=399.94.

### c) 4-[4-Bromo-6-(2-fluoro-6-trifluoromethyl-benzyl)-3-hydroxy-pyridin-2-yl]-4-oxo-butyric acid ethyl ester

The title compound was prepared from 4-[6-(2-Fluoro-6-trifluoromethyl-benzyl)-3-hydroxy-pyridin-2-yl]-4-oxo-butyric acid ethyl ester (see example 60b) and bromine in analogy to example 2c, MS-(+)-ion, M+H=477.76, 479.66.

### d) 4-[6-(2-Fluoro-6-trifluoromethyl-benzyl)-4-cyano-3-hydroxy-pyridin-2-yl]-4-oxo-butyric acid ethyl ester

The title compound was prepared from 4-[4-Bromo-6-(2-fluoro-6-trifluoromethyl-benzyl)-3-hydroxy-pyridin-2-yl]-4-oxo-butyric acid ethyl ester (see example 60c) and zinc(II) cyanide in analogy to example 2b, MS-(+)-ion, M+H=424.95.

### e) 4-[6-(2-Fluoro-6-trifluoromethyl-benzyl)-4-cyano-3-hydroxy-pyridin-2-yl]-4-oxo-butyric acid

The title compound was prepared from 4-[6-(2-Fluoro-6-trifluoromethyl-benzyl)-4-cyano-3-hydroxy-pyridin-2-yl]-4-oxo-butyric acid ethyl ester (see example 60d) in analogy to example 1g, MS-(+)-ion, M+H=396.89.

### Example 61

### 4-[4-Cyano-3-hydroxy-6-(2-methoxy-6-trifluoromethyl-benzyl)-pyridin-2-yl]-4-oxo-butyric acid

### a) 2-Methoxy-6-trifluoromethyl-benzylzinc(II) bromide

The title compound was prepared from 2-Bromomethyl-1-methoxy-3-trifluoromethyl-benzene and zinc dust in analogy to example 39a.

### b) 4-[3-Hydroxy-6-(2-methoxy-6-trifluoromethyl-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-Methoxy-6-trifluoromethyl-benzylzinc(II) bromide in THF (see Example 61a) in analogy to example 10a, MS-(+)-ion, M+H=411.99.

### c) 4-[4-Bromo-3-hydroxy-6-(2-methoxy-6-trifluoromethyl-benzyl)-pyridin-2-yl]-4-oxo-butyric acid ethyl ester

The title compound was prepared from 4-[3-Hydroxy-6-(2-methoxy-6-trifluoromethyl-benzyl)-pyridin-2-yl]-4-oxo-butyric acid ethyl ester (see example 61b) and bromine in analogy to example 2c, MS-(+)-ion, M+H=489.81, 491.76.

### d) 4-[4-Cyano-3-hydroxy-6-(2-methoxy-6-trifluoromethyl-benzyl)-pyridin-2-yl]-4-oxo-butyric acid ethyl ester

The title compound was prepared from 4-[4-Bromo-3-hydroxy-6-(2-methoxy-6-trifluoromethyl-benzyl)-pyridin-2-yl]-4-oxo-butyric acid ethyl ester (see example 61c) and zinc(II) cyanide in analogy to example 2b, MS-(+)-ion, M+H=436.95.

### e) 4-[4-Cyano-3-hydroxy-6-(2-methoxy-6-trifluoromethyl-benzyl)-pyridin-2-yl]-4-oxo-butyric acid

The title compound was prepared from 4-[4-Cyano-3-hydroxy-6-(2-methoxy-6-trifluoromethyl-benzyl)-pyridin-2-yl]-4-oxo-butyric acid ethyl ester (see example 61d) in analogy to example 1g, MS-(+)-ion, M+H=408.94.

### Example 62

### 4-[4-Cyano-3-hydroxy-6-(2-methyl-6-trifluoromethyl-benzyl)-pyridin-2-yl]-4-oxo-butyric acid

### a) 2-Methyl-6-trifluoromethyl-benzylzinc(II) bromide

The title compound was prepared from 2-Bromomethyl-1-methyl-3-trifluoromethyl-benzene and zinc dust in analogy to example 39a.

### b) 4-[3-Hydroxy-6-(2-methyl-6-trifluoromethyl-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2-Methyl-6-trifluoromethyl-benzylzinc(II) bromide in THF (see Example 62a) in analogy to example 10a. ¹H NMR (CDCl₃, 200 MHz) δ=11.41 (s, 1H), 7.58 (d, J=7.4 Hz, 1H), 7.42-7.16 (m, 3H), 6.98 (d, J=8.8 Hz, 1H), 4.33 (s, 2H), 4.16 (q, J=7.0 Hz, 2H), 3.46 (t, J=6.6 Hz, 1H), 2.67 (t, J=6.6 Hz, 1H), 2.25 (s, 3H), 1.27 (t, J=7.0 Hz, 3H).

### c) 4-[4-Bromo-3-hydroxy-6-(2-methyl-6-trifluoromethyl-benzyl)-pyridin-2-yl]-4-oxo-butyric acid ethyl ester

The title compound was prepared from 4-[3-Hydroxy-6-(2-methyl-6-trifluoromethyl-benzyl)-pyridin-2-yl]-4-oxo-butyric acid ethyl ester (see example 62b) and bromine in analogy to example 2c, MS-(+)-ion, M+H=473.86, 475.81.

### d) 4-[4-Cyano-3-hydroxy-6-(2-methyl-6-trifluoromethyl-benzyl)-pyridin-2-yl]-4-oxo-butyric acid ethyl ester

The title compound was prepared from 4-[4-Bromo-3-hydroxy-6-(2-methyl-6-trifluoromethyl-benzyl)-pyridin-2-yl]-4-oxo-butyric acid ethyl ester (see example 62c) and zinc(II) cyanide in analogy to example 2b, MS-(+)-ion, M+H=421.00.

### e) 4-[4-Cyano-3-hydroxy-6-(2-methyl-6-trifluoromethyl-benzyl)-pyridin-2-yl]-4-oxo-butyric acid

The title compound was prepared from 4-[4-Cyano-3-hydroxy-6-(2-methyl-6-trifluoromethyl-benzyl)-pyridin-2-yl]-4-oxo-butyric acid ethyl ester (see example 62d) in analogy to example 1g, MS-(+)-ion, M+H=392.94.

### Example 63

### 4-[4-Cyano-6-(2,5-dichloro-benzyl)-3-hydroxy-pyridin-2-yl]-4-oxo-butyric acid

### a) 2,5-Dichloro-benzylzinc(II) bromide

The title compound was prepared from 2-Bromomethyl-1,4-dichloro-benzene and zinc dust in analogy to example 39a.

### b) 4-[6-(2,5-Dichloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2,5-Dichloro-benzylzinc(II) bromide in THF (see Example 63a) in analogy to example 10a, MS-(+)-ion, M+H=381.94, 383.84.

### c) 4-[4-Bromo-6-(2,5-dichloro-benzyl)-3-hydroxy-pyridin-2-yl]-4-oxo-butyric acid ethyl ester

The title compound was prepared from 4-[6-(2,5-Dichloro-benzyl)-3-hydroxy-pyridin-2-yl]-4-oxo-butyric acid ethyl ester (see example 63b) and bromine in analogy to example 2c, MS-(+)-ion, M+H=461.70.

### d) 4-[4-Cyano-6-(2,5-dichloro-benzyl)-3-hydroxy-pyridin-2-yl]-4-oxo-butyric acid ethyl ester

The title compound was prepared from 4-[4-Bromo-6-(2,5-dichloro-benzyl)-3-hydroxy-pyridin-2-yl]-4-oxo-butyric acid ethyl ester (see example 63c) and zinc(II) cyanide in analogy to example 2b, MS-(+)-ion, M+H=406.89, 408.84.

### e) 4-[4-Cyano-6-(2,5-dichloro-benzyl)-3-hydroxy-pyridin-2-yl]-4-oxo-butyric acid

The title compound was prepared from 4-[4-Cyano-6-(2,5-dichloro-benzyl)-3-hydroxy-pyridin-2-yl]-4-oxo-butyric acid ethyl ester (see example 63d) in analogy to example 1g, MS-(+)-ion, M+H=378.89, 380.74.

### Example 64

### 4-[4-Cyano-6-(2,4-dichloro-benzyl)-3-hydroxy-pyridin-2-yl]-4-oxo-butyric acid

### a) 2,4-Dichloro-benzylzinc(II) bromide

The title compound was prepared from 1-Bromomethyl-2,4-dichloro-benzene and zinc dust in analogy to example 39a.

### b) 4-[6-(2,4-Dichloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2,4-Dichloro-benzylzinc(II) bromide in THF (see Example 64a) in analogy to example 10a, MS-(+)-ion, M+H=381.94, 383.84.

### c) 4-[4-Bromo-6-(2,4-dichloro-benzyl)-3-hydroxy-pyridin-2-yl]-4-oxo-butyric acid ethyl ester

The title compound was prepared from 4-[6-(2,4-Dichloro-benzyl)-3-hydroxy-pyridin-2-yl]-4-oxo-butyric acid ethyl ester (see example 64b) and bromine in analogy to example 2c, MS-(+)-ion, M+H=461.70.

### d) 4-[4-Cyano-6-(2,4-dichloro-benzyl)-3-hydroxy-pyridin-2-yl]-4-oxo-butyric acid ethyl ester

The title compound was prepared from 4-[4-Bromo-6-(2,4-dichloro-benzyl)-3-hydroxy-pyridin-2-yl]-4-oxo-butyric acid ethyl ester (see example 64c) and zinc(II) cyanide in analogy to example 2b, MS-(+)-ion, M+H=406.94, 408.84.

### e) 4-[4-Cyano-6-(2,4-dichloro-benzyl)-3-hydroxy-pyridin-2-yl]-4-oxo-butyric acid

The title compound was prepared from 4-[4-Cyano-6-(2,4-dichloro-benzyl)-3-hydroxy-pyridin-2-yl]-4-oxo-butyric acid ethyl ester (see example 64d) in analogy to example 1g, MS-(+)-ion, M+H=378.89, 380.79.

### Example 65

### 4-[4-Cyano-6-(2,3-dichloro-benzyl)-3-hydroxy-pyridin-2-yl]-4-oxo-butyric acid

### a) 2,3-Dichloro-benzylzinc(II) bromide

The title compound was prepared from 1-Bromomethyl-2,3-dichloro-benzene and zinc dust in analogy to example 39a.

### b) 4-[6-(2,3-Dichloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester (see Example 2a) and 2,3-Dichloro-benzylzinc(II) bromide in THF (see Example 65a) in analogy to example 10a, MS-(+)-ion, M+H=381.94, 383.84.

### c) 4-[4-Bromo-6-(2,3-dichloro-benzyl)-3-hydroxy-pyridin-2-yl]-4-oxo-butyric acid ethyl ester

The title compound was prepared from 4-[6-(2,3-Dichloro-benzyl)-3-hydroxy-pyridin-2-yl]-4-oxo-butyric acid ethyl ester (see example 65b) and bromine in analogy to example 2c, MS-(+)-ion, M+H=461.75.

### d) 4-[4-Cyano-6-(2,3-dichloro-benzyl)-3-hydroxy-pyridin-2-yl]-4-oxo-butyric acid ethyl ester

The title compound was prepared from 4-[4-Bromo-6-(2,3-dichloro-benzyl)-3-hydroxy-pyridin-2-yl]-4-oxo-butyric acid ethyl ester (see example 65c) and zinc(II) cyanide in analogy to example 2b, MS-(+)-ion, M+H=406.94, 408.79.

### e) 4-[4-Cyano-6-(2,3-dichloro-benzyl)-3-hydroxy-pyridin-2-yl]-4-oxo-butyric acid

The title compound was prepared from 4-[4-Cyano-6-(2,3-dichloro-benzyl)-3-hydroxy-pyridin-2-yl]-4-oxo-butyric acid ethyl ester (see example 65d) in analogy to example 1g, MS-(+)-ion, M+H=378.89, 380.74.

### Example 66

### 4-[4-Cyano-3-hydroxy-6-(3-trifluoromethoxy-phenyl)-pyridin-2-yl]-4-oxo-butyric acid

### a) 3-Hydroxy-pyridine-2-carboxylic acid ethyl ester

To the solution of 3-hydroxy-pyridine-2-carboxylic acid (15.0 g, 108 mmol) in anhydrous ethanol (300 mL) was added 98% sulfuric acid (17.0 mL, 324 mmol, 3.0 eq.). The reaction was refluxed for 24 hours. After the solvents were evaporated, the residue was dissolved in 300 mL of water, neutralized with saturated sodium bicarbonate solution and extracted with ethyl acetate (200 mL×3). The combined organics were washed with brine (200 mL), dried over sodium sulfate, filtered and evaporated in vacuo to afford the title compound; MS-(+)-ion, M+1=168.18.

### b) 3-Benzyloxy-pyridine-2-carboxylic acid ethyl ester

Benzyl bromide (7.35 mL, 10.57 g, 60.7 mmol, 1.4 eq.) was added to a mixture of 3-hydroxy-pyridine-2-carboxylic acid ethyl ester (7.25 g, 46.5 mmol, 1.0 eq., see Example 1a) and cesium carbonate (17.0 g, 52.1 mmol, 1.2 eq.) in anhydrous DMF (60 mL) at room temperature. After 3 hours at room temperature, TLC shows the completion of the reaction. The reaction mixture was diluted with water (200 mL) and extracted with ethyl acetate (4×150 mL). The combined extracts were washed with brine (2×200 mL), dried over sodium sulfate, filtered and evaporated in vacuo to afford the crude product. This material was purified by flash chromatography eluting with ethyl acetate/hexane (0%-70%) to give the title compound. ¹H NMR (CDCl₃, 200 MHz) δ=8.29 (t, J=2.8 Hz, 1H), 7.44-7.24 (m, 7H), 5.20 (s, 2H), 4.46 (q, J=7.0 Hz, 2H), 1.40 (t, J=7.0 Hz, 3H).

### c) [2-(3-Benzyloxy-pyridin-2-yl)-2-oxo-ethyl]-phosphonic acid dimethyl ester

At −78° C., to a solution of dimethyl methylphosphonate (10.0 mL, 92 mmol, 2.2 eq.) in THF (200 mL) was added n-BuLi (2.5 M in hexanes, 33.5 mL, 83.6 mmol, 2.0 eq) over 20 min under N₂ atmosphere. After 30 min, a solution of 3-benzyloxy-pyridine-2-carboxylic acid ethyl ester (11.41 g, 92 mmol, see Example 1b) in THF (50 mL) was added slowly over 20 min. After stirring for 1 h at −78° C., the mixture was treated with half saturated aq. NH₄Cl (200 mL) and extracted with ethyl acetate (4×150 mL). The combined extracts were dried over sodium sulfate, filtered and evaporated in vacuo to afford the crude product. This material was purified by flash chromatography eluting with MeOH/DCM (0%-8%) to give the title compound. ¹H NMR (200 MHz, CDCl₃) δ=8.27 (dd, J=3.2 Hz, 2.4 Hz, 1H), 7.46-7.26 (m, 7H), 5.23 9 (s, 2H), 3.98 (d, J=22.2 Hz, 2H), 3.77 (d, J=1.2 Hz, 3H), 3.71 (d, J=1.2 Hz, 3H). MS-(+)-ion, M+1=336.35.

### d) 4-(3-Benzyloxy-pyridin-2-yl)-4-oxo-but-2-enoic acid ethyl ester

To a solution of [2-(3-benzyloxy-pyridin-2-yl)-2-oxo-ethyl]-phosphonic acid dimethyl ester (10.0 g, 30.0 mmol, see Example 1c) in THF (120 mL) at −78° C. was added t-BuOK (1 M in THF, 36 mmol, 36 mL, 1.2 eq). After stirred at −78° C. for 10 min, 8.6 mL of ethyl glyoxalate (50 wt % in toluene, 12.24 g, 60.0 mmol, 2.0 eq) was added dropwise in 20 min via a dropping funnel. The mixture was stirred at −78° C. for 3 h, then was treated with half saturated aq. NH₄Cl (200 mL) and extracted with ethyl acetate (4×150 mL). The combined extracts were dried over sodium sulfate, filtered and evaporated in vacuo to afford the crude product. This material was purified by flash chromatography eluting with EtOAc/hexane (0%-30%) to give the title compound. ¹H NMR (200 MHz, CDCl₃) δ=8.27 (t, J=7.8 Hz, 1H), 8.11 (d, J=19.5 Hz, 1H), 7.48-7.32 (m, 7H), 6.85 (d, J=19.5 Hz, 1H), 5.23 (s, 2H), 4.28 (q, J=9.0 Hz, 2H), 1.34 (t, J=9.0 Hz, 3H). MS-(+)-ion, M+23=344.40.

### e) 4-(3-Hydroxy-pyridin-2-yl)-4-oxo-butyric acid ethyl ester

To a solution of 4-(3-benzyloxy-pyridin-2-yl)-4-oxo-but-2-enoic acid ethyl ester (3.3 g, 10.6 mmol, see Example 1d) in ethyl acetate (60 mL) was added Pd/C (200 mg, 0.01 eq, 10 wt. %, wet, contains ˜51% water). The mixture was vacuumed/refilled with hydrogen gas three times. After stirring for 16 hours at room temperature, the reaction mixture was filtered through celite. The filtrate was evaporated in vacuo to afford the crude product. This material was purified by flash chromatography eluting with EtOAc/hexane (0%-20%) to give the title compound. ¹H NMR (200 MHz, CDCl₃) δ=11.6 (s, 1H), 8.23 (dd, J=4.4 Hz, 1.6 Hz, 1H), 7.41-7.35 (m, 2H), 4.16 (q, J=7.4 Hz, 2H), 3.62 (t, J=6.6 Hz, 2H), 2.75 (t, J=6.6 Hz, 2H), 1.26 (t, J=7.4 Hz, 3H). MS-(+)-ion, M+1=223.96.

### f) 4-(6-Bromo-3-hydroxy-pyridin-2-yl)-4-oxo-butyric acid ethyl ester

To the solution of 4-(3-hydroxy-pyridin-2-yl)-4-oxo-butyric acid ethyl ester (2.0 g, 9.0 mmol) in DCM (9 mL) was added 90 mL H₂O. Bromine (0.51 mL, 1.1 eq, 9.9 mmol) was added slowly over 5 min to the above mixture at room temperature. The reaction flask was wrapped in aluminum foil. After 1 hour at room temperature, a second portion of bromine (0.10 mL, 0.2 eq, 1.8 mmol) was added to the reaction as TLC shows starting material remains. After another 2 hours, the reaction was quenched with 100 mL saturated NaHSO₃ aqueous solution and extracted with DCM (100 mL×3). The combined organics were washed with brine (200 mL) and dried over sodium sulfate, filtered and evaporated in vacuo to afford the crude product. This material was purified by flash chromatography eluting with ethyl acetate/hexane (0%-10%) to give the title compound. ¹H NMR (CDCl₃, 200 MHz) δ=11.58 (s, 1H), 7.53 (d, J=9.0 Hz, 1H), 7.23 (d, J=9.0 Hz, 1H, merged with CHCl₃), 4.16 (q, J=7.4 Hz, 2H), 3.56 (t, J=6.2 Hz, 2H), 2.73 (t, J=6.2 Hz, 2H), 1.26 (t, J=7.4 Hz, 3H). MS-(+)-ion, M+H=303.77.

### g) 4-[3-Hydroxy-6-(3-trifluoromethoxy-phenyl)-pyridin-2-yl]-4-oxo-butyric acid ethyl ester

The title compound was prepared from 4-(6-bromo-3-hydroxy-pyridin-2-yl)-4-oxo-butyric acid ethyl ester (66f) and 3-trifluoromethoxybenzeneboronic acid in analogy to example 9a to give the title compound: MS (m/z) 384 (M+1)+.

### h) 4-[4-Bromo-3-hydroxy-6-(3-trifluoromethoxy-phenyl)-pyridin-2-yl]-4-oxo-butyric acid ethyl ester

The title compound was prepared from 4-[3-hydroxy-6-(3-trifluoromethoxy-phenyl)-pyridin-2-yl]-4-oxo-butyric acid ethyl ester and bromine in analogy to example 2c to give the title compound: MS (m/z) 462, 464 (M+1)+.

### i) 4-[4-Cyano-3-hydroxy-6-(3-trifluoromethoxy-phenyl)-pyridin-2-yl]-4-oxo-butyric acid ethyl ester

To a solution of 4-[4-bromo-3-hydroxy-6-(3-trifluoromethoxy-phenyl)-pyridin-2-yl]-4-oxo-butyric acid ethyl ester (185 mg, 0.4 mmol) in DMAC (5.7 mL) at room temperature were added Zn(CN)₂ (93.7 mg, 0.8 mmol), Pd₂(dba)₃ (36.6 mg, 0.04 mmol), dppf (443 mg, 0.08 mmol), and Zn dust (7.8 mg, 0.12 mmol) to provide a suspension. The reaction mixture was allowed to stir at 100° C. for 3-4 hours. After cooling to rt, the reaction mixture was diluted with EtOAc (50 mL) and filtered through a celite plug, rinsing; with EtOAc (50 mL). The organic layers were washed with a 0.1 N HCl solution and water, dried over MgSO₄, concentrated in vacuo and purified by ISCO over silica gel, eluting with 5-35% EtOAc/hexanes to give the product. MS (m/z) 409.1 (M+1)⁺.

### j) 4-[4-Cyano-3-hydroxy-6-(3-trifluoromethoxy-phenyl)-pyridin-2-yl]-4-oxo-butyric acid

To a solution of 4-[4-cyano-3-hydroxy-6-(3-trifluoromethoxy-phenyl)-pyridin-2-yl]-4-oxo-butyric acid ethyl ester (77 mg, 0.18 mmol) in THF (2.0 mL) and water (0.7 mL) at room temperature was added lithium hydroxide monohydrate (31.7 mg, 0.75 mmol) to give a suspension. The reaction mixture was stirred at room temperature. After 24 h, the mixture was concentrated in vacuo to give a residue. The residue was dissolved in water (25 mL) and extracted with EtOAc (3×15 mL). The aqueous solution was acidified with a 1N HCl solution, filtered, washed with water, and dried to give product. MS (m/z) 381.1 (M+1)⁺.

### Example 67

### 4-[4-Cyano-3-hydroxy-6-(4-trifluoromethoxy-phenyl)-pyridin-2-yl]-4-oxo-butyric acid

### a) 4-[3-Hydroxy-6-(4-trifluoromethoxy-phenyl)-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 4-trifluoronethoxybenzeneboronic acid in analogy to example 9a to give the title compound: MS (m/z) 384 (M+1)⁺.

### b) 4-[4-Bromo-3-hydroxy-6-(4-trifluoromethoxy-phenyl)-pyridin-2-yl]-4-oxo-butyric acid ethyl ester

The title compound was prepared from 4-[3-hydroxy-6-(4-trifluoromethoxy-phenyl)-pyridin-2-yl]-4-oxo-butyric acid ethyl ester and bromine in analogy to example 2c to give the title compound: MS (m/z) 462, 464 (M+1)⁺.

### c) 4-[4-Cyano-3-hydroxy-6-(4-trifluoromethoxy-phenyl)-pyridin-2-yl]-4-oxo-butyric acid ethyl ester

To a solution of 4-[4-bromo-3-hydroxy-6-(4-trifluoromethoxy-phenyl)-pyridin-2-yl]-4-oxo-butyric acid ethyl ester (213 mg, 0.46 mmol) in DMAC (6.5 mL) at room temperature were added Zn(CN)₂ 108 mg, 0.92 mmol), Pd₂(dba)₃ (42.1 mg, 0.046 mmol), dppf (51 mg, 0.092 mmol), and Zn dust (9 mg, 0.13 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 409.1 (M+1)⁺.

### d) 4-[4-Cyano-3-hydroxy-6-(4-trifluoromethoxy-phenyl)-pyridin-2-yl]-4-oxo-butyric acid

To a solution of 4-[4-cyano-3-hydroxy-6-(4-trifluoromethoxy-phenyl)-pyridin-2-yl]-4-oxo-butyric acid ethyl ester (117 mg, 0.28 mmol) in THF (3.0 mL) and water (1 mL) at room temperature was added lithium hydroxide monohydrate (48 mg, 1.15 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 379.2 (M−1)⁺.

### Example 68

### 4-(4-Cyano-3-hydroxy-6-naphthalen-1-yl-pyridin-2-yl)-4-oxo-butyric acid

### a) 4-(3-Hydroxy-6-naphthalen-1-yl-pyridin-2-yl)-4-oxo-butyric acid ethyl ester

The title compound was prepared from 4-(6-bromo-3-hydroxy-pyridin-2-yl)-4-oxo-butyric acid ethyl ester (prepared from 66f) and 1-naphthalenboronic acid in analogy to example 9a to give the title compound: MS (m/z) 350 (M+1)⁺.

### b) 4-(4-Bromo-3-hydroxy-6-naphthalen-1-yl-pyridin-2-yl)-4-oxo-butyric acid ethyl ester

The title compound was prepared from 4-(3-hydroxy-6-naphthalen-1-yl-pyridin-2-yl)-4-oxo-butyric acid ethyl ester and bromine in analogy to example 2c to give the title compound: MS (m/z) 428, 430 (M+1)⁺.

### c) 4-(4-Cyano-3-hydroxy-6-naphthalen-1-yl-pyridin-2-yl)-4-oxo-butyric acid ethyl ester

To a solution of 4-(4-bromo-3-hydroxy-6-naphthalen-1-yl-pyridin-2-yl)-4-oxo-butyric acid ethyl ester (150 mg, 0.35 mmol) in DMAC (5 mL) at room temperature were added Zn(CN)₂ (82 mg, 0.7 mmol), Pd₂(dba)₃ (32 mg, 0.035 mmol), dppf (38.8 mg, 0.07 mmol, and Zn dust (6.8 mg, 0.11 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 375.2 (M+1)⁺.

### d) 4-(4-Cyano-3-hydroxy-6-naphthalen-1-yl-pyridin-2-yl)-4-oxo-butyric acid

To a solution of 4-(4-cyano-3-hydroxy-6-naphthalen-1-yl-pyridin-2-yl)-4-oxo-butyric acid ethyl ester (75.7 mg, 0.2 mmol) in THF (2.1 mL) and water (0.7 mL) at room temperature was added lithium hydroxide monohydrate (34 mg, 0.8 mmol) to give a suspension. The reaction mixture was stirred at room temperature. After 24 h, the mixture was concentrated in vacuo to give a residue. The residue was dissolved in water (25 mL) and extracted with EtOAc (3×15 mL). The aqueous solution was acidified with a 1N HCl solution, filtered, washed with water, and dried to give product. MS (m/z) 345.2 (M−1)⁺.

### Example 69

### 4-[4-Cyano-6-(2-fluoro-phenyl)-3-hydroxy-pyridin-2-yl]-4-oxo-butyric acid

### a) 4-[6-(2-Fluoro-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-fluorobenzeneboronic acid in analogy to example 9a to give the title compound: MS (m/z) 318 (M+1)⁺.

### b) 4-[4-Bromo-6-(2-fluoro-phenyl)-3-hydroxy-pyridin-2-yl]-4-oxo-butyric acid ethyl ester

The title compound was prepared from 4-[6-(2-fluoro-phenyl)-3-hydroxy-pyridin-2-yl]-4-oxo-butyric acid ethyl ester and bromine in analogy to example 2c to give the title compound: MS (m/z) 396, 398 (M+1)⁺.

### c) 4-[4-Cyano-6-(2-fluoro-phenyl)-3-hydroxy-pyridin-2-yl]-4-oxo-butyric acid ethyl ester

To a solution of 4-[4-bromo-6-(2-fluoro-phenyl)-3-hydroxy-pyridin-2-yl]-4-oxo-butyric acid ethyl ester (182 mg, 0.46 mmol) in DMAC (6.6 mL) at room temperature were added Zn(CN)₂ (107 mg, 0.92 mmol), Pd₂(dba)₃ (42 mg, 0.046 mmol), dppf (51 mg, 0.092 mmol) and Zn dust (8.9 mg, 0.13 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 343.1 (M+1)⁺.

### d) 4-[4-Cyano-6-(2-fluoro-phenyl)-3-hydroxy-pyridin-2-yl]-4-oxo-butyric acid

To a solution of 4-[4-cyano-6-(2-fluoro-phenyl)-3-hydroxy-pyridin-2-yl]-4-oxo-butyric acid ethyl ester (105 mg, 0.31 mmol) in THF (3.3 mL) and water (1.1 mL) at room temperature was added lithium hydroxide monohydrate (52 mg, 1.22 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 313.2 (M−1)⁺.

### Example 70

### 4-[6-(2-Chloro-phenyl)-4-cyano-3-hydroxy-pyridin-2-yl]-4-oxo-butyric acid

### a) 4-[6-(2-Chloro-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chlorobenzeneboronic acid in analogy to example 9a to give the title compound: MS (m/z) 334 (M+1)⁺.

### b) 4-[4-Bromo-6-(2-chloro-phenyl)-3-hydroxy-pyridin-2-yl]-4-oxo-butyric acid ethyl ester

The title compound was prepared from 4-[6-(2-chloro-phenyl)-3-hydroxy-pyridin-2-yl]-4-oxo-butyric acid ethyl ester and bromine in analogy to example 2c to give the title compound: MS (m/z) 412, 414 (M+1)⁺.

### c) 4-[6-(2-Chloro-phenyl)-4-cyano-3-hydroxy-pyridin-2-yl]-4-oxo-butyric acid ethyl ester

To a solution of 4-[4-bromo-6-(2-chloro-phenyl)-3-hydroxy-pyridin-2-yl]-4-oxo-butyric acid ethyl ester (93.6 mg, 0.22 mmol) in DMAC (3.2 mL) at room temperature were added Zn(CN)₂ (53.2 mg, 0.45 mmol), Pd₂(dba)₃ (21 mg, 0.022 mmol), dppf 125.2 mg, 0.045 mmol), and Zn dust (4.5 mg, 007 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 359.1, 361.1 (M+1)⁺.

### d) 4-[6-(2-Chloro-phenyl)-4-cyano-3-hydroxy-pyridin-2-yl]-4-oxo-butyric acid

To a solution of 4-[6-(2-chloro-phenyl)-4-cyano-3-hydroxy-pyridin-2-yl]-4-oxo-butyric acid ethyl ester (55 mg, 0.15 mmol) in THF (1.6 mL) and water (0.55 mL) at room temperature was added lithium hydroxide monohydrate (25.8 mg, 0.61 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 329.1, 331.1 (M−1)⁺.

### Example 71

### 4-(4-Cyano-3-hydroxy-6-o-tolyl-pyridin-2-yl)-4-oxo-butyric acid

### a) 4-(3-Hydroxy-6-o-tolyl-pyridin-2-yl)-4-oxo-butyric acid ethyl ester

The title compound was prepared from 4-(6-bromo-3-hydroxy-pyridin-2-yl)-4-oxo-butyric acid ethyl ester (prepared from 66f) and 2-methylbenzeneboronic acid in analogy to example 9a to give the title compound: MS (m/z) 314 (M+1)⁺.

### b) 4-(4-Bromo-3-hydroxy-6-o-tolyl-pyridin-2-yl)-4-oxo-butyric acid ethyl ester

The title compound was prepared from 4-(3-hydroxy-6-o-tolyl-pyridin-2-yl)-4-oxo-butyric acid ethyl ester and bromine in analogy to example 2c to give the title compound: MS (m/z) 392, 394 (M+1)⁺.

### c) 4-(4-Cyano-3-hydroxy-6-o-tolyl-pyridin-2-yl)-4-oxo-butyric acid ethyl ester

To a solution of 4-(4-bromo-3-hydroxy-6-o-tolyl-pyridin-2-yl)-4-oxo-butyric acid ethyl ester (148 mg, 0.37 mmol) in DMAC (5.3 mL) at room temperature were added Zn(CN)_{z}(87 mg, 0.74 mmol), Pd₂(dba)₃ (34 mg, 0.037 mmol), dppf (41 mg, 0.074 mmol), and Zn dust (7.2 mg, 0.11 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 339.2 (M+1)⁺.

### d) 4-(4-Cyano-3-hydroxy-6-o-tolyl-pyridin-2-yl)-4-oxo-butyric acid

To a solution of 4-(4-cyano-3-hydroxy-6-o-tolyl-pyridin-2-yl)-4-oxo-butyric acid ethyl ester (97.1 mg, 0.28 mmol) in THF (3.1 mL) and water (1.0 mL) at room temperature was added lithium hydroxide monohydrate (48 mg, 1.15 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 309.2 (M−1)⁺.

### Example 72

### 4-(4-Cyano-3-hydroxy-6-m-tolyl-pyridin-2-yl)-4-oxo-butyric acid

### a) 4-(3-Hydroxy-6-m-tolyl-pyridin-2-yl)-4-oxo-butyric acid ethyl ester

The title compound was prepared from 4-(6-bromo-3-hydroxy-pyridin-2-yl)-4-oxo-butyric acid ethyl ester (prepared from 66f) and 3-methylbenzeneboronic acid in analogy to example 9a to give the title compound: MS (m/z) 314 (M+1)⁺.

### b) 4-(4-Bromo-3-hydroxy-6-m-tolyl-pyridin-2-yl)-4-oxo-butyric acid ethyl ester

The title compound was prepared from 4-(3-hydroxy-6-m-tolyl-pyridin-2-yl)-4-oxo-butyric acid ethyl ester and bromine in analogy to example 2c to give the title compound: MS (m/z) 392, 394 (M+1)⁺.

### c) 4-(4-Cyano-3-hydroxy-6-m-tolyl-pyridin-2-yl)-4-oxo-butyric acid ethyl ester

To a solution of 4-(4-bromo-3-hydroxy-6-m-tolyl-pyridin-2-yl)-4-oxo-butyric acid ethyl ester (147 mg, 0.37 mmol) in DMAC (5.3 mL) at room temperature were added Zn(CN)₂ (88 mg, 0.74 mmol), Pd₂(dba)₃, (34 mg, 0.037 mmol), dppf (42 mg, 0.074 mmol), and Zn dust (7.3 mg, 0.11 mmol) to give a suspension. The reaction mixture was allowed to stir at 100° C. for 3-4 horns. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 339.2 (M+1)⁺.

### d) 4-(4-Cyano-3-hydroxy-6-m-tolyl-pyridin-2-yl)-4-oxo-butyric acid

To a solution of 4-(4-cyano-3-hydroxy-6-m-tolyl-pyridin-2-yl)-4-oxo-butyric acid ethyl ester (80.2 mg, 0.28 mmol) in THF (2.5 mL) and water (0.85 mL) at room temperature was added lithium hydroxide monohydrate (39.8 mg, 0.95 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 309.2 (M−1)⁺.

### Example 73

### 4-(4-Cyano-3-hydroxy-6-p-tolyl-pyridin-2-yl)-4-oxo-butyric acid

### a) 4-(3-Hydroxy-6-p-tolyl-pyridin-2-yl)-4-oxo-butyric acid ethyl ester

The title compound was prepared from 4-(6-bromo-3-hydroxy-pyridin-2-yl)-4-oxo-butyric acid ethyl ester (prepared from 66f) and 4-methylbzeneboronic acid in analogy to example 9a to give the title compound: MS (m/z) 314 (M+1)⁺.

### b) 4-(4-Bromo-3-hydroxy-6-p-tolyl-pyridin-2-yl)-4-oxo-butyric acid ethyl ester

The title compound was prepared from 4-(3-hydroxy-6-p-tolyl-pyridin-2-yl)-4-oxo-butyric acid ethyl ester and bromine in analogy to example 2c to give the title compound: MS (m/z) 392, 394 (M+1)⁺.

### c) 4-(4-Cyano-3-hydroxy-6-p-tolyl-pyridin-2-yl)-4-oxo-butyric acid ethyl ester

To a solution of 4-(4-bromo-3-hydroxy-6-p-tolyl-pyridin-2-yl)-4-oxo-butyric acid ethyl ester (63 mg, 0.16 mmol) in DMAC (2.3 mL) at room temperature were added Zn(CN)₂ (38 mg, 0.32 mmol), Pd₂(dba)₃ (15 mg, 0.016 mmol), dppf (18 mg, 0.032 mmol), and Zn dust (3.1 mg, 0.05 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 339.2 (M+1)⁺.

### d) 4-(4-Cyano-3-hydroxy-6-p-tolyl-pyridin-2-yl)-4-oxo-butyric acid

To a solution of 4-(4-cyano-3-hydroxy-6-p-tolyl-pyridin-2-yl)-4-oxo-butyric acid ethyl ester (36.3 mg, 0.107 mmol) in THF (1.15 mL) and water (0.35 mL) at room temperature was added lithium hydroxide monohydrate (18 mg, 0.42 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 309.2 (M−1)⁺.

### Example 74

### 4-(6-Biphenyl-3-yl-4-cyano-3-hydroxy-pyridin-2-yl)-4-oxo-butyric acid

### a) 4-(6-Biphenyl-3-yl-3-hydroxy-pyridin-2-yl)-4-oxo-butyric acid ethyl ester

The title compound was prepared from 4-(6-bromo-3-hydroxy-pyridin-2-yl)-4-oxo-butyric acid ethyl ester (prepared from 66f) and 3-biphenylboronic acid in analogy to example 9a to give the title compound: MS (m/z) 376 (M+1)⁺.

### b) 4-(6-Biphenyl-3-yl-4-bromo-3-hydroxy-pyridin-2-yl)-4-oxo-butyric acid ethyl ester

The title compound was prepared from 4-(6-biphenyl-3-yl-3-hydroxy-pyridin-2-yl)-4-oxo-butyric acid ethyl ester and bromine in analogy to example 2c to give the title compound: MS (m/z) 454, 456 (M+1)⁺.

### c) 4-(6-Biphenyl-3-yl-4-cyano-3-hydroxy-pyridin-2-yl)-4-oxo-butyric acid ethyl ester

To a solution of 4-(6-biphenyl-3-yl-4-bromo-3-hydroxy-pyridin-2-yl)-4-oxo-butyric acid ethyl ester (121 mg, 0.266 mmol) in DMAC (3.8 mL) at room temperature were added Zn(CN)₂ (62 mg, 0.53 mmol), Pd₂(dba)t (24.3 mg, 0.0266 mmol), dppf (29.5 mg, 0.053 mmol), and Zn dust (5.2 mg, 0.08 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 401.1 (M+1)⁺.

### d) 4-(6-Biphenyl-3-yl-4-cyano-3-hydroxy-pyridin-2-yl)-4-oxo-butyric acid

To a solution of 4-(6-biphenyl-3-yl-4-cyano-3-hydroxy-pyridin-2-yl)-4-oxo-butyric acid ethyl ester (70.2 mg, 0.175 mmol) in THF (1.8 mL) and water (0.6 mL) at room temperature was added lithium hydroxide monohydrate (29.5 mg, 0.7 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 373.1 (M+1)⁺.

### Example 75

### 4-(6-Biphenyl-4-yl-4-cyano-3-hydroxy-pyridin-2-yl)-4-oxo-butyric acid

### a) 4-(6-Biphenyl-4-yl-3-hydroxy-pyridin-2-yl)-4-oxo-butyric acid ethyl ester

The title compound was prepared from 4-(6-bromo-3-hydroxy-pyridin-2-yl)-4-oxo-butyric acid ethyl ester (prepared from 66f) and 4-biphenylboronic acid in analogy to example 9a to give the title compound: MS (m/z) 376 (M+1)⁺.

### b) 4-(6-Biphenyl-4-yl-4-bromo-3-hydroxy-pyridin-2-yl)-4-oxo-butyric acid ethyl ester

The title compound was prepared from 4-(6-biphenyl-4-yl-3-hydroxy-pyridin-2-yl)-4-oxo-butyric acid ethyl ester and bromine in analogy to example 2c to give the title compound: MS (m/z) 454, 456 (M+1)⁺.

### c) 4-(6-Biphenyl-4-yl-4-cyano-3-hydroxy-pyridin-2-yl)-4-oxo-butyric acid ethyl ester

To a solution of 4-(6-biphenyl-4-yl-4-bromo-3-hydroxy-pyridin-2-yl)-4-oxo-butyric acid ethyl ester (146 mg, 0.32 mmol) in DMAC (4.6 mL) at room temperature were added Zn(CN)₂ (75.2 mg, 0.64 mmol), Pd₂(dba (29.3 rug, 0.032 mmol dppf (35 mg, 0.064 mmol), and Zn dust (6.2 mg, 0.096 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 401.1 (M+1)⁺.

### d) 4-(6-Biphenyl-4-yl-4-cyano-3-hydroxy-pyridin-2-yl)-4-oxo-butyric acid

To a solution of 4-(6-biphenyl-4-yl-4-cyano-3-hydroxy-pyridin-2-yl)-4-oxo-butyric acid ethyl ester (88.4 mg, 0.19 mmol) in THF (2.1 mL) and water (0.7 mL) at room temperature was added lithium hydroxide monohydrate (32.7 mg, 0.78 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 373.1 (M+1)⁺.

### Example 76

### 4-(4-Cyano-3-hydroxy-6-phenoxy-pyridin-2-yl)-4-oxo-butyric acid

### a) 4-(3-Hydroxy-6-phenoxy-pyridin-2-yl)-4-oxo-butyric acid ethyl ester

To a solution of 4-(6-bromo-3-hydroxy-pyridin-2-yl)-4-oxo-butyric acid ethyl ester (965 mg, 3.19 mmol, prepared from 1f) in DMAC (16 mL) at room temperature were added PhOH (1.05 g, 11.18 mmol), Cs₂CO₃ (5.2 g, 15.95 mmol, CuCl (31.6 mg, 0.319 mmol), and 2,2,6,6-tetramethyl-3,5-heptanedione (118 mg, 0.638 mmol) to give a suspension. The reaction mixture was allowed to stir at 150° C. for 2 hours. After cooling to room temperature, the reaction mixture was diluted with EtOAc (100 mL) and filtered through a celite plug, rinsing with EtOAc (100 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 316.2 (M+1)⁺.

### b) 4-(4-Bromo-3-hydroxy-6-phenoxy-pyridin-2-yl)-4-oxo-butyric acid ethyl ester

To a solution of 4-(3-hydroxy-6-phenoxy-pyridin-2-yl)-4-oxo-butyric acid ethyl ester (651 mg, 2.07 mmol) in CHCl₃ (21 mL) at room temperature was added sodium acetate (272 mg, 3.1 mmol) and bromine (495 mg, 3.1 mmol) to give a suspension. The reaction mixture was stirred at room temperature. After 24 h, the mixture was diluted with EtOAc (200 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 394.0, 396.0 (M−1)⁺.

### c) 4-(4-Cyano-3-hydroxy-6-phenoxy-pyridin-2-yl)-4-oxo-butyric acid ethyl ester

To a solution of 4-(4-bromo-3-hydroxy-6-phenoxy-pyridin-2-yl)-4-oxo-butyric acid ethyl ester (159 mg, 0.4 mmol) in DMAC (5.5 mL) at room temperature were added Zn(CN)₂ (94 mg, 0.8 mmol), Pd₂(dba)₃ (36 mg, 0.04 mmol), dppf (44 mg, 0.08 mmol) and Zn dust (7.8 mg, 0.12 mmol to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic lavers were washed with a 0.1 N HCl solution and water. The dried extract (MgSO₄) was concentrated in vacuo and purified by ISCO over silica gel, eluting with 5-35% EtOAc/hexanes to give product. MS (m/z) 341.2 (M+1)⁺.

### d) 4-(4-Cyano-3-hydroxy-6-phenoxy-pyridin-2-yl)-4-oxo-butyric acid

To a solution of 4-(4-cyano-3-hydroxy-6-phenoxy-pyridin-2-yl)-4-oxo-butyric acid ethyl ester (82 mg, 0.24 mmol) in THF (2.6 mL) and water (0.85 mL) at room temperature was added lithium hydroxide monohydrate (41 mg, 0.96 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 311.2 (M−1)⁺.

### Example 77

### 4-(4-Cyano-3-hydroxy-6-naphthalen-2-yl-pyridin-2-yl)-4-oxo-butyric acid

### a) 4-(3-Hydroxy-6-naphthalen-2-yl-pyridin-2-yl)-4-oxo-butyric acid ethyl ester

To a solution of 4-(6-bromo-3-hydroxy-pyridin-2-yl)-4-oxo-butyric acid ethyl ester (172 mg, 0.57 mmol, prepared from if) in toluene (3.8 mL) and H₂O (20 mg) at room temperature were added 2-naphthylboronic acid (147 mg, 0.85 mmol), K₃PO₄ (242 mg, 1.14 mmol), Pd(OAc)₂ (2.5 mg, 0.01 mmol) and S-Phos (11.7 mg, 0.03 mmol) to give a suspension. The reaction mixture was allowed to stir at 100° C. for 18 h. After cooling to room temperature, the reaction mixture was diluted with EtOAc (100 mL) and filtered through a celite plug, rinsing with EtOAc (100 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 350.2 (M+1)⁺.

### b) 4-(4-Bromo-3-hydroxy-6-naphthalen-2-yl-pyridin-2-yl)-4-oxo-butyric acid ethyl ester

To a solution of 4-(3-hydroxy-6-naphthalen-2-yl-pyridin-2-yl)-4-oxo-butyric acid ethyl ester (169 mg, 0.48 mmol) in CHCl₃ (4.8 mL) at room temperature was added sodium acetate (59.6 mg, 0.72 mmol) and bromine (116 mg, 0.72 mmol) to give a suspension. The reaction mixture was stirred at room temperature. After 24 h, the mixture was diluted with EtOAc (100 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 428.1, 430.0 (M+1)⁺.

### c) 4-(4-Cyano-3-hydroxy-6-naphthalen-2-yl-pyridin-2-yl)-4-oxo-butyric acid ethyl ester

To a solution of 4-(4-bromo-3-hydroxy-6-naphthalen-2-yl-pyridin-2-yl)-4-oxo-butyric acid ethyl ester (42.3 mg, 0.098 mmol) in DMAC (1.4 mL) at room temperature were added Zn(CN)₂ (23.1 mg, 0.196 mmol), Pd₂(dba)₃ (9 mg, 0.0098 mmol), dppf (11-mg, 0.0196 mmol), and Zn dust (2 mg, 0.03 mmol) to give a suspension. The reaction mixture was allowed to stir at 100° C. for 3-4 hours. After cooling to room temperature, the reaction mixture was diluted with EtOAc (30 mL) and filtered through a celite plug, rinsing with EtOAc (30 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 375.1 (M+1)⁺.

### d) 4-(4-Cyano-3-hydroxy-6-naphthalen-2-yl-pyridin-2-yl)-4-oxo-butyric acid

To a solution of 4-(4-cyano-3-hydroxy-6-naphthalen-2-yl-pyridin-2-yl)-4-oxo-butyric acid ethyl ester (14.2 mg, 0.04 mmol) in THF (0.57 mL) and water (0.2 mL) at room temperature was added lithium hydroxide monohydrate (6.4 mg, 0.15 mmol) to give a suspension. The reaction mixture was stirred at room temperature. After 24 h, the mixture was concentrated in vacuo to give residue. The residue was dissolved in water (15 mL) and extracted with EtOAc (3×5 mL). The aqueous solution was acidified with a 1N HCl solution, filtered, washed with water, and dried to give product. MS (m/z) 345.1 (M−1)⁺.

### Example 78

### 4-(4-Cyano-3-hydroxy-6-phenylsulfanyl-pyridin-2-yl)-4-oxo-butyric acid

### a) 4-(3-Hydroxy-6-phenylsulfanyl-pyridin-2-yl)-4-oxo-butyric acid ethyl ester

To a solution of 4-(6-bromo-3-hydroxy-pyridin-2-yl)-4-oxo-butyric acid ethyl ester (652 mg, 2.15 mmol, prepared from if) in dioxane (22 mL) at room temperature were added PhSH (356 mg, 3.23 mmol), N,N-Diisopropylethylamine (555 mg, 4.3 mmol), Pd₂(dba)₃ (98 mg, 0.11 mmol), and Xantphos (124 mg, 0.215 mmol) to give a suspension. The reaction mixture was allowed to stir at 100° C. for 3 h. After cooling to room temperature, the reaction mixture was diluted with EtOAc (100 mL) and filtered through a celite plug, rinsing with EtOAc (100 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 332.1 (M+1)⁺.

### b) 4-(4-Bromo-3-hydroxy-6-phenylsulfanyl-pyridin-2-yl)-4-oxo-butyric acid ethyl ester

To a solution of 4-(3-hydroxy-6-phenylsulfanyl-pyridin-2-yl)-4-oxo-butyric acid ethyl ester (321 mg, 0.96 mmol) in CHCl₃ (9.6 mL) at room temperature was added sodium acetate (119 mg, 1.45 mmol) and bromine (232 mg, 1.45 mmol) to give a suspension. The reaction mixture was stirred at room temperature. After 24 h, the mixture was diluted with EtOAc (100 ml). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 410.0, 412.0 (M+1)⁺.

### c) 4-(4-Cyano-3-hydroxy-6-phenylsulfanyl-pyridin-2-yl)-4-oxo-butyric acid ethyl ester

To a solution of 4-(4-bromo-3-hydroxy-6-phenylsulfanyl-pyridin-2-yl)-4-oxo-butyric acid ethyl ester (254 mg, 0.61 mmol) in DMAC (8 mL) at room temperature were added Zn(CN)₂ (145 mg, 1.23 mmol), Pd₂(dba)₃ (57 mg, 0.061 mmol), dppf (69 mg, 0.12 mmol), and Zn dust (12 mg, 0.18 mmol) to give a suspension. The reaction mixture was allowed to stir at 100° C. for 3-4 hours. After cooling to room temperature, the reaction mixture was diluted with EtOAc (80 mL) and filtered through a celite plug, rinsing with EtOAc (80 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 357.1 (M+1)⁺.

### d) 4-(4-Cyano-3-hydroxy-6-phenylsulfanyl-pyridin-2-yl)-4-oxo-butyric acid

To a solution of 4-(4-cyano-3-hydroxy-6-phenylsulfanyl-pyridin-2-yl)-4-oxo-butyric acid ethyl ester (37.6 mg, 0.105 mmol) in THF (1.1 mL) and water (0.4 mL) at room temperature was added lithium hydroxide monohydrate (17.7 mg, 0.42 mmol) to give a suspension. The reaction mixture was stirred at room temperature. After 24 h, the mixture was concentrated in vacuo to give residue. The residue was dissolved in water (15 mL) and extracted with EtOAc (3×5 mL). The aqueous solution was acidified with a 1N HCl solution, filtered, washed with water, and dried to give product. MS (m/z) 327.0 (M−1)⁺.

### Example 79

### 4-[4-Cyano-6-(4-fluoro-phenoxy)-3-hydroxy-pyridin-2-yl]-4-oxo-butyric acid

### a) 4-[6-(4-Fluoro-phenoxy)-3-hydroxy-pyridin-2-yl]-4-oxo-butyric acid ethyl ester

To a solution of 4-(6-bromo-3-hydroxy-pyridin-2-yl)-4-oxo-butyric acid ethyl ester (379 mg, 1.25 mmol, prepared from 66f) in DMAC (16 mL) at room temperature were added 4-fluoro-phenol (492 mg, 4.39 mmol), Cs₂CO₃ (2 g, 6.27 mmol), CuCl (12.4 mg, 0.125 mmol), and 2,2,6,6-tetramethyl-3,5-heptanedione (46 mg, 0.25 mmol) to give a suspension. The reaction mixture was allowed to stir at 150° C. for 2 hours. After cooling to room temperature, the reaction mixture was diluted with EtOAc (100 mL) and filtered through a celite plug, rinsing with EtOAc (100 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 334.0 (M+1)⁺.

### b) 4-[4-Bromo-6-(4-fluoro-phenoxy)-3-hydroxy-pyridin-2-yl]-4-oxo-butyric acid ethyl ester

To a solution of 4-[6-(4-fluoro-phenoxy)-3-hydroxy-pyridin-2-yl]-4-oxo-butyric acid ethyl ester (95 mg, 0.28 mmol) in CHCl₃ (2.8 mL) at room temperature was added sodium acetate (35 mg, 0.42 mmol) and bromine (68 mg, 0.42 mmol) to give a suspension. The reaction mixture was stirred at room temperature. After 24 h, the mixture was diluted with EtOAc (100 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 411.6, 413.8 (M+1)⁺.

### c) 4-[4-Cyano-6-(4-fluoro-phenoxy)-3-hydroxy-pyridin-2-yl]-4-oxo-butyric acid ethyl ester

To a solution of 4-[4-bromo-6-(4-fluoro-phenoxy)-3-hydroxy-pyridin-2-yl]-4-oxo-butyric acid ethyl ester (50.6 mg, 0.12 mmol) in DMAC (1.7 mL) at room temperature were added Zn(CN)₂ (29 mg, 0.24 mmol), Pd₂(dba): (11.2 mg, 0.012 mmol), dppf (13.6 mg, 0.024 mmol), and Zn dust (2.3 rug, 0.036 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 357.1 (M−1)⁺.

### d) 4-[4-Cyano-6-(4-fluoro-phenoxy)-3-hydroxy-pyridin-2-yl]-4-oxo-butyric acid

To a solution of 4-[4-cyano-6-(4-fluoro-phenoxy)-3-hydroxy-pyridin-2-yl]-4-oxo-butyric acid ethyl ester (14.6 mg, 0.04 mmol) in THF (0.75 mL) and water (0.25 mL) at room temperature was added lithium hydroxide monohydrate (6.85 mg, 0.16 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 329.0 (M−1)⁺.

### Example 80

### 4-[4-Cyano-6-(3-fluoro-2-methyl-phenyl)-3-hydroxy-pyridin-2-yl]-4-oxo-butyric acid

### a) 4-[6-(3-Fluoro-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 3-fluoro-2-methylbenzeneboronic acid in analogy to example 9a to give the title compound: MS (m/z) 332 (M+1)⁺.

### b) 4-[4-Bromo-6-(3-fluoro-2-methyl-phenyl)-3-hydroxy-pyridin-2-yl]-4-oxo-butyric acid ethyl ester

The title compound was prepared from 4-[6-(3-fluoro-2-methyl-phenyl)-3-hydroxy-pyridin-2-yl]-4-oxo-butyric acid ethyl ester and bromine in analogy to example 2c to give the title compound: MS (m/z) 410, 412 (M+1)⁺.

### c) 4-[4-Cyano-6-(3-fluoro-2-methyl-phenyl)-3-hydroxy-pyridin-2-yl]-4-oxo-butyric acid ethyl ester

To a solution of 4-[4-bromo-6-(3-fluoro-2-methyl-phenyl)-3-hydroxy-pyridin-2-yl]-4-oxo-butyric acid ethyl ester (146 mg, 0.36 mmol) in DMAC (5 mL) at room temperature were added Zn(CN)₂ (83.3 mg, 0.72 mmol), Pd₂(dba)₃ (32.5 mg, 0.036 mmol), dppf (39.4 mg, 0.072 mmol), and Zn dust (6.9 mg, 0.11 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 357.0 (M+1)⁺.

### d) 4-[4-Cyano-6-(3-fluoro-2-methyl-phenyl)-3-hydroxy-pyridin-2-yl]-4-oxo-butyric acid

To a solution of 4-[4-cyano-6-(3-fluoro-2-methyl-phenyl)-3-hydroxy-pyridin-2-yl]-4-oxo-butyric acid ethyl ester (79.5 mg, 0.22 mmol) in THF (2.4 mL) and water (0.7 mL) at room temperature was added lithium hydroxide monohydrate (37.5 mg, 0.89 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 329.0 (M+1)⁺.

### Example 81

### 4-[6-(2-Chloro-4-methoxy-phenyl)-4-cyano-3-hydroxy-pyridin-2-yl]-4-oxo-butyric acid

### a) 4-[6-(2-Chloro-4-methoxy-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-4-methoxyphenylboronic acid in analogy to example 9a to give the title compound: MS (m/z) 364 (M+1)⁺.

### b) 4-[4-Bromo-6-(2-chloro-4-methoxy-phenyl)-3-hydroxy-pyridin-2-yl]-4-oxo-butyric acid ethyl ester

The title compound was prepared from 4-[6-(2-chloro-4-methoxy-phenyl)-3-hydroxy-pyridin-2-yl]-4-oxo-butyric acid ethyl ester and bromine in analogy to example 2c to give the title compound: MS (m/z) 442, 444 (M+1)⁺.

### c) 4-[6-(2-Chloro-4-methoxy-phenyl)-4-cyano-3-hydroxy-pyridin-2-yl]-4-oxo-butyric acid ethyl ester

To a solution of 4-[4-bromo-6-(2-chloro-4-methoxy-phenyl)-3-hydroxy-pyridin-2-yl]-4-oxo-butyric acid ethyl ester (105 mg, 0.23 mmol) in DMAC (3.3 mL) at room temperature were added Zn(CN)₂ (55.6 in. 0.46 mmol), Pd₂(dba)₃ (21 mg, 0.023 mmol), dppf (25 mg, 0.046 mmol), and Zn dust (4.5 mg, 0.07 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCO solution and water. The dried extract (MgSO₄) was concentrated in vacuo and purified by ISCO over silica gel, eluting with 5-35% EtOAc/hexanes to give product. MS (m/z) 389.0 (M+1)⁺.

### d) 4-[6-(2-Chloro-4-methoxy-phenyl)-4-cyano-3-hydroxy-pyridin-2-yl]-4-oxo-butyric acid

To a solution of 4-[6-(2-chloro-4-methoxy-phenyl)-4-cyano-3-hydroxy-pyridin-2-yl]-4-oxo-butyric acid ethyl ester (53.2 mg, 0.137 mmol) in THF (1.5 mL) and water (0.5 mL) at room temperature was added lithium hydroxide monohydrate (23 mg, 0.54 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 359.0 (M−1)⁺.

### Example 82

### 4-[4-Cyano-6-(5-fluoro-2-methyl-phenyl)-3-hydroxy-pyridin-2-yl]-4-oxo-butyric acid

### a) 4-[6-(5-Fluoro-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 5-fluoro-2-methylphenylboronic acid in analogy to example 9a to give the title compound: MS (m/z) 332 (M+1)⁺.

### b) 4-[4-Bromo-6-(5-fluoro-2-methyl-phenyl)-3-hydroxy-pyridin-2-yl]-4-oxo-butyric acid ethyl ester

The title compound was prepared from 4-[6-(5-fluoro-2-methyl-phenyl)-3-hydroxy-pyridin-2-yl]-4-oxo-butyric acid ethyl ester and bromine in analogy to example 2c to give the title compound: MS (m/z) 410, 412 (M+1)⁺.

### c) 4-[4-Cyano-6-(5-fluoro-2-methyl-phenyl)-3-hydroxy-pyridin-2-yl]-4-oxo-butyric acid ethyl ester

To a solution of 4-[4-bromo-6-(5-fluoro-2-methyl-phenyl)-3-hydroxy-pyridin-2-yl]-4-oxo-butyric acid ethyl ester (143.9 mg, 0.35 mmol) in DMAC (5 mL) at room temperature were added Zn(CN)₂ (82 rug, 0.7 mmol), Pd₂(dba), (32 rug, 0.035 mmol dppf (38 mg, 0.07 mmol), and Zn dust (6.8 mg, 0.11 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 357.0 (M+1)⁺.

### d) 4-[4-Cyano-6-(5-fluoro-2-methyl-phenyl)-3-hydroxy-pyridin-2-yl]-4-oxo-butyric acid

To a solution of 4-[4-cyano-6-(5-fluoro-2-methyl-phenyl)-3-hydroxy-pyridin-2-yl]-4-oxo-butyric acid ethyl ester (72.2 mg, 0.2 mmol) in THF (2.1 mL) and water (0.7 mL) at room temperature was added lithium hydroxide monohydrate (34 mg, 0.81 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 327.0 (M−1)⁺.

### Example 83

### 4-[6-(2-Chloro-4-fluoro-phenyl)-4-cyano-3-hydroxy-pyridin-2-yl]-4-oxo-butyric acid

### a) 4-[6-(2-Chloro-4-fluoro-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-4-fluorophenylboronic acid in analogy to example 9a to give the title compound: MS (m/z) 352 (M+1)⁺.

### b) 4-[4-Bromo-6-(2-chloro-4-fluoro-phenyl)-3-hydroxy-pyridin-2-yl]-4-oxo-butyric acid ethyl ester

The title compound was prepared from 4-[6-(2-chloro-4-fluoro-phenyl)-3-hydroxy-pyridin-2-yl]-4-oxo-butyric acid ethyl ester and bromine in analogy to example 2c to give the title compound: MS (m/z) 430, 432 (M+1)⁺.

### c) 4-[6-(2-Chloro-4-fluoro-phenyl)-4-cyano-3-hydroxy-pyridin-2-yl]-4-oxo-butyric acid ethyl ester

To a solution of 4-[4-bromo-6-(2-chloro-4-fluoro-phenyl)-3-hydroxy-pyridin-2-yl]-4-oxo-butyric acid ethyl ester (100 mg, 0.23 mmol) in DMAC (3.3 mL) at room temperature were added Zn(CN)₂ (54 mg, 0.46 mmol), Pd₂(dba)₃ (21 mg, 0.023 mmol), dppf (25 mg, 0.046 mmol), and Zn dust (4.5 mg, 0.07 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 375.1 (M−1)⁺.

### d) 4-[6-(2-Chloro-4-fluoro-phenyl)-4-cyano-3-hydroxy-pyridin-2-yl]-4-oxo-butyric acid

To a solution of 4-[6-(2-chloro-4-fluoro-phenyl)-4-cyano-3-hydroxy-pyridin-2-yl]-4-oxo-butyric acid ethyl ester (21.1 mg, 0.056 mmol) in THF (0.75 mL) and water (0.25 mL) at room temperature was added lithium hydroxide monohydrate (9.4 mg, 0.22 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 347.0 (M−1)⁺.

### Example 84

### 4-[6-(2-Chloro-4-methyl-phenyl)-4-cyano-3-hydroxy-pyridin-2-yl]-4-oxo-butyric acid

### a) 4-[6-(2-Chloro-4-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-4-methylphenylboronic acid in analogy to example 9a to give the title compound: MS (m/z) 348 (M+1)⁺.

### b) 4-[4-Bromo-6-(2-chloro-4-methyl-phenyl)-3-hydroxy-pyridin-2-yl]-4-oxo-butyric acid ethyl ester

The title compound was prepared from 4-[6-(2-chloro-4-methyl-phenyl)-3-hydroxy-pyridin-2-yl]-4-oxo-butyric acid ethyl ester and bromine in analogy to example 2c to give the title compound: MS (m/z) 426, 428 (M+1)⁺.

### c) 4-[6-(2-Chloro-4-methyl-phenyl)-4-cyano-3-hydroxy-pyridin-2-yl]-4-oxo-butyric acid ethyl ester

To a solution of 4-[4-bromo-6-(2-chloro-4-methyl-phenyl)-3-hydroxy-pyridin-2-yl]-4-oxo-butyric acid ethyl ester (124 mg, 0.29 mmol) in DMAC (4.1 mL) at room temperature were added Zn(CN); (67.9 mg, 0.58 mmol), Pd₂(dba)₃ (27 mg, 0.029 mmol), dppf (32 mg, 0.058 mmol), and Zn dust (5.6 mg, 0.08 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 372.8, 374.8 (M−1)⁺.

### d) 4-[6-(2-Chloro-4-methyl-phenyl)-4-cyano-3-hydroxy-pyridin-2-yl]-4-oxo-butyric acid

To a solution of 4-[6-(2-chloro-4-methyl-phenyl)-4-cyano-3-hydroxy-pyridin-2-yl]-4-oxo-butyric acid ethyl ester (67.4 mg, 0.18 mmol) in THF (1.9 mL) and water (0.65 mL) at room temperature was added lithium hydroxide monohydrate (30 mg, 0.72 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 344.9, 346.6 (M+1)⁺.

### Example 85

### 4-[4-Cyano-6-(2-ethyl-phenyl)-3-hydroxy-pyridin-2-yl]-4-oxo-butyric acid

### a) 4-[6-(2-Ethyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-ethylphenylboronic acid in analogy to example 9a to give the title compound: MS (m/z) 328 (M+1)⁺.

### b) 4-[4-Bromo-6-(2-ethyl-phenyl)-3-hydroxy-pyridin-2-yl]-4-oxo-butyric acid ethyl ester

The title compound was prepared from 4-[6-(2-ethyl-phenyl)-3-hydroxy-pyridin-2-yl]-4-oxo-butyric acid ethyl ester and bromine in analogy to example 2c to give the title compound: MS (m/z) 406, 408 (M+1)⁺.

### c) 4-[4-Cyano-6-(2-ethyl-phenyl)-3-hydroxy-pyridin-2-yl]-4-oxo-butyric acid ethyl ester

To a solution of 4-[4-bromo-6-(2-ethyl-phenyl)-3-hydroxy-pyridin-2-yl]-4-oxo-butyric acid ethyl ester (116 mg, 0.28 mmol) in DMAC (4 mL) at room temperature were added Zn(CN)₂ (69 mg, 0.57 mmol), Pd₂(dba)₃ (26 mg, 0.028 mmol), dppf (31 mg, 0.057 mmol), and Zn dust (5.5 mg, 0.08 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 353.0 (M+1)⁺.

### d) 4-[4-Cyano-6-(2-ethyl-phenyl)-3-hydroxy-pyridin-2-yl]-4-oxo-butyric acid

To a solution of 4-[4-cyano-6-(2-ethyl-phenyl)-3-hydroxy-pyridin-2-yl]-4-oxo-butyric acid ethyl ester (69.1 mg, 0.19 mmol) in THF (2.1 mL) and water (0.7 mL) at room temperature was added lithium hydroxide monohydrate (33 mg, 0.78 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 324.9 (M+1)⁺.

### Example 86

### 4-[4-Cyano-6-(4-fluoro-2-methyl-phenyl)-3-hydroxy-pyridin-2-yl]-4-oxo-butyric acid

### a) 4-[6-(4-Fluoro-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 4-fluoro-2-methylphenylboronic acid in analogy to example 9a to give the title compound: MS (m/z) 332 (M+1)⁺.

### b) 4-[6-(4-Fluoro-2-methyl-phenyl)-3-hydroxy-pyridin-2-yl]-4-oxo-butyric acid ethyl ester

The title compound was prepared from 4-[6-(4-fluoro-2-methyl-phenyl)-3-hydroxy-pyridin-2-yl]-4-oxo-butyric acid ethyl ester and bromine in analogy to example 2c to give the title compound: MS (m/z) 410, 412 (M+1)⁺.

### c) 4-[4-Cyano-6-(4-fluoro-2-methyl-phenyl)-3-hydroxy-pyridin-2-yl]-4-oxo-butyric acid ethyl ester

To a solution of 4-[4-bromo-6-(4-fluoro-2-methyl-phenyl)-3-hydroxy-pyridin-2-yl]-4-oxo-butyric acid ethyl ester (137 mg, 0.33 mmol) in DMAC (4.7 mL) at room temperature were added Zn(CN)₂ (78 mg, 0.66 mmol), Pd₂(dbaⱼ(30 mg, 0.033 mmol), dppf (37 mg, 0.066 mmol), and Zn dust (6.4 mg, 0.1 mmol) to give a suspension. The reaction mixture was allowed to stir at 100° C. for 3-hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 357.0 (M+1)⁺.

### d) 4-[4-Cyano-6-(4-fluoro-2-methyl-phenyl)-3-hydroxy-pyridin-2-yl]-4-oxo-butyric acid

To a solution of 4-[4-cyano-6-(4-fluoro-2-methyl-phenyl)-3-hydroxy-pyridin-2-yl]-4-oxo-butyric acid ethyl ester (79 mg, 0.22 mmol) in THF (2.3 mL) and water (0.75 mL) at room temperature was added lithium hydroxide monohydrate (37 mg, 0.88 mmol) to give a suspension. The reaction mixture was stirred at room temperature. After 24 h, the mixture was concentrated in vacuo to give residue. The residue was dissolved in water (25 mL) and extracted with EtOAc (3×15 mL). The aqueous solution was acidified with a 1N HCl solution, filtered, washed with water, and dried to give product. MS (m/z) 329.0 (M+1)⁺.

### Example 87

### 4-[4-Cyano-6-(2-cyano-4-fluoro-phenyl)-3-hydroxy-pyridin-2-yl]-4-oxo-butyric acid

### a) 4-[6-(2-Chloro-4-fluoro-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-4-fluorophenylboronic acid in analogy to example 9a to give the title compound: MS (m/z) 352 (M+1)⁺.

### b) 4-[4-Bromo-6-(2-chloro-4-fluoro-phenyl)-3-hydroxy-pyridin-2-yl]-4-oxo-butyric acid ethyl ester

The title compound was prepared from 4-[6-(2-chloro-4-fluoro-phenyl)-3-hydroxy-pyridin-2-yl]-4-oxo-butyric acid ethyl ester and bromine in analogy to example 2c to give the title compound: MS (m/z) 430, 432 (M+1)⁺.

### c) 4-[4-Cyano-6-(2-cyano-4-fluoro-phenyl)-3-hydroxy-pyridin-2-yl]-4-oxo-butyric acid ethyl ester

To a solution of 4-[4-bromo-6-(2-chloro-4-fluoro-phenyl)-3-hydroxy-pyridin-2-yl]-4-oxo-butyric acid ethyl ester (100 mg, 0.23 mmol) in DMAC (3.3 mL) at room temperature were added Zn(CN)₂ (54 rug, 0.46 mmol), Pd₂(dba)₃ (21 mg, 0.023 mmol x dppf (25 mg, 0.046 mmol), and Zn dust (4.5 mg, 0.07 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 366.1 (M−1)⁺.

### d) 4-[4-Cyano-6-(2-cyano-4-fluoro-phenyl)-3-hydroxy-pyridin-2-yl]-4-oxo-butyric acid

To a solution of 4-[4-cyano-6-(2-cyano-4-fluoro-phenyl)-3-hydroxy-pyridin-2-yl]-4-oxo-butyric acid ethyl ester (11.9 mg, 0.032 mmol) in THF (0.75 mL) and water (0.25 mL) at room temperature was added lithium hydroxide monohydrate (5.5 mg, 0.13 mmol) to give a suspension. The reaction mixture was stirred at room temperature. After 24 h, the mixture was concentrated in vacuo to give residue. The residue was dissolved in water (15 mL) and extracted with EtOAc (3×10 mL). The aqueous solution was acidified with a 1N HCl solution, filtered, washed with water, and dried to give product. MS (m/z) 338.0 (M−1)⁺.

### Example 88

### 4-[4-Cyano-6-(2-fluoro-6-methyl-phenyl)-3-hydroxy-pyridin-2-yl]-4-oxo-butyric acid

### a) 4-[6-(2-Fluoro-6-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-fluoro-6-methylphenylboronic acid in analogy to example 9a to give the title compound: MS (m/z) 332 (M+1)⁺.

### b) 4-[4-Bromo-6-(2-fluoro-6-methyl-phenyl)-3-hydroxy-pyridin-2-yl]-4-oxo-butyric acid ethyl ester

The title compound was prepared from 4-[6-(2-fluoro-6-methyl-phenyl)-3-hydroxy-pyridin-2-yl]-4-oxo-butyric acid ethyl ester and bromine in analogy to example 2c to give the title compound: MS (m/z) 410, 412 (M+1)⁺.

### c) 4-[4-Cyano-6-(2-fluoro-6-methyl-phenyl)-3-hydroxy-pyridin-2-yl]-4-oxo-butyric acid ethyl ester

To a solution of 4-[4-bromo-6-(2-fluoro-6-methyl-phenyl)-3-hydroxy-pyridin-2-yl]-4-oxo-butyric acid ethyl ester (107 mg, 0.26 mmol) in DMAC (3.3 mL) at room temperature were added Zn(CN)₂ (61 mg, 0.56 mmol), Pd₂(dba)₃ (24 mg, 0.026 mmol), dppf (29 mg, 0.056 mmol), and Zn dust (5 mg, 0.08 mmol) to give a suspension. The reaction mixture was allowed to stir at 100° C. for 3-4 hours. After cooling to room temperature, the reaction mixture was diluted with EtOAc (50 uL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 357.0 (M+1)⁺.

### d) 4-[4-Cyano-6-(2-fluoro-6-methyl-phenyl)-3-hydroxy-pyridin-2-yl]-4-oxo-butyric acid

To a solution of 4-[4-cyano-6-(2-fluoro-6-methyl-phenyl)-3-hydroxy-pyridin-2-yl]-4-oxo-butyric acid ethyl ester (73 mg, 0.2 mmol) in THF (2.1 mL) and water (0.75 mL) at room temperature was added lithium hydroxide monohydrate (34 mg, 0.82 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 329.0 (M+1)⁺.

### Example 89

### 4-[6-(3-Chloro-2-methyl-phenyl)-4-cyano-3-hydroxy-pyridin-2-yl]-4-oxo-butyric acid

### a) 4-[6-(3-Chloro-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 3-chloro-2-methylphenylboronic acid in analogy to example 9a to give the title compound: MS (m/z) 348 (M+1)⁺.

### b) 4-[4-Bromo-6-(3-chloro-2-methyl-phenyl)-3-hydroxy-pyridin-2-yl]-4-oxo-butyric acid ethyl ester

The title compound was prepared from 4-[6-(3-chloro-2-methyl-phenyl)-3-hydroxy-pyridin-2-yl]-4-oxo-butyric acid ethyl ester and bromine in analogy to example 2c to give the title compound: MS (m/z) 426, 428 (M+1)⁺.

### c) 4-[6-(3-Chloro-2-methyl-phenyl)-4-cyano-3-hydroxy-pyridin-2-yl]-4-oxo-butyric acid ethyl ester

To a solution of 4-[4-bromo-6-(3-chloro-2-methyl-phenyl)-3-hydroxy-pyridin-2-yl]-4-oxo-butyric acid ethyl ester (148 mg, 0.34 mmol) in DMAC (4.9 mL) at room temperature were added Zn(CN)₂ (81.3 mg, 0.69 mmol, Pd₂(dba)₃ (31 mg, 0.034 mmol), dppf (38 mg, 0.069 mmol), and Zn dust (6.6 mg, 0.1 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 373.0 (M+1)⁺.

### d) 4-[6-(3-Chloro-2-methyl-phenyl)-4-cyano-3-hydroxy-pyridin-2-yl]-4-oxo-butyric acid

To a solution of 4-[6-(3-chloro-2-methyl-phenyl)-4-cyano-3-hydroxy-pyridin-2-yl]-4-oxo-butyric acid ethyl ester (91 mg, 0.24 mmol) in THF (2.6 mL) and water (0.82 mL) at room temperature was added lithium hydroxide monohydrate (41 mg, 0.97 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 343.0 (M−1)⁺.

### Example 90

### 4-[6-(4-Chloro-2-methyl-phenyl)-4-cyano-3-hydroxy-pyridin-2-yl]-4-oxo-butyric acid

### a) 4-[6-(4-Chloro-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 4-chloro-2-methylphenylboronic acid in analogy to example 9a to give the title compound: MS (m/z) 348 (M+1)⁺.

### b) 4-[4-Bromo-6-(4-chloro-2-methyl-phenyl)-3-hydroxy-pyridin-2-yl]-4-oxo-butyric acid ethyl ester

The title compound was prepared from 4-[6-(4-chloro-2-methyl-phenyl)-3-hydroxy-pyridin-2-yl]-4-oxo-butyric acid ethyl ester and bromine in analogy to example 2c to give the title compound: MS (m/z) 426, 428 (M+1)⁺.

### c) 4-[6-(4-Chloro-2-methyl-phenyl)-4-cyano-3-hydroxy-pyridin-2-yl]-4-oxo-butyric acid ethyl ester

To a solution of 4-[4-bromo-6-(4-chloro-2-methyl-phenyl)-3-hydroxy-pyridin-2-yl]-4-oxo-butyric acid ethyl ester (128 mg, 0.3 mmol) in DMAC (4.3 mL) at room temperature were added Zn(CN)₂ (703 mg, 0.6 mmol), Pd₂(dba)₃ (27 mg, 0.03 mmol), dppf (33 mg, 0.06 mmol), and Zn dust (5.8 mg, 0.09 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 373.0 (M+1)⁺.

### d) 4-[6-(4-Chloro-2-methyl-phenyl)-4-cyano-3-hydroxy-pyridin-2-yl]-4-oxo-butyric acid

To a solution of 4-[6-(4-chloro-2-methyl-phenyl)-4-cyano-3-hydroxy-pyridin-2-yl]-4-oxo-butyric acid ethyl ester (75 mg, 0.2 mmol) in THF (2.1 mL) and water (0.75 mL) at room temperature was added lithium hydroxide monohydrate (34 mg, 0.8 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 345.0 (M+1)⁺.

### Example 91

### 4-[6-(5-Chloro-2-methyl-phenyl)-4-cyano-3-hydroxy-pyridin-2-yl]-4-oxo-butyric acid

### a) 4-[6-(5-Chloro-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 5-chloro-2-methylphenylboronic acid in analogy to example 9a to give the title compound: MS (m/z) 348 (M+1)⁺.

### b) 4-[4-Bromo-6-(5-chloro-2-methyl-phenyl)-3-hydroxy-pyridin-2-yl]-4-oxo-butyric acid ethyl ester

The title compound was prepared from 4-[6-(5-chloro-2-methyl-phenyl)-3-hydroxy-pyridin-2-yl]-4-oxo-butyric acid ethyl ester and bromine in analogy to example 2c to give the title compound: MS (m/z) 426, 428 (M+1)⁺.

### c) 4-[6-(5-Chloro-2-methyl-phenyl)-4-cyano-3-hydroxy-pyridin-2-yl]-4-oxo-butyric acid ethyl ester

To a solution of 4-[4-bromo-6-(5-chloro-2-methyl-phenyl)-3-hydroxy-pyridin-2-yl]-4-oxo-butyric acid ethyl ester (121 mg, 0.28 mmol) in DMAC (4 mL) at room temperature were added Zn(CN)₂ (66.5 mg, 0.56 mmol), Pd₂(dba)₃ (26 mg, 0,028 mmol), dppf (31 mg, 0.056 mmol), and Zn dust (5.5 mg, 0.08 mmol) to give a suspension. The reaction mixture was allowed to stir at 100° C. for 3-4 hours. After cooling to room temperature, the reaction mixture was diluted with EtOAc (50 ml) and filtered through a celite plug, rinsing with EtOAc 150 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 373.0 (M+1)⁺.

### d) 4-[6-(5-Chloro-2-methyl-phenyl)-4-cyano-3-hydroxy-pyridin-2-yl]-4-oxo-butyric acid

To a solution of 4-[6-(5-chloro-2-methyl-phenyl)-4-cyano-3-hydroxy-pyridin-2-yl]-4-oxo-butyric acid ethyl ester (31 mg, 0.083 mmol) in THF (0.9 mL) and water (0.3 mL) at room temperature was added lithium hydroxide monohydrate (14 mg, 0.33 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 344.9 (M+1)⁺.

### Example 92

### 4-[4-Cyano-6-(2,3-dimethyl-phenyl)-3-hydroxy-pyridin-2-yl]-4-oxo-butyric acid

### a) 4-[6-(2,3-Dimethyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2,3-dimethylphenylboronic acid in analogy to example 9a to give the title compound: MS (m/z) 328 (M+1)⁺.

### b) 4-[4-Bromo-6-(2,3-dimethyl-phenyl)-3-hydroxy-pyridin-2-yl]-4-oxo-butyric acid ethyl ester

The title compound was prepared from 4-[6-(2,3-dimethyl-phenyl)-3-hydroxy-pyridin-2-yl]-4-oxo-butyric acid ethyl ester and bromine in analogy to example 2c to give the title compound: MS (m/z) 406, 408 (M+1)⁺.

### c) 4-[4-Cyano-6-(2,3-dimethyl-phenyl)-3-hydroxy-pyridin-2-yl]-4-oxo-butyric acid ethyl ester

To a solution of 4-[4-bromo-6-(2,3-dimethyl-phenyl)-3-hydroxy-pyridin-2-yl]-4-oxo-butyric acid ethyl ester (130 mg, 0.32 mmol) in DMAC (4.6 mL) at room temperature were added Zn(CN)₂ (75 rug, 0.64 mmol), Pd₂(dba)₃ (293 mg, 0,032 mmol), dppf (35 mg, 0.064 mmol), and Zn dust (6.2 ag, 0.09 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 353.0 (M+1)⁺.

### d) 4-[4-Cyano-6-(2,3-dimethyl-phenyl)-3-hydroxy-pyridin-2-yl]-4-oxo-butyric acid

To a solution of 4-[4-cyano-6-(2,3-dimethyl-phenyl)-3-hydroxy-pyridin-2-yl]-4-oxo-butyric acid ethyl ester (74 mg, 0.21 mmol) in THF (2.2 mL) and water (0.8 mL) at room temperature was added lithium hydroxide monohydrate (35 mg, 0.84 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 325.0 (M+1)⁺.

### Example 93

### 4-[4-Cyano-6-(2,4-dimethyl-phenyl)-3-hydroxy-pyridin-2-yl]-4-oxo-butyric acid

### a) 4-[6-(2,4-Dimethyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2,4-dimethylphenylboronic acid in analogy to example 9a to give the title compound: MS (m/z) 328 (M+1)⁺.

### b) 4-[4-Bromo-6-(2,4-dimethyl-phenyl)-3-hydroxy-pyridin-2-yl]-4-oxo-butyric acid ethyl ester

The title compound was prepared from 4-[6-(2,4-dimethyl-phenyl)-3-hydroxy-pyridin-2-yl]-4-oxo-butyric acid ethyl ester and bromine in analogy to example 2c to give the title compound: MS (m/z) 406, 408 (M+1)⁺.

### c) 4-[4-Cyano-6-(2,4-dimethyl-phenyl)-3-hydroxy-pyridin-2-yl]-4-oxo-butyric acid ethyl ester

To a solution of 4-[4-bromo-6-(2,4-dimethyl-phenyl)-3-hydroxy-pyridin-2-yl]-4-oxo-butyric acid ethyl ester (130 mg, 0.32 mmol) in DMAC (4.6 mL) at room temperature were added Zn(CN)₂ (75 mg, 0.64 mmol) Pd₂(dba)₃ (29.3 mg, 0.032 mmol), dppf (35 mg, 0.064 mmol), and Zn dust (6.2 mg, 0.09 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 353.0 (M+1)⁺.

### d) 4-[4-Cyano-6-(2,4-dimethyl-phenyl)-3-hydroxy-pyridin-2-yl]-4-oxo-butyric acid

To a solution of 4-[4-cyano-6-(2,4-dimethyl-phenyl)-3-hydroxy-pyridin-2-yl]-4-oxo-butyric acid ethyl ester (50.1 mg, 0.14 mmol) in THF (1.5 mL) and water (0.5 mL) at room temperature was added lithium hydroxide monohydrate (24 mg, 0.56 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 325.0 (M+1)⁺.

### Example 94

### 4-[4-Cyano-6-(2,5-dimethyl-phenyl)-3-hydroxy-pyridin-2-yl]-4-oxo-butyric acid

### a) 4-[6-(2,5-Dimethyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2,5-dimethylphenylboronic acid in analogy to example 9a to give the title compound: MS (m/z) 328 (M+1)⁺.

### b) 4-[4-Bromo-6-(2,5-dimethyl-phenyl)-3-hydroxy-pyridin-2-yl]-4-oxo-butyric acid ethyl ester

The title compound was prepared from 4-[6-(2,5-dimethyl-phenyl)-3-hydroxy-pyridin-2-yl]-4-oxo-butyric acid ethyl ester and bromine in analogy to example 2c to give the title compound: MS (m/z) 406, 408 (M+1)⁺.

### c) 4-[4-Cyano-6-(2,5-dimethyl-phenyl)-3-hydroxy-pyridin-2-yl]-4-oxo-butyric acid ethyl ester

To a solution of 4-[4-bromo-6-(2,5-dimethyl-phenyl)-3-hydroxy-pyridin-2-yl]-4-oxo-butyric acid ethyl ester (135 mg, 0.33 mmol) in DMAC (4.7 mL) at room temperature were added Zn(CN)₂ (77.8 mg, 0.66 mmol), Pd₂(dba)₃ (30.2 mg, 0.033 mmol), dppf (37 mg, 0.066 mmol), and Zn dust (64 mg, 0.1 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 353.0 (M+1)⁺.

### d) 4-[4-Cyano-6-(2,5-dimethyl-phenyl)-3-hydroxy-pyridin-2-yl]-4-oxo-butyric acid

To a solution of 4-[4-cyano-6-(2,5-dimethyl-phenyl)-3-hydroxy-pyridin-2-yl]-4-oxo-butyric acid ethyl ester (78 mg, 0.22 mmol) in THF (2.35 mL) and water (0.8 mL) at room temperature was added lithium hydroxide monohydrate (37 mg, 0.88 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 325.0 (M+1)⁺.

### Example 95

### 4-[4-Cyano-3-hydroxy-6-(2-methyl-3-trifluoromethyl-phenyl)-pyridin-2-yl]-4-oxo-butyric acid

### a) 4-[3-Hydroxy-6-(2-methyl-3-trifluoromethyl-phenyl)-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-methyl-3-trifluorophenylboronic acid in analogy to example 9a to give the title compound: MS (m/z) 382 (M+1)⁺.

### b) 4-[4-Bromo-3-hydroxy-6-(2-methyl-3-trifluoromethyl-phenyl)-pyridin-2-yl]-4-oxo-butyric acid ethyl ester

The title compound was prepared from 4-[3-hydroxy-6-(2-methyl-3-trifluoromethyl-phenyl)-pyridin-2-yl]-4-oxo-butyric acid ethyl ester and bromine in analogy to example 2c to give the title compound: MS (m/z) 460, 462 (M+1)⁺.

### c) 4-[4-Cyano-3-hydroxy-6-(2-methyl-3-trifluoromethyl-phenyl)-pyridin-2-yl]-4-oxo-butyric acid ethyl ester

To a solution of 4-[4-bromo-3-hydroxy-6-(2-methyl-3-trifluoromethyl-phenyl)-pyridin-2-yl]-4-oxo-butyric acid ethyl ester (138 mg, 0.3 mmol) in DMAC (4.2 mL) at room temperature were added Zn(CN)₂ (70.2 mg, 0.6 mmol), Pd₂(dba)₃ (27.5 mg, 0.03 mmol), dppf (33 mg, 0.06 mmol), and Zn dust (5.9 mg, 0.09 mmol) to give a suspension. The reaction mixture was allowed to stir at 100° C. for 34 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 407.0 (M+1)⁺.

### d) 4-[4-Cyano-3-hydroxy-6-(2-methyl-3-trifluoromethyl-phenyl)-pyridin-2-yl]-4-oxo-butyric acid

To a solution of 4-[4-cyano-3-hydroxy-6-(2-methyl-3-trifluoromethyl-phenyl)-pyridin-2-yl]-4-oxo-butyric acid ethyl ester (55 mg, 0.135 mmol) in THF (1.5 mL) and water (0.5 mL) at room temperature was added lithium hydroxide monohydrate (23 mg, 0.54 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 377.0 (M−1)⁺.

### Example 96

### 4-[4-Cyano-3-hydroxy-6-(2-methyl-4-trifluoromethyl-phenyl)-pyridin-2-yl]-4-oxo-butyric acid

### a) 4-[3-Hydroxy-6-(2-methyl-4-trifluoromethyl-phenyl)-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-methyl-4-trifluoromethyl-phenylboronic acid in analogy to example 9a to give the title compound: MS (m/z) 382 (M+1)⁺.

### b) 4-[4-Bromo-3-hydroxy-6-(2-methyl-4-trifluoromethyl-phenyl)-pyridin-2-yl]-4-oxo-butyric acid ethyl ester

The title compound was prepared from 4-[3-hydroxy-6-(2-methyl-4-trifluoromethyl-phenyl)-pyridin-2-yl]-4-oxo-butyric acid ethyl ester and bromine in analogy to example 2c to give the title compound: MS (m/z) 460, 462 (M+1)⁺.

### c) 4-[4-Cyano-3-hydroxy-6-(2-methyl-4-trifluoromethyl-phenyl)-pyridin-2-yl]-4-oxo-butyric acid ethyl ester

To a solution of 4-[4-bromo-3-hydroxy-6-(2-methyl-4-trifluoromethyl-phenyl)-pyridin-2-yl]-4-oxo-butyric acid ethyl ester (133 mg, 0.29 mmol) in DMAC (4.1 mL) at room temperature were added Zn(CN)₂ (68 mg, 0.58 mmol), Pd₂(dba)₃ (265 mg, 0.029 mmol) dppf (32 mg, 0.058 mmol), and Zn dust (5.6 mg, 0.09 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 407.0 (M+1)⁺.

### d) 4-[4-Cyano-3-hydroxy-6-(2-methyl-4-trifluoromethyl-phenyl)-pyridin-2-yl]-4-oxo-butyric acid

To a solution of 4-[4-cyano-3-hydroxy-6-(2-methyl-4-trifluoromethyl-phenyl)-pyridin-2-yl]-4-oxo-butyric acid ethyl ester (70 mg, 0.17 mmol) in THF (1.8 mL) and water (0.6 mL) at room temperature was added lithium hydroxide monohydrate (29 mg, 0.69 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 377.0 (M−1)⁺.

### Example 97

### 4-[4-Cyano-3-hydroxy-6-(2-methyl-5-trifluoromethyl-phenyl)-pyridin-2-yl]-4-oxo-butyric acid

### a) 4-[3-Hydroxy-6-(2-methyl-5-trifluoromethyl-phenyl)-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-methyl-5-trifluoromethyl-phenylboronic acid in analogy to example 9a to give the title compound: MS (m/z) 382 (M+1)⁺.

### b) 4-[4-Bromo-3-hydroxy-6-(2-methyl-5-trifluoromethyl-phenyl)-pyridin-2-yl]-4-oxo-butyric acid ethyl ester

The title compound was prepared from 4-[3-hydroxy-6-(2-methyl-5-trifluoromethyl-phenyl)-pyridin-2-yl]-4-oxo-butyric acid ethyl ester and bromine in analogy to example 2c to give the title compound: MS (m/z) 460, 462 (M+1)⁺.

### c) 4-[4-Cyano-3-hydroxy-6-(2-methyl-5-trifluoromethyl-phenyl)-pyridin-2-yl]-4-oxo-butyric acid ethyl ester

To a solution of 4-[4-bromo-3-hydroxy-6-(2-methyl-5-trifluoromethyl-phenyl)-pyridin-2-yl]-4-oxo-butyric acid ethyl ester (127 mg, 0.28 mmol) in DMAC (3.9 mL) at room temperature were added Zn(CN)₂ (65 mg, 0.56 moil), Pd₂(dba)₃ (25.2 mg, 0.028 mmo), dppf (30.6 mg, 0,056 mmol), and Zn dust (5.4 mg, 0.08 mmol) to give a suspension. The reaction mixture was allowed to stir at 100° C. for 3-4 hours. After cooling to room temperature, the reaction mixture was diluted with EtOAc (50 ml) and filtered through a celite plug, rinsing with EtOAc 150 mL E The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 406.9 (M+1)⁺.

### d) 4-[4-Cyano-3-hydroxy-6-(2-methyl-5-trifluoromethyl-phenyl)-pyridin-2-yl]-4-oxo-butyric acid

To a solution of 4-[4-cyano-3-hydroxy-6-(2-methyl-5-trifluoromethyl-phenyl)-pyridin-2-yl]-4-oxo-butyric acid ethyl ester (64.5 mg, 0.16 mmol) in THF (1.7 mL) and water (0.6 mL) at room temperature was added lithium hydroxide monohydrate (27 mg, 0.63 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 377.0 (M−1)⁺.

### Example 98

### 4-[4-Cyano-6-(3-cyano-2-methyl-phenyl)-3-hydroxy-pyridin-2-yl]-4-oxo-butyric acid

### a) 4-[6-(3-Cyano-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 3-cyano-2-methylphenylboronic acid in analogy to example 9a to give the title compound: MS (m/z) 339 (M+1)⁺.

### b) 4-[4-Bromo-6-(3-cyano-2-methyl-phenyl)-3-hydroxy-pyridin-2-yl]-4-oxo-butyric acid ethyl ester

The title compound was prepared from 4-[6-(3-cyano-2-methyl-phenyl)-3-hydroxy-pyridin-2-yl]-4-oxo-butyric acid ethyl ester and bromine in analogy to example 2c to give the title compound: MS (m/z) 417, 419 (M+1)⁺.

### c) 4-[4-Cyano-6-(3-cyano-2-methyl-phenyl)-3-hydroxy-pyridin-2-yl]-4-oxo-butyric acid ethyl ester

To a solution of 4-[4-bromo-6-(3-cyano-2-methyl-phenyl)-3-hydroxy-pyridin-2-yl]-4-oxo-butyric acid ethyl ester (125 mg, 0.3 mmol) in DMAC (4.3 mL) at room temperature were added Zn(CN)₂ (7.1 mg, 0.6 mmol), Pd₂(dba), (27 mg, 0.03 mmol), dppf (33 mg, 0.06 mmol), and Zn dust (5.8 mg, 009 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 363.9 (M+1)⁺.

### d) 4-[4-Cyano-6-(3-cyano-2-methyl-phenyl)-3-hydroxy-pyridin-2-yl]-4-oxo-butyric acid

To a solution of 4-[4-cyano-6-(3-cyano-2-methyl-phenyl)-3-hydroxy-pyridin-2-yl]-4-oxo-butyric acid ethyl ester (62.6 mg, 0.17 mmol) in THF (1.8 mL) and water (0.6 mL) at room temperature was added lithium hydroxide monohydrate (29 mg, 0.69 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 334.0 (M−1)⁺.

### Example 99

### 4-[4-Cyano-6-(4-cyano-2-methyl-phenyl)-3-hydroxy-pyridin-2-yl]-4-oxo-butyric acid

### a) 4-[6-(4-Cyano-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 4-cyano-2-methylphenylboronic acid in analogy to example 9a to give the title compound: MS (m/z) 339 (M+1)⁺.

### b) 4-[4-Bromo-6-(4-cyano-2-methyl-phenyl)-3-hydroxy-pyridin-2-yl]-4-oxo-butyric acid ethyl ester

The title compound was prepared from 4-[6-(4-cyano-2-methyl-phenyl)-3-hydroxy-pyridin-2-yl]-4-oxo-butyric acid ethyl ester and bromine in analogy to example 2c to give the title compound: MS (m/z) 417, 419 (M+1)⁺.

### c) 4-[4-Cyano-6-(4-cyano-2-methyl-phenyl)-3-hydroxy-pyridin-2-yl]-4-oxo-butyric acid ethyl ester

To a solution of 4-[4-bromo-6-(4-cyano-2-methyl-phenyl)-3-hydroxy-pyridin-2-yl]-4-oxo-butyric acid ethyl ester (103 mg, 0.25 mmol) in DMAC (3.5 mL) at room temperature were added Zn(CN)₂ (58 mg, 0.5 mmol), Pd₂(dba)₃ (22.6 mg, 0.025 mmol), dppf (27 mg, 0.05 mmol), and Zn dust (4.8 m, 0.075 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCO solution and water. The dried extract (MgSO₄) was concentrated in vacuo and purified by ISCO over silica gel, eluting with 5-35% EtOAc/hexanes to give product. MS (m/z) 364.0 (M+1)⁺.

### d) 4-[4-Cyano-6-(4-cyano-2-methyl-phenyl)-3-hydroxy-pyridin-2-yl]-4-oxo-butyric acid

To a solution of 4-[4-cyano-6-(4-cyano-2-methyl-phenyl)-3-hydroxy-pyridin-2-yl]-4-oxo-butyric acid ethyl ester (56 mg, 0.15 mmol) in THF (1.6 mL) and water (0.5 mL) at room temperature was added lithium hydroxide monohydrate (26 mg, 0.62 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 334.0 (M−1)⁺.

### Example 100

### 4-[4-Cyano-6-(5-cyano-2-methyl-phenyl)-3-hydroxy-pyridin-2-yl]-4-oxo-butyric acid

### a) 4-[6-(5-Cyano-2-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 5-cyano-2-methylphenylboronic acid in analogy to example 9a to give the title compound: MS (m/z) 339 (M+1)⁺.

### b) 4-[4-Bromo-6-(5-cyano-2-methyl-phenyl)-3-hydroxy-pyridin-2-yl]-4-oxo-butyric acid ethyl ester

The title compound was prepared from 4-[6-(5-cyano-2-methyl-phenyl)-3-hydroxy-pyridin-2-yl]-4-oxo-butyric acid ethyl ester and bromine in analogy to example 2c to give the title compound: MS (m/z) 417, 419 (M+1)⁺.

### c) 4-[4-Cyano-6-(5-cyano-2-methyl-phenyl)-3-hydroxy-pyridin-2-yl]-4-oxo-butyric acid ethyl ester

To a solution of 4-[4-bromo-6-(5-cyano-2-methyl-phenyl)-3-hydroxy-pyridin-2-yl]-4-oxo-butyric acid ethyl ester (138 mg, 0.33 mmol) in DMAC (4.7 mL) at room temperature were added Zn(CN)₂ (77 mg, 0.66 mmol), Pd₂(dba)₃ (30.2 mg, 0.033 mmol), dppf 37 mg, 0.066 mmol), and Zn dust (6.4 mg, 0.1 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 364.0 (M+1)⁺.

### d) 4-[4-Cyano-6-(5-cyano-2-methyl-phenyl)-3-hydroxy-pyridin-2-yl]-4-oxo-butyric acid

To a solution of 4-[4-cyano-6-(5-cyano-2-methyl-phenyl)-3-hydroxy-pyridin-2-yl]-4-oxo-butyric acid ethyl ester (78 mg, 0.21 mmol) in THF (2.3 mL) and water (0.85 mL) at room temperature was added lithium hydroxide monohydrate (36 mg, 0.85 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 334.0 (M−1)⁺.

### Example 101

### 4-[6-(2-Chloro-3-methyl-phenyl)-4-cyano-3-hydroxy-pyridin-2-yl]-4-oxo-butyric acid

### a) 4-[6-(2-Chloro-3-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-3-methylphenylboronic acid in analogy to example 9a to give the title compound: MS (m/z) 348 (M+1)⁺.

### b) 4-[4-Bromo-6-(2-chloro-3-methyl-phenyl)-3-hydroxy-pyridin-2-yl]-4-oxo-butyric acid ethyl ester

The title compound was prepared from 4-[6-(2-chloro-3-methyl-phenyl)-3-hydroxy-pyridin-2-yl]-4-oxo-butyric acid ethyl ester and bromine in analogy to example 2c to give the title compound: MS (m/z) 426, 428 (M+1)⁺.

### c) 4-[6-(2-Chloro-3-methyl-phenyl)-4-cyano-3-hydroxy-pyridin-2-yl]-4-oxo-butyric acid ethyl ester

To a solution of 4-[4-bromo-6-(2-chloro-3-methyl-phenyl)-3-hydroxy-pyridin-2-yl]-4-oxo-butyric acid ethyl ester (140 mg, 0.33 mmol) in DMAC (4.5 mL) at room temperature were added Zn(CN)₂ (77 mg, 0.66 mmol), Pd₂(dba)₃ (29 mg, 0.033 mmol), dppf (35 mg, 0.066 mmol), and Zn dust (6.2 mg, 0.1 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL), The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 373.0 (M+1)⁺.

### d) 4-[6-(2-Chloro-3-methyl-phenyl)-4-cyano-3-hydroxy-pyridin-2-yl]-4-oxo-butyric acid

To a solution of 4-[6-(2-chloro-3-methyl-phenyl)-4-cyano-3-hydroxy-pyridin-2-yl]-4-oxo-butyric acid ethyl ester (54.6 mg, 0.14 mmol) in THF (1.5 mL) and water (0.6 mL) at room temperature was added lithium hydroxide monohydrate (24.6 mg, 0.58 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 344.9 (M+1)⁺.

### Example 102

### 4-[6-(2-Chloro-5-methyl-phenyl)-4-cyano-3-hydroxy-pyridin-2-yl]-4-oxo-butyric acid

### a) 4-[6-(2-Chloro-5-methyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-5-methylphenylboronic acid in analogy to example 9a to give the title compound: MS (m/z) 348 (M+1)⁺.

### b) 4-[4-Bromo-6-(2-chloro-5-methyl-phenyl)-3-hydroxy-pyridin-2-yl]-4-oxo-butyric acid ethyl ester

The title compound was prepared from 4-[6-(2-chloro-5-methyl-phenyl)-3-hydroxy-pyridin-2-yl]-4-oxo-butyric acid ethyl ester and bromine in analogy to example 2c to give the title compound: MS (m/z) 426, 428 (M+1)⁺.

### c) 4-[6-(2-Chloro-5-methyl-phenyl)-4-cyano-3-hydroxy-pyridin-2-yl]-4-oxo-butyric acid ethyl ester

To a solution of 4-[4-bromo-6-(2-chloro-5-methyl-phenyl)-3-hydroxy-pyridin-2-yl]-4-oxo-butyric acid ethyl ester (139 mg, 0.32 mmol) in DMAC (4.6 mL) at room temperature were added Zn(CN)₂ (76 mg, 0.64 mmol), Pd₂(dba)₃ (29.8 mg 0,032 mmol), dppf (36.1 mg, 0.064 mmol), and Zn dust (6.3 mg, 0.09 mmol) to give a suspension. The reaction mixture was allowed to stir at 100° C. for 3-4 hours. After cooling to room temperature, the reaction mixture was diluted with EtOAc (50 ml and filtered through a celite plug, rinsing with EtOAc 150 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 372.9 (M+1)⁺.

### d) 4-[6-(2-Chloro-5-methyl-phenyl)-4-cyano-3-hydroxy-pyridin-2-yl]-4-oxo-butyric acid

To a solution of 4-[6-(2-chloro-5-methyl-phenyl)-4-cyano-3-hydroxy-pyridin-2-yl]-4-oxo-butyric acid ethyl ester (35 mg, 0.094 mmol) in THF (1 mL) and water (0.5 mL) at room temperature was added lithium hydroxide monohydrate (15.8 mg, 0.37 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 343.0, 345.0 (M−1)⁺.

### Example 103

### 4-[6-(2-Chloro-3-trifluoromethyl-phenyl)-4-cyano-3-hydroxy-pyridin-2-yl]-4-oxo-butyric acid

### a) 4-[6-(2-Chloro-3-trifluoromethyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-3-trifuoromethylphenylboronic acid in analogy to example 9a to give the title compound: MS (m/z) 402 (M+1)⁺.

### b) 4-[4-Bromo-6-(2-chloro-3-trifluoromethyl-phenyl)-3-hydroxy-pyridin-2-yl]-4-oxo-butyric acid ethyl ester

The title compound was prepared from 4-[6-(2-chloro-3-trifluoromethyl-phenyl)-3-hydroxy-pyridin-2-yl]-4-oxo-butyric acid ethyl ester and bromine in analogy to example 2c to give the title compound: MS (m/z) 478, 480 (M−1)⁺.

### c) 4-[6-(2-Chloro-3-trifluoromethyl-phenyl)-4-cyano-3-hydroxy-pyridin-2-yl]-4-oxo-butyric acid ethyl ester

To a solution of 4-[4-bromo-6-(2-chloro-3-trifluoromethyl-phenyl)-3-hydroxy-pyridin-2-yl]-4-oxo-butyric acid ethyl ester (145 mg, 0.3 mmol) in DMAC (4.2 mL) at room temperature were added Zn(CN)₂ (70.7 mg, 0.6 mmol), Pd₂(dba)₃ (27.5 mg, 0.03 mmol), dppf (33 mg, 0.06 mmol), and Zn dust (5.8 mg, 0.09 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc 150 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 426.9, 428.6 (M+1)⁺.

### d) 4-[6-(2-Chloro-3-trifluoromethyl-phenyl)-4-cyano-3-hydroxy-pyridin-2-yl]-4-oxo-butyric acid

To a solution of 4-[6-(2-chloro-3-trifluoromethyl-phenyl)-4-cyano-3-hydroxy-pyridin-2-yl]-4-oxo-butyric acid ethyl ester (65 mg, 0.15 mmol) in THF (1.6 mL) and water (0.5 mL) at room temperature was added lithium hydroxide monohydrate (25.6 mg, 0.61 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 397.0, 399.0 (M−1)⁺.

### Example 104

### 4-(5-Cyano-4-hydroxy-biphenyl-3-yl)-4-oxo-butyric acid

### a) 1-(4-Hydroxy-biphenyl-3-yl)-ethanone

A solution was prepared of 1-(5-bromo-2-hydroxy-phenyl)-ethanone (Combi-blocks, 6.2 g, 0.028 mol), 2% Pd(OAc)₂ (126 mg, 0.56 mmol), S-Phos (575 mg, 1.4 mmol), phenylboronic acid (5.1 g, 0.042 mol), and K₃PO₄ (11.92 g, 0.056 mol) in toluene (140 mL) and water (1 mL). The reaction mixture was stirred at 100° C. overnight. After cooling to room temperature, the reaction mixture was diluted with EtOAc (500 mL) and washed with 0.1 N HCl and water. The dried extract (MgSO₄) was concentrated in vacuo and purified by ISCO over silica gel, eluting with 2-50% EtOAc/hexanes, to give product. MS (m/z) 213.0 (M+1)⁺.

### b) 1-[4-(tert-Butyl-dimethyl-silanyloxy)-biphenyl-3-yl]-ethanone

To a solution of 1-(4-hydroxy-biphenyl-3-yl)-ethanone (3.78 g, 17.83 mmol) in DMF (35 mL) at room temperature was added imidazole (1.45 g, 21.39 mmol) and TBSCl (2.94 g, 19.61 mmol). The reaction mixture was stirred at room temperature overnight. The reaction mixture was diluted with Et₂O (500 mL) and washed with a NaHCO₃ solution and water. The dried extract (MgSO₄) was concentrated in vacuo and purified by ISCO over silica gel, eluting with 2-35% EtOAc/hexanes, to give product. MS (m/z) 327.0 (M+1)⁺.

### c) 4-(4-Hydroxy-biphenyl-3-yl)-4-oxo-butyric acid ethyl ester

To a solution of 1-[4-(tert-butyl-dimethyl-silanyloxy)-biphenyl-3-yl]-ethanone (5.1 g, 15.64 mmol) in THF (31 mL) and DMPU (8.1 mL) was added a solution of LiHMDS (20.3 mL, 1 M solution in THF, 20.3 mmol) at −60° C. under argon. After 10 minutes of stirring at −60° C., ethyl bromoacetate (3.48 mL, 31.28 mmol) was added. The mixture was stirred at −60° C. for an additional 10 minutes, then warmed to room temperature for 4h, quenched with water, and extracted with EtOAc (500 mL). The extract was washed with 0.1 N HCl solution and water. The dried extract (MgSO₄) was concentrated in vacuo and purified by ISCO over silica gel, eluting with 2-35% EtOAc/hexanes, to give product. MS (m/z) 298.9 (M+1)⁺.

### d) 4-(5-Bromo-4-hydroxy-biphenyl-3-yl)-4-oxo-butyric acid ethyl ester

To a solution of 4-(4-hydroxy-biphenyl-3-yl)-4-oxo-butyric acid ethyl ester (202 mg, 0.67 mmol) in CHCl₃ (6.7 mL) at room temperature was added sodium acetate (83 mg, 1.01 mmol) and bromine (162 mg, 1.01 mmol) to give a suspension. The reaction mixture was stirred at room temperature. After 24 h, the mixture was diluted with EtOAc (100 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 376.1, 378.8 (M+1)⁺.

### e) 4-(5-Cyano-4-hydroxy-biphenyl-3-yl)-4-oxo-butyric acid ethyl ester

To a solution of 4-(5-bromo-4-hydroxy-biphenyl-3-yl)-4-oxo-butyric acid ethyl ester (178 mg, 0.47 mmol) in DMAC (6.7 mL) at room temperature were added Zn(CN)_(110 rug, 0.94 mmol), Pd₂′dba (43 mg, 0.047 mmol) dppf (52 mg, 0.094 mmol, and Zn dust (9.1 mg, 0.14 m−1 mol) to give a suspension. The reaction mixture was allowed to stir at 100° C. for 3-4 hours. After cooling to room temperature, the reaction mixture was diluted with EtOAc (100 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 322.0 (M−1)⁺.

### f) 4-(5-Cyano-4-hydroxy-biphenyl-3-yl)-4-oxo-butyric acid

To a solution of 4-(5-cyano-4-hydroxy-biphenyl-3-yl)-4-oxo-butyric acid ethyl ester (86 mg, 0.26 mmol) in THF (2.8 mL) and water (1 mL) at room temperature was added lithium hydroxide monohydrate (45 mg, 1.06 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 294.0 (M−1)⁺.

### Example 105

### 4-[4-Cyano-6-(2-cyano-5-trifluoromethyl-phenyl)-3-hydroxy-pyridin-2-yl]-4-oxo-butyric acid

### a) 4-[6-(2-Chloro-5-trifluoromethyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-5-trifuoromethylphenylboronic acid in analogy to example 9a to give the title compound: MS (m/z) 402 (M+1)⁺.

### b) 4-[4-Bromo-6-(2-chloro-5-trifluoromethyl-phenyl)-3-hydroxy-pyridin-2-yl]-4-oxo-butyric acid ethyl ester

The title compound was prepared from 4-[6-(2-chloro-5-trifluoromethyl-phenyl)-3-hydroxy-pyridin-2-yl]-4-oxo-butyric acid ethyl ester and bromine in analogy to example 2c to give the title compound: MS (m/z) 480, 482 (M+1)⁺.

### c) 4-[4-Cyano-6-(2-cyano-5-trifluoromethyl-phenyl)-3-hydroxy-pyridin-2-yl]-4-oxo-butyric acid ethyl ester

To a solution of 4-[4-bromo-6-(2-chloro-5-trifluoromethyl-phenyl)-3-hydroxy-pyridin-2-yl]-4-oxo-butyric acid ethyl ester (132 mg, 0.27 mmol) in DMAC (3.9 mL) at room temperature were added Zn(CN)₂ (64 mg, 0.54 mmol), Pd₂(dba)₃ (25.2 mg, 0.027 mmol), dppf (30 mg, 0.054 mmol), and Zn dust (5.4 mg, 0.08 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 416.1 (M−1)⁺.

### d) 4-[4-Cyano-6-(2-cyano-5-trifluoromethyl-phenyl)-3-hydroxy-pyridin-2-yl]-4-oxo-butyric acid

To a solution of 4-[4-cyano-6-(2-cyano-5-trifluoromethyl-phenyl)-3-hydroxy-pyridin-2-yl]-4-oxo-butyric acid ethyl ester (42.2 mg, 0.1 mmol) in THF (1.1 mL) and water (0.4 mL) at room temperature was added lithium hydroxide monohydrate (17 mg, 0.4 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 388.0 (M−1)⁺.

### Example 106

### 4-[6-(2-Chloro-5-methoxy-phenyl)-4-cyano-3-hydroxy-pyridin-2-yl]-4-oxo-butyric acid

### a) 4-[6-(2-Chloro-5-methoxy-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-5-methoxyphenylboronic acid in analogy to example 9a to give the title compound: MS (m/z) 364 (M+1)⁺.

### b) 4-[4-Bromo-6-(2-chloro-5-methoxy-phenyl)-3-hydroxy-pyridin-2-yl]-4-oxo-butyric acid ethyl ester

The title compound was prepared from 4-[6-(2-chloro-5-methoxy-phenyl)-3-hydroxy-pyridin-2-yl]-4-oxo-butyric acid ethyl ester and bromine in analogy to example 2c to give the title compound: MS (m/z) 442, 444 (M+1)⁺.

### c) 4-[6-(2-Chloro-5-methoxy-phenyl)-4-cyano-3-hydroxy-pyridin-2-yl]-4-oxo-butyric acid ethyl ester

To a solution of 4-[4-bromo-6-(2-chloro-5-methoxy-phenyl)-3-hydroxy-pyridin-2-yl]-4-oxo-butyric acid ethyl ester (135 mg, 0.3 mmol) in DMAC (4.2 mL) at room temperature were added Zn(CN)₂ (71.5 mg, 0.6 mmol), Pd₂(dba)₃ (28 mg, 0.03 mmol), dppf (34 mg, 0.06 mmol), and Zn dust (6 mg, 0.09 mmol) to give a suspension. The reaction mixture was allowed to stir at 100° C. for 3-4 hours. After cooling to room temperature, the reaction mixture was diluted with EtOAc (50 mL) and filtered through celite plug, rinsing with EtOAc (50 mL) The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 389.0, 391.0 (M+1)⁺.

### d) 4-[6-(2-Chloro-5-methoxy-phenyl)-4-cyano-3-hydroxy-pyridin-2-yl]-4-oxo-butyric acid

To a solution of 4-[6-(2-chloro-5-methoxy-phenyl)-4-cyano-3-hydroxy-pyridin-2-yl]-4-oxo-butyric acid ethyl ester (76.4 mg, 0.19 mmol) in THF (2.1 mL) and water (0.75 mL) at room temperature was added lithium hydroxide monohydrate (33 mg, 0.78 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 359.0, 361.0 (M−1)⁺.

### Example 107

### 4-[6-(2-Chloro-3-fluoro-phenyl)-4-cyano-3-hydroxy-pyridin-2-yl]-4-oxo-butyric acid

### a) 4-[6-(2-Chloro-3-fluoro-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-3-fluorophenylboronic acid in analogy to example 9a to give the title compound: MS (m/z) 352 (M+1)⁺.

### b) 4-[4-Bromo-6-(2-chloro-3-fluoro-phenyl)-3-hydroxy-pyridin-2-yl]-4-oxo-butyric acid ethyl ester

The title compound was prepared from 4-[6-(2-chloro-3-fluoro-phenyl)-3-hydroxy-pyridin-2-yl]-4-oxo-butyric acid ethyl ester and bromine in analogy to example 2c to give the title compound: MS (m/z) 430, 432 (M+1)⁺.

### c) 4-[6-(2-Chloro-3-fluoro-phenyl)-4-cyano-3-hydroxy-pyridin-2-yl]-4-oxo-butyric acid ethyl ester

To a solution of 4-[4-bromo-6-(2-chloro-3-fluoro-phenyl)-3-hydroxy-pyridin-2-yl]-4-oxo-butyric acid ethyl ester (133 mg, 0.31 mmol) in DMAC (4.4 mL) at room temperature were added Zn(CN)₂ (723 mg, 0.62 mmol), Pd₂(dba)₃ (28 mg, 0.031 mmol, dppf (34 mg, 0.062 mmol), and Zn dust (6 mg, 0.09 mmol) to give a suspension. The reaction mixture was allowed to stir at 100° C. for 3-4 hours After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 375.0 (M−1)⁺.

### d) 4-[6-(2-Chloro-3-fluoro-phenyl)-4-cyano-3-hydroxy-pyridin-2-yl]-4-oxo-butyric acid

To a solution of 4-[6-(2-chloro-3-fluoro-phenyl)-4-cyano-3-hydroxy-pyridin-2-yl]-4-oxo-butyric acid ethyl ester (61.6 mg, 0.16 mmol) in THF (1.8 mL) and water (0.6 mL) at room temperature was added lithium hydroxide monohydrate (27 mg, 0.65 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 346.9, 348.9 (M−1)⁺.

### Example 108

### 4-[6-(2-Chloro-5-fluoro-phenyl)-4-cyano-3-hydroxy-pyridin-2-yl]-4-oxo-butyric acid

### a) 4-[6-(2-Chloro-5-fluoro-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-5-fluorophenylboronic acid in analogy to example 9a to give the title compound: MS (m/z) 352 (M+1)⁺.

### b) 4-[4-Bromo-6-(2-chloro-5-fluoro-phenyl)-3-hydroxy-pyridin-2-yl]-4-oxo-butyric acid ethyl ester

The title compound was prepared from 4-[6-(2-chloro-5-fluoro-phenyl)-3-hydroxy-pyridin-2-yl]-4-oxo-butyric acid ethyl ester and bromine in analogy to example 2c to give the title compound: MS (m/z) 430, 432 (M+1)⁺.

### c) 4-[6-(2-Chloro-5-fluoro-phenyl)-4-cyano-3-hydroxy-pyridin-2-yl]-4-oxo-butyric acid ethyl ester

To a solution of 4-[4-bromo-6-(2-chloro-5-fluoro-phenyl)-3-hydroxy-pyridin-2-yl]-4-oxo-butyric acid ethyl ester (142 mg, 0.33 mmol) in DMAC (4.7 mL) at room temperature were added Zn(CN)₂ (77.3 mg, 0.66 mmol), Pd₂(dbas (30.2 mg, 0,033 mmol), dppf (36.6 mg, 0.066 mmol), and Zn dust (6.4 mg, 0.1 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCO solution and water. The dried extract (MgSO₄) was concentrated in vacuo and purified by ISCO over silica gel, eluting with 5-35% EtOAc/hexanes to give product. MS (m/z) 377.0 (M+1)⁺.

### d) 4-[6-(2-Chloro-5-fluoro-phenyl)-4-cyano-3-hydroxy-pyridin-2-yl]-4-oxo-butyric acid

To a solution of 4-[6-(2-chloro-5-fluoro-phenyl)-4-cyano-3-hydroxy-pyridin-2-yl]-4-oxo-butyric acid ethyl ester (77.3 mg, 0.20 mmol) in THF (2.2 mL) and water (0.8 mL) at room temperature was added lithium hydroxide monohydrate (34.5 mg, 0.82 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 346.9, 348.9 (M−1)⁺.

### Example 109

### 4-[4-Cyano-6-(4-fluoro-naphthalen-1-yl)-3-hydroxy-pyridin-2-yl]-4-oxo-butyric acid

### a) 4-[6-(4-Fluoro-naphthalen-1-yl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and (4-fluoronaphthalen-1-yl)boronic acid in analogy to example 9a to give the title compound: MS (m/z) 368 (M+1)⁺.

### b) 4-[4-Bromo-6-(4-fluoro-naphthalen-1-yl)-3-hydroxy-pyridin-2-yl]-4-oxo-butyric acid ethyl ester

The title compound was prepared from 4-[6-(4-fluoro-naphthalen-1-yl)-3-hydroxy-pyridin-2-yl]-4-oxo-butyric acid ethyl ester and bromine in analogy to example 2c to give the title compound: MS (m/z) 446, 448 (M+1)⁺.

### c) 4-[4-Cyano-6-(4-fluoro-naphthalen-1-yl)-3-hydroxy-pyridin-2-yl]-4-oxo-butyric acid ethyl ester

To a solution of 4-[4-bromo-6-(4-fluoro-naphthalen-1-yl)-3-hydroxy-pyridin-2-yl]-4-oxo-butyric acid ethyl ester (148 mg, 0.33 mmol) in DMAC (4.7 mL) at room temperature were added Zn(CN)₂ (77.6 mg, 0.66 mmol), Pd₂(dba), (30.2 mg, 0.033 mmol), dppf (36.6 mg, 0.066 mmol), and Zn dust (6.4 mg, 0.1 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 391.0 (M−1)⁺.

### d) 4-[4-Cyano-6-(4-fluoro-naphthalen-1-yl)-3-hydroxy-pyridin-2-yl]-4-oxo-butyric acid

To a solution of 4-[4-cyano-6-(4-fluoro-naphthalen-1-yl)-3-hydroxy-pyridin-2-yl]-4-oxo-butyric acid ethyl ester (52.5 mg, 0.13 mmol) in THF (1.4 mL) and water (0.5 mL) at room temperature was added lithium hydroxide monohydrate (22.5 mg, 0.53 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 363.0 (M−1)⁺.

### Example 110

### 4-[6-(4-Chloro-naphthalen-1-yl)-4-cyano-3-hydroxy-pyridin-2-yl]-4-oxo-butyric acid

### a) 4-[6-(4-Chloro-naphthalen-1-yl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and (4-chloronaphthalen-1-yl)boronic acid in analogy to example 9a to give the title compound: MS (m/z) 384 (M+1)⁺.

### b) 4-[4-Bromo-6-(4-chloro-naphthalen-1-yl)-3-hydroxy-pyridin-2-yl]-4-oxo-butyric acid ethyl ester

The title compound was prepared from 4-[6-(4-chloro-naphthalen-1-yl)-3-hydroxy-pyridin-2-yl]-4-oxo-butyric acid ethyl ester and bromine in analogy to example 2c to give the title compound: MS (m/z) 462, 464 (M+1)⁺.

### c) 4-[6-(4-Chloro-naphthalen-1-yl)-4-cyano-3-hydroxy-pyridin-2-yl]-4-oxo-butyric acid ethyl ester

To a solution of 4-[4-bromo-6-(4-chloro-naphthalen-1-yl)-3-hydroxy-pyridin-2-yl]-4-oxo-butyric acid ethyl ester (141 mg, 0.305 mmol) in DMAC (4.3 mL) at room temperature were added Zn(CN)₂ (714 mg, 0.61 mmol), Pd₂(dba)₃ (27.9 mg, 0.0305 mmol), dppf (33.8 mg, 0.061 mmol), and Zn dust (5.9 mg, 0.09 mmol)) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL), The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 409.0, 411.0 (M+1)⁺.

### d) 4-[6-(4-Chloro-naphthalen-1-yl)-4-cyano-3-hydroxy-pyridin-2-yl]-4-oxo-butyric acid

To a solution of 4-[6-(4-chloro-naphthalen-1-yl)-4-cyano-3-hydroxy-pyridin-2-yl]-4-oxo-butyric acid ethyl ester (53.3 mg, 0.13 mmol) in THF (1.4 mL) and water (0.5 mL) at room temperature was added lithium hydroxide monohydrate (21.9 mg, 0.52 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 380.9, 382.8 (M+1)⁺.

### Example 111

### 4-[4-Cyano-3-hydroxy-6-(4-phenyl-naphthalen-1-yl)-pyridin-2-yl]-4-oxo-butyric acid

### a) 4-[3-Hydroxy-6-(4-phenyl-naphthalen-1-yl)-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and (4-phenylnaphthalen-1-yl)boronic acid in analogy to example 9a to give the title compound: MS (m/z) 426 (M+1)⁺.

### b) 4-[4-Bromo-3-hydroxy-6-(4-phenyl-naphthalen-1-yl)-pyridin-2-yl]-4-oxo-butyric acid ethyl ester

The title compound was prepared from 4-[3-hydroxy-6-(4-phenyl-naphthalen-1-yl)-pyridin-2-yl]-4-oxo-butyric acid ethyl ester and bromine in analogy to example 2c to give the title compound: MS (m/z) 504, 506 (M+1)⁺.

### c) 4-[4-Cyano-3-hydroxy-6-(4-phenyl-naphthalen-1-yl)-pyridin-2-yl]-4-oxo-butyric acid ethyl ester

To a solution of 4-[4-bromo-3-hydroxy-6-(4-phenyl-naphthalen-1-yl)-pyridin-2-yl]-4-oxo-butyric acid ethyl ester (141 mg, 0.28 mmol) in DMAC (4 mL) at room temperature were added Zn(CN)₂ (65 mg, 0.56 mmol), Pd₂(dba)₃ (25.5 mg, 0,028 mmol), dppf (30.9 mg, 0.056 mmol), and Zn dust (5.4 mg, 0.08 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc 150 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 451.0 (M+1)⁺.

### d) 4-[4-Cyano-3-hydroxy-6-(4-phenyl-naphthalen-1-yl)-pyridin-2-yl]-4-oxo-butyric acid

To a solution of 4-[4-cyano-3-hydroxy-6-(4-phenyl-naphthalen-1-yl)-pyridin-2-yl]-4-oxo-butyric acid ethyl ester (62.8 mg, 0.13 mmol) in THF (1.5 mL) and water (0.5 mL) at room temperature was added lithium hydroxide monohydrate (22 mg, 0.55 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 421.4 (M−1)⁺.

### Example 112

### 4-(4-Cyano-3-hydroxy-6-quinolin-5-yl-pyridin-2-yl)-4-oxo-butyric acid

### a) 4-(3-Hydroxy-6-quinolin-5-yl-pyridin-2-yl)-4-oxo-butyric acid ethyl ester

The title compound was prepared from 4-(6-bromo-3-hydroxy-pyridin-2-yl)-4-oxo-butyric acid ethyl ester (prepared from 66f) and 5-quinolineboronic acid in analogy to example 9a to give the title compound: MS (m/z) 351 (M+1)⁺.

### b) 4-(4-Bromo-3-hydroxy-6-quinolin-5-yl-pyridin-2-yl)-4-oxo-butyric acid ethyl ester

The title compound was prepared from 4-(3-hydroxy-6-quinolin-5-yl-pyridin-2-yl)-4-oxo-butyric acid ethyl ester and bromine in analogy to example 2c to give the title compound: MS (m/z) 429, 431 (M+1)⁺.

### c) 4-(4-Cyano-3-hydroxy-6-quinolin-5-yl-pyridin-2-yl)-4-oxo-butyric acid ethyl ester

To a solution of 4-(4-bromo-3-hydroxy-6-quinolin-5-yl-pyridin-2-yl)-4-oxo-butyric acid ethyl ester (141 mg, 0.32 mmol) in DMAC (4.6 mL) at room temperature were added Zn(CN)₂ (77 mg, 0.64 mmol), Pd₂(dba)₃ (30 mg, 0.032 mmol), dppf (36 mg, 0.064 mmol), and Zn dust (6.4 mg, 0.09 mmol) to give a suspension. The reaction mixture was allowed to stir at 100° C. for 3-4 hours. After cooling to room temperature, the reaction mixture was diluted with EtOAc (50 ml) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 376.0 (M+1)⁺.

### d) 4-(4-Cyano-3-hydroxy-6-quinolin-5-yl-pyridin-2-yl)-4-oxo-butyric acid

To a solution of 4-(4-cyano-3-hydroxy-6-quinolin-5-yl-pyridin-2-yl)-4-oxo-butyric acid ethyl ester (23.5 mg, 0.062 mmol) in THF (0.75 mL) and water (0.25 mL) at room temperature was added lithium hydroxide monohydrate (10.5 mg, 0.25 mmol) to give a suspension. The reaction mixture was stirred at room temperature. After 24 h, the mixture was concentrated in vacuo to give residue. The residue was dissolved in water (10 mL) and extracted with EtOAc (3×10 mL). The aqueous solution was acidified with a 1N HCl solution, filtered, washed with water, and dried to give product. MS (m/z) 347.9 (M+1)⁺.

### Example 113

### 4-[4-Cyano-3-hydroxy-6-(4-methyl-naphthalen-1-yl)-pyridin-2-yl]-4-oxo-butyric acid

### a) 4-[3-Hydroxy-6-(4-methyl-naphthalen-1-yl)-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and (4-methylnaphthalen-1-yl)boronic acid in analogy to example 9a to give the title compound: MS (m/z) 364 (M+1)⁺.

### b) 4-[4-Bromo-3-hydroxy-6-(4-methyl-naphthalen-1-yl)-pyridin-2-yl]-4-oxo-butyric acid ethyl ester

The title compound was prepared from 4-[3-hydroxy-6-(4-methyl-naphthalen-1-yl)-pyridin-2-yl]-4-oxo-butyric acid ethyl ester and bromine in analogy to example 2c to give the title compound: MS (m/z) 442, 444 (M+1)⁺.

### c) 4-[4-Cyano-3-hydroxy-6-(4-methyl-naphthalen-1-yl)-pyridin-2-yl]-4-oxo-butyric acid ethyl ester

To a solution of 4-[4-bromo-3-hydroxy-6-(4-methyl-naphthalen-1-yl)-pyridin-2-yl]-4-oxo-butyric acid ethyl ester (143 mg, 0.32 mmol) in DMAC (4.6 mL) at room temperature were added Zn(CN)₂ (76 mg, 0.64 mmol), Pd₂(dba)₃ (30 mg 0.032 mmol), dppf (36 mg, 0.064 mmol), and Zn dust (6.4 mg, 0.09 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 389.0 (M+1)⁺.

### d) 4-[4-Cyano-3-hydroxy-6-(4-methyl-naphthalen-1-yl)-pyridin-2-yl]-4-oxo-butyric acid

To a solution of 4-[4-cyano-3-hydroxy-6-(4-methyl-naphthalen-1-yl)-pyridin-2-yl]-4-oxo-butyric acid ethyl ester (68.6 mg, 0.17 mmol) in THF (1.9 mL) and water (0.6 mL) at room temperature was added lithium hydroxide monohydrate (29.7 mg, 0.71 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 359.1 (M−1)⁺.

### Example 114

### 4-[6-(2-Chloro-3-methoxy-phenyl)-4-cyano-3-hydroxy-pyridin-2-yl]-4-oxo-butyric acid

### a) 4-[6-(2-Chloro-3-methoxy-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-3-methoxyphenylboronic acid in analogy to example 9a to give the title compound: MS (m/z) 364 (M+1)⁺.

### b) 4-[4-Bromo-6-(2-chloro-3-methoxy-phenyl)-3-hydroxy-pyridin-2-yl]-4-oxo-butyric acid ethyl ester

The title compound was prepared from 4-[6-(2-chloro-3-methoxy-phenyl)-3-hydroxy-pyridin-2-yl]-4-oxo-butyric acid ethyl ester and bromine in analogy to example 2c to give the title compound: MS (m/z) 442, 444 (M+1)⁺.

### c) 4-[6-(2-Chloro-3-methoxy-phenyl)-4-cyano-3-hydroxy-pyridin-2-yl]-4-oxo-butyric acid ethyl ester

To a solution of 4-[4-bromo-6-(2-chloro-3-methoxy-phenyl)-3-hydroxy-pyridin-2-yl]-4-oxo-butyric acid ethyl ester (105 mg, 0.23 mmol) in DMAC (3.2 mL) at room temperature were added Zn(CN)₂ (56 mg, 0.46 mmol, Pd₂(dba)₃ (21 mg, 0.023 mmol), dppf (26 mg, 0.046 mmol), and Zn dust (4.5 mg, 0.07 mmol) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 388.9, 390.6 (M+1)⁺.

### d) 4-[6-(2-Chloro-3-methoxy-phenyl)-4-cyano-3-hydroxy-pyridin-2-yl]-4-oxo-butyric acid

To a solution of 4-[6-(2-chloro-3-methoxy-phenyl)-4-cyano-3-hydroxy-pyridin-2-yl]-4-oxo-butyric acid ethyl ester (54 mg, 0.13 mmol) in THF (1.5 mL) and water (0.5 mL) at room temperature was added lithium hydroxide monohydrate (23.4 mg, 0.56 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 359.1 (M−1)⁺.

### Example 115

### 4-[6-(2-Chloro-4-trifluoromethyl-phenyl)-4-cyano-3-hydroxy-pyridin-2-yl]-4-oxo-butyric acid

### a) 4-[6-(2-Chloro-4-trifluoromethyl-phenyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (prepared from 66f) and 2-chloro-4-trifluoromethylphenylboronic acid in analogy to example 9a to give the title compound: MS (m/z) 402 (M+1)⁺.

### b) 4-[4-Bromo-6-(2-chloro-4-trifluoromethyl-phenyl)-3-hydroxy-pyridin-2-yl]-4-oxo-butyric acid ethyl ester

The title compound was prepared from 4-[6-(2-chloro-4-trifluoromethyl-phenyl)-3-hydroxy-pyridin-2-yl]-4-oxo-butyric acid ethyl ester and bromine in analogy to example 2c to give the title compound: MS (m/z) 480, 482 (M+1)⁺.

### c) 4-[6-(2-Chloro-4-trifluoromethyl-phenyl)-4-cyano-3-hydroxy-pyridin-2-yl]-4-oxo-butyric acid ethyl ester

To a solution of 4-[4-bromo-6-(2-chloro-4-trifluoromethyl-phenyl)-3-hydroxy-pyridin-2-yl]-4-oxo-butyric acid ethyl ester (148 mg, 0.31 mmol) in DMAC (4.4 mL) at room temperature were added Zn(CN)₂ (72 mg, 0.62 mmol), Pd₂(dba)₃ (28.2 mg, 0.031 mmol), dppf (34.1 mg, 0062 mmol), and Zn dust (6 mg, 0.09 mmol) to give a suspension. The reaction mixture was allowed to stir at 100° C. for 3-4 hours. After cooling to room temperature, the reaction mixture was diluted with EtOAc (50 ml) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 425.0, 427.0 (M+1)⁺.

### d) 4-[6-(2-Chloro-4-trifluoromethyl-phenyl)-4-cyano-3-hydroxy-pyridin-2-yl]-4-oxo-butyric acid

To a solution of 4-[6-(2-chloro-4-trifluoromethyl-phenyl)-4-cyano-3-hydroxy-pyridin-2-yl]-4-oxo-butyric acid ethyl ester (42 mg, 0.098 mmol) in THF (1.05 mL) and water (0.35 mL) at room temperature was added lithium hydroxide monohydrate (16 mg, 0.39 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 397.0, 399.0 (M−1)⁺.

### Example 116

### 4-(4-Cyano-3-hydroxy-6-quinolin-8-yl-pyridin-2-yl)-4-oxo-butyric acid

### a) 4-(3-Hydroxy-6-quinolin-8-yl-pyridin-2-yl)-4-oxo-butyric acid ethyl ester

The title compound was prepared from 4-(6-bromo-3-hydroxy-pyridin-2-yl)-4-oxo-butyric acid ethyl ester (prepared from 66f) and 8-quinolinylboronic acid in analogy to example 9a to give the title compound: MS (m/z) 351 (M+1)⁺.

### b) 4-(4-Bromo-3-hydroxy-6-quinolin-8-yl-pyridin-2-yl)-4-oxo-butyric acid ethyl ester

The title compound was prepared from 4-(3-hydroxy-6-quinolin-8-yl-pyridin-2-yl)-4-oxo-butyric acid ethyl ester and bromine in analogy to example 2c to give the title compound: MS (m/z) 429, 431 (M+1)⁺.

### c) 4-(4-Cyano-3-hydroxy-6-quinolin-8-yl-pyridin-2-yl)-4-oxo-butyric acid ethyl ester

To a solution of 4-(4-bromo-3-hydroxy-6-quinolin-8-yl-pyridin-2-yl)-4-oxo-butyric acid ethyl ester (150 mg, 0.35 mmol) in DMAC (5 mL) at room temperature were added Zn(CN)₂ (82 rug, 0.7 mmol), Pd₂(dba)₃ (32 mg 0.035 mmol), dppf (39 mg, 0.07 mmol), and Zn dust (6.8 mg, 0.11 mmo) to give a suspension. The reaction mixture was allowed to stir at 100° C. for 3-4 hours. After cooling to room temperature, the reaction mixture was diluted with EtOAc (50 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 376.0 (M+1)⁺.

### d) 4-(4-Cyano-3-hydroxy-6-quinolin-8-yl-pyridin-2-yl)-4-oxo-butyric acid

To a solution of 4-(4-cyano-3-hydroxy-6-quinolin-8-yl-pyridin-2-yl)-4-oxo-butyric acid ethyl ester (59.4 mg, 0.16 mmol) in THF (1.7 mL) and water (0.6 mL) at room temperature was added lithium hydroxide monohydrate (27 mg, 0.63 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 347.9 (M+1)⁺.

### Example 117

### 4-(5-Benzyl-3-cyano-2-hydroxy-phenyl)-4-oxo-butyric acid

### a) 1-[5-Bromo-2-(tert-butyl-dimethyl-silanyloxy)-phenyl]-ethanone

To a solution of 1-(5-bromo-2-hydroxy-phenyl)-ethanone (Combi-blocks, 5.08 g, 23.6 mmol) in DMF (47 mL) at room temperature was added imidazole (1.93 g, 28 mmol) and TBSCl (3.89 g, 25.6 mmol). The reaction mixture was stirred at room temperature overnight. The reaction mixture was diluted with Et₂O (500 mL) and washed with a NaHCO₃ solution and water. The dried extract (MgSO₄) was concentrated in vacuo and purified by ISCO over silica gel, eluting with 2-35% EtOAc/hexanes, to give product. MS (m/z) 328.9, 330.8 (M+1)⁺.

### b) 4-(5-Bromo-2-hydroxy-phenyl)-4-oxo-butyric acid ethyl ester

To a solution of 1-[5-bromo-2-(tert-butyl-dimethyl-silanyloxy)-phenyl]-ethanone (5.3 g, 16.1 mmol) in THF (32 mL) and DMPU (8.35 mL) was added a solution of LiHMDS (20.9 mL, 1 M solution in THF, 20.9 mmol) at −60° C. under argon. After 10 minutes of stirring at −60° C., ethyl bromoacetate (3.48 mL, 31.28 mmol) was added. The mixture was stirred at −60° C. for additional 10 minutes, then warmed to room temperature for 4 h, quenched with water, and extracted with EtOAc (500 mL). The extract was washed with 0.1 N HCl solution and water. The dried extract (MgSO₄) was concentrated in vacuo and purified by ISCO over silica gel, eluting with 2-35% EtOAc/hexanes, to give product. MS (m/z) 300.9, 302.0 (M+1)⁺.

### c) 4-(5-Benzyl-2-hydroxy-phenyl)-4-oxo-butyric acid ethyl ester

A flask was charged with 4-(5-bromo-2-hydroxy-phenyl)-4-oxo-butyric acid ethyl ester (415 mg, 1.37 mmol), palladium acetate (15.37 mg, 0.068 mmol), and S-Phos (56.2 mg, 0.137 mmol). The flask was cycled with nitrogen and vacuum, then 5.5 mL of a benzylzinc bromide solution (0.5 M in THF) was added. The reaction mixture was stirred at room temperature for 16 h, then quenched with water, and extracted with EtOAc (100 mL). The dried extract (MgSO₄) was concentrated in vacuo and purified by ISCO over silica gel, eluting with 2-35% EtOAc/hexanes, to give product. MS (m/z) 312.9 (M+1)⁺.

### d) 4-(5-Benzyl-3-bromo-2-hydroxy-phenyl)-4-oxo-butyric acid ethyl ester

To a solution of 4-(5-benzyl-2-hydroxy-phenyl)-4-oxo-butyric acid ethyl ester (373 mg, 1.19 mmol) in CHCl₃ (11 mL) at room temperature was added sodium acetate (147 mg, 1.79 mmol) and bromine (286 mg, 1.79 mmol) to give a suspension. The reaction mixture was stirred at room temperature. After 24 h, the mixture was diluted with EtOAc (100 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 391.0, 393.0 (M+1)⁺.

### e) 4-(5-Benzyl-3-cyano-2-hydroxy-phenyl)-4-oxo-butyric acid ethyl ester

To a solution of 4-(5-benzyl-3-bromo-2-hydroxy-phenyl)-4-oxo-butyric acid ethyl ester (156 mg, 0.4 mmol) in DMAC (5.7 mL) at room temperature were added Zn(CN)₂ (94 mg, 0.8 mmol, Pd₂(dba)₃ (37 mg, 0.04 mmol) dppf (44 mg, 0.08 mmol), and Zn dust (7.8 rug, 0.12 mmol) to give a suspension. The reaction mixture was allowed to stir at 100° C. for 3-4 hours. After cooling to room temperature, the reaction mixture was diluted with EtOAc (100 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 336.1 (M−1)⁺.

### f) 4-(5-Benzyl-3-cyano-2-hydroxy-phenyl)-4-oxo-butyric acid

To a solution of 4-(5-benzyl-3-cyano-2-hydroxy-phenyl)-4-oxo-butyric acid ethyl ester (65.1 mg, 0.19 mmol) in THF (2 mL) and water (0.75 mL) at room temperature was added lithium hydroxide monohydrate (32 mg, 0.77 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 308.0 (M−1)⁺.

### Example 118

### 4-(4′-Chloro-5-cyano-4-hydroxy-biphenyl-3-yl)-4-oxo-butyric acid

### a) 4-(4′-Chloro-4-hydroxy-biphenyl-3-yl)-4-oxo-butyric acid ethyl ester

A solution was prepared of 4-(5-bromo-2-hydroxy-phenyl)-4-oxo-butyric acid ethyl ester (203 mg, 0.67 mol, prepared from example 117b), 2% Pd(OAc)₂ (3 mg, 0.013 mmol), S-Phos (14 mg, 0.033 mmol), 4-chlorophenylboronic acid (158 mg, 1.01 mol), and K₃PO₄ (285 mg, 1.34 mol) in toluene (4.5 mL) and water (18 mg). The reaction mixture was stirred at 100° C. overnight. After cooling to room temperature, the reaction mixture was diluted with EtOAc (100 mL) and washed with 0.1 N HCl and water. The dried extract (MgSO₄) was concentrated in vacuo and purified by ISCO over silica gel, eluting with 2-50% EtOAc/hexanes, to give product. MS (m/z) 332.3, 334.0 (M+1)⁺.

### b) 4-(5-Bromo-4′-chloro-4-hydroxy-biphenyl-3-yl)-4-oxo-butyric acid ethyl ester

To a solution of 4-(4′-Chloro-4-hydroxy-biphenyl-3-yl)-4-oxo-butyric acid ethyl ester (192 mg, 0.57 mmol) in CHCl₃ (5.7 mL) at room temperature was added sodium acetate (93.5 mg, 1.1 mmol) and bromine (102 mg, 0.63 mmol) to give a suspension. The reaction mixture was stirred at room temperature. After 24 h, the mixture was diluted with EtOAc (100 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 410.9, 412.9 (M+1)⁺.

### c) 4-(4′-Chloro-5-cyano-4-hydroxy-biphenyl-3-yl)-4-oxo-butyric acid ethyl ester

To a solution of 4-(5-bromo-4′-chloro-4-hydroxy-biphenyl-3-yl)-4-oxo-butyric acid ethyl ester (100 mg, 0.24 mmol) in DMAC (3.4 mL) at room temperature were added Zn(CN)₂ (57 mg, 0.48 mmol), Pd₂(dba)₃ (22 mg, 0.024 mmol), dppf (27 mg, 0.048 mmol), and Zn dust (4.6 mg, 0.07 mmol) to give a suspension. The reaction mixture was allowed to stir at 100° C. for 3-4 hours. After cooling to room temperature, the reaction mixture was diluted with EtOAc (100 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 355.9 (M−1)⁺.

### d) 4-(4′-Chloro-5-cyano-4-hydroxy-biphenyl-3-yl)-4-oxo-butyric acid

To a solution of 4-(4′-chloro-5-cyano-4-hydroxy-biphenyl-3-yl)-4-oxo-butyric acid ethyl ester (63 mg, 0.17 mmol) in THF (1.8 mL) and water (0.6 mL) at room temperature was added lithium hydroxide monohydrate (30 mg, 0.7 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 328.0 (M−1)⁺.

### Example 119

### 4-(2′-Chloro-5-cyano-4-hydroxy-biphenyl-3-yl)-4-oxo-butyric acid

### a) 4-(2′-Chloro-4-hydroxy-biphenyl-3-yl)-4-oxo-butyric acid ethyl ester

A solution was prepared of 4-(5-bromo-2-hydroxy-phenyl)-4-oxo-butyric acid ethyl ester (201 mg, 0.66 mol, prepared from example 117b), 2% Pd(OAc)₂ (3 mg, 0.013 mmol), S-Phos (14 mg, 0.033 mmol), 2-chlorophenylboronic acid (156 mg, 1.0 mol), and K₃PO₄ (280 mg, 1.32 mol) in toluene (4.5 mL) and water (24 mg). The reaction mixture was stirred at 100° C. overnight. After cooling to room temperature, the reaction mixture was diluted with EtOAc (100 mL) and washed with 0.1 N HCl and water. The dried extract (MgSO₄) was concentrated in vacuo and purified by ISCO over silica gel, eluting with 2-50% EtOAc/hexanes, to give product. MS (m/z) 331.0, 333.0 (M−1)⁺.

### b) 4-(5-Bromo-2′-chloro-4-hydroxy-biphenyl-3-yl)-4-oxo-butyric acid ethyl ester

To a solution of 4-(2′-chloro-4-hydroxy-biphenyl-3-yl)-4-oxo-butyric acid ethyl ester (201 mg, 0.6 mmol) in CHCl₃ (5.9 mL) at room temperature was added sodium acetate (98 mg, 1.2 mmol) and bromine (106 mg, 0.66 mmol) to give a suspension. The reaction mixture was stirred at room temperature. After 24 h, the mixture was diluted with EtOAc (100 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 410.9, 412.0 (M−1)⁺.

### c) 4-(2′-Chloro-5-cyano-4-hydroxy-biphenyl-3-yl)-4-oxo-butyric acid ethyl ester

To a solution of 4-(5-bromo-2′-chloro-4-hydroxy-biphenyl-3-yl)-4-oxo-butyric acid ethyl ester (190 mg, 0.46 mmol) in DMAC (6.5 mL) at room temperature were added Zn(CN)₂ (108 mg, 0.92 mmol), Pd₂(dba)₃ (42 mg, 0.046 mmol), dppf (51 mg, 0.092 mmol), and Zn dust (9 mg, 0.14 mmol) to give a suspension. The reaction mixture was allowed to stir at 100° C. for 3-4 hours. After cooling to room temperature, the reaction mixture was diluted with EtOAc (100 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 355.9 (M−1)⁺.

### d) 4-(2′-Chloro-5-cyano-4-hydroxy-biphenyl-3-yl)-4-oxo-butyric acid

To a solution of 4-(2′-chloro-5-cyano-4-hydroxy-biphenyl-3-yl)-4-oxo-butyric acid ethyl ester (51.6 mg, 0.14 mmol) in THF (1.5 mL) and water (0.6 mL) at room temperature was added lithium hydroxide monohydrate (24.2 mg, 0.57 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 328.0 (M−1)⁺.

### Example 120

### 4-(3′-Chloro-5-cyano-4-hydroxy-biphenyl-3-yl)-4-oxo-butyric acid

### a) 4-(3′-Chloro-4-hydroxy-biphenyl-3-yl)-4-oxo-butyric acid ethyl ester

A solution was prepared of 4-(5-bromo-2-hydroxy-phenyl)-4-oxo-butyric acid ethyl ester (190 mg, 0.63 mol, prepared from example 117b), 2% Pd(OAc)₂ (2.8 mg, 0.012 mmol), S-Phos (13 mg, 0.031 mmol), 3-chlorophenylboronic acid (148 mg, 0.94 mol), and K₃PO₄ (268 mg, 1.26 mol) in toluene (4.2 mL) and water (23 mg). The reaction mixture was stirred at 100° C. overnight. After cooling to room temperature, the reaction mixture was diluted with EtOAc (100 mL) and washed with 0.1 N HCl and water. The dried extract (MgSO₄) was concentrated in vacuo and purified by ISCO over silica gel, eluting with 2-50% EtOAc/hexanes, to give product. MS (m/z) 333.0 (M+1)⁺.

### b) 4-(5-Bromo-3′-chloro-4-hydroxy-biphenyl-3-yl)-4-oxo-butyric acid ethyl ester

To a solution of 4-(3′-chloro-4-hydroxy-biphenyl-3-yl)-4-oxo-butyric acid ethyl ester (118 mg, 0.33 mmol) in CHCl₃ (3.3 mL) at room temperature was added sodium acetate (54 mg, 0.66 mmol) and bromine (59 mg, 0.37 mmol) to give a suspension. The reaction mixture was stirred at room temperature. After 24 h, the mixture was diluted with EtOAc (100 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 410.9, 412.9 (M−1)⁺.

### c) 4-(3′-Chloro-5-cyano-4-hydroxy-biphenyl-3-yl)-4-oxo-butyric acid ethyl ester

To a solution of 4-(5-bromo-3′-chloro-4-hydroxy-biphenyl-3-yl)-4-oxo-butyric acid ethyl ester (118 mg, 0.28 mmol) in DMAC (4.1 mL) at room temperature were added Zn(CN)₂ (67 mg, 0.56 mmol), Pd₂(dba, (26 mg, 0.028 mmol), dppf (31 mg, 0.056 mmol), and Zn dust (5.6 mg, 0.08 mmol) to give a suspension. The reaction mixture was allowed to stir at 100° C. for 3-4 hours. After cooling to room temperature, the reaction mixture was diluted with EtOAc (100 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 356.0 (M−1)⁺.

### d) 4-(3′-Chloro-5-cyano-4-hydroxy-biphenyl-3-yl)-4-oxo-butyric acid

To a solution of 4-(3′-chloro-5-cyano-4-hydroxy-biphenyl-3-yl)-4-oxo-butyric acid ethyl ester (42 mg, 0.11 mmol) in THF (1.2 mL) and water (0.4 mL) at room temperature was added lithium hydroxide monohydrate (20 mg, 0.44 mmol) to give a suspension. The reaction mixture was stirred at room temperature. After 24 h, the mixture was concentrated in vacuo to give residue. The residue was dissolved in water (15 mL) and extracted with EtOAc (3×25 mL). The aqueous solution was acidified with a 1N HCl solution, filtered, washed with water, and dried to give product. MS (m/z) 327.9 (M−1)⁺.

### Example 121

### 4-[3-Cyano-5-(2,6-dichloro-benzyl)-2-hydroxy-phenyl]-4-oxo-butyric acid

### a) 4-[5-(2,6-Dichloro-benzyl)-2-hydroxy-phenyl]-4-oxo-butyric acid ethyl ester

A flask was charged with 4-(5-bromo-2-hydroxy-phenyl)-4-oxo-butyric acid ethyl ester (403 mg, 1.33 mmol, prepared from example 117b), palladium acetate (14.9 mg, 0.066 mmol) and S-Phos (54.5 mg, 0.133 mmol). The flask was cycled with nitrogen and vacuum, then 5.4 mL of a 2,6-dichlorobenzylzinc bromide solution (0.5 M in THF) was added. The reaction mixture was stirred at room temperature for 16 h, quenched with water, and extracted with EtOAc (100 mL). The dried extract (MgSO₄) was concentrated in vacuo and purified by ISCO over silica gel, eluting with 2-35% EtOAc/hexanes, to give product. MS (m/z) 381.0, 382.8 (M+1)⁺.

### b) 4-[3-Bromo-5-(2,6-dichloro-benzyl)-2-hydroxy-phenyl]-4-oxo-butyric acid ethyl ester

To a solution of 4-[5-(2,6-dichloro-benzyl)-2-hydroxy-phenyl]-4-oxo-butyric acid ethyl ester (500 mg, 1.31 mmol) in CHCl₃ (13 mL) at room temperature was added sodium acetate (161 mg, 1.96 mmol) and bromine (230 mg, 1.44 mmol) to give a suspension. The reaction mixture was stirred at room temperature. After 24 h, the mixture was diluted with EtOAc (100 mL). The organic layers were washed with a Na₂S₂O₃ solution and water. The dried extract (MgSO₄) was concentrated in vacuo and purified by ISCO over silica gel, eluting with 5-35% EtOAc/hexanes to give product. MS (m/z) 460.0, 461.0 (M+1)⁺.

### c) 4-[3-Cyano-5-(2,6-dichloro-benzyl)-2-hydroxy-phenyl]-4-oxo-butyric acid ethyl ester

To a solution of 4-[3-bromo-5-(2,6-dichloro-benzyl)-2-hydroxy-phenyl]-4-oxo-butyric acid ethyl ester (155 mg, 0.33 mmol) in DMAC (4.7 mL) at room temperature were added Zn(CN)₂ (79 mg, 0.66 mmol), Pd₂(dba)₃ (30 mg, 0.033 mmol), dppf (37 rug, 0.066 mmol and Zn dust (6.4 mg, 0.1 mmol) to give a suspension. The reaction mixture was allowed to stir at 100° C. for 3-4 hours. After cooling to room temperature, the reaction mixture was diluted with EtOAc (100 mL) and filtered through a celite plug, rinsing with EtOAc (50 mL). The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 404.0, 406.0 (M−1)⁺.

### d) 4-[3-Cyano-5-(2,6-dichloro-benzyl)-2-hydroxy-phenyl]-4-oxo-butyric acid

To a solution of 4-[3-cyano-5-(2,6-dichloro-benzyl)-2-hydroxy-phenyl]-4-oxo-butyric acid ethyl ester (72.9 mg, 0.18 mmol) in THF (1.9 mL) and water (0.6 mL) at room temperature was added lithium hydroxide monohydrate (30 mg, 0.72 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 376.0, 378.0 (M−1)⁺.

### Example 122

### 4-[3-Cyano-5-(2,6-dimethyl-benzyl)-2-hydroxy-phenyl]-4-oxo-butyric acid

### a) 4-[5-(2,6-Dimethyl-benzyl)-2-hydroxy-phenyl]-4-oxo-butyric acid ethyl ester

Zinc powder (496 mg, 7.64 mmol) was suspended in anhydrous THF (4 mL) under N₂, and then 1,2-dibromoethane (28.1 mg, 0.15 mmol) and TMSCl (132 mg, 1.21 mmol) was added. The mixture was heated at 65° C. for 30 min. After the mixture was cooled to 0° C., a solution of 2,6-dimethyl benzyl bromide (760 mg, 3.82 mmol) in anhydrous THF (5 mL) was added dropwise. The resulting mixture was stirred at room temperature for 2 h.

A flask was charged with 4-(5-bromo-2-hydroxy-phenyl)-4-oxo-butyric acid ethyl ester (286 mg, 0.95 mmol, prepared from example 117b), palladium acetate (11 mg, 0.05 mmol) and S-Phos (39.1 mg, 0.095 mmol). The flask was cycled with nitrogen and vacuum, and then the dimethyl benzyl bromide solution in THF was added. The reaction mixture was stirred at room temperature for 16 h, quenched with water, and extracted with EtOAc (100 mL). The dried extract (MgSO₄) was concentrated in vacuo and purified by ISCO over silica gel, eluting with 2-35% EtOAc/hexanes, to give product. MS (m/z) 341.1 (M+1)⁺.

### b) 4-[3-Bromo-5-(2,6-dimethyl-benzyl)-2-hydroxy-phenyl]-4-oxo-butyric acid ethyl ester

To a solution of 4-[5-(2,6-dimethyl-benzyl)-2-hydroxy-phenyl]-4-oxo-butyric acid ethyl ester (321 mg, 0.94 mmol) in CHCl₃ (9.4 mL) at room temperature was added sodium acetate (115 mg, 1.4 mmol) and bromine (166 mg, 1.03 mmol) to give a suspension. The reaction mixture was stirred at room temperature. After 24 h, the mixture was diluted with EtOAc (100 mL). The organic layers were washed with a Na S₂O₃ solution and water. The dried extract (MgSO₄) was concentrated in vacuo and purified by ISCO over silica gel, eluting with 5-35% EtOAc/hexanes to give product. MS (m/z) 418.9, 420.9 (M+1)⁺.

### c) 4-[3-Cyano-5-(2,6-dimethyl-benzyl)-2-hydroxy-phenyl]-4-oxo-butyric acid ethyl ester

To a solution of 4-[3-bromo-5-(2,6-dimethyl-benzyl)-2-hydroxy-phenyl]-4-oxo-butyric acid ethyl ester (161 mg, 0.39 mmol) in DMAC (5.5 mL) at room temperature were added Zn(CN)₂ (90 mg, 0.78 mmol), Pd₂(dba)₃ (35 mg, 0.039 mmol), dppf (43 mg, 0.078 mmol), and Zn dust (7.5 mug, 0.12 mmol) to give a suspension. The reaction mixture was allowed to stir at 100° C. for 3-4 hours. After cooling to room temperature, the reaction mixture was diluted with EtOAc (100 mill) and filtered through a celite plug, rinsing with EtOAc (50 mL), The organic layers were washed with a 0.1 N HCl solution and water. The dried extract (MgSO₄) was concentrated in vacuo and purified by ISCO over silica gel, eluting with 5-35% EtOAc/hexanes to give product. MS (m/z) 364.1 (M−1)⁺.

### d) 4-[3-Cyano-5-(2,6-dimethyl-benzyl)-2-hydroxy-phenyl]-4-oxo-butyric acid

To a solution of 4-[3-Cyano-5-(2,6-dimethyl-benzyl)-2-hydroxy-phenyl]-4-oxo-butyric acid ethyl ester (50.4 mg, 0.14 mmol) in THF (1.5 mL) and water (0.5 mL) at room temperature was added lithium hydroxide monohydrate (23 mg, 0.55 mmol) to give a suspension. The reaction mixture was stirred at room temperature. After 24 h, the mixture was concentrated in vacuo to give residue. The residue was dissolved in water (15 mL) and extracted with EtOAc (3×15 mL). The aqueous solution was acidified with a 1N HCl solution, filtered, washed with water, and dried to give product. MS (m/z) 336.1 (M−1)⁺.

### Example 123

### 4-[6-(2-Chloro-6-methyl-benzyl)-4-cyano-3-hydroxy-pyridin-2-yl]-4-oxo-butyric acid

### a) (2-Chloro-6-methyl-phenyl)-methanol

To a solution of 2-chloro-6-methyl benzoic acid (1g, 5.86 mmol) in THF (10 mL) was added 1M BH₃ (17.6 mL, 17.6 mmol) in THF at 0° C. under N₂ with ice-cooling. The mixture was allowed to warm to room temperature and then refluxed for overnight. The reaction mixture was then cooled to 0° C. and quenched with MeOH, followed by 1M HCl. The mixture was extracted with DCM, the organic layer was separated, dried over Na₂SO₄, and concentrated under vacuum to give crude, which was purified by column chromatography using 0-5% ethyl acetate in hexane as eluent to give the title compound. MS-(+)-ion, M−OH=139.55. ¹H NMR (CDCl₃, 400 MHz) δ=7.24-7.22 (m, 1H), 7.15-7.09 (m, 2H), 4.84 (s, 2H), 2.46 (s, 3H).

### b) 2-Bromomethyl-1-chloro-3-methyl-benzene

To a solution of (2-chloro-6-methyl-phenyl)-methanol (0.720 g, 4.59 mmol) in DCM (10 mL) was added PPh₃ (1.68g, 6.42 mmol) and NBS (1.14g, 6.42 mmol) at 0° C. under N₂. The mixture was stirred at room temperature for 8h. Upon reaction completion, the resulting mixture was diluted with water and extracted with ethyl acetate. The organic layer was separated, dried over Na₂SO₄, and concentrated under vacuum to give crude, which was purified by column chromatography using 0-5% ethyl acetate in hexane as eluent to give the title compound. ¹H NMR (CDCl₃, 400 MHz) δ=7.26-7.24 (m, 1H, merged with CHCl₃), 7.16-7.09 (m, 2H), 4.69 (s, 2H), 2.46 (s, 3H).

### c) 2-chloro-6-methyl-benzylzinc(II) bromide

The title compound was prepared from 2-bromomethyl-1-chloro-3-methyl-benzene (see Example 123b) and zinc dust in analogy to example 39a.

### d) 4-[6-(2-Chloro-6-methyl-benzyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (see Example 2a) and 2-chloro-6-methyl-benzylzinc(II) bromide in THF in analogy to example 10a, MS-(+)-ion, M+H=381.94, 361.98.

### e) 4-[4-Bromo-6-(2-chloro-6-methyl-benzyl)-3-hydroxy-pyridin-2-yl]-4-oxo-butyric acid ethyl ester

The title compound was prepared from 4-[6-(2-chloro-6-methyl-benzyl)-3-hydroxy-pyridin-2-yl]-4-oxo-butyric acid ethyl ester and bromine in analogy to example 2c, MS-(+)-ion, M+H=441.80.

### f) 4-[6-(2-Chloro-6-methyl-benzyl)-4-cyano-3-hydroxy-pyridin-2-yl]-4-oxo-butyric acid ethyl ester

The title compound was prepared from 4-[4-bromo-6-(2-chloro-6-methyl-benzyl)-3-hydroxy-pyridin-2-yl]-4-oxo-butyric acid ethyl ester and zinc(II) cyanide in analogy to example 2b, MS-(+)-ion, M+H=386.99.

### g) 4-[6-(2-Chloro-6-methyl-benzyl)-4-cyano-3-hydroxy-pyridin-2-yl]-4-oxo-butyric acid

The title compound was prepared from 4-[6-(2-chloro-6-methyl-benzyl)-4-cyano-3-hydroxy-pyridin-2-yl]-4-oxo-butyric acid ethyl ester in analogy to example 1g, MS-(+)-ion, M+H=358.93.

### Example 124

### 4-[4-Cyano-3-hydroxy-6-(2,4,6-trimethyl-benzyl)-pyridin-2-yl]-4-oxo-butyric acid

### a) 2,4,6-Trimethyl-benzylzinc(II) bromide

The title compound was prepared from 2-bromomethyl-1,3,5-trimethyl-benzene and zinc dust in analogy to example 39a.

### b) 4-[3-Hydroxy-6-(2,4,6-trimethyl-benzyl)-pyridin-2-yl]-4-oxo-butyric acid ethyl ester

The title compound was prepared from 4-(6-bromo-3-hydroxy-pyridin-2-yl)-4-oxo-butyric acid ethyl ester (see Example 2a) and 2,4,6-trimethyl-benzylzinc(II) bromide in THF in analogy to example 10a, MS-(+)-ion, M+H=356.03.

### c) 4-[4-Bromo-3-hydroxy-6-(2,4,6-trimethyl-benzyl)-pyridin-2-yl]-4-oxo-butyric acid ethyl ester

To a solution of 4-[3-hydroxy-6-(2,4,6-trimethyl-benzyl)-pyridin-2-yl]-4-oxo-butyric acid ethyl ester (142 mg, 0.4 mmol) in methanol (10 mL) was added N-bromosuccinimide (71 mg, 0.4 mmol). The mixture was stirred at room temperature for 2h. Silica gel was added to the reaction. The mixture was concentrated under vacuum to give crude product, which was purified by column chromatography using 0-10% ethyl acetate in hexane as eluent to give the title compound. MS-(+)-ion, M+H=433.95, 435.90.

### d) 4-[4-Cyano-3-hydroxy-6-(2,4,6-trimethyl-benzyl)-pyridin-2-yl]-4-oxo-butyric acid ethyl ester

The title compound was prepared from 4-[4-bromo-3-hydroxy-6-(2,4,6-trimethyl-benzyl)-pyridin-2-yl]-4-oxo-butyric acid ethyl ester and zinc(II) cyanide in analogy to example 2b, MS-(+)-ion, M+H=414.99.

### e) 4-[4-Cyano-3-hydroxy-6-(2,4,6-trimethyl-benzyl)-pyridin-2-yl]-4-oxo-butyric acid

The title compound was prepared from 4-[4-cyano-3-hydroxy-6-(2,4,6-trimethyl-benzyl)-pyridin-2-yl]-4-oxo-butyric acid ethyl ester in analogy to example 1g, MS-(+)-ion, M+H=353.03.

### Example 125

### 4-[6-(2-Chloro-6-methoxy-benzyl)-4-cyano-3-hydroxy-pyridin-2-yl]-4-oxo-butyric acid

### a) (2-Chloro-6-methoxyl-phenyl)-methanol

The title compound was prepared from 2-chloro-6-methoxyl benzoic acid and BH₃ in analogy to example 123a. MS-(+)-ion, M-OH=155.45. ¹H NMR (CDCl₃, 400 MHz) δ=7.20 (t, J=8.2 Hz, 1H), 7.01 (d, J=8.2 Hz, 1H), 6.82 (d, J=8.2 Hz, 1H), 4.88 (s, 2H), 3.88 (s, 3H).

### b) 2-Bromomethyl-1-chloro-3-methoxyl-benzene

To a solution of (2-chloro-6-methoxyl-phenyl)-methanol (0.500g, 2.89 mmol) in DCM (10 mL) was added PBr₃ (0.27 mL, 2.92 mmol) in DCM (10 mL) at −10° C. under N₂. The mixture was stirred at 0° C. for 6h. Upon reaction completion, the resulting mixture was diluted with water, quenched with aq. NaHCO₃ solution, and extracted with ethyl acetate. The organic layer was separated, dried over Na₂SO₄, and concentrated under vacuum to give crude product, which was purified by column chromatography using 0-5% ethyl acetate in hexane as eluent to give the title compound. ¹H NMR (CDCl₃, 400 MHz) δ=7.21 (t, J=8.0 Hz, 1H), 7.01 (d, J=8.0 Hz, 1H), 6.81 (d, J=8.0 Hz, 1H), 4.72 (s, 2H), 3.91 (s, 3H).

### c) 2-chloro-6-methoxyl-benzylzinc(II) bromide

The title compound was prepared from 2-bromomethyl-1-chloro-3-methoxy-benzene and zinc dust in analogy to example 39a.

### d) 4-[6-(2-Chloro-6-methoxy-benzyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (see Example 2a) and 2-chloro-6-methoxyl-benzylzinc(II) bromide in THF in analogy to example 10a, MS-(+)-ion, M+H=377.94.

### e) 4-[4-Bromo-6-(2-chloro-6-methoxy-benzyl)-3-hydroxy-pyridin-2-yl]-4-oxo-butyric acid ethyl ester

The title compound was prepared from 4-[6-(2-chloro-6-methoxy-benzyl)-3-hydroxy-pyridin-2-yl]-4-oxo-butyric acid ethyl ester and N-bromosuccinimide in analogy to example 124c, MS-(+)-ion, M+H=457.75.

### f) 4-[6-(2-Chloro-6-methoxy-benzyl)-4-cyano-3-hydroxy-pyridin-2-yl]-4-oxo-butyric acid ethyl ester

The title compound was prepared from 4-[4-bromo-6-(2-chloro-6-methoxy-benzyl)-3-hydroxy-pyridin-2-yl]-4-oxo-butyric acid ethyl ester and zinc(II) cyanide in analogy to example 2b, MS-(+)-ion, M+H=402.99.

### g) 4-[6-(2-Chloro-6-methoxy-benzyl)-4-cyano-3-hydroxy-pyridin-2-yl]-4-oxo-butyric acid

The title compound was prepared from 4-[6-(2-chloro-6-methoxyl-benzyl)-4-cyano-3-hydroxy-pyridin-2-yl]-4-oxo-butyric acid ethyl ester in analogy to example 1g, MS-(+)-ion, M+H=374.94.

### Example 126

### 4-[4-Cyano-6-(2,6-dichloro-4-fluoro-benzyl)-3-hydroxy-pyridin-2-yl]-4-oxo-butyric acid

### a) (2,6-Dichloro-4-fluoro-phenyl)-methanol

To a solution of 2,6-dichloro-4-fluoro-benzaldehyde (1.0 g, 5.2 mmol) in methanol (20 mL) was added sodium borohydride (0.3 g, 7.8 mmol) under N₂ with ice-cooling. 30 min later, the mixture was quenched by the addition of NH₄Cl aqueous solution (20 mL). The mixture was then concentrated under reduced pressure to remove most of the methanol. The residue was extracted with ethyl acetate (20 mL×3). The combined extracts were washed with brine (50 mL), dried over sodium sulfate, filtered and evaporated in vacuo to afford the product. MS-(+)-ion, M-OH=176.98. ¹H NMR (CDCl₃, 200 MHz) δ=7.11 (d, J=8.2 Hz, 2H), 4.92 (s, 2H).

### b) 2-Bromomethyl-1,3-dichloro-5-fluoro-benzene

To a solution of (2,6-dichloro-4-fluoro-phenyl)-methanol (1.01 g, 5.2 mmol) in CH₂C₁₂ (15 mL) was added PBr₃ (4.41 g, 5.2 mmol) at 0° C. After 2 h at 0° C., the mixture was poured into ice-water and extracted with CH₂Cl₂. The combined organic layers were washed with saturated aq. NaHCO₃, brine, dried (Na₂SO₄), concentrated under reduced pressure, and purified by flash column chromatography to give the title compound. ¹H NMR (CDCl₃, 200 MHz) δ=7.12 (d, J=7.8 Hz, 2H), 4.72 (s, 2H).

### c) 2,6-Dichloro-4-fluoro-benzylzinc(II) bromide

The title compound was prepared from 2-bromomethyl-1,3-dichloro-5-fluoro-benzene and zinc dust in analogy to example 39a.

### d) 4-[6-(2,6-Dichloro-4-fluoro-benzyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (see Example 2a) and 2,6-dichloro-4-fluoro-benzylzinc(II) bromide in THF in analogy to example 10a, MS-(+)-ion, M+H=399.94, 401.79.

### e) 4-[4-Bromo-6-(2,6-dichloro-4-fluoro-benzyl)-3-hydroxy-pyridin-2-yl]-4-oxo-butyric acid ethyl ester

The title compound was prepared from 4-[6-(2,6-dichloro-4-fluoro-benzyl)-3-hydroxy-pyridin-2-yl]-4-oxo-butyric acid ethyl ester and bromine in analogy to example 2c, MS-(+)-ion, M+H=479.76.

### f) 4-[4-Cyano-6-(2,6-dichloro-4-fluoro-benzyl)-3-hydroxy-pyridin-2-yl]-4-oxo-butyric acid ethyl ester

The title compound was prepared from 4-[4-bromo-6-(2,6-dichloro-4-fluoro-benzyl)-3-hydroxy-pyridin-2-yl]-4-oxo-butyric acid ethyl ester and zinc(II) cyanide in analogy to example 2b, MS-(+)-ion, M+H=424.90, 426.75.

### g) 4-[4-Cyano-6-(2,6-dichloro-4-fluoro-benzyl)-3-hydroxy-pyridin-2-yl]-4-oxo-butyric acid

The title compound was prepared from 4-[4-cyano-6-(2,6-dichloro-4-fluoro-benzyl)-3-hydroxy-pyridin-2-yl]-4-oxo-butyric acid ethyl ester in analogy to example 1g, MS-(+)-ion, M+H=396.89.

### Example 127

### 4-[4-Cyano-6-(2,6-dichloro-4-methyl-benzyl)-3-hydroxy-pyridin-2-yl]-4-oxo-butyric acid

### a) (2,6-Dichloro-4-methyl-phenyl)-methanol

The title compound was prepared from 2,6-dichloro-4-methyl-benzaldehyde and sodium borohydride in analogy to example 126a. MS-(+)-ion, M-OH=172.90. ¹H NMR (CDCl₃, 200 MHz) δ=7.15 (s, 2H), 4.92 (d, J=6.2 Hz, 2H), 2.31 (s, 3H), 2.01 (t, J=6.2 Hz, 1H).

### b) 2-Bromomethyl-1,3-dichloro-5-methyl-benzene

The title compound was prepared from (2,6-dichloro-4-methyl-phenyl)-methanol and PBr₃ in analogy to example 126b. MS-(+)-ion, M-OH=172.90. ¹H NMR (CDCl₃, 200 MHz) δ=7.15 (s, 2H), 4.74 (s, 2H), 2.31 (s, 3H).

### c) 2,6-Dichloro-4-methyl-benzylzinc(II) bromide

The title compound was prepared from 2-bromomethyl-1,3-dichloro-5-methyl-benzene and zinc dust in analogy to example 39a.

### d) 4-[6-(2,6-Dichloro-4-methyl-benzyl)-3-hydroxy-pyridin-2-yl]-4-oxo-butyric acid ethyl ester

The title compound was prepared from 4-(6-bromo-3-hydroxy-pyridin-2-yl)-4-oxo-butyric acid ethyl ester (see Example 2a) and 2,6-dichloro-4-methyl-benzylzinc(II) bromide in THF in analogy to example 10a, MS-(+)-ion, M+H=395.94, 397.84.

### e) 4-[4-Bromo-6-(2,6-dichloro-4-methyl-benzyl)-3-hydroxy-pyridin-2-yl]-4-oxo-butyric acid ethyl ester

The title compound was prepared from 4-[6-(2,6-dichloro-4-methyl-benzyl)-3-hydroxy-pyridin-2-yl]-4-oxo-butyric acid ethyl ester and bromine in analogy to example 2c, MS-(+)-ion, M+H=475.76.

### f) 4-[4-Cyano-6-(2,6-dichloro-4-methyl-benzyl)-3-hydroxy-pyridin-2-yl]-4-oxo-butyric acid ethyl ester

The title compound was prepared from 4-[4-bromo-6-(2,6-dichloro-4-methyl-benzyl)-3-hydroxy-pyridin-2-yl]-4-oxo-butyric acid ethyl ester and zinc(II) cyanide in analogy to example 2b, MS-(+)-ion, M+H=420.95, 422.95.

### g) 4-[4-Cyano-6-(2,6-dichloro-4-methyl-benzyl)-3-hydroxy-pyridin-2-yl]-4-oxo-butyric acid

The title compound was prepared from 4-[4-cyano-6-(2,6-dichloro-4-methyl-benzyl)-3-hydroxy-pyridin-2-yl]-4-oxo-butyric acid ethyl ester in analogy to example 1g, MS-(+)-ion, M+H=392.89, 394.79.

### Example 128

### 4-[4-Cyano-6-(2,6-dichloro-benzoylamino)-3-hydroxy-pyridin-2-yl]-4-oxo-butyric acid

### a) 4-[3-Benzyloxy-4-bromo-6-(2,6-dichloro-benzoylamino)-pyridin-2-yl]-4-oxo-butyric acid ethyl ester

The title compound was prepared from 4-(3-Benzyloxy-4,6-dibromo-pyridin-2-yl)-4-oxo-butyric acid ethyl ester and 2,6-dichloro-benzamide in analogy to example 157a. MS-(+)-ion, M+H=580.82.

### b) 4-[4-Cyano-6-(2,6-dichloro-benzoylamino)-3-hydroxy-pyridin-2-yl]-4-oxo-butyric acid ethyl ester

The title compound was prepared from 4-[3-benzyloxy-4-bromo-6-(2,6-dichloro-benzoylamino)-pyridin-2-yl]-4-oxo-butyric acid ethyl ester and zinc(II) cyanide in analogy to example 2b. MS-(+)-ion, M+H=435.90.

### c) 4-[4-Cyano-6-(2,6-dichloro-benzoylamino)-3-hydroxy-pyridin-2-yl]-4-oxo-butyric acid

The title compound was prepared from 4-[4-Cyano-6-(2,6-dichloro-benzoylamino)-3-hydroxy-pyridin-2-yl]-4-oxo-butyric acid ethyl ester in analogy to example 1g. MS-(+)-ion, M+H=407.89.

### Example 129

### 4-[4-Cyano-6-(2,6-dimethyl-benzoylamino)-3-hydroxy-pyridin-2-yl]-4-oxo-butyric acid

### a) 4-[4-Cyano-6-(2,6-dimethyl-benzoylamino)-3-hydroxy-pyridin-2-yl]-4-oxo-butyric acid ethyl ester

The title compound was prepared from 4-[3-Benzyloxy-4-cyano-6-(2,6-dimethyl-benzoylamino)-pyridin-2-yl]-4-oxo-butyric acid ethyl ester (see Example 36b) and thioanisole in analogy to example 158a. MS-(+)-ion, M+H=396.04.

### b) 4-[4-Cyano-6-(2,6-dimethyl-benzoylamino)-3-hydroxy-pyridin-2-yl]-4-oxo-butyric acid

The title compound was prepared from 4-[4-Cyano-6-(2,6-dimethyl-benzoylamino)-3-hydroxy-pyridin-2-yl]-4-oxo-butyric acid ethyl ester (see example 37a) in analogy to example 143g. MS-(+)-ion, M+H=382.04.

### Example 130

### 4-{4-Cyano-6-[(2,6-dimethyl-benzoyl)-N-methyl-amino]-3-hydroxy-pyridin-2-yl}-4-oxo-butyric acid

### a) 4-[3-Benzyloxy-4-bromo-6-(2,6-dimethyl-benzoylamino)-pyridin-2-yl]-4-oxo-butyric acid ethyl ester

The title compound was prepared from 4-(3-Benzyloxy-4,6-dibromo-pyridin-2-yl)-4-oxo-butyric acid ethyl ester and 2,6-dimethyl-benzamide in analogy to example 23a. MS-(+)-ion, M+H=538.97, 540.87.

### b) 4-[3-Benzyloxy-4-cyano-6-(2,6-dimethyl-benzoylamino)-pyridin-2-yl]-4-oxo-butyric acid ethyl ester

The title compound was prepared from 4-[3-Benzyloxy-4-bromo-6-(2,6-dimethyl-benzoylamino)-pyridin-2-yl]-4-oxo-butyric acid ethyl ester and zinc(II) cyanide in analogy to example 43f. MS-(+)-ion, M+H=486.11.

### c) 4-{3-Benzyloxy-4-cyano-6-[(2,6-dimethyl-benzoyl)-N-methyl-amino]-pyridin-2-yl}-4-oxo-butyric acid

### ethyl ester

The title compound was prepared from 4-(6-Benzoylamino-3-benzyloxy-4-cyano-pyridin-2-yl)-4-oxo-butyric acid ethyl ester (see example 36b) and methyl iodide in analogy to example 159a. MS-(+)-ion, M+H=500.11.

### d) 4-{4-Cyano-6-[(2,6-dimethyl-benzoyl)-N-methyl-amino]-3-hydroxy-pyridin-2-yl}-4-oxo-butyric acid ethyl ester

The title compound was prepared from 4-{3-Benzyloxy-4-cyano-6-[(2,6-dimethyl-benzoyl)-N-methyl-amino]-pyridin-2-yl}-4-oxo-butyric acid ethyl ester (see Example 36c) and thioanisole in analogy to example 158a. MS-(+)-ion, M+H=410.09.

### e) 4-{4-Cyano-6-[(2,6-dimethyl-benzoyl)-N-methyl-amino]-3-hydroxy-pyridin-2-yl}-4-oxo-butyric acid

The title compound was prepared from 4-{4-Cyano-6-[(2,6-dimethyl-benzoyl)-N-methyl-amino]-3-hydroxy-pyridin-2-yl}-4-oxo-butyric acid ethyl ester (see example 36d) in analogy to example 143g. MS-(+)-ion, M+H=382.04.

### Example 131

### 4-[3-Cyano-2-hydroxy-5-(2,4,6-trichloro-benzyl)-phenyl]-4-oxo-butyric acid

### a) 1-[5-Bromo-2-(tert-butyl-dimethyl-silanyloxy)-phenyl]-ethanone

To a solution of 1-(5-bromo-2-hydroxy-phenyl)-ethanone (Combi-blocks, 5.08 g, 23.6 mmol) in DMF (47 mL) at r.t was added imidazole (1.93 g, 28 mmol) and TBSCl (3.89 g, 25.6 mmol). The reaction mixture was stirred at r.t. for overnight. After diluted with Et₂O (500 mL), washed by NaHCO₃ solution and water, The dried extract (MgSO₄) was concentrated in vacuo and purified by ISCO over silica gel, eluting with 2-35% EtOAc/hexanes, to give product: MS (m/z) 328.9, 330.8 (M+1)⁺.

### b) 4-(5-Bromo-2-hydroxy-phenyl)-4-oxo-butyric acid ethyl ester

To a solution of 1-[5-bromo-2-(tert-butyl-dimethyl-silanyloxy)-phenyl]-ethanone (5.3 g, 16.1 mmol) in THF (32 mL) and DMPU (8.35 ml) was added a solution of LiHMDS (20.9 mL, 1 M solution in THF, 20.9 mmol) at −60° C. under argon. After 10 minutes of stirring at −60° C. to the above mixture, ethyl bromoacetate (3.48 mL, 31.28 mmol) was added. The mixture was stirred at −60° C. for additional 10 min, then warmed to r.t for 4h. quenched by water, extracted with EtOAc (500 mL). The extract was washed with 0.1 N HCl solution and water. The dried extract (MgSO₄) was concentrated in vacuo and purified by ISCO over silica gel, eluting with 2-35% EtOAc/hexanes, to give product: MS (m/z) 300.9, 302.0 (M+1)⁺.

### c) (2,4,6-Trichloro-phenyl)-methanol

To a solution of 2, 4, 6-Trichloro benzoic acid (1 g, 4.43 mmol) in THF (60 mL) was added 1M BH₃ (13.3 mL, 13.30 mmol) in THF at 0° C. under N₂ with ice-cooling. The mixture was allowed to warm to room temperature and then reflux for overnight. Reaction was completed; reaction mixture was cooled to 0° C. and quenched with MeOH followed by 1 M HCl, The mixture was extracted with DCM, organic layer was separated, dried over Na₂SO₄, concentrated under vacuo to get crude. Attempts to purify by column chromatography were unsuccessful due to low solubility. The title compound was obtained and was used without purification.

### d) 2-Bromomethyl-1,3,5-trichloro-benzene

To a solution of crude (2,4,6-Trichloro-phenyl)-methanol (1 g, 4.72 mmol) in DCM (20 mL) was added PBr₃ (0.45 ml, 4.77 mmol) in DCM (20 ml) at −10° C. under N₂. The mixture was stirred at 0° C. for 6h; Reaction was completed; The resulting mixture was diluted with water and quenched with Aq. NaHCO₃ solution, extracted with ethyl acetate, organic layer was separated, dried over Na₂SO₄, concentrated under vacuo to get crude; purified by column chromatography using 0-5% ethyl acetate in hexane as eluent gave the title compound. ¹H NMR (CDCl₃, 400 MHz): δ=7.36 (s, 2H), 4.70 (s, 2H).

### e) 4-[2-Hydroxy-5-(2,4,6-trichloro-benzyl)-phenyl]-4-oxo-butyric acid ethyl ester

A flask charged with Zn dust (343 mg, 5.28 mmol) in THF (3 mL) was stirred at 65° C. was added 1,2-dibromoethane (18.6 mg, 0.1 mmol) and chlorotrimethylsilane (92 mg, 0.84 mmol). The suspension was stirred at 65° C. for 30 min. After cooled to r.t, 2-bromomethyl-1,3,5-trichloro-benzene (728 mg, 2.65 mmol, 14d) in THF (2.3 mL) was added, the mixture was allowed to continue to stir at r.t. for 2h to form 1,3,5-trichlorobenzylzinc bromide solution.

Charge a flask with 4-(5-bromo-2-hydroxy-phenyl)-4-oxo-butyric acid ethyl ester (200 mg, 0.66 mmol), palladium acetate (7.4 mg, 0.033 mmol) and S-Phos (27 mg, 0.066 mmol). Cycle the flask with nitrogen and vacuum, added 5.3 ml of 1,3,5-trichlorobenzylzinc bromide solution (0.5 M in THF). The reaction mixture was stirred at r.t. for 16 h, quenched by water, extracted with EtOAc (100 mL). The dried extract (MgSO₄) was concentrated in vacuo and purified by ISCO over silica gel, eluting with 2-35% EtOAc/hexanes, to give product: MS (m/z) 415.4, 416.8 (M+1)⁺.

### f) 4-[3-Bromo-2-hydroxy-5-(2,4,6-trichloro-benzyl)-phenyl]-4-oxo-butyric acid ethyl ester

To a solution of 4-[2-Hydroxy-5-(2,4,6-trichloro-benzyl)-phenyl]-4-oxo-butyric acid ethyl ester (175 mg, 0.42 mmol) in CHCl₃ (4.2 mL) at r.t. was added sodium acetate (52 mg, 0.63 mmol) and bromine (100 mg, 0.63 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 490.8, 492.8, 494.7 (M−1)⁺.

### g) 4-[3-Cyano-2-hydroxy-5-(2,4,6-trichloro-benzyl)-phenyl]-4-oxo-butyric acid ethyl ester

To a solution of 4-[3-Bromo-2-hydroxy-5-(2,4,6-trichloro-benzyl)-phenyl]-4-oxo-butyric acid ethyl ester (129 mg, 0.26 mmol) in DMAC (3.7 mL) at r.t. were added Zn(CN)₂ (61.6 mg, 0.52 mmol), Pd₂(dba)₃ (23.8 mg, 0.026 mmol), dppf (29 mg, 0.052 mmol), and Zn dust (5 mg, 0.078 mmol) to give suspension solution, the reaction mixture was allowed to stir at 100° C. for 3-4 hours. After cooled to rt, diluted with EtOAc (100 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 437.9, 440.0, 441.9 (M−1)⁺.

### h) 4-[3-Cyano-2-hydroxy-5-(2,4,6-trichloro-benzyl)-phenyl]-4-oxo-butyric acid

To a solution of 4-[3-Cyano-2-hydroxy-5-(2,4,6-trichloro-benzyl)-phenyl]-4-oxo-butyric acid ethyl ester (77.8 mg, 0.17 mmol) in THF (1.8 mL) and water (0.6 mL) at r.t. was added lithium hydroxide monohydrate (29.7 mg, 0.71 mmol) to give suspension solution, the reaction mixture was stirred at r.t. After 24 h, the mixture was concentrated in vacuo to give residue as a solid. The solid was dissolved in water (15 mL), extracted with EtOAc (3×15 mL). The aqueous solution was acidified by 1N HCl solution, filtered, washed with water, dried to give product: MS (m/z) 409.9, 411.9, 413.8 (M−1)⁺.

### Example 132

### 4-(3-Cyano-2-hydroxy-5-phenoxy-phenyl)-4-oxo-butyric acid

### a) Acetic acid 4-phenoxy-phenyl ester

A solution of 4-phenoxy-phenol (9 g, 0.048 mol) in pyridine (26 mL) was treated with acetic anhydride (4.76 mL). The reaction mixture was stirred at room temperature overnight. The mixture was partitioned between DCM and 10% HCl solution, and the resulting mixture was stirred for 1 h. The organic phase was washed with 10% HCl and water until pH=7. The organic layer was dried and the solvent was evaporated to afford acetic acid 4-phenoxy-phenyl ester.

### b) 1-(2-Hydroxy-5-phenoxy-phenyl)-ethanone

A mixture of acetic acid 4-phenoxy-phenyl ester (10.8 g, 0.047 mol) and AlCl₃) (12.6 g, 0.094 mol) was stirred at 120° C. for 20-30 minutes. The mixture was cooled to 60-80° C. and 0.1 N HCl solution was added. The resulting mixture was extracted with EtOAc (500 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 229.0 (M+1)⁺.

### c) 1-[2-(tert-Butyl-dimethyl-silanyloxy)-5-phenoxy-phenyl]-ethanone

To a solution of 1-(2-Hydroxy-5-phenoxy-phenyl)-ethanone (452 mg, 1.98 mmol) in DMF (3.9 mL) at r.t was added imidazole (161 mg, 2.37 mmol) and TBSCl (327 mg, 2.18 mmol). The reaction mixture was stirred at r.t. for overnight. After diluted with Et₂O (100 mL), washed by NaHCO₃ solution and water, The dried extract (MgSO₄) was concentrated in vacuo and purified by ISCO over silica gel, eluting with 2-35% EtOAc/hexanes, to give product: MS (m/z) 343.1 (M+1)⁺.

### d) 4-(2-Hydroxy-5-phenoxy-phenyl)-4-oxo-butyric acid ethyl ester

To a solution of 1-[2-(tert-Butyl-dimethyl-silanyloxy)-5-phenoxy-phenyl]-ethanone (651 mg, 1.9 mmol) in THF (3.8 mL) and DMPU (0.98 ml) was added a solution of LiHMDS (2.47 mL, 1 M solution in THF, 2.47 mmol) at −60° C. under argon. After 10 minutes of stirring at −60° C. to the above mixture, ethyl bromoacetate (0.42 mL, 3.8 mmol) was added. The mixture was stirred at −60° C. for additional 10 min, then warmed to r.t for 4h. quenched by water, extracted with EtOAc (100 mL). The dried extract (MgSO₄) was concentrated in vacuo and purified by ISCO over silica gel, eluting with 2-35% EtOAc/hexanes, to give product: MS (m/z) 315.0 (M+1)⁺.

### e) 4-(3-Bromo-2-hydroxy-5-phenoxy-phenyl)-4-oxo-butyric acid ethyl ester

To a solution of the 4-(2-Hydroxy-5-phenoxy-phenyl)-4-oxo-butyric acid ethyl ester (137 mg, 0.43 mmol) in CHCl₃ (4.3 mL) at r.t. was added sodium acetate (53 mg, 0.65 mmol) and bromine (105 mg, 0.65 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 392.8, 395.1 (M+1)⁺.

### f) 4-(3-Cyano-2-hydroxy-5-phenoxy-phenyl)-4-oxo-butyric acid ethyl ester

To a solution of 4-(3-Bromo-2-hydroxy-5-phenoxy-phenyl)-4-oxo-butyric acid ethyl ester (116 mg, 0.3 mmol) in DMAC (4.2 mL) at r.t. were added Zn(CN)₂ (69 mg, 0.59 mmol), Pd₂(dba)₃ (27 mg, 0.03 mmol), dppf (32 mg, 0.06 mmol), and Zn dust (5.8 mg, 0.09 mmol) to give suspension solution, the reaction mixture was allowed to stir at 100° C. for 3-4 hours. After cooled to rt, diluted with EtOAc (100 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 338.0 (M−1)⁺.

### g) 4-(3-Cyano-2-hydroxy-5-phenoxy-phenyl)-4-oxo-butyric acid

To a solution of 4-(3-Cyano-2-hydroxy-5-phenoxy-phenyl)-4-oxo-butyric acid ethyl ester (37 mg, 0.11 mmol) in THF (1.2 mL) and water (0.4 mL) at r.t. was added lithium hydroxide monohydrate (18 mg, 0.4 mmol) to give suspension solution, the reaction mixture was stirred at r.t. After 24 h, the mixture was concentrated in vacuo to give residue as a solid. The solid was dissolved in water (15 mL), extracted with EtOAc (3×25 mL). The aqueous solution was acidified by 1N HCl solution, filtered, washed with water, dried to give product: MS (m/z) 310.0 (M−1)⁺.

### Example 133

### 4-[2-Cyano-6-(2,6-dimethyl-benzyl)-3-hydroxy-pyridin-4-yl]-4-oxo-butyric acid

### a) 4-[2-Cyano-6-(2,6-dimethyl-benzyl)-3-hydroxy-pyridin-4-yl]-4-oxo-butyric acid ethyl ester

The title compound was prepared from 4-(6-Bromo-2-cyano-3-hydroxy-pyridin-4-yl)-4-oxo-butyric acid ethyl ester and 2,6-Dimethyl-benzylzinc(II) bromide in THF in analogy to example 143d, MS-(+)-ion, M+H=367.03.

### b) 4-[2-Cyano-6-(2,6-dimethyl-benzyl)-3-hydroxy-pyridin-4-yl]-4-oxo-butyric acid

The title compound was prepared from 4-[2-Cyano-6-(2,6-dimethyl-benzyl)-3-hydroxy-pyridin-4-yl]-4-oxo-butyric acid ethyl ester in analogy to example 143g. MS-(+)-ion, M+H=339.03.

### Example 134

### 4-[3-Cyano-5-(2,6-dichloro-3-fluoro-benzyl)-2-hydroxy-phenyl]-4-oxo-butyric acid

### a) (2,6-Dichloro-3-fluoro-phenyl)-methanol

To a solution of 2,6-Dichloro-3-fluoro benzoic acid (5g, 23.92 mmol) in THF (50 mL) was added 1M BH₃ (72 mL, 71.76 mmol) in THF at 0° C. under N₂ with ice-cooling. The mixture was allowed to warm to room temperature and then reflux for overnight. Reaction was completed; reaction mixture was cooled to 0° C. and quenched with MeOH followed by 1 M HCl, The mixture was extracted with DCM, organic layer was separated, dried over Na₂SO₄, concentrated under vacuo to get crude; purified by column chromatography using 0-5% ethyl acetate in hexane as eluent gave the title compound. ¹H NMR (CDCl₃, 400 MHz): δ=7.29-7.35 (m, 1H), 7.09 (t, J=8.4 Hz, 1H), 4.97 (s, 2H).

### b) 2-Bromomethyl-1,3-dichloro-4-fluoro-benzene

To a solution of (2,6-Dichloro-3-fluoro-phenyl)-methanol (0.40 g, 2.04 mmol) in DCM (8 mL) was added PBr₃ (0.2 ml, 2.06 mmol) in DCM (8 ml) at −10° C. under N₂. The mixture was stirred at 0° C. for 6h; Reaction was completed; The resulting mixture was diluted with water and quenched with Aq. NaHCO₃ solution, extracted with ethyl acetate, organic layer was separated, dried over Na₂SO₄, concentrated under vacuo to get crude product. The procedures were repeated using 4.0 g of 2,6-Dichloro-3-fluoro-phenyl)-methanol, and the two batches of crude product were combined and purified by column chromatography using 0-5% ethyl acetate in hexane to give the title compound. ¹H NMR (CDCl₃, 400 MHz): δ=7.29-7.35 (m, 1H), 7.10 (t, J=8.0 Hz, 1H), 4.74 (s, 2H).

### c) 4-[5-(2,6-Dichloro-3-fluoro-benzyl)-2-hydroxy-phenyl]-4-oxo-butyric acid ethyl ester

A flask charged with Zn dust (353 mg, 5.44 mmol) in THF (3 mL) was stirred at 65° C. was added 1,2-dibromoethane (20.4 mg, 0.1 mmol) and chlorotrimethylsilane (0.11 mL, 0.87 mmol). The suspension was stirred at 65° C. for 30 min. After cooled to r.t, 2-bromomethyl-1,3-dichloro-4-fluoro-benzene (702 mg, 2.72 mmol, 134b) in THF (2.4 mL) was added, the mixture was allowed to continue to stir at r.t. for 2h to form 1,3,-dichloro-4-fluorobenzylzinc bromide solution.

Charge a flask with 4-(5-bromo-2-hydroxy-phenyl)-4-oxo-butyric acid ethyl ester (205 mg, 0.68 mmol, prepared in the same manner as 131b), palladium acetate (7.6 mg, 0.034 mmol) and S-Phos (27.9 mg, 0.068 mmol). Cycle the flask with nitrogen and vacuum, added 5.3 ml of 1,3,-dichloro-4-fluorobenzylzinc bromide solution (0.5 M in THF). The reaction mixture was stirred at r.t. for 16 h, quenched by water, extracted with EtOAc (100 mL). The dried extract (MgSO₄) was concentrated in vacuo and purified by ISCO over silica gel, eluting with 2-35% EtOAc/hexanes, to give product: MS (m/z) 399.1, 401.2 (M+1)⁺.

### d) 4-[3-Bromo-5-(2,6-dichloro-3-fluoro-benzyl)-2-hydroxy-phenyl]-4-oxo-butyric acid ethyl ester

To a solution of 4-[5-(2,6-Dichloro-3-fluoro-benzyl)-2-hydroxy-phenyl]-4-oxo-butyric acid ethyl ester (268 mg, 0.67 mmol) in CHCl₃ (6.7 mL) at r.t. was added sodium acetate (82 mg, 1.0 mmol) and bromine (118 mg, 0.73 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 476.8, 478.7 (M−1)⁺.

### e) 4-[3-Cyano-5-(2,6-dichloro-3-fluoro-benzyl)-2-hydroxy-phenyl]-4-oxo-butyric acid ethyl ester

To a solution of 4-[3-Bromo-5-(2,6-dichloro-3-fluoro-benzyl)-2-hydroxy-phenyl]-4-oxo-butyric acid ethyl ester (125 mg, 0.26 mmol) in DMAC (3.7 mL) at r.t. were added Zn(CN)₂ (61 mg, 0.52 mmol), Pd₂(dba)₃ (23.8 mg, 0.026 mmol), dppf (29 mg, 0.052 mmol), and Zn dust (5 mg, 0.078 mmol) to give suspension solution, the reaction mixture was allowed to stir at 100° C. for 3-4 hours. After cooled to rt, diluted with EtOAc (100 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 422.0, 424.0 (M−1)⁺.

### f) 4-[3-Cyano-5-(2,6-dichloro-3-fluoro-benzyl)-2-hydroxy-phenyl]-4-oxo-butyric acid

To a solution of 4-[3-Cyano-5-(2,6-dichloro-3-fluoro-benzyl)-2-hydroxy-phenyl]-4-oxo-butyric acid ethyl ester (66 mg, 0.16 mmol) in THF (1.7 mL) and water (0.5 mL) at r.t. was added lithium hydroxide monohydrate (26 mg, 0.62 mmol) to give suspension solution, the reaction mixture was stirred at r.t. After 24 h, the mixture was concentrated in vacuo to give residue as a solid. The solid was dissolved in water (15 mL), extracted with EtOAc (3×15 mL). The aqueous solution was acidified by 1N HCl solution, filtered, washed with water, dried to give product: MS (m/z) 394.0, 396.0 (M−1)⁺.

### Example 135

### 4-(3-Cyano-2-hydroxy-5-phenethyl-phenyl)-4-oxo-butyric acid

### a) 4-(2-Hydroxy-5-phenylethynyl-phenyl)-4-oxo-butyric acid ethyl ester

To a solution of 4-(5-bromo-2-hydroxy-phenyl)-4-oxo-butyric acid ethyl ester (211 mg, 0.7 mmol, prepared in the same manner as 14b) in DMF (3.5 mL) at r.t. were added tributyl-phenylethynyl-stannane (548 mg, 1.4 mmol) and PdCl₂(PPh₃)₂ (49.1 mg, 0.07 mmol) to give suspension solution, the reaction mixture was allowed to stir at 120° C. for 1 hours. After cooled to rt, diluted with EtOAc (100 mL) and filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 321.0 (M−1)⁺.

### b) 4-(2-Hydroxy-5-phenethyl-phenyl)-4-oxo-butyric acid ethyl ester

To a solution of 4-(2-Hydroxy-5-phenylethynyl-phenyl)-4-oxo-butyric acid ethyl ester (162 mg, 0.5 mmol) in EtOAC and EtOH (1:1, 24 mL) at r.t. were added Pd/C (30 mg) to give suspension solution, the reaction mixture was allowed to stir at r.t under hydrogen atmosphere for 2 hours, filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 327.0 (M+1)⁺.

### c) 4-(3-Bromo-2-hydroxy-5-phenethyl-phenyl)-4-oxo-butyric acid ethyl ester

To a solution of 4-(2-Hydroxy-5-phenethyl-phenyl)-4-oxo-butyric acid ethyl ester (123 mg, 0.37 mmol) in CHCl₃ (3.7 mL) at r.t. was added sodium acetate (46 mg, 0.56 mmol) and bromine (90 mg, 0.56 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 405.0, 406.3 (M+1)⁺.

### d) 4-(3-Cyano-2-hydroxy-5-phenethyl-phenyl)-4-oxo-butyric acid ethyl ester

To a solution of 4-(3-Bromo-2-hydroxy-5-phenethyl-phenyl)-4-oxo-butyric acid ethyl ester (121 mg, 0.29 mmol) in DMAC (3.7 mL) at r.t. were added Zn(CN)₂ (70 mg, 0.59 mmol), Pd₂(dba)₃ (27.2 mg, 0.029 mmol), dppf (33 mg, 0.058 mmol), and Zn dust (5.8 mg, 0.089 mmol) to give suspension solution, the reaction mixture was allowed to stir at 100° C. for 3-4 hours. After cooled to rt, diluted with EtOAc (100 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 350.0 (M−1)⁺.

### e) 4-(3-Cyano-2-hydroxy-5-phenethyl-phenyl)-4-oxo-butyric acid

To a solution of 4-(3-Cyano-2-hydroxy-5-phenethyl-phenyl)-4-oxo-butyric acid ethyl ester (47.9 mg, 0.13 mmol) in THF (1.5 mL) and water (0.5 mL) at r.t. was added lithium hydroxide monohydrate (22.9 mg, 0.54 mmol) to give suspension solution, the reaction mixture was stirred at r.t. After 24 h, the mixture was concentrated in vacuo to give residue as a solid. The solid was dissolved in water (15 mL), extracted with EtOAc (3×15 mL). The aqueous solution was acidified by 1N HCl solution, filtered, washed with water, dried to give product: MS (m/z) 322.0 (M−1)⁺.

### Example 136

### 4-[5-(2-Chloro-phenoxy)-3-cyano-2-hydroxy-phenyl]-4-oxo-butyric acid

### a) 4-(2-Benzyloxy-5-bromo-phenyl)-4-oxo-butyric acid ethyl ester

To a solution of 4-(5-bromo-2-hydroxy-phenyl)-4-oxo-butyric acid ethyl ester (385 mg, 1.27 mmol, prepared in the same manner as 131b) in acetone (6.3 mL) at r.t. were added benzyl bromide (547 mg, 3.19 mmol) and potassium carbonate (1.05 g, 7.6 mmol) to give suspension solution, the reaction mixture was allowed to stir at r.t for overnight, filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 389.2, 391.1 (M−1)⁺.

### b) 4-[2-Benzyloxy-5-(2-chloro-phenoxy)-phenyl]-4-oxo-butyric acid ethyl ester

To a solution of 4-(2-Benzyloxy-5-bromo-phenyl)-4-oxo-butyric acid ethyl ester (458 mg, 1.17 mmol) in DMF (5.8 mL) at r.t. were added 2-chloro-phenol (225 mg, 1.75 mmol), Cs₂CO₃ (498 mg, 2.34 mmol), CuCl (11.6 mg, 0.117 mmol), and 2,2,6,6-tetramethyl-3,5-heptanedione (43 mg, 0.23 mmol) to give suspension solution, the reaction mixture was allowed to stir at 150° C. for 1-2 hours. After cooled to rt, diluted with EtOAc (100 mL) and filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 439.1 (M+1)⁺.

### c) 4-[5-(2-Chloro-phenoxy)-2-hydroxy-phenyl]-4-oxo-butyric acid ethyl ester

A flask charged with 4-[2-Benzyloxy-5-(2-chloro-phenoxy)-phenyl]-4-oxo-butyric acid ethyl ester (68 mg, 0.15 mmol) in TFA (1 mL) was added thioanisole (192 mg, 1.5 mmol). The suspension was stirred at 65° C. for 2 h. After cooled to rt, diluted with EtOAc (100 mL) and filtered through a celite plug rinsing with EtOAc (100 mL). The organic layers were washed with NaHCO₃ solution and water. The dried extract (MgSO₄) was concentrated in vacuo and purified by ISCO over silica gel, eluting with 5-35% EtOAc/hexanes to give product: MS (m/z) 346.7, 347.1 (M−1)⁺.

### d) 4-[3-Bromo-5-(2-chloro-phenoxy)-2-hydroxy-phenyl]-4-oxo-butyric acid ethyl ester

To a solution of the 4-[5-(2-Chloro-phenoxy)-2-hydroxy-phenyl]-4-oxo-butyric acid ethyl ester (49.7 mg, 0.14 mmol) in CHCl₃ (1.4 mL) at r.t. was added sodium acetate (17 mg, 0.21 mmol) and bromine (33.6 mg, 0.21 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 424.9, 426.9, 428.9 (M−1)⁺.

### e) 4-[5-(2-Chloro-phenoxy)-3-cyano-2-hydroxy-phenyl]-4-oxo-butyric acid ethyl ester

To a solution of the 4-[3-Bromo-5-(2-chloro-phenoxy)-2-hydroxy-phenyl]-4-oxo-butyric acid ethyl ester (43 mg, 0.1 mmol) in DMAC (1.4 mL) at r.t. were added Zn(CN)₂ (23.5 mg, 0.2 mmol), Pd₂(dba)₃ (9.1 mg, 0.01 mmol), dppf (11 mg, 0.02 mmol), and Zn dust (2 mg, 0.03 mmol) to give suspension solution, the reaction mixture was allowed to stir at 100° C. for 3-4 hours. After cooled to rt, diluted with EtOAc (50 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 372.0 (M−1)⁺.

### f) 4-[5-(2-Chloro-phenoxy)-3-cyano-2-hydroxy-phenyl]-4-oxo-butyric acid

To a solution of 4-[5-(2-Chloro-phenoxy)-3-cyano-2-hydroxy-phenyl]-4-oxo-butyric acid ethyl ester (23.7 mg, 0.063 mmol) in THF (0.75 mL) and water (0.25 mL) at r.t. was added lithium hydroxide monohydrate (10.7 mg, 0.25 mmol) to give suspension solution, the reaction mixture was stirred at r.t. After 24 h, the mixture was concentrated in vacuo to give residue as a solid. The solid was dissolved in water (25 mL), extracted with EtOAc (3×15 mL). The aqueous solution was acidified by 1N HCl solution, filtered, washed with water, dried to give product: MS (m/z) 343.9, 345.9 (M−1)⁺.

### Example 137

### 4-[5-(4-Chloro-phenoxy)-3-cyano-2-hydroxy-phenyl]-4-oxo-butyric acid

### a) 4-[2-Benzyloxy-5-(4-chloro-phenoxy)-phenyl]-4-oxo-butyric acid ethyl ester

To a solution of 4-(2-Benzyloxy-5-bromo-phenyl)-4-oxo-butyric acid ethyl ester (406 mg, 1.03 mmol, prepared in the same manner as 131b) in DMF (5.1 mL) at r.t. were added 4-chloro-phenol (199 mg, 1.55 mmol), Cs₂CO₃ (671 mg, 2.06 mmol), CuCl (10.2 mg, 0.1 mmol), and 2,2,6,6-tetramethyl-3,5-heptanedione (38 mg, 0.2 mmol) to give suspension solution, the reaction mixture was allowed to stir at 150° C. for 1-2 hours. After cooled to rt, diluted with EtOAc (100 mL) and filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 438.6 (M+1)⁺.

### b) 4-[5-(4-Chloro-phenoxy)-2-hydroxy-phenyl]-4-oxo-butyric acid ethyl ester

A flask charged with 4-[2-Benzyloxy-5-(4-chloro-phenoxy)-phenyl]-4-oxo-butyric acid ethyl ester (162 mg, 0.36 mmol) in TFA (3.6 mL) was added thioanisole (0.4 mL, 3.6 mmol). The suspension was stirred at 65° C. for 2 h. After cooled to rt, diluted with EtOAc (100 mL) and filtered through a celite plug rinsing with EtOAc (100 mL). The organic layers were washed with NaHCO₃ solution and water. The dried extract (MgSO₄) was concentrated in vacuo and purified by ISCO over silica gel, eluting with 5-35% EtOAc/hexanes to give product: MS (m/z) 348.2 (M+1)⁺.

### c) 4-[3-Bromo-5-(4-chloro-phenoxy)-2-hydroxy-phenyl]-4-oxo-butyric acid ethyl ester

To a solution of the 4-[5-(4-Chloro-phenoxy)-2-hydroxy-phenyl]-4-oxo-butyric acid ethyl ester (105 mg, 0.3 mmol) in CHCl₃ (3 mL) at r.t. was added sodium acetate (36.9 mg, 0.45 mmol) and bromine (72 mg, 0.45 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 428.0 (M+1)⁺.

### d) 4-[5-(4-Chloro-phenoxy)-3-cyano-2-hydroxy-phenyl]-4-oxo-butyric acid ethyl ester

To a solution of the 4-[3-Bromo-5-(4-chloro-phenoxy)-2-hydroxy-phenyl]-4-oxo-butyric acid ethyl ester (53.2 mg, 0.12 mmol) in DMAC (1.7 mL) at r.t. were added Zn(CN)₂ (29.1 mg, 0.25 mmol), Pd₂(dba)₃ (11.5 mg, 0.012 mmol), dppf (13.8 mg, 0.024 mmol), and Zn dust (2.4 mg, 0.03 mmol) to give suspension solution, the reaction mixture was allowed to stir at 100° C. for 3-4 hours. After cooled to rt, diluted with EtOAc (50 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 372.0 (M−1)⁺.

### e) 4-[5-(4-Chloro-phenoxy)-3-cyano-2-hydroxy-phenyl]-4-oxo-butyric acid

To a solution of 4-[5-(4-Chloro-phenoxy)-3-cyano-2-hydroxy-phenyl]-4-oxo-butyric acid ethyl ester (20.5 mg, 0.055 mmol) in THF (0.75 mL) and water (0.25 mL) at r.t. was added lithium hydroxide monohydrate (9.2 mg, 0.21 mmol) to give suspension solution, the reaction mixture was stirred at r.t. After 24 h, the mixture was concentrated in vacuo to give residue as a solid. The solid was dissolved in water (25 mL), extracted with EtOAc (3×15 mL). The aqueous solution was acidified by 1N HCl solution, filtered, washed with water, dried to give product: MS (m/z) 343.9, 345.9 (M−1)⁺.

### Example 138

### 4-[5-(3-Chloro-phenoxy)-3-cyano-2-hydroxy-phenyl]-4-oxo-butyric acid

### a) 4-[2-Benzyloxy-5-(3-chloro-phenoxy)-phenyl]-4-oxo-butyric acid ethyl ester

To a solution of 4-(2-Benzyloxy-5-bromo-phenyl)-4-oxo-butyric acid ethyl ester (431 mg, 1.1 mmol, prepared in the same manner as 131b) in DMF (5.5 mL) at r.t. were added 3-chloro-phenol (212 mg, 1.65 mmol), Cs₂CO₃ (718 mg, 2.2 mmol), CuCl (11 mg, 0.11 mmol), and 2,2,6,6-tetramethyl-3,5-heptanedione (40 mg, 0.22 mmol) to give suspension solution, the reaction mixture was allowed to stir at 150° C. for 1-2 hours. After cooled to rt, diluted with EtOAc (100 mL) and filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 439.0 (M+1)⁺.

### b) 4-[5-(3-Chloro-phenoxy)-2-hydroxy-phenyl]-4-oxo-butyric acid ethyl ester

A flask charged with 4-[2-Benzyloxy-5-(3-chloro-phenoxy)-phenyl]-4-oxo-butyric acid ethyl ester (148 mg, 0.34 mmol) in TFA (3.4 mL) was added thioanisole (0.4 mL, 3.4 mmol). The suspension was stirred at 65° C. for 2 h. After cooled to rt, diluted with EtOAc (100 mL) and filtered through a celite plug rinsing with EtOAc (100 mL). The organic layers were washed with NaHCO₃ solution and water. The dried extract (MgSO₄) was concentrated in vacuo and purified by ISCO over silica gel, eluting with 5-35% EtOAc/hexanes to give product: MS (m/z) 348.2 (M+1)⁺.

### c) 4-[3-Bromo-5-(3-chloro-phenoxy)-2-hydroxy-phenyl]-4-oxo-butyric acid ethyl ester

To a solution of the 4-[5-(3-Chloro-phenoxy)-2-hydroxy-phenyl]-4-oxo-butyric acid ethyl ester (42.5 mg, 0.12 mmol) in CHCl₃ (1.2 mL) at r.t. was added sodium acetate (14 mg, 0.18 mmol) and bromine (29.3 mg, 0.18 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 426.9, 428.8 (M−1)⁺.

### d) 4-[5-(3-Chloro-phenoxy)-3-cyano-2-hydroxy-phenyl]-4-oxo-butyric acid ethyl ester

To a solution of the 4-[3-Bromo-5-(3-chloro-phenoxy)-2-hydroxy-phenyl]-4-oxo-butyric acid ethyl ester (46 mg, 0.11 mmol) in DMAC (1.5 mL) at r.t. were added Zn(CN)₂ (25.2 mg, 0.21 mmol), Pd₂(dba)₃ (9.8 mg, 0.011 mmol), dppf (11.8 mg, 0.022 mmol), and Zn dust (2.1 mg, 0.03 mmol) to give suspension solution, the reaction mixture was allowed to stir at 100° C. for 3-4 hours. After cooled to rt, diluted with EtOAc (50 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 372.0 (M−1)⁺.

### e) 4-[5-(3-Chloro-phenoxy)-3-cyano-2-hydroxy-phenyl]-4-oxo-butyric acid

To a solution of 4-[5-(3-Chloro-phenoxy)-3-cyano-2-hydroxy-phenyl]-4-oxo-butyric acid ethyl ester (18.5 mg, 0.049 mmol) in THF (0.75 mL) and water (0.25 mL) at r.t. was added lithium hydroxide monohydrate (8.3 mg, 0.20 mmol) to give suspension solution, the reaction mixture was stirred at r.t. After 24 h, the mixture was concentrated in vacuo to give residue as a solid. The solid was dissolved in water (25 mL), extracted with EtOAc (3×15 mL). The aqueous solution was acidified by 1N HCl solution, filtered, washed with water, dried to give product: MS (m/z) 343.9, 345.9 (M−1)⁺.

### Example 139

### 4-(3-Cyano-2-hydroxy-5-p-tolyloxy-phenyl)-4-oxo-butyric acid

### a) 4-(2-Benzyloxy-5-p-tolyloxy-phenyl)-4-oxo-butyric acid ethyl ester

To a solution of 4-(2-Benzyloxy-5-bromo-phenyl)-4-oxo-butyric acid ethyl ester (475 mg, 1.21 mmol, prepared in the same manner as 131b) in DMF (6 mL) at r.t. were added 4-methyl-phenol (197 mg, 1.82 mmol), Cs₂CO₃ (788 mg, 2.42 mmol), CuCl (12 mg, 0.12 mmol), and 2,2,6,6-tetramethyl-3,5-heptanedione (45 mg, 0.24 mmol) to give suspension solution, the reaction mixture was allowed to stir at 150° C. for 1-2 hours. After cooled to rt, diluted with EtOAc (100 mL) and filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 419.0 (M+1)⁺.

### b) 4-(2-Hydroxy-5-p-tolyloxy-phenyl)-4-oxo-butyric acid ethyl ester

A flask charged with 4-(2-Benzyloxy-5-p-tolyloxy-phenyl)-4-oxo-butyric acid ethyl ester (132 mg, 0.32 mmol) in TFA (3 mL) was added thioanisole (0.37 mL, 3.2 mmol). The suspension was stirred at 65° C. for 2 h. After cooled to rt, diluted with EtOAc (100 mL) and filtered through a celite plug rinsing with EtOAc (100 mL). The organic layers were washed with NaHCO₃ solution and water. The dried extract (MgSO₄) was concentrated in vacuo and purified by ISCO over silica gel, eluting with 5-35% EtOAc/hexanes to give product: MS (m/z) 329.0 (M+1)⁺.

### c) 4-(3-Bromo-2-hydroxy-5-p-tolyloxy-phenyl)-4-oxo-butyric acid ethyl ester

To a solution of the 4-(2-Hydroxy-5-p-tolyloxy-phenyl)-4-oxo-butyric acid ethyl ester (80.1 mg, 0.24 mmol) in CHCl₃ (2.4 mL) at r.t. was added sodium acetate (30 mg, 0.36 mmol) and bromine (58 mg, 0.36 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 405.1, 407.0 (M−1)⁺.

### d) 4-(3-Cyano-2-hydroxy-5-p-tolyloxy-phenyl)-4-oxo-butyric acid ethyl ester

To a solution of the 4-(3-Bromo-2-hydroxy-5-p-tolyloxy-phenyl)-4-oxo-butyric acid ethyl ester (71.8 mg, 0.17 mmol) in DMAC (2.5 mL) at r.t. were added Zn(CN)₂ (41.2 mg, 0.35 mmol), Pd₂(dba)₃ (16.1 mg, 0.017 mmol), dppf (19.5 mg, 0.034 mmol), and Zn dust (3.4 mg, 0.051 mmol) to give suspension solution, the reaction mixture was allowed to stir at 100° C. for 3-4 hours. After cooled to rt, diluted with EtOAc (50 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 352.0 (M−1)⁺.

### e) 4-(3-Cyano-2-hydroxy-5-p-tolyloxy-phenyl)-4-oxo-butyric acid

To a solution of 4-(3-Cyano-2-hydroxy-5-p-tolyloxy-phenyl)-4-oxo-butyric acid ethyl ester (20.2 mg, 0.057 mmol) in THF (0.75 mL) and water (0.25 mL) at r.t. was added lithium hydroxide monohydrate (9.6 mg, 0.22 mmol) to give suspension solution, the reaction mixture was stirred at r.t. After 24 h, the mixture was concentrated in vacuo to give residue as a solid. The solid was dissolved in water (25 mL), extracted with EtOAc (3×15 mL). The aqueous solution was acidified by 1N HCl solution, filtered, washed with water, dried to give product: MS (m/z) 324.0 (M−1)⁺.

### Example 140

### 4-(3-Cyano-2-hydroxy-5-o-tolyloxy-phenyl)-4-oxo-butyric acid

### a) 4-(2-Benzyloxy-5-o-tolyloxy-phenyl)-4-oxo-butyric acid ethyl ester

To a solution of 4-(2-Benzyloxy-5-bromo-phenyl)-4-oxo-butyric acid ethyl ester (478 mg, 1.22 mmol, prepared in the same manner as 131b) in DMF (6.1 mL) at r.t. were added 2-methyl-phenol (198 mg, 1.83 mmol), Cs₂CO₃ (795 mg, 2.44 mmol), CuCl (12 mg, 0.12 mmol), and 2,2,6,6-tetramethyl-3,5-heptanedione (45 mg, 0.24 mmol) to give suspension solution, the reaction mixture was allowed to stir at 150° C. for 1-2 hours. After cooled to rt, diluted with EtOAc (100 mL) and filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 419.0 (M+1)⁺.

### b) 4-(2-Hydroxy-5-o-tolyloxy-phenyl)-4-oxo-butyric acid ethyl ester

A flask charged with 4-(2-Benzyloxy-5-o-tolyloxy-phenyl)-4-oxo-butyric acid ethyl ester (79.6 mg, 0.19 mmol) in TFA (2 mL) was added thioanisole (0.23 mL, 1.9 mmol). The suspension was stirred at 65° C. for 2 h. After cooled to rt, diluted with EtOAc (100 mL) and filtered through a celite plug rinsing with EtOAc (100 mL). The organic layers were washed with NaHCO₃ solution and water. The dried extract (MgSO₄) was concentrated in vacuo and purified by ISCO over silica gel, eluting with 5-35% EtOAc/hexanes to give product: MS (m/z) 329.0 (M+1)⁺.

### c) 4-(3-Bromo-2-hydroxy-5-o-tolyloxy-phenyl)-4-oxo-butyric acid ethyl ester

To a solution of the 4-(2-Hydroxy-5-o-tolyloxy-phenyl)-4-oxo-butyric acid ethyl ester (53 mg, 0.16 mmol) in CHCl₃ (1.6 mL) at r.t. was added sodium acetate (19.7 mg, 0.24 mmol) and bromine (38.7 mg, 0.24 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 404.9, 406.6 (M−1)⁺.

### d) 4-(3-Cyano-2-hydroxy-5-o-tolyloxy-phenyl)-4-oxo-butyric acid ethyl ester

To a solution of the 4-(3-Bromo-2-hydroxy-5-o-tolyloxy-phenyl)-4-oxo-butyric acid ethyl ester (53.5 mg, 0.13 mmol) in DMAC (1.8 mL) at r.t. were added Zn(CN)₂ (30.7 mg, 0.26 mmol), Pd₂(dba)₃ (11.9 mg, 0.013 mmol), dppf (14 mg, 0.026 mmol), and Zn dust (2.5 mg, 0.04 mmol) to give suspension solution, the reaction mixture was allowed to stir at 100° C. for 3-4 hours. After cooled to rt, diluted with EtOAc (50 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 352.0 (M−1)⁺.

### e) 4-(3-Cyano-2-hydroxy-5-o-tolyloxy-phenyl)-4-oxo-butyric acid

To a solution of 4-(3-Cyano-2-hydroxy-5-o-tolyloxy-phenyl)-4-oxo-butyric acid ethyl ester (17.9 mg, 0.05 mmol) in THF (0.75 mL) and water (0.25 mL) at r.t. was added lithium hydroxide monohydrate (8.5 mg, 0.2 mmol) to give suspension solution, the reaction mixture was stirred at r.t. After 24 h, the mixture was concentrated in vacuo to give residue as a solid. The solid was dissolved in water (25 mL), extracted with EtOAc (3×15 mL). The aqueous solution was acidified by 1N HCl solution, filtered, washed with water, dried to give product: MS (m/z) 324.0 (M−1)⁺.

### Example 141

### 4-[3-Cyano-2-hydroxy-5-(4-methoxy-phenoxy)-phenyl]-4-oxo-butyric acid

### a) 4-[2-Benzyloxy-5-(4-methoxy-phenoxy)-phenyl]-4-oxo-butyric acid ethyl ester

To a solution of 4-(2-Benzyloxy-5-bromo-phenyl)-4-oxo-butyric acid ethyl ester (952 mg, 2.43 mmol, prepared in the same manner as 131b) in DMF (12 mL) at r.t. were added 4-methoxy-phenol (452 mg, 3.65 mmol), Cs₂CO₃ (1.58 g, 4.86 mmol), CuCl (24 mg, 0.24 mmol), and 2,2,6,6-tetramethyl-3,5-heptanedione (89 mg, 0.48 mmol) to give suspension solution, the reaction mixture was allowed to stir at 150° C. for 1-2 hours. After cooled to rt, diluted with EtOAc (100 mL) and filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 435.1 (M+1)⁺.

### b) 4-[2-Hydroxy-5-(4-methoxy-phenoxy)-phenyl]-4-oxo-butyric acid ethyl ester

A flask charged with 4-[2-Benzyloxy-5-(4-methoxy-phenoxy)-phenyl]-4-oxo-butyric acid ethyl ester (233 mg, 0.53 mmol) in TFA (5 mL) was added thioanisole (0.64 mL, 5.3 mmol). The suspension was stirred at 65° C. for 2 h. After cooled to rt, diluted with EtOAc (100 mL) and filtered through a celite plug rinsing with EtOAc (100 mL). The organic layers were washed with NaHCO₃ solution and water. The dried extract (MgSO₄) was concentrated in vacuo and purified by ISCO over silica gel, eluting with 5-35% EtOAc/hexanes to give product: MS (m/z) 345.0 (M+1)⁺.

### c) 4-[3-Bromo-2-hydroxy-5-(4-methoxy-phenoxy)-phenyl]-4-oxo-butyric acid ethyl ester

To a solution of the 4-[2-Hydroxy-5-(4-methoxy-phenoxy)-phenyl]-4-oxo-butyric acid ethyl ester (168 mg, 0.48 mmol) in CHCl₃ (4.8 mL) at r.t. was added sodium acetate (59.8 mg, 0.73 mmol) and bromine (117 mg, 0.73 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 421.0, 423.0 (M−1)⁺.

### d) 4-[3-Cyano-2-hydroxy-5-(4-methoxy-phenoxy)-phenyl]-4-oxo-butyric acid ethyl ester

To a solution of the 4-[3-Bromo-2-hydroxy-5-(4-methoxy-phenoxy)-phenyl]-4-oxo-butyric acid ethyl ester (71 mg, 0.16 mmol) in DMAC (2.2 mL) at r.t. were added Zn(CN)₂ (39.2 mg, 0.33 mmol), Pd₂(dba)₃ (15 mg, 0.016 mmol), dppf (18 mg, 0.032 mmol), and Zn dust (3.1 mg, 0.048 mmol) to give suspension solution, the reaction mixture was allowed to stir at 100° C. for 3-4 hours. After cooled to rt, diluted with EtOAc (50 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 368.0 (M−1)⁺.

### e) 4-[3-Cyano-2-hydroxy-5-(4-methoxy-phenoxy)-phenyl]-4-oxo-butyric acid

To a solution of 4-[3-Cyano-2-hydroxy-5-(4-methoxy-phenoxy)-phenyl]-4-oxo-butyric acid ethyl ester (21.5 mg, 0.058 mmol) in THF (0.75 mL) and water (0.25 mL) at r.t. was added lithium hydroxide monohydrate (9.8 mg, 0.23 mmol) to give suspension solution, the reaction mixture was stirred at r.t. After 24 h, the mixture was concentrated in vacuo to give residue as a solid. The solid was dissolved in water (25 mL), extracted with EtOAc (3×15 mL). The aqueous solution was acidified by 1N HCl solution, filtered, washed with water, dried to give product: MS (m/z) 340.0 (M−1)⁺.

### Example 142

### 4-[3-Cyano-5-(4-fluoro-phenoxy)-2-hydroxy-phenyl]-4-oxo-butyric acid

### a) 4-[2-Benzyloxy-5-(4-fluoro-phenoxy)-phenyl]-4-oxo-butyric acid ethyl ester

To a solution of 4-(2-Benzyloxy-5-bromo-phenyl)-4-oxo-butyric acid ethyl ester (956 mg, 2.44 mmol, prepared in the same manner as 131b) in DMF (12 mL) at r.t. were added 4-fluoro-phenol (413 mg, 3.66 mmol), Cs₂CO₃ (1.59 g, 4.8 mmol), CuCl (24 mg, 0.24 mmol), and 2,2,6,6-tetramethyl-3,5-heptanedione (89.7 mg, 0.48 mmol) to give suspension solution, the reaction mixture was allowed to stir at 150° C. for 1-2 hours. After cooled to rt, diluted with EtOAc (100 mL) and filtered through a celite plug rinsing with EtOAc (100 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 445.1 (M+Na)⁺.

### b) 4-[5-(4-Fluoro-phenoxy)-2-hydroxy-phenyl]-4-oxo-butyric acid ethyl ester

A flask charged with 4-[2-Benzyloxy-5-(4-fluoro-phenoxy)-phenyl]-4-oxo-butyric acid ethyl ester (430 mg, 1.01 mmol) in TFA (10 mL) was added thioanisole (1.2 mL, 10.1 mmol). The suspension was stirred at 65° C. for 2 h. After cooled to rt, diluted with EtOAc (100 mL) and filtered through a celite plug rinsing with EtOAc (100 mL). The organic layers were washed with NaHCO₃ solution and water. The dried extract (MgSO₄) was concentrated in vacuo and purified by ISCO over silica gel, eluting with 5-35% EtOAc/hexanes to give product: MS (m/z) 331.0 (M−1)⁺.

### c) 4-[3-Bromo-5-(4-fluoro-phenoxy)-2-hydroxy-phenyl]-4-oxo-butyric acid ethyl ester

To a solution of the 4-[5-(4-fluoro-phenoxy)-2-hydroxy-phenyl]-4-oxo-butyric acid ethyl ester (124 mg, 0.38 mmol) in CHCl₃ (3.8 mL) at r.t. was added sodium acetate (47.3 mg, 0.57 mmol) and bromine (92 mg, 0.57 mmol) to give suspension solution, the reaction mixture was stirred at r.t. After 24 h, the mixture was diluted with EtOAc (100 mL). The organic layers were washed with Na₂S₂O₃ solution and water. The dried extract (MgSO₄) was concentrated in vacuo and purified by ISCO over silica gel, eluting with 5-35% EtOAc/hexanes to give product: MS (m/z) 408.9, 410.9 (M−1)⁺.

### d) 4-[3-Cyano-5-(4-fluoro-phenoxy)-2-hydroxy-phenyl]-4-oxo-butyric acid ethyl ester

To a solution of the 4-[3-Bromo-5-(4-fluoro-phenoxy)-2-hydroxy-phenyl]-4-oxo-butyric acid ethyl ester (116 mg, 0.28 mmol) in DMAC (4 mL) at r.t. were added Zn(CN)₂ (66 mg, 0.56 mmol), Pd₂(dba)₃ (25.6 mg, 0.028 mmol), dppf (31 mg, 0.056 mmol), and Zn dust (5.5 mg, 0.084 mmol) to give suspension solution, the reaction mixture was allowed to stir at 100° C. for 3-4 hours. After cooled to rt, diluted with EtOAc (50 mL) and filtered through a celite plug rinsing with EtOAc (50 mL). The organic layers were washed with 0.1 N HCl solution and water. The dried extract (MgSO₄) was concentrated in vacuo and purified by ISCO over silica gel, eluting with 5-35% EtOAc/hexanes to give product: MS (m/z) 356.0 (M−1)⁺.

### e) 4-[3-Cyano-5-(4-fluoro-phenoxy)-2-hydroxy-phenyl]-4-oxo-butyric acid

To a solution of 4-[5-(4-fluoro-phenoxy)-3-cyano-2-hydroxy-phenyl]-4-oxo-butyric acid ethyl ester (48.9 mg, 0.13 mmol) in THF (1.5 mL) and water (0.5 mL) at r.t. was added lithium hydroxide monohydrate (23 mg, 0.55 mmol) to give suspension solution, the reaction mixture was stirred at r.t. After 24 h, the mixture was concentrated in vacuo to give residue as a solid. The solid was dissolved in water (25 mL), extracted with EtOAc (3×15 mL). The aqueous solution was acidified by 1N HCl solution, filtered, washed with water, dried to give product: MS (m/z) 328.0 (M−1)⁺.

### Example 143

### 4-[4-Cyano-6-(2,6-dichloro-4-trifluoromethoxy-benzyl)-3-hydroxy-pyridin-2-yl]-4-oxo-butyric acid

### a) (2,6-Dichloro-4-trifluoromethoxy-phenyl)-methanol

To a solution of 2,6-Dichloro-4-trifluoromethoxy-benzaldehyde (1.0 g, 3.9 mmol) in methanol (20 mL) was added sodium borohydride (0.3 g, 7.8 mmol) under N₂ with ice-cooling. 30 min later, the mixture was quenched by the NH₄Cl aqueous solution (20 mL). The mixture was then concentrated under reduced pressure to remove most of the methanol. The residue was extracted with ethyl acetate (20×3 mL). The combined extracts were washed with brine (50 mL), dried over sodium sulfate, filtered and evaporated in vacuo to afford the product. MS-(+)-ion, M-OH=242.81. 1H NMR (CDCl₃, 200 mHz) δ=7.26 (s, 2H), 4.94 (s, 2H).

### b) 2-Bromomethyl-1,3-dichloro-5-trifluoromethoxy-benzene

To a solution of (2,6-Dichloro-4-trifluoromethoxy-phenyl)-methanol (1.01 g, 3.9 mmol) in CH₂Cl₂ (15 mL) was added PBr₃ (1.05 g, 3.9 mmol) at 0° C. After 2 h at 0° C., and the mixture was poured into ice-water and extracted with CH₂Cl₂. The combined organic layers were washed with saturated aq. NaHCO₃, brine, dried (Na₂SO₄), concentrated under reduced pressure, and purified by flash column chromatography to give the title compound. ¹H NMR (CDCl₃, 200 mHz) 8=7.26 (s, 2H), 4.72 (s, 2H).

### c) 2,6-Dichloro-4-trifluoromethoxy-benzylzinc(II) bromide

At 65° C., to a suspension mixture of zinc dust (260 mg, 4 mmol, Sigma, catalog #209988, <10 uM) in dry THF (5 mL) was added 1,2-dibromoethane (7 uL, 0.08 mmol) under nitrogen atmosphere, followed by the addition of chlorotrimethylsilane (41 uL, 0.32 mmol). (Note: Bubbles will be observed after 10-20 seconds the addition of chlorotrimethylsilane, which indicates the initiation of the reaction. If no bubbles formed, additional chlorotrimethylsilane should be added until the observation of bubbles). The mixture was then stirred at 65° C. for another 30 min. After cooling to room temperature, the THF solution (2 mL) of 2-Bromomethyl-1,3-dichloro-5-trifluoromethoxy-benzene (0.66g, 2.0 mmol) was dropwise added, and the suspension was stirred at room temperature for another 1 h. The reaction mixture was directly used in the next step.

### d) 4-[6-(2,6-Dichloro-4-trifluoromethoxy-benzyl)-3-hydroxy-pyridin-2-yl]-4-oxo-butyric acid ethyl ester

At 0° C., to a solution of 4-(6-Bromo-3-hydroxy-pyridin-2-yl)-4-oxo-butyric acid ethyl ester (151 mg, 0.5 mmol), Pd(OAc)₂ (5.6 mg, 0.025 mmol) and S-Phos (21 mg, 0.05 mmol) in dry THF (3 mL) was added dropwise a solution of 2,6-Dichloro-4-trifluoromethoxy-benzylzinc(II) bromide in THF (2.0 mmol) under nitrogen atmosphere. The reaction mixture was stirred at room temperature for 18 hours, then quenched by half saturated NH₄Cl aqueous solution (20 mL) and extracted with ethyl acetate (20 mL×3). The combined organic layers were dried (Na₂SO₄), concentrated under reduced pressure, and purified by flash column chromatography to give the 4-(6-Benzyl-3-hydroxy-pyridin-2-yl)-4-oxo-butyric acid ethyl ester. MS-(+)-ion, M+H=465.85.

### e) 4-[4-Bromo-6-(2,6-dichloro-4-trifluoromethoxy-benzyl)-3-hydroxy-pyridin-2-yl]-4-oxo-butyric acid ethyl ester

At room temperature, to a solution of 4-[6-(2,6-Dichloro-4-trifluoromethoxy-benzyl)-3-hydroxy-pyridin-2-yl]-4-oxo-butyric acid ethyl ester (210 mg, 0.45 mmol) and sodium acetate (74 mg, 0.9 mmol) in anhydrous CHCl₃(5 mL) was added bromine (35 uL, 108 mg, 0.68 mmol). The reaction flask was wrapped by aluminum foil. After 20 hrs at room temperature, the reaction was quenched by 15 mL saturated NaHSO₃ aqueous solution and extracted with DCM (15 mL×3). The combined organics were washed with brine (20 mL) and dried over sodium sulfate, filtered and evaporated in vacuo to afford the crude product. This material was purified by flash chromatography eluting with ethyl acetate/hexane (0%-10%) to give the title compound. MS-(+)-ion, M+H=545.77.

### f) 4-[4-Cyano-6-(2,6-dichloro-4-trifluoromethoxy-benzyl)-3-hydroxy-pyridin-2-yl]-4-oxo-butyric acid ethyl ester

The mixture of 4-[4-Bromo-6-(2,6-dichloro-4-trifluoromethoxy-benzyl)-3-hydroxy-pyridin-2-yl]-4-oxo-butyric acid ethyl ester (210 mg, 0.38 mmol), zinc cyanide (89 mg, 0.76 mmol), tris(dibenzylideneacetone)dipalladium(0) (35 mg, 0.038 mmol), 1,1′-Bis(diphenylphosphino)ferrocene (dppf, 43 mg, 0.076 mmol), and zinc dust (7.4 mg, 0.12 mmol) in anhydrous dimethylacetamide (3 mL) was heated at 100° C. under N₂ atmosphere for 3 hours. After cooling down to room temperature, the reaction mixture was diluted with water (20 mL) and ethyl acetate (20 mL). 2 mL 1 N HCl was added to the mixture following by stirring for 30 min at room temperature. The organic layer was collected. The aqueous layer was further extracted with ethyl acetate (2×20 mL). The combined extracts were washed with brine (30 mL), dried over sodium sulfate, filtered and evaporated in vacuo to afford the crude product. This material was purified by flash chromatography eluting with ethyl acetate/hexane (0%-20%) to give the title compound. MS-(+)-ion, M+H=490.86.

### g) 4-[4-Cyano-6-(2,6-dichloro-4-trifluoromethoxy-benzyl)-3-hydroxy-pyridin-2-yl]-4-oxo-butyric acid

At room temperature, to a solution of 4-[4-Cyano-6-(2,6-dichloro-4-trifluoromethoxy-benzyl)-3-hydroxy-pyridin-2-yl]-4-oxo-butyric acid ethyl ester (65 mg, 0.13 mmol) in THF/H₂O (3 mL/1 mL) was added lithium hydroxide monohydrate (27 mg, 0.65 mmol). After 20 hours at room temperature, the reaction was diluted in 15 mL water and extracted with ethyl acetate (10 mL×3). The resulting aqueous layer was treated with 1N HCl to pH=4, followed by extraction with ethyl acetate (10 mL×3). Combined organics were washed with brine and dried over sodium sulfate, filtered and evaporated in vacuo to afford the title compound. MS-(+)-ion, M+H=462.80.

### Example 144

### 4-[4-Cyano-3-hydroxy-6-(2,4,6-trichloro-benzyl)-pyridin-2-yl]-4-oxo-butyric acid

### a) (2,4,6-Trichloro-phenyl)-methanol

To a solution of 2,4,6-Trichloro benzoic acid (1 g, 4.43 mmol) in THF (60 mL) was added 1M BH₃ (13.3 mL, 13.30 mmol) in THF at 0° C. under N₂ with ice-cooling. The mixture was allowed to warm to room temperature and then reflux for overnight. Reaction was completed; reaction mixture was cooled to 0° C. and quenched with MeOH followed by 1 M HCl, The mixture was extracted with DCM, organic layer was separated, dried over Na₂SO₄, concentrated under vacuo to get crude. Attempts to purify by column chromatography were unsuccessful due to low solubility. The title compound was obtained and was used without purification.

### b) 2-Bromomethyl-1,3,5-trichloro-benzene

To a solution of crude (2,4,6-Trichloro-phenyl)-methanol (1 g, 4.72 mmol) in DCM (20 mL) was added PBr₃ (0.45 ml, 4.77 mmol) in DCM (20 ml) at −10° C. under N₂. The mixture was stirred at 0° C. for 6h; Reaction was completed; The resulting mixture was diluted with water and quenched with Aq. NaHCO₃ solution, extracted with ethyl acetate, organic layer was separated, dried over Na₂SO₄, concentrated under vacuo to get crude; purified by column chromatography using 0-5% ethyl acetate in hexane as eluent gave the title compound. ¹H NMR (CDCl₃, 400 MHz): δ=7.36 (s, 2H), 4.70 (s, 2H).

### c) 2,4,6-Trichloro-benzylzinc(II) bromide

The title compound was prepared from 2-bromomethyl-1,3,5-trichloro-benzene and zinc dust in analogy to example 4c.

### d) 4-[3-Hydroxy-6-(2,4,6-trichloro-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,4,6-trichloro-benzylzinc(II) bromide in THF in analogy to example 143d. MS-(+)-ion, M+H=417.84.

### e) 4-[4-Bromo-3-hydroxy-6-(2,4,6-trichloro-benzyl)-pyridin-2-yl]-4-oxo-butyric acid ethyl ester

The title compound was prepared from 4-[3-Hydroxy-6-(2,4,6-trichloro-benzyl)-pyridin-2-yl]-4-oxo-butyric acid ethyl ester and bromine in analogy to example 143f. MS-(+)-ion, M+H=495.66.

### f) 4-[4-Cyano-3-hydroxy-6-(2,4,6-trichloro-benzyl)-pyridin-2-yl]-4-oxo-butyric acid ethyl ester

The title compound was prepared from 4-[4-Bromo-3-hydroxy-6-(2,4,6-trichloro-benzyl)-pyridin-2-yl]-4-oxo-butyric acid ethyl ester (see example 5e) and zinc(II) cyanide in analogy to example 4f. MS-(+)-ion, M+H=442.80.

### g) 4-[4-Cyano-3-hydroxy-6-(2,4,6-trichloro-benzyl)-pyridin-2-yl]-4-oxo-butyric acid

The title compound was prepared 4-[4-Cyano-3-hydroxy-6-(2,4,6-trichloro-benzyl)-pyridin-2-yl]-4-oxo-butyric acid ethyl ester in analogy to example 143g. MS-(+)-ion, M+H=412.84.

### Example 145

### 4-[4-Cyano-6-(2,6-dichloro-4-trifluoromethyl-benzyl)-3-hydroxy-pyridin-2-yl]-4-oxo-butyric acid

### a) (2,6-Dichloro-4-trifluoromethyl-phenyl)-methanol

The title compound was prepared from 2,6-Dichloro-4-trifluoromethyl-benzaldehyde and sodium borohydride in analogy to example 143a. 1H NMR (CDCl₃, 200 mHz) 8=7.60 (s, 2H), 4.99 (s, 2H).

### b) 2-Bromomethyl-1,3-dichloro-5-trifluoromethyl-benzene

The title compound was prepared from (2,6-Dichloro-4-trifluoromethyl-phenyl)-methanol and PBr₃ in analogy to example 143b. 1H NMR (CDCl₃, 200 mHz) 8=7.60 (s, 2H), 4.76 (s, 2H).

### c) 2,6-Dichloro-4-trifluoromethyl-benzylzinc(II) bromide

The title compound was prepared from 2-Bromomethyl-1,3-dichloro-5-trifluoromethyl-benzene and zinc dust in analogy to example 143c.

### d) 4-[6-(2,6-Dichloro-4-trifluoromethyl-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,6-Dichloro-4-trifluoromethyl-benzylzinc(II) bromide in THF in analogy to example 143d. MS-(+)-ion, M+H=449.90.

### e) 4-[4-Bromo-6-(2,6-dichloro-4-trifluoromethyl-benzyl)-3-hydroxy-pyridin-2-yl]-4-oxo-butyric acid ethyl ester

The title compound was prepared from 4-[6-(2,6-Dichloro-4-trifluoromethyl-benzyl)-3-hydroxy-pyridin-2-yl]-4-oxo-butyric acid ethyl ester and bromine in analogy to example 143e. MS-(+)-ion, M+H=529.71.

### f) 4-[4-Cyano-6-(2,6-dichloro-4-trifluoromethyl-benzyl)-3-hydroxy-pyridin-2-yl]-4-oxo-butyric acid ethyl ester

The title compound was prepared from 4-[4-Bromo-6-(2,6-dichloro-4-trifluoromethyl-benzyl)-3-hydroxy-pyridin-2-yl]-4-oxo-butyric acid ethyl ester and zinc(II) cyanide in analogy to example 143f. MS-(+)-ion, M+H=474.86.

### g) 4-[4-Cyano-6-(2,6-dichloro-4-trifluoromethyl-benzyl)-3-hydroxy-pyridin-2-yl]-4-oxo-butyric acid

The title compound was prepared from 4-[4-Cyano-6-(2,6-dichloro-4-trifluoromethyl-benzyl)-3-hydroxy-pyridin-2-yl]-4-oxo-butyric acid ethyl ester in analogy to example 143g. MS-(+)-ion, M+H=446.80.

### Example 146

### 4-[6-(2-Benzyl-benzyl)-4-cyano-3-hydroxy-pyridin-2-yl]-4-oxo-butyric acid

### a) (2-Benzyl-phenyl)-methanol

To a solution of 2-Benzyl benzoic acid (1 g, 4.71 mmol) in THF (60 mL) was added 1M BH₃ (14 mL, 14.13 mmol) in THF at 0° C. under N₂ with ice-cooling. The mixture was allowed to warm to room temperature and then reflux for overnight. Reaction was completed; reaction mixture was cooled to 0° C. and quenched with MeOH followed by 1 M HCl, The mixture was extracted with DCM, organic layer was separated, dried over Na₂SO₄, concentrated under vacuo to get crude; purified by column chromatography using 0-5% ethyl acetate in hexane as eluent gave the title compound. ¹H NMR (CDCl₃, 400 MHz): δ=7.41-7.44 (m, 1H), 7.24-7.32 (m, 4H), 7.12-7.24 (m, 4H), 4.66 (s, 2H), 4.10 (s, 2H).

### b) 1-Benzyl-2-bromomethyl-benzene

To a solution of (2-Benzyl-phenyl)-methanol (1.0 g, 5.04 mmol) in DCM (20 mL) was added PBr₃ (0.5 ml, 5.09 mmol) in DCM (20 ml) at −10° C. under N₂. The mixture was stirred at 0° C. for 6h; Reaction was completed; The resulting mixture was diluted with water and quenched with Aq. NaHCO₃ solution, extracted with ethyl acetate, organic layer was separated, dried over Na₂SO₄, concentrated under vacuo to get crude; purified by column chromatography using 0-5% ethyl acetate in hexane as eluent gave the title compound. ¹H NMR (CDCl₃, 400 MHz): δ=7.12-7.38 (m, 9H), 4.46 (s, 2H), 4.17 (s, 2H).

### c) 2-Benzyl-benzylzinc(II) bromide

The title compound was prepared from 1-Benzyl-2-bromomethyl-benzene and zinc dust in analogy to example 143c.

### d) 4-[6-(2-Benzyl-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2-Benzyl-benzylzinc(II) bromide in THF in analogy to example 143d. MS-(+)-ion, M+H=404.09.

### e) 4-[6-(2-Benzyl-benzyl)-4-bromo-3-hydroxy-pyridin-2-yl]-4-oxo-butyric acid ethyl ester

The title compound was prepared from 4-[6-(2-Benzyl-benzyl)-3-hydroxy-pyridin-2-yl]-4-oxo-butyric acid ethyl ester and bromine in analogy to example 143e. MS-(+)-ion, M+H=495.66.

### f) 4-[6-(2-Benzyl-benzyl)-4-cyano-3-hydroxy-pyridin-2-yl]-4-oxo-butyric acid ethyl ester

The title compound was prepared from 4-[6-(2-Benzyl-benzyl)-4-bromo-3-hydroxy-pyridin-2-yl]-4-oxo-butyric acid ethyl ester and zinc(II) cyanide in analogy to example 143f. MS-(+)-ion, M+H=429.10.

### g) 4-[6-(2-Benzyl-benzyl)-4-cyano-3-hydroxy-pyridin-2-yl]-4-oxo-butyric acid

The title compound was prepared 4-[6-(2-Benzyl-benzyl)-4-cyano-3-hydroxy-pyridin-2-yl]-4-oxo-butyric acid ethyl ester in analogy to example 143g. MS-(+)-ion, M+H=401.04.

### Example 147

### 4-[4-Cyano-6-(2,6-dichloro-4-methoxy-benzyl)-3-hydroxy-pyridin-2-yl]-4-oxo-butyric acid

### a) (2,6-Dichloro-4-methoxy-phenyl)-methanol

The title compound was prepared from 2,6-dichloro-4-methoxy-benzaldehyde and sodium borohydride in analogy to example 143a. 1H NMR (CDCl₃, 200 mHz) 8=6.88 (s, 2H), 4.88 (s, 2H), 3.80 (s, 3H).

### b) 2-Bromomethyl-1,3-dichloro-5-methoxy-benzene

The title compound was prepared from (2,6-Dichloro-4-methoxy-phenyl)-methanol and PBr₃ in analogy to example 143b. 1H NMR (CDCl₃, 200 mHz) δ=6.89 (s, 2H), 4.74 (s, 2H), 3.80 (s, 3H).

### c) 2,6-Dichloro-4-methoxy-benzylzinc(II) bromide

The title compound was prepared from 2-Bromomethyl-1,3-dichloro-5-methoxy-benzene and zinc dust in analogy to example 143c.

### d) 4-[6-(2,6-Dichloro-4-methoxy-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,6-Dichloro-4-methoxy-benzylzinc(II) bromide in THF in analogy to example 143d. MS-(+)-ion, M+H=411.89.

### e) 4-[4-Bromo-6-(2,6-dichloro-4-methoxy-benzyl)-3-hydroxy-pyridin-2-yl]-4-oxo-butyric acid ethyl ester

To a solution of 4-[6-(2,6-Dichloro-4-methoxy-benzyl)-3-hydroxy-pyridin-2-yl]-4-oxo-butyric acid ethyl ester (316 mg, 0.77 mmol) in methanol (15 mL) was added N-bromosuccinimide (137 mg, 0.77 mmol). The mixture was stirred at room temperature for 2h. Silica gel was added to the reaction. The mixture was concentrated under vacuo to get crude; purified by column chromatography using 0-10% ethyl acetate in hexane as eluent gave the title compound. MS-(+)-ion, M+H=491.71.

### f) 4-[4-Cyano-6-(2,6-dichloro-4-methoxy-benzyl)-3-hydroxy-pyridin-2-yl]-4-oxo-butyric acid ethyl ester

The title compound was prepared from 4-[4-Bromo-6-(2,6-dichloro-4-methoxy-benzyl)-3-hydroxy-pyridin-2-yl]-4-oxo-butyric acid ethyl ester and zinc(II) cyanide in analogy to example 143f. MS-(+)-ion, M+H=436.95.

### g) 4-[4-Cyano-6-(2,6-dichloro-4-methoxy-benzyl)-3-hydroxy-pyridin-2-yl]-4-oxo-butyric acid

The title compound was prepared from 4-[4-Cyano-6-(2,6-dichloro-4-methoxy-benzyl)-3-hydroxy-pyridin-2-yl]-4-oxo-butyric acid ethyl ester in analogy to example 143g. MS-(+)-ion, M+H=408.94.

### Example 148

### 4-[4-Cyano-6-(2-fluoro-6-methyl-benzyl)-3-hydroxy-pyridin-2-yl]-4-oxo-butyric acid

### a) 2-fluoro-6-methyl-benzylzinc(II) bromide

The title compound was prepared from 2-Bromomethyl-1-fluoro-3-methyl-benzene and zinc dust in analogy to example 143c.

### b) 4-[6-(2-Fluoro-6-methyl-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2-fluoro-6-methyl-benzylzinc(II) bromide in THF (see Example 9a) in analogy to example 143d. MS-(+)-ion, M+H=346.03.

### c) 4-[4-Bromo-6-(2-fluoro-6-methyl-benzyl)-3-hydroxy-pyridin-2-yl]-4-oxo-butyric acid ethyl ester

The title compound was prepared from 4-[6-(2-Fluoro-6-methyl-benzyl)-3-hydroxy-pyridin-2-yl]-4-oxo-butyric acid ethyl ester and bromine in analogy to example 143e. MS-(+)-ion, M+H=425.96.

### d) 4-[4-Cyano-6-(2-fluoro-6-methyl-benzyl)-3-hydroxy-pyridin-2-yl]-4-oxo-butyric acid ethyl ester

The title compound was prepared from 4-[4-Bromo-6-(2-fluoro-6-methyl-benzyl)-3-hydroxy-pyridin-2-yl]-4-oxo-butyric acid ethyl ester and zinc(II) cyanide in analogy to example 143f. MS-(+)-ion, M+H=371.03.

### e) 4-[4-Cyano-6-(2-fluoro-6-methyl-benzyl)-3-hydroxy-pyridin-2-yl]-4-oxo-butyric acid

The title compound was prepared from 4-[4-Cyano-6-(2-fluoro-6-methyl-benzyl)-3-hydroxy-pyridin-2-yl]-4-oxo-butyric acid ethyl ester in analogy to example 143g. MS-(+)-ion, M+H=343.08.

### Example 149

### 4-[4-Cyano-6-(2,6-dichloro-3-fluoro-benzyl)-3-hydroxy-pyridin-2-yl]-4-oxo-butyric acid

### a) (2,6-Dichloro-3-fluoro-phenyl)-methanol

To a solution of 2,6-Dichloro-3-fluoro benzoic acid (5g, 23.92 mmol) in THF (50 mL) was added 1M BH₃ (72 mL, 71.76 mmol) in THF at 0° C. under N₂ with ice-cooling. The mixture was allowed to warm to room temperature and then reflux for overnight. Reaction was completed; reaction mixture was cooled to 0° C. and quenched with MeOH followed by 1 M HCl, The mixture was extracted with DCM, organic layer was separated, dried over Na₂SO₄, concentrated under vacuo to get crude; purified by column chromatography using 0-5% ethyl acetate in hexane as eluent gave the title compound. ¹H NMR (CDCl₃, 400 MHz): δ=7.29-7.35 (m, 1H), 7.09 (t, J=8.4 Hz, 1H), 4.97 (s, 2H).

### b) 2-Bromomethyl-1,3-dichloro-4-fluoro-benzene

To a solution of (2,6-Dichloro-3-fluoro-phenyl)-methanol (0.40 g, 2.04 mmol) in DCM (8 mL) was added PBr₃ (0.2 ml, 2.06 mmol) in DCM (8 ml) at −10° C. under N₂. The mixture was stirred at 0° C. for 6h; Reaction was completed; The resulting mixture was diluted with water and quenched with Aq. NaHCO₃ solution, extracted with ethyl acetate, organic layer was separated, dried over Na₂SO₄, concentrated under vacuo to get crude product. The procedures were repeated using 4.0g of 2,6-Dichloro-3-fluoro-phenyl)-methanol, and the two batches of crude product were combined and purified by column chromatography using 0-5% ethyl acetate in hexane to give the title compound. ¹H NMR (CDCl₃, 400 MHz): δ=7.29-7.35 (m, 1H), 7.10 (t, J=8.0 Hz, 1H), 4.74 (s, 2H).

### c) 2,4-Dichloro-3-fluoro-benzylzinc(II) bromide

The title compound was prepared from 2-Bromomethyl-1,3-dichloro-4-fluoro-benzene and zinc dust in analogy to example 143c.

### d) 4-[6-(2,6-Dichloro-3-fluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,4-Dichloro-3-fluoro-benzylzinc(II) bromide in THF in analogy to example 143d. MS-(+)-ion, M+H=399.99.

### e) 4-[4-Bromo-6-(2,6-dichloro-3-fluoro-benzyl)-3-hydroxy-pyridin-2-yl]-4-oxo-butyric acid ethyl ester

The title compound was prepared from 4-[6-(2,6-Dichloro-3-fluoro-benzyl)-3-hydroxy-pyridin-2-yl]-4-oxo-butyric acid ethyl ester and bromine in analogy to example 143e. MS-(+)-ion, M+H=479.71.

### f) 4-[4-Cyano-6-(2,6-dichloro-3-fluoro-benzyl)-3-hydroxy-pyridin-2-yl]-4-oxo-butyric acid ethyl ester

The title compound was prepared from 4-[4-Bromo-6-(2,6-dichloro-3-fluoro-benzyl)-3-hydroxy-pyridin-2-yl]-4-oxo-butyric acid ethyl ester and zinc(II) cyanide in analogy to example 143f. MS-(+)-ion, M+H=424.90.

### g) 4-[4-Cyano-6-(2,6-dichloro-3-fluoro-benzyl)-3-hydroxy-pyridin-2-yl]-4-oxo-butyric acid

The title compound was prepared 4-[4-Cyano-6-(2,6-dichloro-3-fluoro-benzyl)-3-hydroxy-pyridin-2-yl]-4-oxo-butyric acid ethyl ester in analogy to example 143g. MS-(+)-ion, M+H=396.84.

### Example 150

### 4-[4-Cyano-6-(2-fluoro-6-methoxy-benzyl)-3-hydroxy-pyridin-2-yl]-4-oxo-butyric acid

### a) 2-fluoro-6-methoxy-benzylzinc(II) bromide

The title compound was prepared from 2-Bromomethyl-1-fluoro-3-methyl-benzene and zinc dust in analogy to example 143c.

### b) 4-[6-(2-Fluoro-6-methoxy-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2-fluoro-6-methoxy-benzylzinc(II) bromide in THF in analogy to example 143d. MS-(+)-ion, M+H=362.08.

### c) 4-[4-Bromo-6-(2-fluoro-6-methoxy-benzyl)-3-hydroxy-pyridin-2-yl]-4-oxo-butyric acid ethyl ester

The title compound was prepared from 4-[6-(2-Fluoro-6-methoxy-benzyl)-3-hydroxy-pyridin-2-yl]-4-oxo-butyric acid ethyl ester and N-bromosuccinimide in analogy to 147e. MS-(+)-ion, M+H=441.95.

### d) 4-[4-Cyano-6-(2-fluoro-6-methoxy-benzyl)-3-hydroxy-pyridin-2-yl]-4-oxo-butyric acid ethyl ester

The title compound was prepared from 4-[4-Bromo-6-(2-fluoro-6-methoxy-benzyl)-3-hydroxy-pyridin-2-yl]-4-oxo-butyric acid ethyl ester and zinc(II) cyanide in analogy to example 143f. MS-(+)-ion, M+H=387.14.

### e) 4-[4-Cyano-6-(2-fluoro-6-methoxy-benzyl)-3-hydroxy-pyridin-2-yl]-4-oxo-butyric acid

The title compound was prepared from 4-[4-Cyano-6-(2-fluoro-6-methoxy-benzyl)-3-hydroxy-pyridin-2-yl]-4-oxo-butyric acid ethyl ester in analogy to example 143g. MS-(+)-ion, M+H=359.03.

### Example 151

### 4-[4-Cyano-6-(4-fluoro-2,6-dimethyl-benzyl)-3-hydroxy-pyridin-2-yl]-4-oxo-butyric acid

### a) 4-Fluoro-2,6-dimethyl-benzaldehyde

The title compound was prepared from 4-Fluoro-2,6-dimethyl-benzaldehyde and sodium borohydride in analogy to example 143a. 1H NMR (CDCl₃, 200 mHz) 8=6.74 (d, J=9.6 Hz, 2H), 4.70 (s, 2H), 2.42 (s, 6H).

### b) 2-Bromomethyl-5-fluoro-1,3-dimethyl-benzene

The title compound was prepared from 4-Fluoro-2,6-dimethyl-benzaldehyde and PBr₃ in analogy to example 143b. 1H NMR (CDCl₃, 200 mHz) 8=6.74 (d, J=9.2 Hz, 2H), 4.53 (s, 2H), 2.40 (s, 6H).

### c) 2,6-Dimethyl-4-fluoro-benzylzinc(II) bromide

The title compound was prepared from 2-Bromomethyl-5-fluoro-1,3-dimethyl-benzene and zinc dust in analogy to example 143c.

### d) 4-[6-(2,6-Dimethyl-4-fluoro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,6-Dimethyl-4-fluoro-benzylzinc(II) bromide in THF in analogy to example 143d. MS-(+)-ion, M+H=360.13.

### e) 4-[4-Bromo-6-(4-fluoro-2,6-dimethyl-benzyl)-3-hydroxy-pyridin-2-yl]-4-oxo-butyric acid ethyl ester

The title compound was prepared from 4-[6-(4-fluoro-2,6-dimethyl-benzyl)-3-hydroxy-pyridin-2-yl]-4-oxo-butyric acid ethyl ester and N-Bromosuccinimide in analogy to example 147e. MS-(+)-ion, M+H=439.90.

### f) 4-[4-Cyano-6-(4-fluoro-2,6-dimethyl-benzyl)-3-hydroxy-pyridin-2-yl]-4-oxo-butyric acid ethyl ester

The title compound was prepared from 4-[4-Bromo-6-(4-fluoro-2,6-dimethyl-benzyl)-3-hydroxy-pyridin-2-yl]-4-oxo-butyric acid ethyl ester and zinc(II) cyanide in analogy to example 143f. MS-(+)-ion, M+H=385.14.

### g) 4-[4-Cyano-6-(4-fluoro-2,6-dimethyl-benzyl)-3-hydroxy-pyridin-2-yl]-4-oxo-butyric acid

The title compound was prepared from 4-[4-Cyano-6-(4-fluoro-2,6-dimethyl-benzyl)-3-hydroxy-pyridin-2-yl]-4-oxo-butyric acid ethyl ester in analogy to example 143g. MS-(+)-ion, M+H=357.03.

### Example 152

### 4-[4-Cyano-6-(4-chloro-2,6-dimethyl-benzyl)-3-hydroxy-pyridin-2-yl]-4-oxo-butyric acid

### a) 4-Chloro-2,6-dimethyl-benzaldehyde

The title compound was prepared from 4-Chloro-2,6-dimethyl-benzaldehyde and sodium borohydride in analogy to example 143a. 1H NMR (CDCl₃, 200 mHz) δ=7.04 (s, 2H), 4.70 (s, 2H), 2.40 (s, 6H).

### b) 2-Bromomethyl-5-chloro-1,3-dimethyl-benzene

The title compound was prepared from 4-Chloro-2,6-dimethyl-benzaldehyde and PBr₃ in analogy to example 143b. 1H NMR (CDCl₃, 200 mHz) δ=7.03 (s, 2H), 4.50 (s, 2H), 2.39 (s, 6H).

### c) 2,6-Dimethyl-4-chloro-benzylzinc(II) bromide

The title compound was prepared from 2-Bromomethyl-5-chloro-1,3-dimethyl-benzene and zinc dust in analogy to example 143c.

### d) 4-[6-(2,6-Dimethyl-4-chloro-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,6-Dimethyl-4-chloro-benzylzinc(II) bromide in THF in analogy to example 143d. MS-(+)-ion, M+H=376.08.

### e) 4-[4-Bromo-6-(4-chloro-2,6-dimethyl-benzyl)-3-hydroxy-pyridin-2-yl]-4-oxo-butyric acid ethyl ester

The title compound was prepared from 4-[6-(4-fluoro-2,6-dimethyl-benzyl)-3-hydroxy-pyridin-2-yl]-4-oxo-butyric acid ethyl ester and N-Bromosuccinimide in analogy to 147e. MS-(+)-ion, M+H=455.85.

### f) 4-[4-Cyano-6-(4-chloro-2,6-dimethyl-benzyl)-3-hydroxy-pyridin-2-yl]-4-oxo-butyric acid ethyl ester

The title compound was prepared from 4-[4-Bromo-6-(4-chloro-2,6-dimethyl-benzyl)-3-hydroxy-pyridin-2-yl]-4-oxo-butyric acid ethyl ester and zinc(II) cyanide in analogy to example 143f. MS-(+)-ion, M+H=401.04.

### g) 4-[4-Cyano-6-(4-chloro-2,6-dimethyl-benzyl)-3-hydroxy-pyridin-2-yl]-4-oxo-butyric acid

The title compound was prepared from 4-[4-Cyano-6-(4-chloro-2,6-dimethyl-benzyl)-3-hydroxy-pyridin-2-yl]-4-oxo-butyric acid ethyl ester in analogy to example 143g. MS-(+)-ion, M+H=372.99.

### Example 153

### 4-[4-Cyano-6-(4-cyano-2,6-dimethyl-benzyl)-3-hydroxy-pyridin-2-yl]-4-oxo-butyric acid

### a) 4-Formyl-3,5-dimethyl-benzonitrile

The title compound was prepared from 4-Bromo-2,6-dimethyl-benzaldehyde and zinc(II) cyanide in analogy to example 143f. ¹H NMR (CDCl₃, 200 MHz) δ=10.82 (s, 1H), 7.39 (s, 2H), 2.62 (s, 6H).

### b) 4-Hydroxymethyl-3,5-dimethyl-benzonitrile

The title compound was prepared from 4-Formyl-3,5-dimethyl-benzonitrile and sodium borohydride in analogy to example 143a. 1H NMR (CDCl₃, 200 MHz) δ=7.33 (s, 2H), 4.76 (s, 2H), 2.45 (s, 6H).

### c) 4-Bromomethyl-3,5-dimethyl-benzonitrile

The title compound was prepared from 4-Hydroxymethyl-3,5-dimethyl-benzonitrile and PBr₃ in analogy to example 143b. ¹H NMR (CDCl₃, 200 mHz) 8=7.33 (s, 2H), 4.50 (s, 2H), 2.44 (s, 6H).

### d) 2,6-Dimethyl-4-nitrile-benzylzinc(II) bromide

The title compound was prepared from 4-Bromomethyl-3,5-dimethyl-benzonitrile and zinc dust in analogy to example 143c.

### e) 4-[6-(4-Cyano-2,6-dimethyl-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,6-Dimethyl-4-nitrile-benzylzinc(II) bromide in THF in analogy to example 143d. MS-(+)-ion, M+H=367.08.

### f) 4-[4-Bromo-6-(4-cyano-2,6-dimethyl-benzyl)-3-hydroxy-pyridin-2-yl]-4-oxo-butyric acid ethyl ester

The title compound was prepared from 4-[6-(4-Cyano-2,6-dimethyl-benzyl)-3-hydroxy-pyridin-2-yl]-4-oxo-butyric acid ethyl ester and bromine in analogy to example 143e. MS-(+)-ion, M+H=446.80.

### g) 4-[4-Cyano-6-(4-cyano-2,6-dimethyl-benzyl)-3-hydroxy-pyridin-2-yl]-4-oxo-butyric acid ethyl ester

The title compound was prepared from 4-[4-Bromo-6-(4-cyano-2,6-dimethyl-benzyl)-3-hydroxy-pyridin-2-yl]-4-oxo-butyric acid ethyl ester and zinc(II) cyanide in analogy to example 143f. MS-(+)-ion, M+H=392.09.

### h) 4-[4-Cyano-6-(4-cyano-2,6-dimethyl-benzyl)-3-hydroxy-pyridin-2-yl]-4-oxo-butyric acid

The title compound was prepared from 4-[4-Cyano-6-(4-cyano-2,6-dimethyl-benzyl)-3-hydroxy-pyridin-2-yl]-4-oxo-butyric acid ethyl ester in analogy to example 143g. MS-(+)-ion, M+H=364.03.

### Example 154

### 4-[4-Cyano-6-(3,5-dimethyl-isoxazol-4-ylmethyl)-3-hydroxy-pyridin-2-yl]-4-oxo-butyric acid

### a) (3,5-Dimethyl-isoxazol-4-yl)-methanol

The title compound was prepared from 3,5-Dimethyl-isoxazole-4-carbaldehyde and sodium borohydride in analogy to example 143a.

### b) 4-Bromomethyl-3,5-dimethyl-isoxazole

The title compound was prepared from (3,5-Dimethyl-isoxazol-4-yl)-methanol and PBr₃ in analogy to example 143b. MS-(+)-ion, M+H=191.95.

### c) (3,5-Dimethyl-isoxazol-4-yl)-methene zinc(II) bromide

The title compound was prepared from 2-Bromomethyl-5-chloro-1,3-dimethyl-benzene and zinc dust in analogy to example 143c.

### d) 4-[6-(3,5-Dimethyl-isoxazol-4-ylmethyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and (3,5-Dimethyl-isoxazol-4-yl)-methene zinc(II) bromide in THF in analogy to example 143d. MS-(+)-ion, M+H=332.98.

### e) 4-[4-Bromo-6-(3,5-dimethyl-isoxazol-4-ylmethyl)-3-hydroxy-pyridin-2-yl]-4-oxo-butyric acid ethyl ester

The title compound was prepared from 4-[6-(3,5-Dimethyl-isoxazol-4-ylmethyl)-3-hydroxy-pyridin-2-yl]-4-oxo-butyric acid ethyl ester and bromine in analogy to example 143e.

### f) 4-[4-Cyano-6-(3,5-dimethyl-isoxazol-4-ylmethyl)-3-hydroxy-pyridin-2-yl]-4-oxo-butyric acid ethyl ester

The title compound was prepared from 4-[4-Bromo-6-(3,5-dimethyl-isoxazol-4-ylmethyl)-3-hydroxy-pyridin-2-yl]-4-oxo-butyric acid ethyl ester and zinc(II) cyanide in analogy to example 143f. MS-(+)-ion, M+H=358.08.

### g) 4-[4-Cyano-6-(3,5-dimethyl-isoxazol-4-ylmethyl)-3-hydroxy-pyridin-2-yl]-4-oxo-butyric acid

The title compound was prepared from 4-[4-Cyano-6-(3,5-dimethyl-isoxazol-4-ylmethyl)-3-hydroxy-pyridin-2-yl]-4-oxo-butyric acid ethyl ester in analogy to example 143g. MS-(+)-ion, M+H=329.98.

### Example 155

### 4-[4-Cyano-6-(2,6-dichloro-3-methyl-benzyl)-3-hydroxy-pyridin-2-yl]-4-oxo-butyric acid

### a) (2,6-Dichloro-3-methyl-phenyl)-methanol

The title compound was prepared from 2,6-Dichloro-3-methyl-benzaldehyde and sodium borohydride in analogy to example 143a. 1H NMR (CDCl₃, 200 mHz) 8=7.27-7.12 (m, 2H), 4.99 (s, 2H), 2.37 (s, 3H).

### b) 2-Bromomethyl-1,3-dichloro-4-methyl-benzene

The title compound was prepared from (2,6-Dichloro-3-methyl-phenyl)-methanol and PBr₃ in analogy to example 143b. 1H NMR (CDCl₃, 200 mHz) δ=7.26-7.16 (m, 2H), 4.80 (s, 2H), 2.37 (s, 3H).

### c) 2,6-Dichloro-3-methyl-benzylzinc(II) bromide

The title compound was prepared from 2-Bromomethyl-1,3-dichloro-4-methyl-benzene and zinc dust in analogy to example 143c.

### d) 4-[6-(2,6-Dichloro-3-methyl-benzyl)-3-hydroxy-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,6-Dichloro-3-methyl-benzylzinc(II) bromide in THF in analogy to example 143d. MS-(+)-ion, M+H=395.94.

### e) 4-[4-Bromo-6-(2,6-dichloro-3-methyl-benzyl)-3-hydroxy-pyridin-2-yl]-4-oxo-butyric acid ethyl ester

The title compound was prepared from 4-[6-(2,6-Dichloro-3-methyl-benzyl)-3-hydroxy-pyridin-2-yl]-4-oxo-butyric acid ethyl ester and bromine in analogy to example 143e. MS-(+)-ion, M+H=475.77.

### f) 4-[4-Cyano-6-(2,6-dichloro-3-methyl-benzyl)-3-hydroxy-pyridin-2-yl]-4-oxo-butyric acid ethyl ester

The title compound was prepared from 4-[4-Bromo-6-(2,6-dichloro-3-methyl-benzyl)-3-hydroxy-pyridin-2-yl]-4-oxo-butyric acid ethyl ester and zinc(II) cyanide in analogy to example 143f. MS-(+)-ion, M+H=422.85.

### g) 4-[4-Cyano-6-(2,6-dichloro-3-methyl-benzyl)-3-hydroxy-pyridin-2-yl]-4-oxo-butyric acid

The title compound was prepared from 4-[4-Cyano-6-(2,6-dichloro-3-methyl-benzyl)-3-hydroxy-pyridin-2-yl]-4-oxo-butyric acid ethyl ester in analogy to example 143g. MS-(+)-ion, M+H=392.89.

### Example 156

### 4-[4-Cyano-3-hydroxy-6-(2,3,6-trichloro-benzyl)-pyridin-2-yl]-4-oxo-butyric acid

### a) (2,3,6-Trichloro-phenyl)-methanol

The title compound was prepared from 2,3,6-Trichloro-benzaldehyde and sodium borohydride in analogy to example 143a. 1H NMR (CDCl₃, 200 mHz) 8=7.40-7.31 (m, 2H), 4.99 (s, 2H).

### b) 2-Bromomethyl-1,3,4-trichloro-benzene

The title compound was prepared from (2,3,6-Trichloro-phenyl)-methanol and PBr₃ in analogy to example 143b. 1H NMR (CDCl₃, 200 mHz) δ=7.40-7.26 (m, 2H), 4.76 (s, 2H).

### c) 2,3,6-Trichloro-benzylzinc(II) bromide

The title compound was prepared from 2-Bromomethyl-1,3,4-trichloro-benzene and zinc dust in analogy to example 143c.

### d) 4-[3-Hydroxy-6-(2,3,6-trichloro-benzyl)-pyridin-2-yl]-4-oxo-butyric acid ethyl ester

The title compound was prepared from 4-(6-Bromo-3-hydroxy-pyridin-2-yl)-4-oxo-butyric acid ethyl ester and 2,3,6-trichloro-benzylzinc(II) bromide in THF in analogy to example 143d. MS-(+)-ion, M+H=417.89.

### e) 4-[4-Bromo-3-hydroxy-6-(2,3,6-trichloro-benzyl)-pyridin-2-yl]-4-oxo-butyric acid ethyl ester

The title compound was prepared from 4-[3-Hydroxy-6-(2,3,6-trichloro-benzyl)-pyridin-2-yl]-4-oxo-butyric acid ethyl ester and bromine in analogy to example 143e. MS-(+)-ion, M+H=495.66.

### f) 4-[4-Cyano-3-hydroxy-6-(2,3,6-trichloro-benzyl)-pyridin-2-yl]-4-oxo-butyric acid ethyl ester

The title compound was prepared from 4-[4-Bromo-3-hydroxy-6-(2,3,6-trichloro-benzyl)-pyridin-2-yl]-4-oxo-butyric acid ethyl ester and zinc(II) cyanide in analogy to example 143f. MS-(+)-ion, M+H=442.85.

### g) 4-[4-Cyano-3-hydroxy-6-(2,3,6-trichloro-benzyl)-pyridin-2-yl]-4-oxo-butyric acid

The title compound was prepared from 4-[4-Cyano-3-hydroxy-6-(2,3,6-trichloro-benzyl)-pyridin-2-yl]-4-oxo-butyric acid ethyl ester in analogy to example 143g. MS-(+)-ion, M+H=414.89.

### Example 157

### 4-(2-Cyano-3-hydroxy-6-phenyl-pyridin-4-yl)-4-oxo-butyric acid

### a) 4-(2-Cyano-3-hydroxy-6-phenyl-pyridin-4-yl)-4-oxo-butyric acid ethyl ester

A round bottom flask was charged with 4-(6-Bromo-2-cyano-3-hydroxy-pyridin-4-yl)-4-oxo-butyric acid ethyl ester (65 mg, 0.2 mmol), phenylboronic acid (37 mg, 0.3 mmol, 1.5 eq), S-Phos (7.0 mg, 0.016 mmol, 0.08 eq,), palladium acetate (3.0 mg, 0.012 mmol, 0.06 eq), and tripotassium phosphate (85 mg, 0.4 mmol, 2 eq). The flask was evacuated and backfilled with nitrogen for three times. Anhydrous toluene (3 mL) and water (7 mg, 0.4 mmol, 2.0 eq) were added to the reaction. The reaction was heated at 100° C. for 2 hrs till LC-MS shows the completion of the reaction. After cooling back to room temperature, the reaction was diluted with water (20 mL) and acidified to pH=4 with 1N HCl. The mixture was extracted with ethyl acetate (3×15 mL). The combined extracts were washed with brine (20 mL), dried over sodium sulfate, filtered and evaporated in vacuo to afford the crude product. This material was purified by flash chromatography eluting with ethyl acetate/hexane (0%-50%) to give the title compound. MS-(+)-ion, M+H=325.00.

### b) 4-(2-Cyano-3-hydroxy-6-phenyl-pyridin-4-yl)-4-oxo-butyric acid

The title compound was prepared from 4-(2-Cyano-3-hydroxy-6-phenyl-pyridin-4-yl)-4-oxo-butyric acid ethyl ester (see example 28a) in analogy to example 143g. MS-(+)-ion, M+H=296.92.

### Example 158

### 4-(6-Benzoylamino-4-cyano-3-hydroxy-pyridin-2-yl)-4-oxo-butyric acid

### a) 4-(6-Benzoylamino-4-cyano-3-hydroxy-pyridin-2-yl)-4-oxo-butyric acid ethyl ester

The solution of 4-(6-Benzoylamino-3-benzyloxy-4-cyano-pyridin-2-yl)-4-oxo-butyric acid ethyl ester (130 mg, 0.28 mmol, see Example 23b) and thioanisole (348 mg, 2.8 mmol) in 2 mL trifluoroacetic acid was stirred at room temperature for 16 hours. The reaction was then slowly quenched by aqueous sodium bicarbonate solution to pH=7-8. The mixture was extracted with ethyl acetate, dried over sodium sulfate, filtered and evaporated in vacuo to afford the crude product. This material was purified by flash chromatography eluting with ethyl acetate/hexane (0%-40%) to give the title compound.

MS-(−)-ion, M−H=367.98.

### b) 4-(6-Benzoylamino-4-cyano-3-hydroxy-pyridin-2-yl)-4-oxo-butyric acid

The title compound was prepared from 4-(6-Benzoylamino-4-cyano-3-hydroxy-pyridin-2-yl)-4-oxo-butyric acid ethyl ester in analogy to example 143g. MS-(+)-ion, M+H=339.98.

### Example 159

### 4-[6-(Benzoyl-N-methyl-amino)-4-cyano-3-hydroxy-pyridin-2-yl]-4-oxo-butyric acid

### a) 4-[6-(Benzoyl-N-methyl-amino)-3-benzyloxy-4-cyano-pyridin-2-yl]-4-oxo-butyric acid ethyl ester

Methyl iodide (28 mg, 0.2 mmol, 2.0 eq.) was added to a mixture of 4-(6-Benzoylamino-3-benzyloxy-4-cyano-pyridin-2-yl)-4-oxo-butyric acid ethyl ester (46 mg, 0.1 mmol, see Example 23b) and potassium carbonate (28 mg, 0.2 mmol, 2.0 eq.) in anhydrous DMF (3 mL) at room temperature. After 18 hours at room temperature, TLC shows the completion of the reaction. The reaction mixture was diluted with water (20 mL) and extracted with ethyl acetate (4×15 mL). The combined extracts were washed with brine (2×20 mL), dried over sodium sulfate, filtered and evaporated in vacuo to afford the crude product. This material was purified by flash chromatography eluting with ethyl acetate/hexane (0%-40%) to give the title compound. MS-(+)-ion, M+H=472.10.

### b) 4-[6-(Benzoyl-N-methyl-amino)-4-cyano-3-hydroxy-pyridin-2-yl]-4-oxo-butyric acid ethyl ester

The title compound was prepared from 4-[6-(Benzoyl-methyl-amino)-3-benzyloxy-4-cyano-pyridin-2-yl]-4-oxo-butyric acid ethyl ester and thioanisole in analogy to example 158a. MS-(+)-ion, M+H=382.09.

### c) 4-[6-(Benzoyl-N-methyl-amino)-4-cyano-3-hydroxy-pyridin-2-yl]-4-oxo-butyric acid

The title compound was prepared from 4-[6-(Benzoyl-methyl-amino)-4-cyano-3-hydroxy-pyridin-2-yl]-4-oxo-butyric acid ethyl ester in analogy to example 143g. MS-(+)-ion, M+H=353.98.

### Example 160

### 4-(6-Benzyl-2-cyano-3-hydroxy-pyridin-4-yl)-4-oxo-butyric acid

### a) 3-Hydroxy-isonicotinic acid ethyl ester

To the solution of 3-Hydroxy-isonicotinic acid (12.5 g, 90 mmol) in anhydrous ethanol (300 mL) was added 98% sulfuric acid (14.5 mL, 270 mmol, 3.0 eq.). The reaction was refluxed for 48 hours. After the solvents were evaporated, the residue was dissolved in 300 ml of water, neutralized with saturated sodium bicarbonate solution and extracted with ethyl acetate (200 mL×3). The combined organics were washed with brine (200 mL), dried over sodium sulfate, filtered and evaporated in vacuo to afford the title compound; MS-(+)-ion, M+1=167.95.

### b) 3-Hydroxy-2-iodo-isonicotinic acid ethyl ester

The mixture of 3-Hydroxy-isonicotinic acid ethyl ester (7.5g, 45 mmol) and sodium carbonate (5.25g, 49.5 mmol, 1.1 eq) in water (400 mL) was stirred at room temperature for 30 min. Iodine (9.65g, 38 mmol, 0.9 eq) was added to the reaction mixture in one portion. After 3 hours at room temperature, the reaction mixture was quenched with 1 N HCl to pH=4. The mixture extracted with ethyl acetate (4×150 mL). The combined extracts were washed with brine (2×200 mL), dried over sodium sulfate, filtered and evaporated in vacuo to afford the crude product. This material was purified by flash chromatography eluting with ethyl acetate/hexane (0%-10%) to give the title compound, 2.95 g. MS-(+)-ion, M+1=293.77.

### c) 2-Cyano-3-hydroxy-isonicotinic acid ethyl ester

The mixture of 3-Hydroxy-2-iodo-isonicotinic acid ethyl ester (2.93 g, 10 mmol), copper (I) cyanide (2.69 g, 30 mmol), in anhydrous dimethylacetamide (50 mL) was heated at 100° C. under N₂ atmosphere for 1 hour. After cooling down to room temperature, the reaction mixture was diluted with water (50 mL) and ethyl acetate (50 mL). 5 mL 1 N HCl was added to the mixture following by stirring for 30 min at room temperature. The organic layer was collected. The aqueous layer was further extracted with ethyl acetate (2×50 mL). The combined extracts were washed with brine (100 mL), dried over sodium sulfate, filtered and evaporated in vacuo to afford the crude product, which was used directly in the next step.

### d) 3-Benzyloxy-2-cyano-isonicotinic acid ethyl ester

Benzyl bromide (1.81 mL, 2.61 g, 15 mmol) was added to a mixture of 2-Cyano-3-hydroxy-isonicotinic acid ethyl ester (1.92 g, 10 mmol) and cesium carbonate (4.24 g, 13 mmol) in anhydrous DMF (50 mL) at room temperature. After 40 hours at room temperature, TLC shows the completion of the reaction. The reaction mixture was diluted with water (100 mL) and extracted with ethyl acetate (4×50 mL). The combined extracts were washed with brine (2×100 mL), dried over sodium sulfate, filtered and evaporated in vacuo to afford the crude product. This material was purified by flash chromatography to give the title compound, 2.92 g. MS-(+)-ion, M+1=282.97.

### e) [2-(3-Benzyloxy-2-cyano-pyridin-4-yl)-2-oxo-ethyl]-phosphonic acid dimethyl ester

At −78° C., to a solution of dimethyl methylphosphonate (3.2 mL, 29.7 mmol, 3.3 eq.) in THF (50 mL) was added sodium bis(trimethylsilyl)amide solution (1.0 M in THF, 27 mL, 83.6 mmol, 3.0 eq) over 10 min under N₂ atmosphere. After 30 min, a solution of 3-benzyloxy-2-cyano-isonicotinic acid ethyl ester (2.54 g, 9.0 mmol) in THF (10 mL) was added slowly over 10 min. After stirring for 1 h at −78° C., the mixture was treated with half saturated aq. NH₄Cl (50 mL) and extracted with ethyl acetate (4×50 mL). The combined extracts were dried over sodium sulfate, filtered and evaporated in vacuo to afford the crude product. This material was purified by flash chromatography to give the title compound. MS-(+)-ion, M+1=361.03.

### f) 4-(3-Benzyloxy-2-cyano-pyridin-4-yl)-4-oxo-but-2-enoic acid ethyl ester

To an ice cooled solution of [2-(3-Benzyloxy-2-cyano-pyridin-4-yl)-2-oxo-ethyl]-phosphonic acid dimethyl ester (2.7 g, 7.5 mmol) in acetonitrile was added ethyl glyoxalate (3.3 mmol, 50% in toluene). After 1 hour at 0° C., the reaction mixture was quenched using saturated aq. solution of ammonium chloride. The mixture was extracted with ethyl acetate (3×30 mL). The combined extracts were washed with brine (20 mL), dried over sodium sulfate, filtered and evaporated in vacuo to afford the crude product. This material was purified by flash chromatography eluting with ethyl acetate/hexane (0%-25%) to give the title compound, 1.09 g. MS-(+)-ion, M+H=337.03.

### g) 4-(2-Cyano-3-hydroxy-pyridin-4-yl)-4-oxo-butyric acid ethyl ester

To a solution of 4-(3-Benzyloxy-2-cyano-pyridin-4-yl)-4-oxo-but-2-enoic acid ethyl ester (1.09 g, 3.25 mmol) in ethyl acetate (50 mL) was added Pd/C (138 mg, 0.02eq, 10 wt. %, wet, contains ˜51% water). The mixture was vacuumed/refilled with hydrogen gas for three times. After stirring for 16 hr at room temperature, the reaction mixture was filtrated off through celite. The filtrate was evaporated in vacuo to afford the crude product. This material was purified by flash chromatography eluting with EtOAc/Hexane (0%-80%) to give the title compound, 270 mg. MS-(−)-ion, M−1=247.01.

### h) 4-(6-Bromo-2-cyano-3-hydroxy-pyridin-4-yl)-4-oxo-butyric acid ethyl ester

To a solution of 4-(2-Cyano-3-hydroxy-pyridin-4-yl)-4-oxo-butyric acid ethyl ester (270 mg, 1.1 mmol) in acetonitrile (10 mL) was added N-bromosuccinimide (195 mg, 1.1 mmol). The mixture was stirred at room temperature for 1 h. Silica gel was added to the reaction. The mixture was concentrated under vacuo to get crude; purified by column chromatography using 0-50% ethyl acetate in hexane as eluent gave the title compound. 1H NMR (CDCl₃, 200 mHz) 8=11.86 (s, 1H), 8.01 (s, 1H), 4.15 (q, J=7.4 Hz, 2H), 3.35 (t, J=6.2 Hz, 2H), 2.82 (t, J=6.2 Hz, 2H), 1.31 (t, J=7.4 Hz, 3H).

### i) 4-(6-Benzyl-2-cyano-3-hydroxy-pyridin-4-yl)-4-oxo-butyric acid ethyl ester

The title compound was prepared from 4-(6-Bromo-2-cyano-3-hydroxy-pyridin-4-yl)-4-oxo-butyric acid ethyl ester and benzylzinc(II) bromide in THF in analogy to example 143d. MS-(+)-ion, M+H=338.98.

### j) 4-(6-Benzyl-2-cyano-3-hydroxy-pyridin-4-yl)-4-oxo-butyric acid

The title compound was prepared from 4-(6-Benzyl-2-cyano-3-hydroxy-pyridin-4-yl)-4-oxo-butyric acid ethyl ester in analogy to example 143g. MS-(+)-ion, M+H=310.97.

### BIOLOGICAL EXAMPLES

### Biological Example 1

Enzymatic activity was determined based on the capture of ¹⁴CO₂ released by the decarboxylation of [1-¹⁴C] αKG (Zhang et al., Anal. Biochem., 1998, vol. 271, pp. 137-142). [1-¹⁴C]alphaketoglutarate (αKG) was from Perkin-Elmer, H3K4me3 and H3K9me3 peptides were from Mimotopes. All other reagents were from Sigma. Reaction mixtures included the following components: Fe(SO₄), [1-¹⁴C] αKG, non-labeled αKG, ascorbate, peptide-substrate (H3K4me3: H-ARTK(me3)QTARKSTGGKAPRKQLA-OH. H3K9me3: H-ARTKQTARK(me3)STGGKAPRKQLA-OH), NaCl, Tween-20 and catalase in 25 mM HEPES buffer, pH 7.4. The enzymatic reactions were initiated by addition of recombinant human KDM5B or KDM4A enzyme (truncated enzyme produced in house by method and using sequence disclosed in Ng et al, Nature 448:87-91, 2007). The reactions were performed in 96-well microtiter plates (20 μL total assay volume) (Greiner #650201). ¹⁴CO₂ was captured on a glass fiber filter paper (Cat. No. IH-201-A, Inotech Biosystems International) soaked with saturated Ba(OH)₂ that was laid on top of the 96-well plate. A microtiter plate sealer film (Thermal Seal cat #T7961100) was applied to the filter paper. The plate and filter paper were sandwiched between two custom made aluminum plates (Advanced Component Manufacturing, Burlingame, CA) and transferred to a 37° C. oven and allowed to incubate for 1 hour. After incubation, the filter paper was dried in a 103° C. oven for 40-60 minutes. To determine percent turnover, aliquots of the reaction mixture were spotted onto the filter paper and the filter paper was dried again. The dry filter paper was exposed to a storage phosphor screen for 24-72 hours and the images recorded using a Typhoon FLA 7000 Imager (Amersham Biosciences, Piscataway, NJ). Integrated spot intensities corresponding to control reactions lacking the enzyme were subtracted from integration results for enzyme containing reactions and data were converted to enzyme dependent percent ¹⁴CO₂ release. All enzymatic reactions were run in duplicates.

For IC₅₀ determination, a 3-fold dilution series of compound was prepared and added to the reaction mixture (final 1% DMSO, v/v) prior to enzyme addition. The final reagent concentrations were: 10 μM Fe(SO₄), 10 μM [1-¹⁴C] αKG, 90 μM non-labeled αKG, 2 mM ascorbate, 50 μM peptide substrate, 75 mM NaCl, 0.01% Tween-20 and ˜2 units/μL catalase. IC₅₀s were determined by non-linear fitting using Grafit version 7.0. Data for compounds disclosed herein is shown in Table 2.

TABLE 2

| No | KDM5B | KDM4A |
|---|---|---|
| 1 | 0.45 | 36 |
| 2 | 0.31 | 19 |
| 3 | 0.11 | 16 |
| 4 | 0.23 | 3.5 |
| 5 | 0.24 | 71 |
| 6 | 0.28 | >40 |
| 7 | 0.21 | >40 |
| 8 | 0.27 | 13 |
| 9 | 0.16 | >40 |
| 10 | 0.16 | >40 |
| 11 | 0.59 | >40 |
| 12 | 0.13 | 16 |
| 13 | 0.14 | 22 |
| 14 | 0.47 | >40 |
| 15 | 0.39 | >40 |
| 16 | 0.21 | >40 |
| 17 | 0.19 | >40 |
| 18 | 0.10 | >40 |
| 19 | 0.23 | >40 |
| 20 | 0.17 | >40 |
| 21 | 0.21 | 29 |
| 22 | 0.18 | 40 |
| 23 | 0.15 | 40 |
| 24 | 0.52 | >40 |
| 25 | 0.33 | >40 |
| 26 | 0.20 | >40 |
| 27 | 0.55 | >40 |
| 28 | 0.20 | >40 |
| 29 | 0.46 | >40 |
| 30 | 0.25 | 17.0 |
| 31 | 0.23 | >40 |
| 32 | 0.24 | 12 |
| 33 | 0.42 | >40 |
| 34 | 0.06 | 12 |
| 35 | 0.05 | 12 |
| 36 | 0.07 | 3.7 |
| 37 | 0.06 | >40 |
| 38 | 0.12 | >40 |
| 39 | 0.13 | >40 |
| 40 | 0.06 | 17 |
| 41 | 0.15 | 22 |
| 42 | 0.21 | 32 |
| 43 | 0.15 | 37 |
| 44 | 0.23 | >40 |
| 45 | 0.54 | >40 |
| 46 | 0.43 | 13 |
| 47 | 0.30 | >40 |
| 48 | 0.31 | >40 |
| 49 | 0.27 | >40 |
| 50 | 0.39 | >40 |
| 51 | 0.20 | >40 |
| 52 | 0.30 | >40 |
| 53 | 0.25 | >40 |
| 54 | 0.23 | 4.9 |
| 55 | 0.22 | 34 |
| 56 | 0.29 | >40 |
| 57 | 0.27 | 28 |
| 58 | 0.18 | 16 |
| 59 | 0.33 | >40 |
| 60 | 0.33 | >40 |
| 61 | 0.57 | >40 |
| 62 | 0.47 | >40 |
| 63 | 0.24 | 36 |
| 64 | 0.26 | >40 |
| 65 | 0.18 | 14 |
| 66 | 0.15 | >40 |
| 67 | 0.24 | >40 |
| 68 | 0.28 | >40 |
| 69 | 0.15 | >40 |
| 70 | 0.30 | >40 |
| 71 | 0.31 | >40 |
| 72 | 0.10 | >40 |
| 73 | 0.11 | >40 |
| 74 | 0.06 | >40 |
| 75 | 0.09 | 20 |
| 76 | 0.19 | >40 |
| 77 | 0.04 | 12 |
| 78 | 0.09 | >40 |
| 79 | 0.23 | >40 |
| 80 | 0.39 | >40 |
| 81 | 0.34 | >40 |
| 82 | 0.35 | >40 |
| 83 | 0.25 | >40 |
| 84 | 0.46 | >40 |
| 85 | 0.45 | >40 |
| 86 | 0.45 | >40 |
| 87 | 0.28 | >40 |
| 88 | 0.71 | >40 |
| 89 | 0.31 | >40 |
| 90 | 0.25 | >40 |
| 91 | 0.17 | >40 |
| 92 | 0.37 | >40 |
| 93 | 0.33 | >40 |
| 94 | 0.27 | >40 |
| 95 | 0.35 | >40 |
| 96 | 0.31 | >40 |
| 97 | 0.30 | >40 |
| 98 | 0.30 | >40 |
| 99 | 0.27 | >40 |
| 100 | 0.43 | >40 |
| 101 | 0.31 | >40 |
| 102 | 0.40 | >40 |
| 103 | 0.38 | >40 |
| 104 | 0.46 | >40 |
| 105 | 0.42 | >40 |
| 106 | 0.35 | >40 |
| 107 | 0.32 | >40 |
| 108 | 0.27 | >40 |
| 109 | 0.25 | >40 |
| 110 | 0.27 | >40 |
| 111 | 0.32 | >40 |
| 112 | 0.39 | >40 |
| 113 | 0.23 | >40 |
| 114 | 0.14 | 10 |
| 115 | 0.23 | >40 |
| 116 | 0.10 | >40 |
| 117 | 0.28 | >40 |
| 118 | 0.16 | >40 |
| 119 | 0.28 | >40 |
| 120 | 0.15 | >40 |
| 121 | 0.08 | >40 |
| 122 | 0.23 | >40 |
| 123 | 0.12 | 3.0 |
| 124 | 0.16 | >40 |
| 125 | 0.08 | 13 |
| 126 | 0.07 | 2.9 |
| 127 | 0.06 | >40 |
| 128 | 0.38 | >40 |
| 129 | 0.56 | >40 |
| 130 | 3.2 | >40 |
| 131 | 0.05 | >40 |
| 132 | 0.31 | >40 |
| 133 | 1.4 | >40 |
| 134 | 0.12 | >40 |
| 135 | 0.37 | >40 |
| 136 | 0.37 | >40 |
| 137 | 0.82 | >40 |
| 138 | 0.39 | >40 |
| 139 | 0.56 | >40 |
| 140 | 0.33 | >40 |
| 141 | 0.25 | >40 |
| 142 | 0.28 | >40 |
| 143 | 0.23 | >40 |
| 144 | 0.18 | 28 |
| 145 | 0.36 | >40 |
| 146 | 0.14 | 39 |
| 147 | 0.16 | >40 |
| 148 | 0.30 | 4.3 |
| 149 | 0.07 | 3.7 |
| 150 | 0.28 | 27 |
| 151 | 0.50 | 1.5 |
| 152 | 0.45 | 9.7 |
| 153 | 0.32 | 17 |
| 154 | 0.26 | 1.4 |
| 155 | 0.18 | 34 |
| 156 | 0.07 | 11 |
| 157 | 1.4 | >40 |
| 158 | 0.08 | >40 |
| 159 | 0.99 | >40 |
| 160 | 2.0 | >40 |

### Biological Example 2

### a) Cell-Based Assay for KDM5 Inhibitors

COS-7 monkey kidney fibroblasts (ATCC, Manassas VA) were seeded into collagen-coated 96-well culture dishes and incubated overnight at 37° C., 5% CO₂ in standard culture medium, e.g., Dulbecco's Modified Eagle Medium containing 10% fetal bovine serum. The next day, cells were transfected with an expression plasmid for Myc-His-tagged KDM5B (Origene, Rockville MD) for 3h before replacing the transfection medium with fresh culture medium and treating with vehicle or compound.

After 18h incubation, the cell culture treatment medium was removed and cell layers were fixed using 4% formaldehyde in Dulbecco's Phosphate Buffered Saline (DPBS), then permeabilized with 0.25% Triton X-100, and then blocked using a suitable blocking agent e.g., Odyssey Blocking Buffer (LICOR, Lincoln NE). Cell layers were then incubated overnight with primary antibodies for detection of MYC (Thermo Fisher Scientific, Waltham MA) and tri-methylated Lysine 4 of Histone 3 (H3K4me3) (Cell Signaling Technology, Danvers MA). The next day cell layers were washed and incubated with appropriate fluorescently labeled secondary antibodies. Finally, nuclei were stained using 4′,6-diamidino-2-phenylindole (DAPI).

### b) Imaging and Analyses of Cell-Based Assay for KDM5 Inhibitors

In one analysis method, fold increases in H3K4me3 levels per cell were assessed. A suitable cell imaging system e.g., Cytation5 (Biotek, Winooski VT) was used to scan cell layers that had been immunostained for MYC and H3K4me3. Scanned objects were gated using DAPI (cells) and binned according to MYC-KDM5B expression levels (low, medium, or high). H3K4me3 levels were quantified per cell. Mean fold increases in H3K4me3 in compound-treated cells relative to vehicle-treated cells were calculated for each MYC-KDM5B expressing bin (low, medium, or high).

In another analysis method, the percentages of MYC-KDM5B-overexpressing cells with H3K4me3 levels above a pre-determined threshold were calculated. A suitable cell imaging system e.g., Incucyte ZOOM (Sartorius, Germany) was used to scan cell layers that had been immunostained for MYC-KDM5B and H3K4me3. Cells highly overexpressing MYC-KDM5B were gated based on MYC staining levels and then the percentages of cells within vehicle- and compound-treated groups with H3K4me3 levels above a set threshold were determined. Compound-dependent increases in percentages of cells above the pre-determined H3K4me3 threshold were calculated.

1. A compound of formula I: 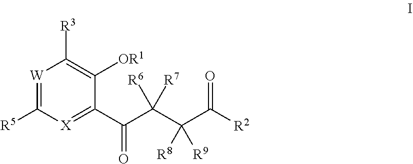
or a pharmaceutically acceptable salt, isotopically enriched analog, stereoisomer, mixture of stereoisomers or prodrug thereof, wherein:
one of W or X is N and the other of W and X is CR⁴; or W and X are CR⁴;
R¹ is hydrogen, —P(O)(OR²⁰)₂, —CH₂P(O)(OR²⁰)₂, —P(O)(R²⁰)(OR²⁰), —CH₂P(O)(R²⁰)(OR²⁰), —P(O)(N(R²⁰)₂)(OR²⁰), —CH₂P(O)(N(R²⁰)₂)(OR²⁰), —P(O)(R²⁰)(N(R²⁰)₂), —CH₂P(O)(R²⁰)(N(R²⁰)₂), —C(O)R²⁰, —C(O)N(R²)(R²²), —CH₂P(O)(N(R²⁰)₂)₂, or —P(O)(N(R²⁰)₂)₂;
R² is —OH, —OCH₂P(O)(OR²⁰)₂, —OCH₂P(O)(R²⁰)(N(R²⁰)₂), —OCH₂P(O)(R²⁰)(OR²⁰), —OCH₂P(O)(N(R²⁰)₂)(OR²⁰), —OCH₂P(O)(N(R²⁰)₂)₂, —N(R²¹)(R²²), —N(R²⁰)C(O)R²⁰, —N(R²⁰)C(O)OR²⁰, —N(R²⁰)C(O)N(R²¹)(R²¹), —N(R²⁰)S(O)₂(R²⁰), —NR²⁰S(O)₂N(R²)(R²²), or —NR²⁰S(O)₂O(R²⁰)
R³ is halo, cyano, or C₁₋₆ haloalkyl;
each R⁴ is independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each alkyl, alkenyl, alkynyl, haloalkyl, cycloalkyl, cycloalkenyl, heterocyclyl, aryl, and heteroaryl of R⁴ is independently optionally substituted with 1-5 R¹⁴;
R⁵ is halo, cyano, -L-C₁₋₆ alkyl, -L-C₁₋₆ haloalkyl, -L-C₃₋₁₀ cycloalkyl, -L-C₃₋₁₀ cycloalkenyl, -L-heterocyclyl, -L-aryl, or -L-heteroaryl; wherein each alkyl, haloalkyl, cycloalkyl, cycloalkenyl, heterocyclyl, aryl, and heteroaryl of R⁵ is independently optionally substituted with 1-5 R¹⁵;
L is a bond, —C₁₋₆ alkylene, —C₁₋₆ heteroalkylene, —O—, —S—, —S(O)—, —S(O)₂—, —NR¹⁶—, —C(O)NR¹⁶—, —NR¹⁶C(O)—, —OC(O)—, or —C(O)O—;
each of R⁶, R⁷, R⁸, and R⁹ are independently hydrogen, deuterium, C₁₋₆ alkyl, or C₁₋₆ haloalkyl;
each of R¹⁴ and R¹⁵ are independently hydroxy, halo, cyano, —NO₂, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, aryl, benzyl, heteroaryl, heterocyclyl, —N(R¹⁶)₂, —C(O)R¹⁶, —C(O)OR¹⁶, —S—R¹⁶, S(O)R¹⁶, —NR¹⁶S(O)R¹⁶, —S(O)N(R¹⁶)₂, —NR¹⁶S(O)N(R¹⁶)₂, —S(O)₂R¹⁶, —NR¹⁶S(O)₂—R¹⁶, —S(O)₂N(R¹⁶)₂, —NR¹⁶S(O)₂N(R¹⁶)₂, —NR¹⁶C(O)N(R¹⁶)₂, —C(O)N(R¹⁶)₂, —NR¹⁶C(O)R¹⁶, —OC(O)N(R¹⁶)₂, or —NR¹⁶C(O)OR¹⁶;
each R¹⁶ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂-6 alkynyl, C₁₋₆ haloalkyl, C₁₋₆ heteroalkyl, C₃₋₁₀ cycloalkyl, heterocyclyl, aryl, or heteroaryl of R¹⁶ is independently optionally substituted with 1-5 halo, cyano, —NO₂, oxo, —SF₅, C₁₋₆ alkyl, C₁₋₆ haloalkyl, C₁₋₆ alkoxy, C₁₋₆ haloalkoxy, C₃₋₁₀ cycloalkyl, aryl, benzyl, heteroaryl, or heterocyclyl;
each R²⁰ is independently hydrogen, C₁₋₆ alkyl, C₂₋₆ alkenyl, C₂₋₆ alkynyl, C₃₋₁₀ cycloalkyl, aryl, heteroaryl, or heterocyclyl; wherein each alkyl, alkenyl, alkynyl, cycloalkyl, aryl, heteroaryl, or heterocyclyl is independently optionally substituted with 1-5 R³⁰;
each R²¹ and R²² is independently hydrogen, C₁₋₆ alkyl, C₂₋₆ alkenyl, C₂₋₆ alkynyl, C₃₋₁₀ cycloalkyl, aryl, heteroaryl, or heterocyclyl; wherein each alkyl, alkenyl, alkynyl, cycloalkyl, aryl, heteroaryl, or heterocyclyl is independently optionally substituted with 1-5 R³⁰ groups, or R²⁰ and R²¹ together with the nitrogen to which they are attached form a heterocyclyl; wherein said heterocyclyl is independently optionally substituted with 1-5 R³⁰;
each R³⁰ is independently oxo, thioxo, hydroxy, halo, —NO₂, —N₃, cyano, C₁₋₆ alkyl, C₂₋₆ alkenyl, C₂₋₆ alkynyl, C₃₋₁₀ cycloalkyl, C₁₋₆ haloalkyl, aryl, heteroaryl, heterocyclyl, —O(C₁₋₆ alkyl), —O(C₂₋₆ alkenyl), —O(C₂₋₆ alkynyl), —O(C₃₋₁₀ cycloalkyl), —O(C₁₋₆ haloalkyl), —O(aryl), —O(heteroaryl), —O(heterocyclyl), —NH₂, —NH(C₁₋₆ alkyl), —NH(C₂₋₆ alkenyl), —NH(C₂₋₆ alkynyl), —NH(C₃₋₁₀ cycloalkyl), —NH(C₁₋₆ haloalkyl), —NH(aryl), —NH(heteroaryl), —NH(heterocyclyl), —N(C₁₋₆ alkyl)₂, —N(C₃₋₁₀ cycloalkyl)₂, —N(C₂₋₆ alkenyl)₂, —N(C₂₋₆ alkynyl)₂, —N(C₃₋₁₀ cycloalkyl)₂, —N(C₁₋₆ haloalkyl)₂, —N(aryl)₂, —N(heteroaryl)₂, —N(heterocyclyl)₂, —N(C₁₋₆ alkyl)(C₃₋₁₀ cycloalkyl), —N(C₁₋₆ alkyl)(C₂₋₆ alkenyl), —N(C₁₋₆ alkyl)(C₂₋₆ alkynyl), —N(C₁₋₆ alkyl)(C₃₋₁₀ cycloalkyl), —N(C₁₋₆ alkyl)(C₁₋₆ haloalkyl), —N(C₁₋₆ alkyl)(aryl), —N(C₁₋₆ alkyl)(heteroaryl), —N(C₁₋₆ alkyl)(heterocyclyl), —C(O)(C₁₋₆ alkyl), —C(O)(C₂₋₆ alkenyl), —C(O)(C₂₋₆ alkynyl), —C(O)(C₃₋₁₀ cycloalkyl), —C(O)(C₁₋₆ haloalkyl), —C(O)(aryl), —C(O)(heteroaryl), —C(O)(heterocyclyl), —C(O)O(C₁₋₆ alkyl), —C(O)O(C₂₋₆ alkenyl), —C(O)O(C₂₋₆ alkynyl), —C(O)O(C₃₋₁₀ cycloalkyl), —C(O)O(C₁₋₆ haloalkyl), —C(O)O(aryl), —C(O)O(heteroaryl), —C(O)O(heterocyclyl), —C(O)NH₂, —C(O)NH(C₁₋₆ alkyl), —C(O)NH(C₂₋₆ alkenyl), —C(O)NH(C₂₋₆ alkynyl), —C(O)NH(C₃₋₁₀ cycloalkyl), —C(O)NH(C₁₋₆ haloalkyl), —C(O)NH(aryl), —C(O)NH(heteroaryl), —C(O)NH(heterocyclyl), —C(O)N(C₁₋₆ alkyl)₂, —C(O)N(C₃₋₁₀ cycloalkyl)₂, —C(O)N(C₂₋₆ alkenyl)₂, —C(O)N(C₂₋₆ alkynyl)₂, —C(O)N(C₃₋₁₀ cycloalkyl)₂, —C(O)N(C₁₋₆ haloalkyl)₂, —C(O)N(aryl)₂, —C(O)N(heteroaryl)₂, —C(O)N(heterocyclyl)₂, —NHC(O)(C₁₋₆ alkyl), —NHC(O)(C₂₋₆ alkenyl), —NHC(O)(C₂₋₆ alkynyl), —NHC(O)(C₃₋₁₀ cycloalkyl), —NHC(O)(C₁₋₆ haloalkyl), —NHC(O)(aryl), —NHC(O)(heteroaryl), —NHC(O)(heterocyclyl), —NHC(O)O(C₁₋₆ alkyl), —NHC(O)O(C₂₋₆ alkenyl), —NHC(O)O(C₂₋₆ alkynyl), —NHC(O)O(C₃₋₁₀ cycloalkyl), —NHC(O)O(C₁₋₆ haloalkyl), —NHC(O)O(aryl), —NHC(O)O(heteroaryl), —NHC(O)O(heterocyclyl), —NHC(O)NH(C₁₋₆ alkyl), —NHC(O)NH(C₂₋₆ alkenyl), —NHC(O)NH(C₂₋₆ alkynyl), —NHC(O)NH(C₃₋₁₀ cycloalkyl), —NHC(O)NH(C₁₋₆ haloalkyl), —NHC(O)NH(aryl), —NHC(O)NH(heteroaryl), —NHC(O)NH(heterocyclyl), —SH, —S(C₁₋₆ alkyl), —S(C₂₋₆ alkenyl), —S(C₂₋₆ alkynyl), —S(C₃₋₁₀ cycloalkyl), —S(C₁₋₆ haloalkyl), —S(aryl), —S(heteroaryl), —S(heterocyclyl), —NHS(O)(C₁₋₆ alkyl), —N(C₁₋₆ alkyl)(S(O)(C₁₋₆ alkyl), —S(O)N(C₁₋₆ alkyl)₂, —S(O)(C₁₋₆ alkyl), —S(O)(NH)(C₁₋₆ alkyl), —S(O)(C₂₋₆ alkenyl), —S(O)(C₂₋₆ alkynyl), —S(O)(C₃₋₁₀ cycloalkyl), —S(O)(C₁₋₆ haloalkyl), —S(O)(aryl), —S(O)(heteroaryl), —S(O)(heterocyclyl), —S(O)₂(C₁₋₆ alkyl), —S(O)₂(C₂₋₆ alkenyl), —S(O)₂(C₂₋₆ alkynyl), —S(O)₂(C₃₋₁₀ cycloalkyl), —S(O)₂(C₁₋₆ haloalkyl), —S(O)₂(aryl), —S(O)₂(heteroaryl), —S(O)₂(heterocyclyl), —S(O)₂NH(C₁₋₆ alkyl), or —S(O)₂N(C₁₋₆ alkyl)₂; wherein each alkyl, cycloalkyl, aryl, heteroaryl, or heterocyclyl of R³⁰ is optionally substituted with one to four halo, C₁₋₆ alkyl, C₁₋₆ haloalkyl, —OH, —NH₂, —NH(C₁₋₆ alkyl), —NH(C₃₋₁₀ cycloalkyl), —NH(C₁₋₆ haloalkyl), —NH(aryl), —NH(heteroaryl), —NH(heterocyclyl), —N(C₁₋₆ alkyl)₂, —N(C₃₋₁₀ cycloalkyl)₂, —NHC(O)(C₃₋₁₀ cycloalkyl), —NHC(O)(C₁₋₆ haloalkyl), —NHC(O)(aryl), —NHC(O)(heteroaryl), —NHC(O)(heterocyclyl), —NHC(O)O(C₁₋₆ alkyl), —NHC(O)O(C₂₋₆ alkynyl), —NHC(O)O(C₃₋₁₀ cycloalkyl), —NHC(O)O(C₁₋₆ haloalkyl), —NHC(O)O(aryl), —NHC(O)O(heteroaryl), —NHC(O)O(heterocyclyl), —NHC(O)NH(C₁₋₆ alkyl), —S(O)(NH)(C₁₋₆ alkyl), S(O)₂(C₁₋₆ alkyl), —S(O)₂(C₃₋₁₀ cycloalkyl), —S(O)₂(C₁₋₆ haloalkyl), —S(O)₂(aryl), —S(O)₂(heteroaryl), —S(O)₂(heterocyclyl), —S(O)₂NH(C₁₋₆ alkyl), —S(O)₂N(C₁₋₆ alkyl)₂, —O(C₃₋₁₀ cycloalkyl), —O(C₁₋₆ haloalkyl), —O(aryl), —O(heteroaryl), —O(heterocyclyl), or —O(C₁₋₆ alkyl);
provided that:
when W and X are both CH, then R⁵ and R³ are not both halo; and
the compound is not 3-bromo-2-hydroxy-5-methoxy-y-oxo-benzenebutanoic acid.

2. The compound of claim 1, or a pharmaceutically acceptable salt, isotopically enriched analog, stereoisomer, mixture of stereoisomers or prodrug thereof, represented by formula II:

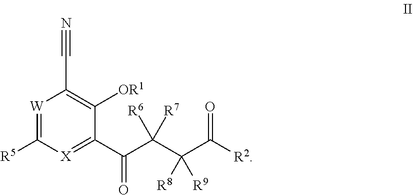

3. The compound of claim 1, wherein X is N and W is CR⁴.

4. The compound of claim 1, wherein W is N and X is CR⁴.

5. The compound of claim 1, wherein X is N and W is CH.

6. The compound of claim 1, wherein W is N and X is CH.

7. The compound of claim 1, wherein W and X are CH.

8. The compound of claim 1, or a pharmaceutically acceptable salt, isotopically enriched analog, stereoisomer, mixture of stereoisomers or prodrug thereof, represented by formula III:

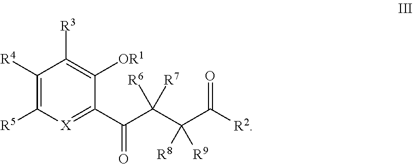

9. The compound of claim 1, wherein R⁶, R⁷, R⁸, and R⁹ are hydrogen.

10. The compound of claim 1, or a pharmaceutically acceptable salt, isotopically enriched analog, stereoisomer, mixture of stereoisomers or prodrug thereof, represented by formula IV:

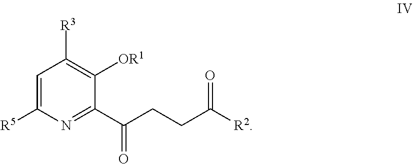

11. The compound of claim 1, or a pharmaceutically acceptable salt, isotopically enriched analog, stereoisomer, mixture of stereoisomers or prodrug thereof, represented by formula V:

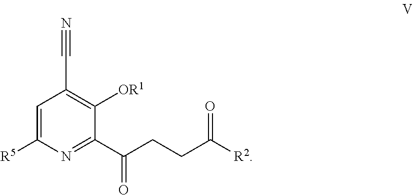

12. The compound of claim 1, wherein R¹ is H.

13. The compound of claim 1, wherein R² is —OH.

14. The compound of claim 1, wherein R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R⁵.

15. The compound of claim 1, wherein R⁵ is -L-aryl or -L-heteroaryl; wherein each aryl and heteroaryl of R is optionally substituted with 1-3 R⁵.

16. The compound of claim 1, wherein L is a bond, —C₁₋₅ alkylene, —C₁₋₅ heteroalkylene, —O—, —S—, —NR¹⁶—, or —C(O)NR¹⁶—; or L is a bond, —C₁₋₅ alkylene, —C₁₋₅ heteroalkylene, —O—, —S—, —S(O)—, —S(O)₂—, —C(O)NR¹⁶—, —NR¹⁶C(O)—, —OC(O)—, or —C(O)O—.

17. The compound of claim 1, wherein L is a bond, methylene, or —O—.

18. The compound according to claim 1, wherein R⁵ is bromo, cyano, —CF₃, —S—CH₃, methyl, phenyl, —O—CH₃, benzyl, 2-pyridinyl, 3-pyridinyl, 4-pyridinyl, 2-methoxy-phenyl, phenethyl, 1-methyl-1H-pyrazol-4-yl, 4-chloro-benzyl, 3-chloro-benzyl, 4-fluoro-phenyl, 3-fluoro-phenyl, 2-chloro-benzyl, 4-chloro-phenyl, 3-chloro-phenyl, naphthalen-2-ylmethyl, cyclohexyl, 4-trifluoromethyl-phenyl, 4-methoxy-benzyl, 3-trifluoromethyl-phenyl, 3-trifluoromethyl-benzyl, 2-chloro-6-fluoro-benzyl, 3,5-dichloro-benzyl, 2,6-dichloro-benzyl, cyclohexylmethyl, naphthalen-1-ylmethyl, 2-methyl-benzyl, 3-methyl-benzyl, 4-methyl-benzyl, 4-trifluoromethyl-benzyl, 4-fluoro-benzyl, 2-trifluoromethyl-benzyl, 3-fluoro-benzyl, 2-fluoro-benzyl, 4-cyano-benzyl, 3-cyano-benzyl, 2-cyano-benzyl, 4-trifluoromethoxy-benzyl, 3-trifluoromethoxy-benzyl, 2-trifluoromethoxy-benzyl, biphenyl-4-ylmethyl, biphenyl-3-ylmethyl, biphenyl-2-ylmethyl, 2,6-difluoro-benzyl, 2,6-dimethyl-benzyl, 2,4,6-trifluoro-benzyl, 3-chloro-2,6-difluoro-benzyl, 2,3,6-trifluoro-benzyl, 2-chloro-6-cyano-benzyl, 2-chloro-6-trifluoromethyl-benzyl, 2-fluoro-6-trifluoromethyl-benzyl, 2-methoxy-6-trifluoromethyl-benzyl, 2-methyl-6-trifluoromethyl-benzyl, 2,5-dichloro-benzyl, 2,4-dichloro-benzyl, 2,3-dichloro-benzyl, 3-trifluoromethoxy-phenyl, 4-trifluoromethoxy-phenyl, naphthalen-1-yl, 2-fluoro-phenyl, 2-chloro-phenyl, 2-methyl-phenyl, 3-methyl-phenyl, 4-methyl-phenyl, biphenyl-3-yl, biphenyl-4-yl, phenoxy, naphthalen-2-yl, phenylsulfanyl, 4-fluoro-phenoxy, 3-fluoro-2-methyl-phenyl, 2-chloro-4-methoxy-phenyl, 5-fluoro-2-methyl-phenyl, 2-chloro-4-fluoro-phenyl, 2-chloro-4-methyl-phenyl, 2-ethyl-phenyl, 4-fluoro-2-methyl-phenyl, 2-cyano-4-fluoro-phenyl, 2-fluoro-6-methyl-phenyl, 3-chloro-2-methyl-phenyl, 4-chloro-2-methyl-phenyl, 5-chloro-2-methyl-phenyl, 2,3-dimethyl-phenyl, 2,4-dimethyl-phenyl, 2,5-dimethyl-phenyl, 2-methyl-3-trifluoromethyl-phenyl, 2-methyl-4-trifluoromethyl-phenyl, 2-methyl-5-trifluoromethyl-phenyl, 3-cyano-2-methyl-phenyl, 4-cyano-2-methyl-phenyl, 5-cyano-2-methyl-phenyl, 2-chloro-3-methyl-phenyl, 2-chloro-5-methyl-phenyl, 2-chloro-3-trifluoromethyl-phenyl, 2-cyano-5-trifluoromethyl-phenyl, 2-chloro-5-methoxy-phenyl, 2-chloro-3-fluoro-phenyl, 2-chloro-5-fluoro-phenyl, 4-fluoro-naphthalen-1-yl, 4-chloro-naphthalen-1-yl, 4-phenyl-naphthalen-1-yl, quinolin-5-yl, 4-methyl-naphthalen-1-yl, 2-chloro-3-methoxy-phenyl, 2-chloro-4-trifluoromethyl-phenyl, quinolin-8-yl, 2-chloro-phenyl, 2-chloro-6-methyl-benzyl, 2,4,6-trimethyl-benzyl, 2-chloro-6-methoxy-benzyl, 2,6-dichloro-4-fluoro-benzyl, 2,6-dichloro-4-methyl-benzyl, 2,4,6-trichloro-benzyl, 2,6-dichloro-3-fluoro-benzyl, 2-chloro-phenoxy, 4-chloro-phenoxy, 3-chloro-phenoxy, p-tolyloxy-phenyl, o-tolyloxy-phenyl, 4-methoxy-phenoxy, 2,6-dichloro-4-trifluoromethoxy-benzyl, 2,6-dichloro-4-trifluoromethyl-benzyl, 2-benzyl-benzyl, 2,6-dichloro-4-methoxy-benzyl, 2-fluoro-6-methyl-benzyl, 2,6-dichloro-3-fluoro-benzyl, 2-fluoro-6-methoxy-benzyl, 4-fluoro-2,6-dimethyl-benzyl, 4-chloro-2,6-dimethyl-benzyl, 4-cyano-2,6-dimethyl-benzyl, 3,5-dimethyl-isoxazol-4-ylmethyl, 2,6-dichloro-3-methyl-benzyl, 2,3,6-trichloro-benzyl, benzoylamino, benzoyl-methyl-amino, (2,6-dimethyl-benzoyl)-methyl-amino, 2,6-dimethyl-benzoylamino, or 2,6-dichloro-benzoylamino.

19. The compound of claim 1, wherein:
one of W or X is N and the other of W and X is CR⁴;
R¹ is H;
R² is H;
R³ is halo, cyano, C₁₋₆ alkyl, or C₁₋₆ haloalkyl;
R⁵ is -L-₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵;
each of R⁶, R⁷, R⁸, and R⁹ is hydrogen; and
L is a bond, —C₁₋₅ alkylene, —C₁₋₅ heteroalkylene, —O—, —S—, —NR¹⁶—, or —C(O)NR¹⁶—.

20. The compound of claim 1, wherein:
X is N and W is CR⁴;
R¹ is H;
R² is H;
R³ is halo, cyano, C₁₋₆ alkyl, or C₁₋₆ haloalkyl;
R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵;
each of R⁶, R⁷, R⁸, and R⁹ is hydrogen; and
L is a bond, —C₁₋₅ alkylene, —C₁₋₅ heteroalkylene, —O—, —S—, —NR¹⁶—, or —C(O)NR¹⁶—.

21. The compound of claim 1, wherein:
X is N and W is CH;
R¹ is H;
R² is H;
R³ is halo, cyano, methyl, or —CF₃;
R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵;
each of R⁶, R⁷, R⁸, and R⁹ is hydrogen; and
L is a bond, methylene, or —O—.

22. The compound of claim 1, wherein:
X and W are CH;
R¹ is H;
R² is H;
R³ is halo, cyano, methyl, or —CF₃;
R⁵ is -L-C₃₋₁₀ cycloalkyl, -L-C₃₋₁₀ cycloalkenyl, -L-heterocyclyl, -L-aryl, or -L-heteroaryl; wherein each cycloalkyl, cycloalkenyl, heterocyclyl, aryl, and heteroaryl of R⁵ is optionally substituted with 1-3 R¹⁵;
each of R⁶, R⁷, R⁸, and R⁹ is hydrogen; and
L is a bond, methylene, or —O—.

23. A compound, or a pharmaceutically acceptable salt, isotopically enriched analog, stereoisomer, mixture of stereoisomers or prodrug thereof, selected from:

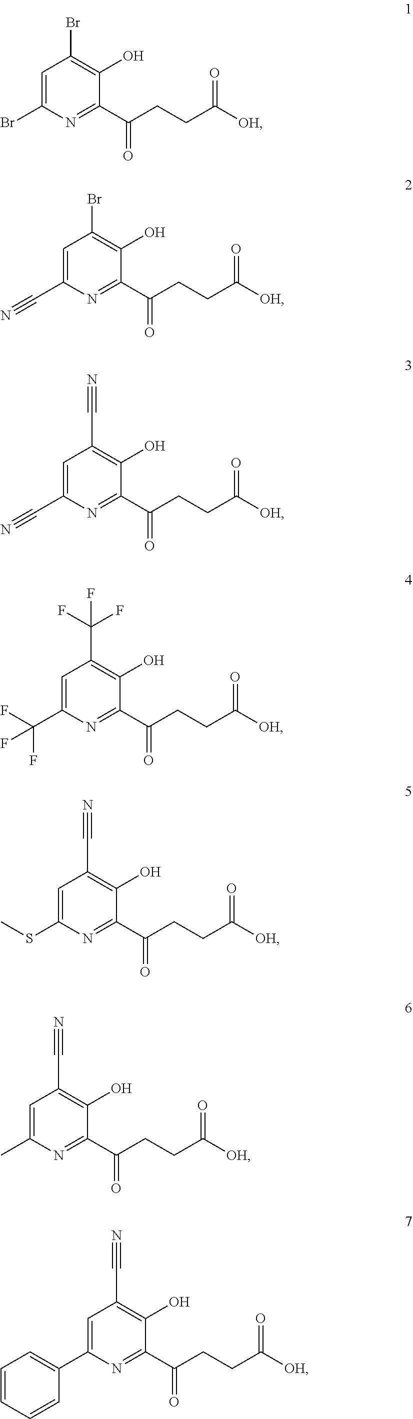

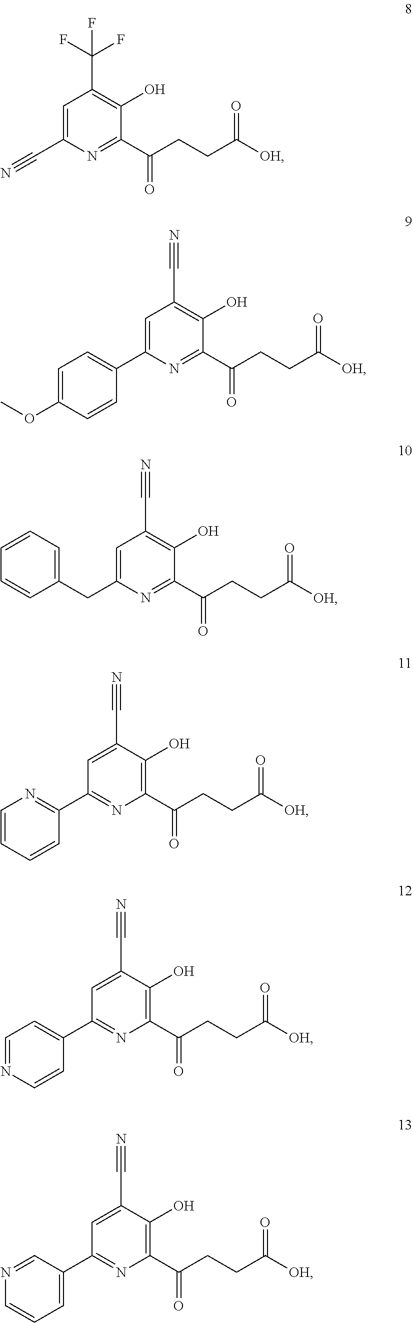 [SMILES: N#Cc1cc(C(F)(F)F)c(O)c(C(=O)CCC(=O)O)n1 ; COc1ccc(-c2cc(C#N)c(O)c(C(=O)CCC(=O)O)n2)cc1 ; N#Cc1cc(Cc2ccccc2)nc(C(=O)CCC(=O)O)c1O ; N#Cc1cc(-c2ccccn2)nc(C(=O)CCC(=O)O)c1O ; N#Cc1cc(-c2ccncc2)nc(C(=O)CCC(=O)O)c1O ; N#Cc1cc(-c2cccnc2)nc(C(=O)CCC(=O)O)c1O]

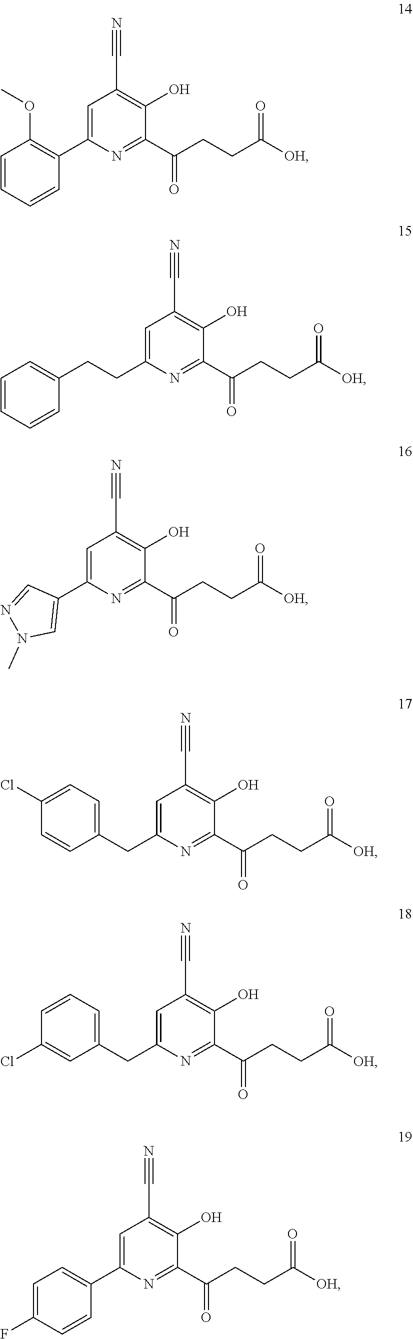 [SMILES: COc1ccccc1-c1cc(C#N)c(O)c(C(=O)CCC(=O)O)n1 ; N#Cc1cc(CCc2ccccc2)nc(C(=O)CCC(=O)O)c1O ; Cn1cc(-c2cc(C#N)c(O)c(C(=O)CCC(=O)O)n2)cn1 ; N#Cc1cc(Cc2ccc(Cl)cc2)nc(C(=O)CCC(=O)O)c1O ; N#Cc1cc(Cc2cccc(Cl)c2)nc(C(=O)CCC(=O)O)c1O ; N#Cc1cc(-c2ccc(F)cc2)nc(C(=O)CCC(=O)O)c1O]

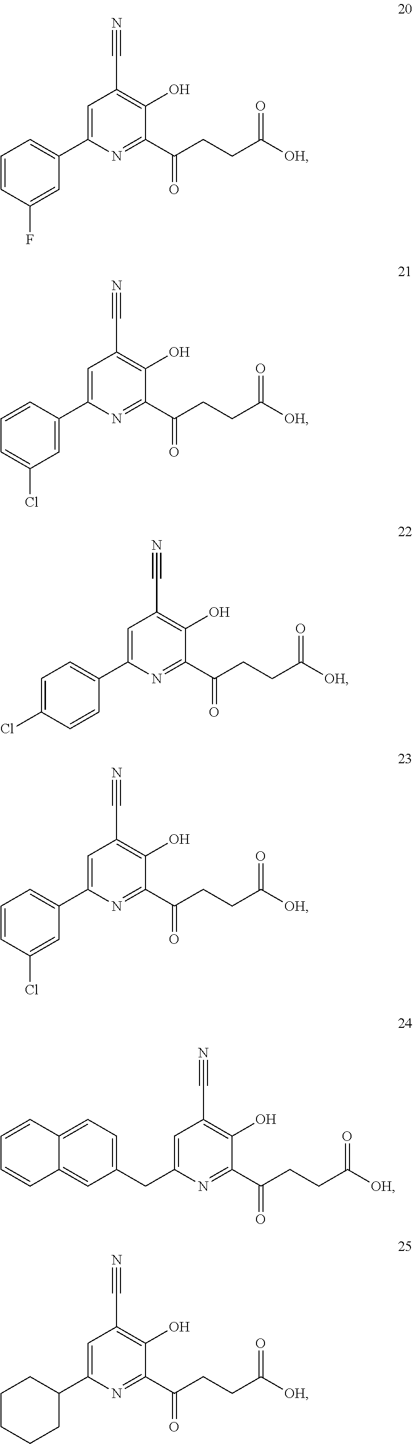 [SMILES: N#Cc1cc(-c2cccc(F)c2)nc(C(=O)CCC(=O)O)c1O ; N#Cc1cc(-c2cccc(Cl)c2)nc(C(=O)CCC(=O)O)c1O ; N#Cc1cc(-c2ccc(Cl)cc2)nc(C(=O)CCC(=O)O)c1O ; N#Cc1cc(-c2cccc(Cl)c2)nc(C(=O)CCC(=O)O)c1O ; N#Cc1cc(Cc2ccc3ccccc3c2)nc(C(=O)CCC(=O)O)c1O ; N#Cc1cc(C2CCCCC2)nc(C(=O)CCC(=O)O)c1O]

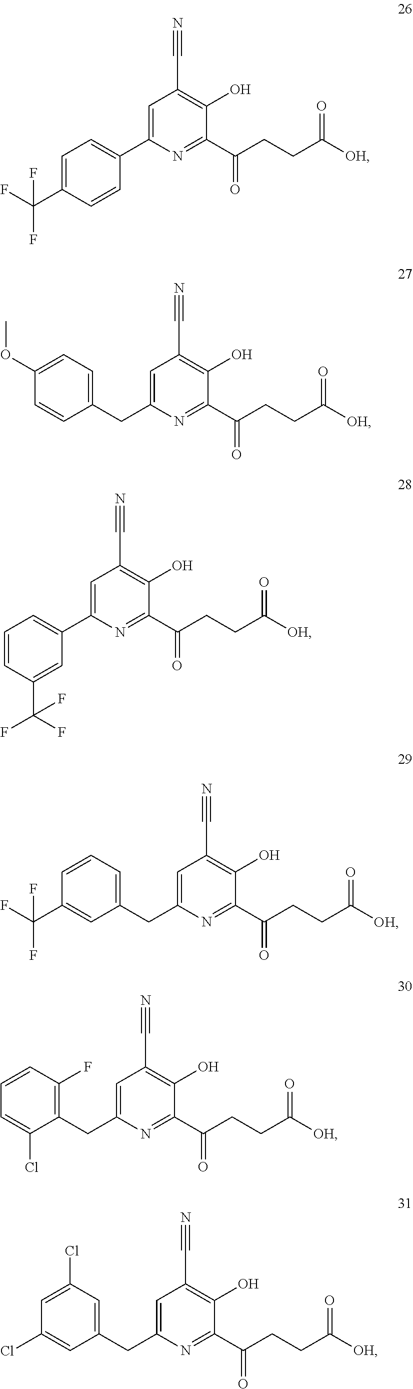 [SMILES: N#Cc1cc(-c2ccc(C(F)(F)F)cc2)nc(C(=O)CCC(=O)O)c1O ; COc1ccc(Cc2cc(C#N)c(O)c(C(=O)CCC(=O)O)n2)cc1 ; N#Cc1cc(-c2cccc(C(F)(F)F)c2)nc(C(=O)CCC(=O)O)c1O ; N#Cc1cc(Cc2cccc(C(F)(F)F)c2)nc(C(=O)CCC(=O)O)c1O ; N#Cc1cc(Cc2c(F)cccc2Cl)nc(C(=O)CCC(=O)O)c1O ; N#Cc1cc(Cc2cc(Cl)cc(Cl)c2)nc(C(=O)CCC(=O)O)c1O]

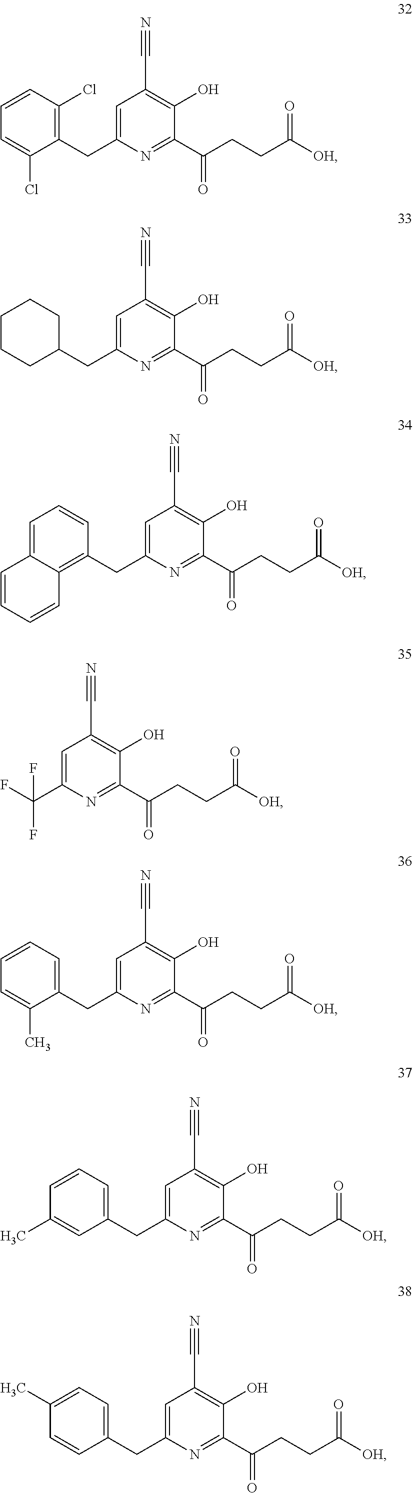

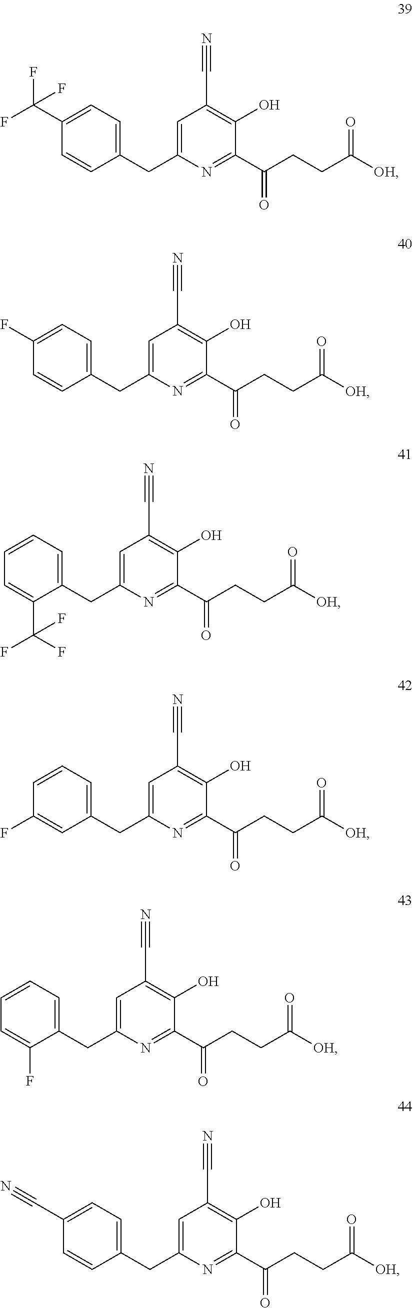 [SMILES: N#Cc1cc(Cc2ccc(C(F)(F)F)cc2)nc(C(=O)CCC(=O)O)c1O ; N#Cc1cc(Cc2ccc(F)cc2)nc(C(=O)CCC(=O)O)c1O ; N#Cc1cc(Cc2ccccc2C(F)(F)F)nc(C(=O)CCC(=O)O)c1O ; N#Cc1cc(Cc2cccc(F)c2)nc(C(=O)CCC(=O)O)c1O ; N#Cc1cc(Cc2ccccc2F)nc(C(=O)CCC(=O)O)c1O ; N#Cc1ccc(Cc2cc(C#N)c(O)c(C(=O)CCC(=O)O)n2)cc1]

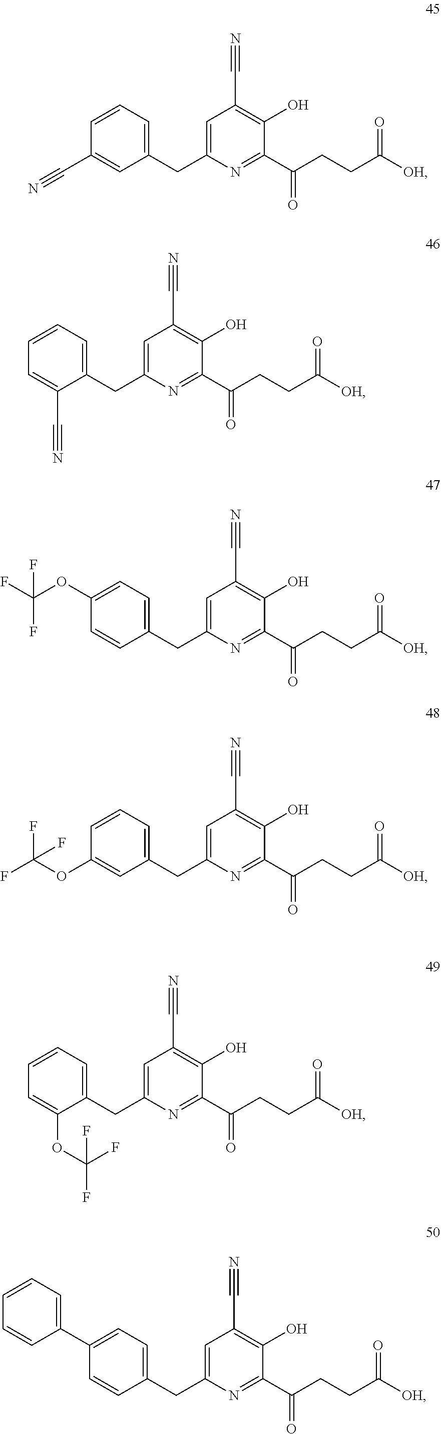 [SMILES: N#Cc1cccc(Cc2cc(C#N)c(O)c(C(=O)CCC(=O)O)n2)c1 ; N#Cc1ccccc1Cc1cc(C#N)c(O)c(C(=O)CCC(=O)O)n1 ; N#Cc1cc(Cc2ccc(OC(F)(F)F)cc2)nc(C(=O)CCC(=O)O)c1O ; N#Cc1cc(Cc2cccc(OC(F)(F)F)c2)nc(C(=O)CCC(=O)O)c1O ; N#Cc1cc(Cc2ccccc2OC(F)(F)F)nc(C(=O)CCC(=O)O)c1O ; N#Cc1cc(Cc2ccc(-c3ccccc3)cc2)nc(C(=O)CCC(=O)O)c1O]

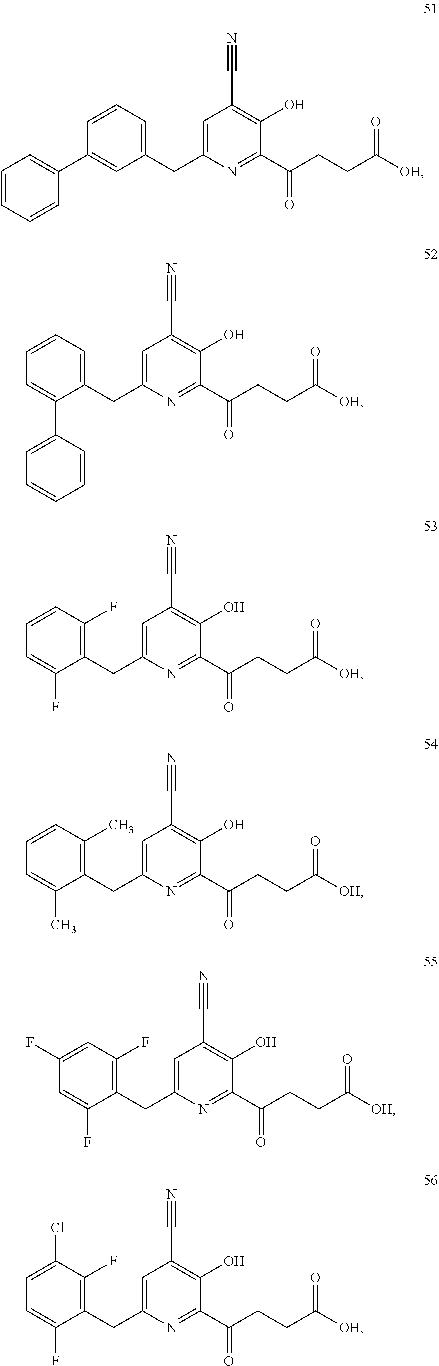 [SMILES: N#Cc1cc(Cc2cccc(-c3ccccc3)c2)nc(C(=O)CCC(=O)O)c1O ; N#Cc1cc(Cc2ccccc2-c2ccccc2)nc(C(=O)CCC(=O)O)c1O ; N#Cc1cc(Cc2c(F)cccc2F)nc(C(=O)CCC(=O)O)c1O ; Cc1cccc(C)c1Cc1cc(C#N)c(O)c(C(=O)CCC(=O)O)n1 ; N#Cc1cc(Cc2c(F)cc(F)cc2F)nc(C(=O)CCC(=O)O)c1O ; N#Cc1cc(Cc2c(F)ccc(Cl)c2F)nc(C(=O)CCC(=O)O)c1O]

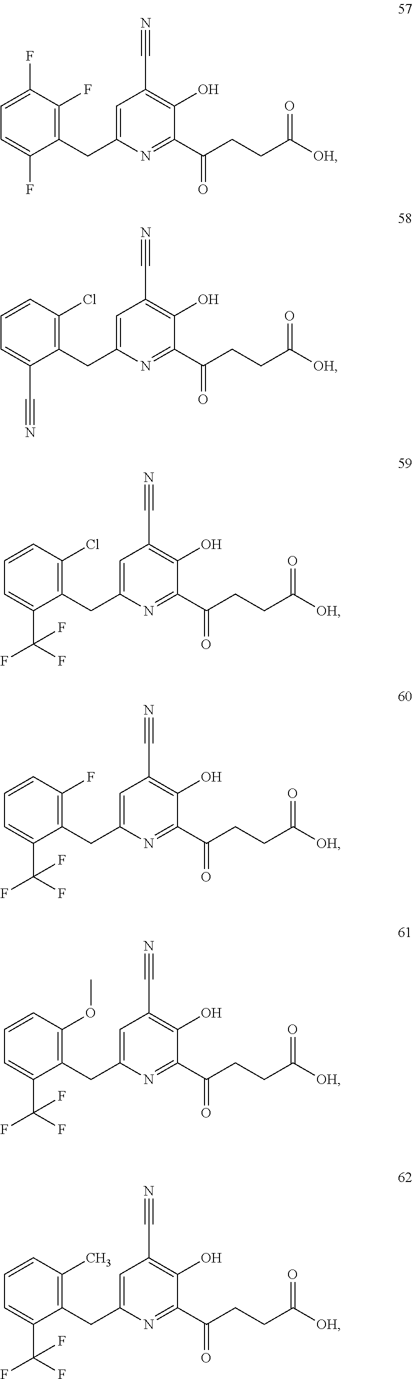 [SMILES: N#Cc1cc(Cc2c(F)ccc(F)c2F)nc(C(=O)CCC(=O)O)c1O ; N#Cc1cc(Cc2c(Cl)cccc2C#N)nc(C(=O)CCC(=O)O)c1O ; N#Cc1cc(Cc2c(Cl)cccc2C(F)(F)F)nc(C(=O)CCC(=O)O)c1O ; N#Cc1cc(Cc2c(F)cccc2C(F)(F)F)nc(C(=O)CCC(=O)O)c1O ; COc1cccc(C(F)(F)F)c1Cc1cc(C#N)c(O)c(C(=O)CCC(=O)O)n1 ; Cc1cccc(C(F)(F)F)c1Cc1cc(C#N)c(O)c(C(=O)CCC(=O)O)n1]

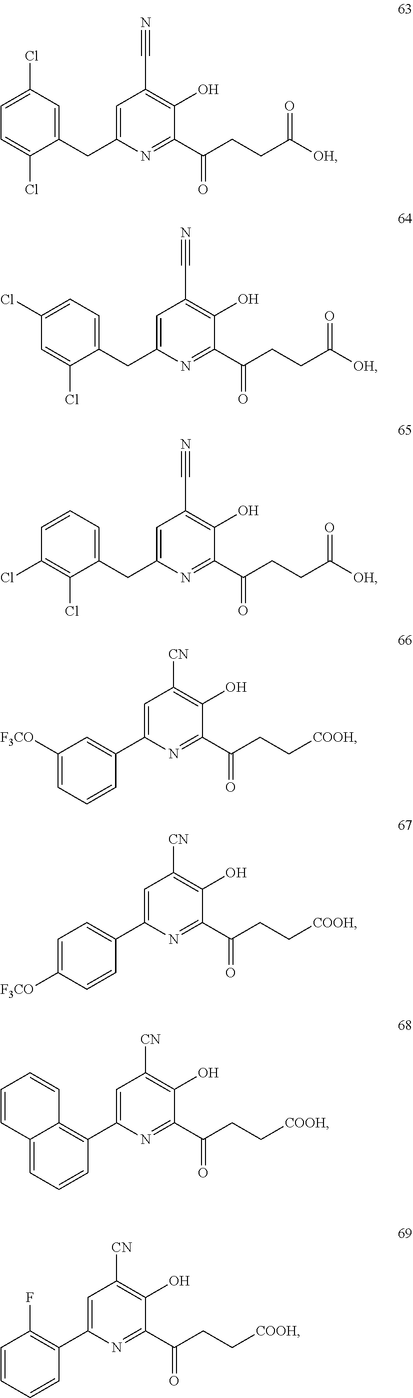

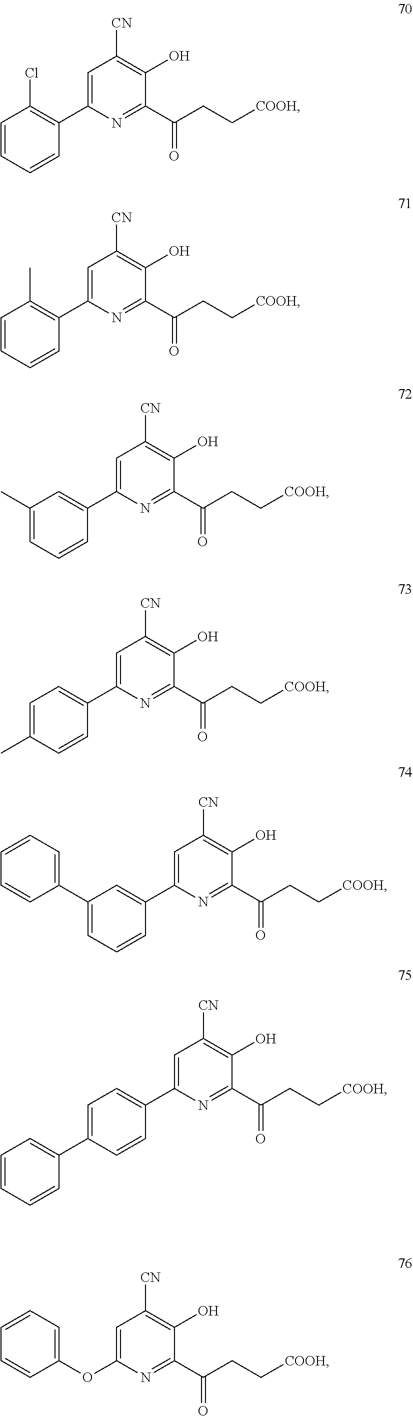

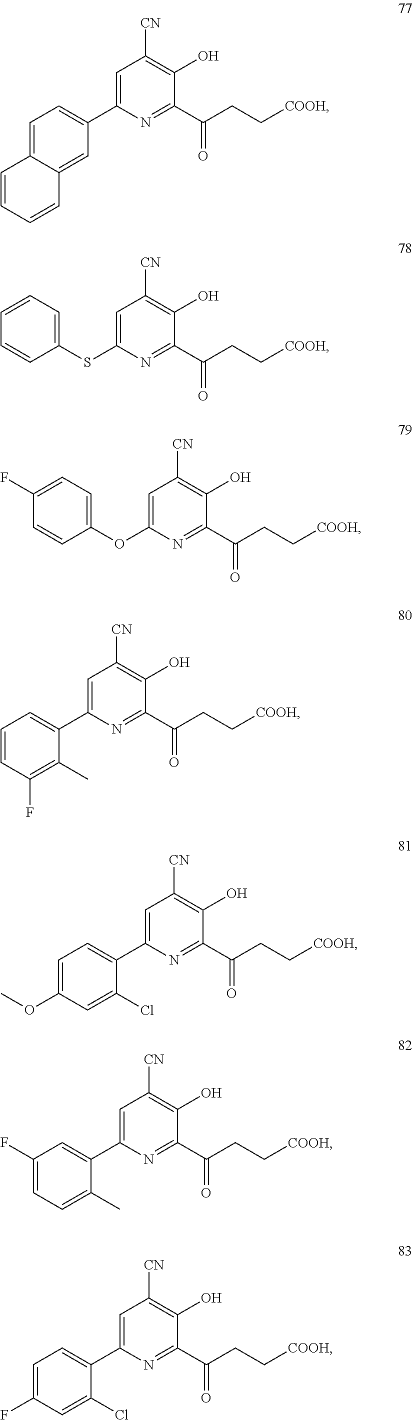

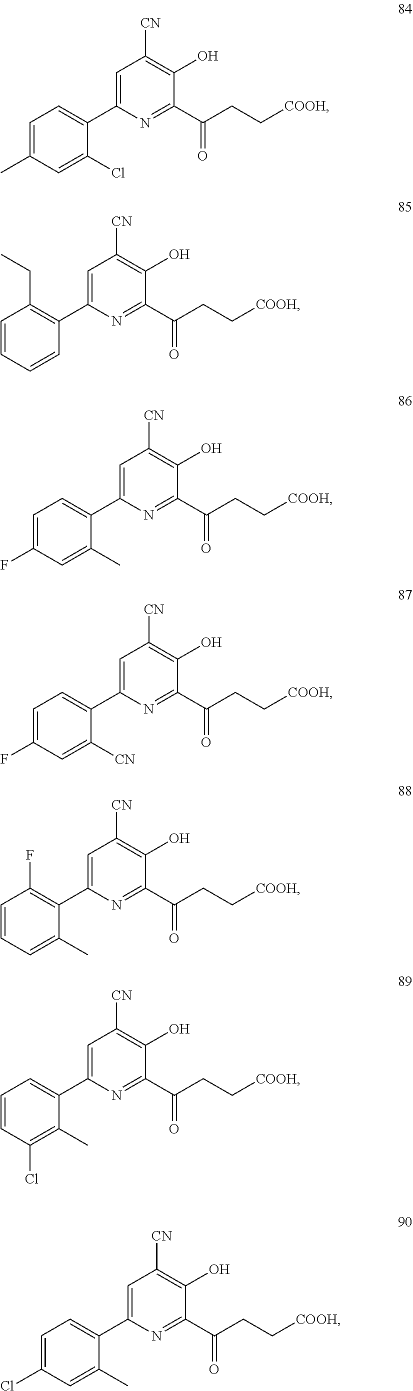

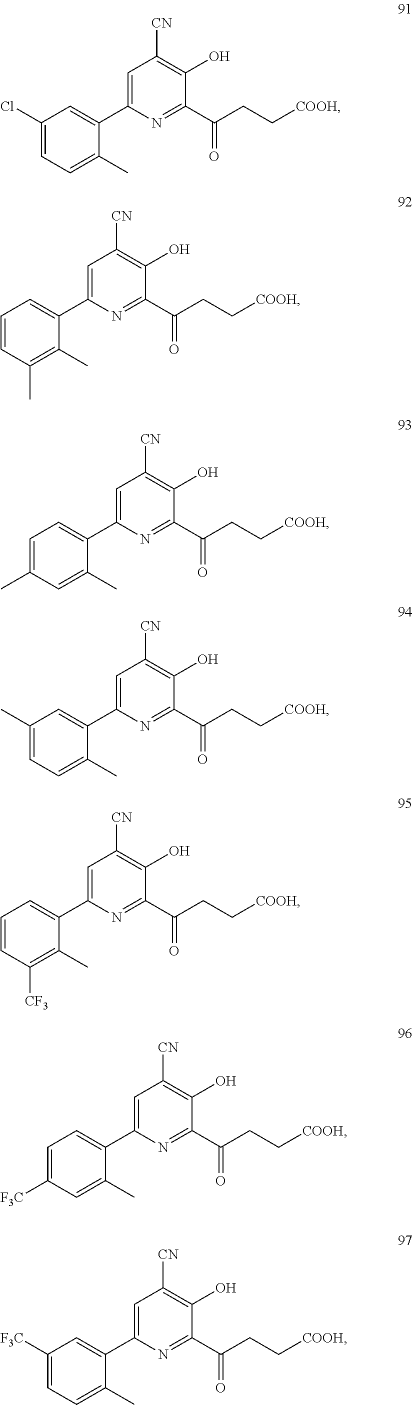

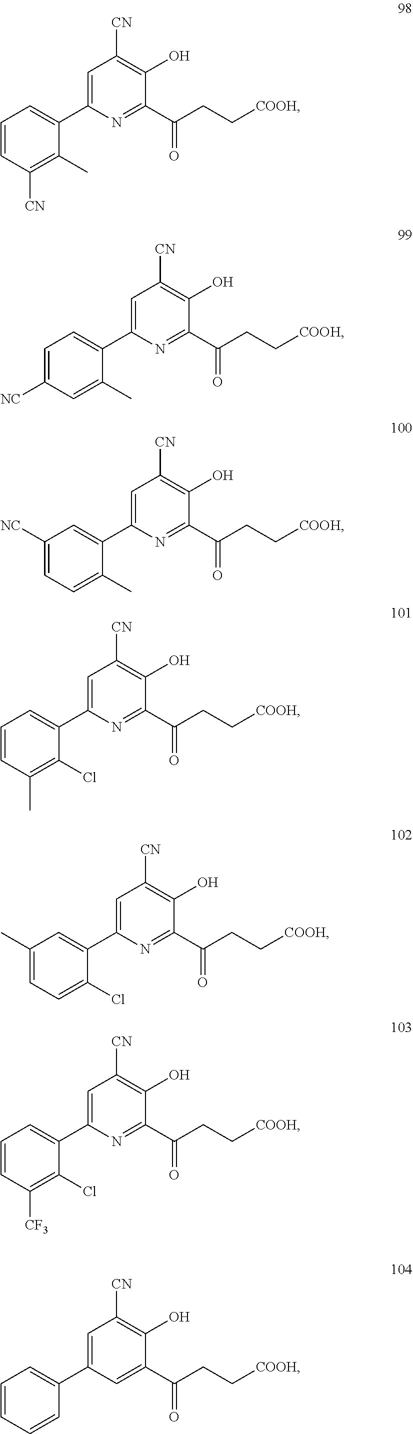

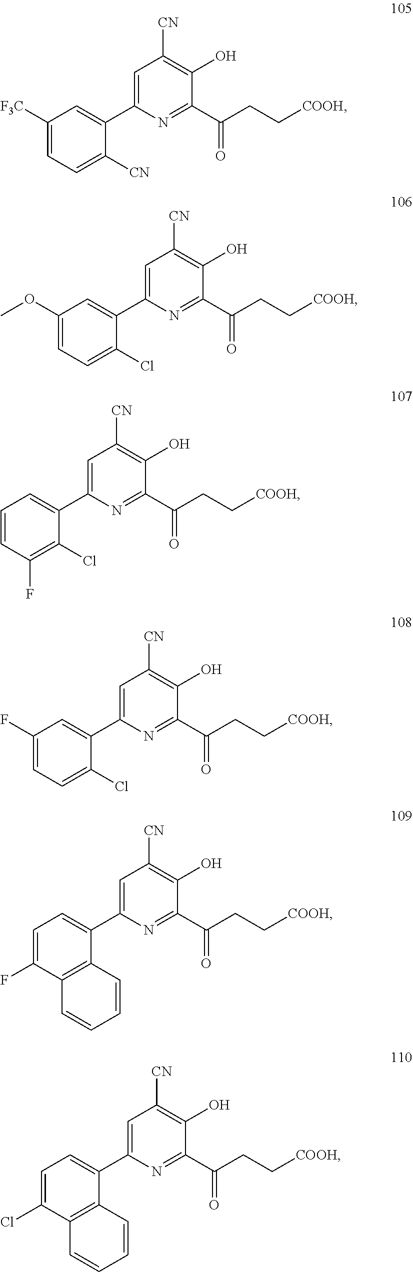 [SMILES: N#Cc1ccc(C(F)(F)F)cc1-c1cc(C#N)c(O)c(C(=O)CCC(=O)O)n1 ; COc1ccc(Cl)c(-c2cc(C#N)c(O)c(C(=O)CCC(=O)O)n2)c1 ; N#Cc1cc(-c2cccc(F)c2Cl)nc(C(=O)CCC(=O)O)c1O ; N#Cc1cc(-c2cc(F)ccc2Cl)nc(C(=O)CCC(=O)O)c1O ; N#Cc1cc(-c2ccc(F)c3ccccc23)nc(C(=O)CCC(=O)O)c1O ; N#Cc1cc(-c2ccc(Cl)c3ccccc23)nc(C(=O)CCC(=O)O)c1O]

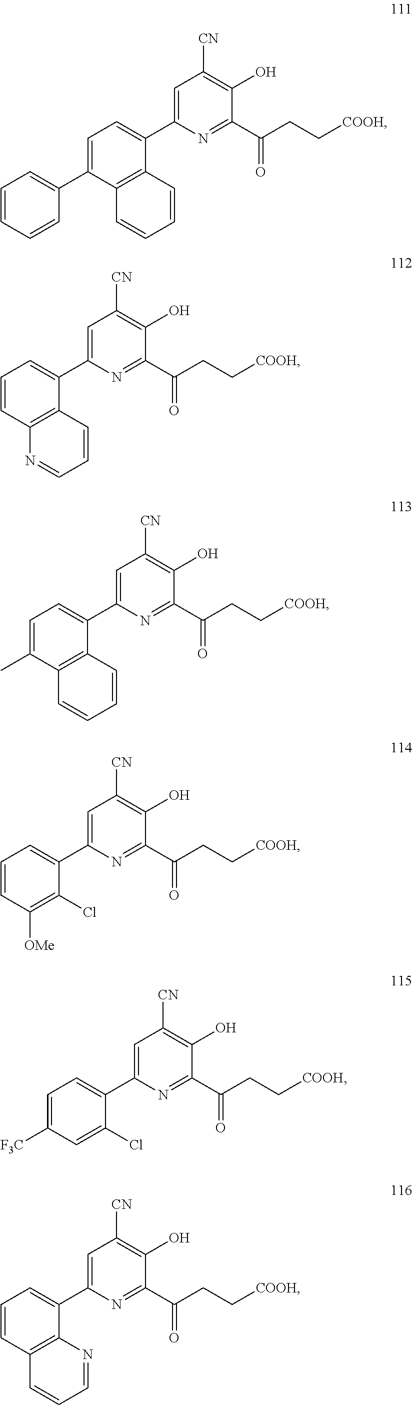 [SMILES: N#Cc1cc(-c2ccc(-c3ccccc3)c3ccccc23)nc(C(=O)CCC(=O)O)c1O ; N#Cc1cc(-c2cccc3ncccc23)nc(C(=O)CCC(=O)O)c1O ; Cc1ccc(-c2cc(C#N)c(O)c(C(=O)CCC(=O)O)n2)c2ccccc12 ; COc1cccc(-c2cc(C#N)c(O)c(C(=O)CCC(=O)O)n2)c1Cl ; N#Cc1cc(-c2ccc(C(F)(F)F)cc2Cl)nc(C(=O)CCC(=O)O)c1O ; N#Cc1cc(-c2cccc3cccnc23)nc(C(=O)CCC(=O)O)c1O]

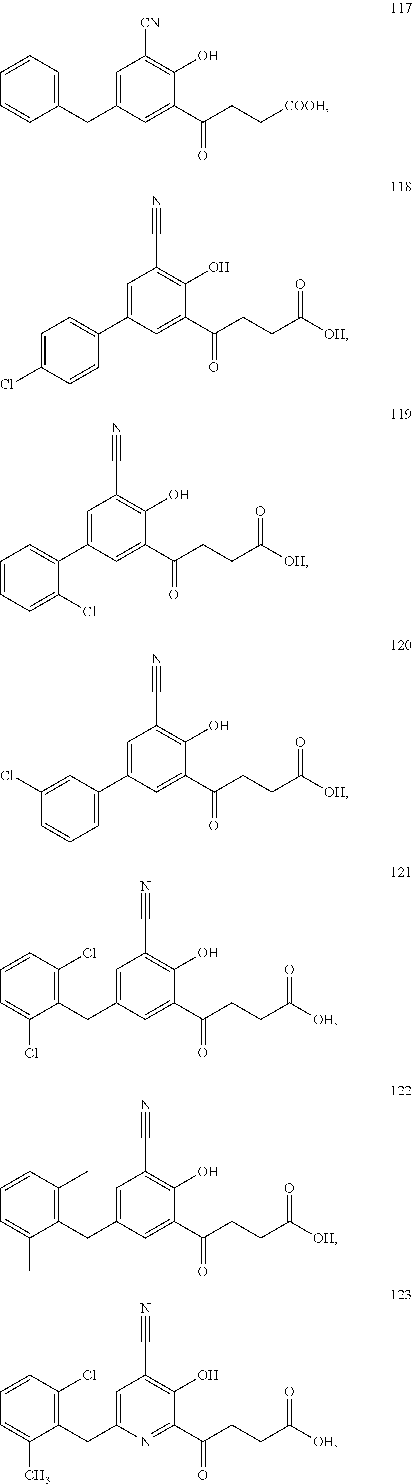

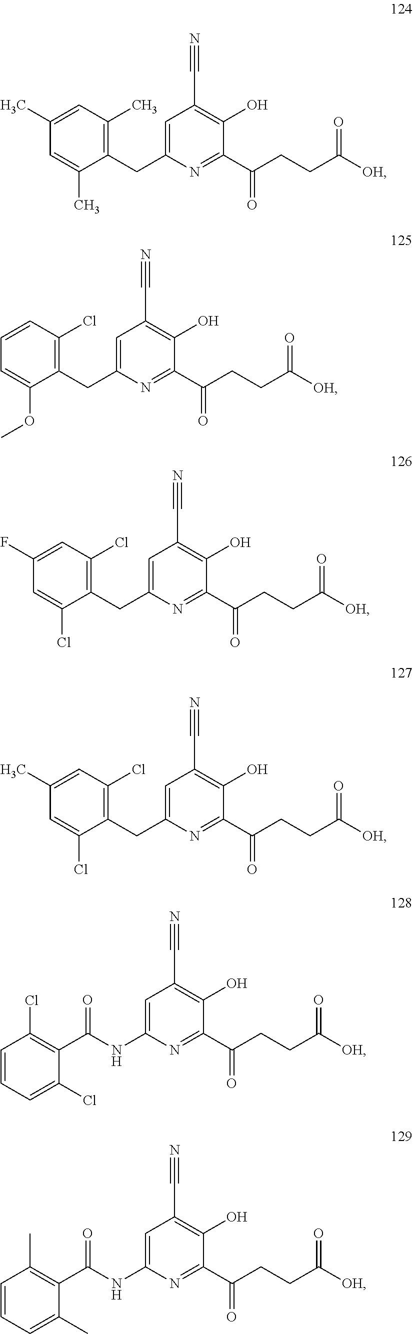 [SMILES: Cc1cc(C)c(Cc2cc(C#N)c(O)c(C(=O)CCC(=O)O)n2)c(C)c1 ; COc1cccc(Cl)c1Cc1cc(C#N)c(O)c(C(=O)CCC(=O)O)n1 ; N#Cc1cc(Cc2c(Cl)cc(F)cc2Cl)nc(C(=O)CCC(=O)O)c1O ; Cc1cc(Cl)c(Cc2cc(C#N)c(O)c(C(=O)CCC(=O)O)n2)c(Cl)c1 ; N#Cc1cc(NC(=O)c2c(Cl)cccc2Cl)nc(C(=O)CCC(=O)O)c1O ; Cc1cccc(C)c1C(=O)Nc1cc(C#N)c(O)c(C(=O)CCC(=O)O)n1]

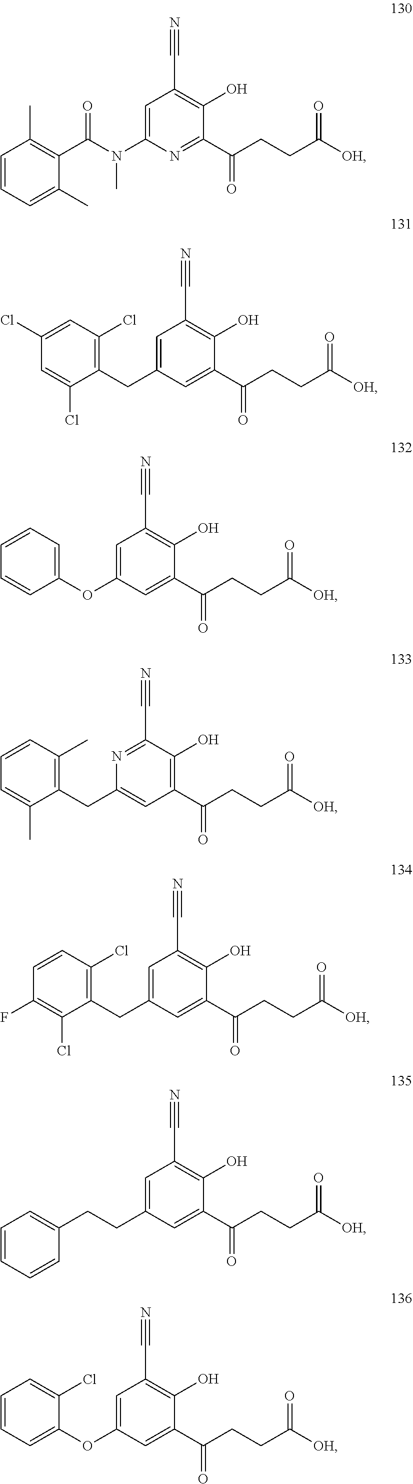

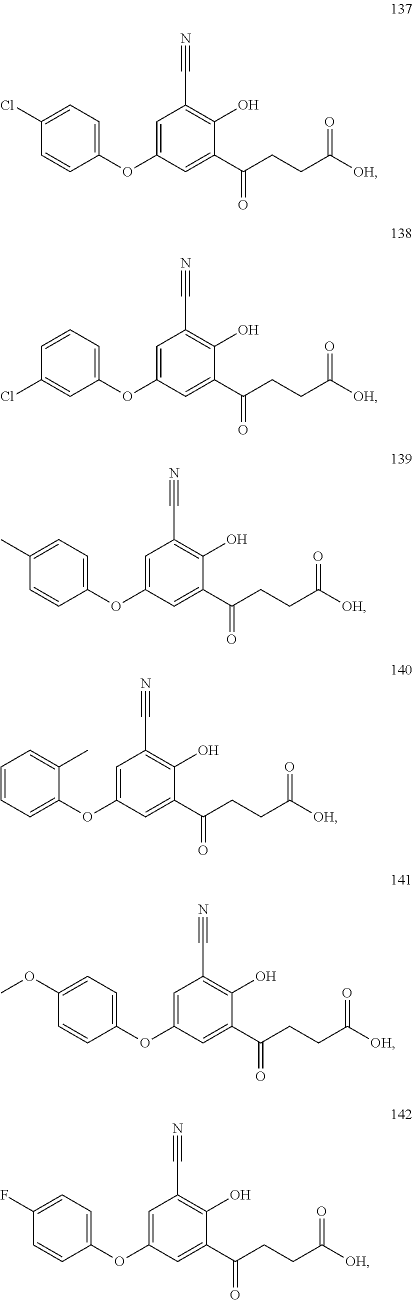 [SMILES: N#Cc1cc(Oc2ccc(Cl)cc2)cc(C(=O)CCC(=O)O)c1O ; N#Cc1cc(Oc2cccc(Cl)c2)cc(C(=O)CCC(=O)O)c1O ; Cc1ccc(Oc2cc(C#N)c(O)c(C(=O)CCC(=O)O)c2)cc1 ; Cc1ccccc1Oc1cc(C#N)c(O)c(C(=O)CCC(=O)O)c1 ; COc1ccc(Oc2cc(C#N)c(O)c(C(=O)CCC(=O)O)c2)cc1 ; N#Cc1cc(Oc2ccc(F)cc2)cc(C(=O)CCC(=O)O)c1O]

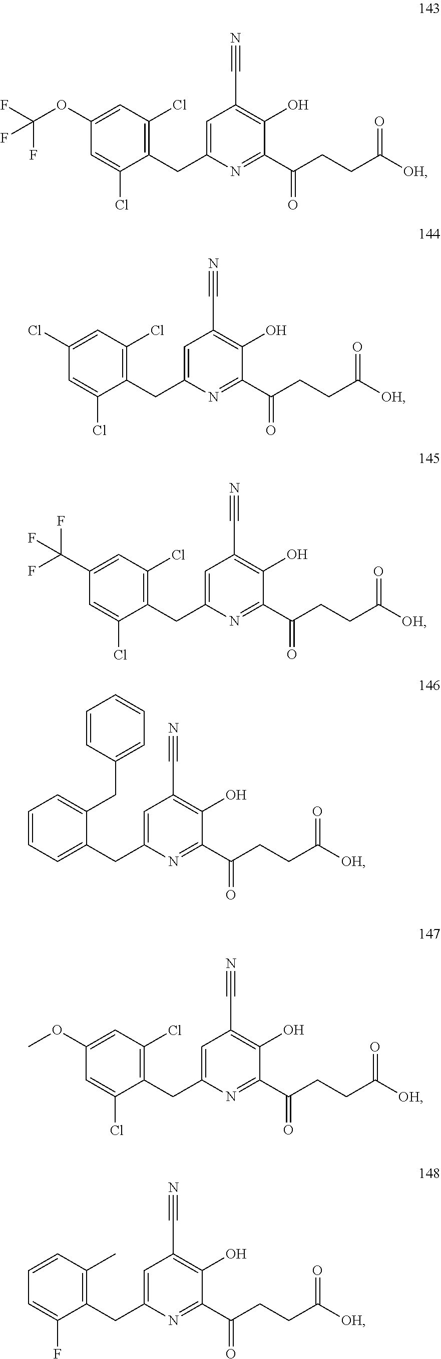

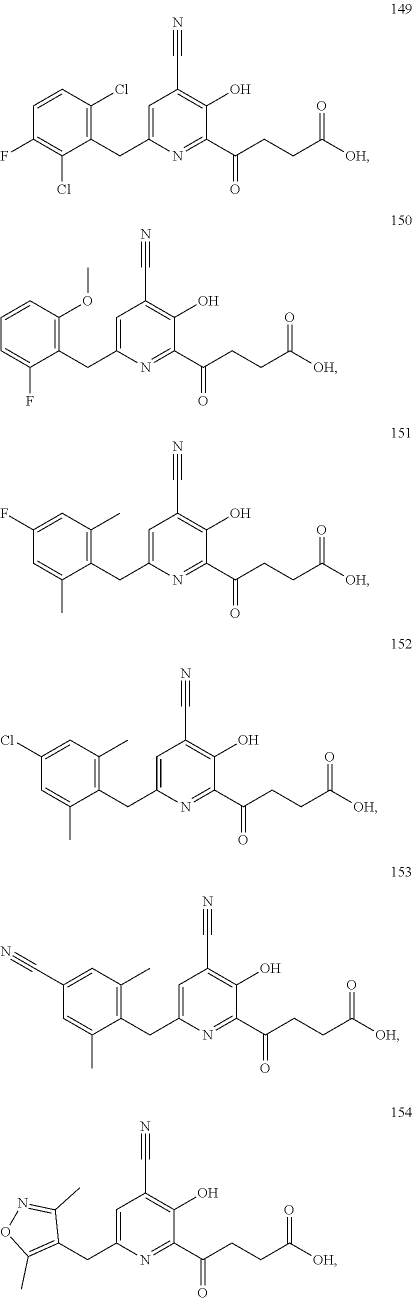 [SMILES: N#Cc1cc(Cc2c(Cl)ccc(F)c2Cl)nc(C(=O)CCC(=O)O)c1O ; COc1cccc(F)c1Cc1cc(C#N)c(O)c(C(=O)CCC(=O)O)n1 ; Cc1cc(F)cc(C)c1Cc1cc(C#N)c(O)c(C(=O)CCC(=O)O)n1 ; Cc1cc(Cl)cc(C)c1Cc1cc(C#N)c(O)c(C(=O)CCC(=O)O)n1 ; Cc1cc(C#N)cc(C)c1Cc1cc(C#N)c(O)c(C(=O)CCC(=O)O)n1 ; Cc1noc(C)c1Cc1cc(C#N)c(O)c(C(=O)CCC(=O)O)n1]

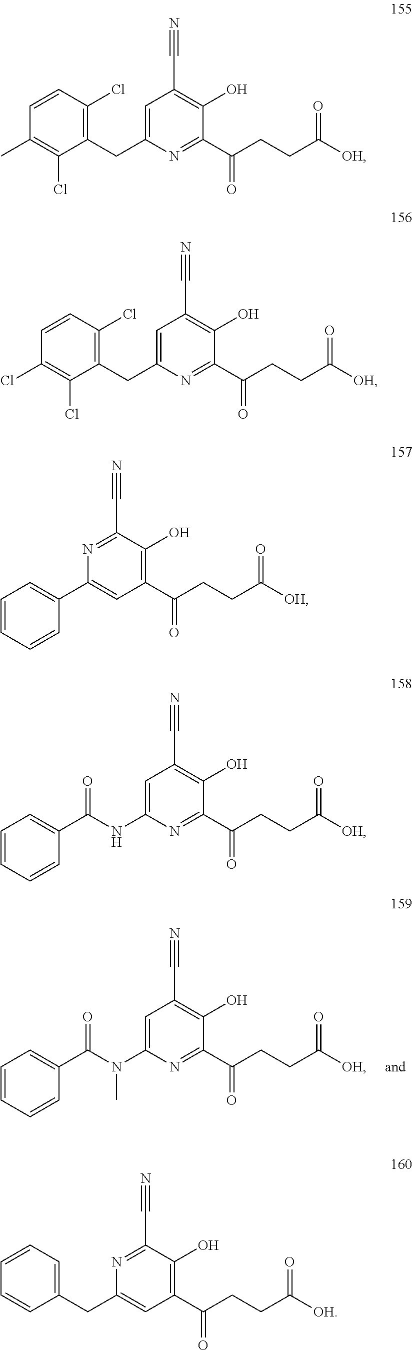

24. A pharmaceutical composition comprising one or more compounds of claim 1 and a pharmaceutically acceptable excipient.

25. The pharmaceutical composition of claim 24, further comprising at least one additional therapeutic agent.

26. A method of inhibiting the activity of histone lysine demethylase, the method comprising bringing into contact histone lysine demethylase and an inhibitory-effective amount of a pharmaceutical composition of claim 24.

27-40. (canceled)
